(19) United States
(12) Patent Application Publication
(10) Pub. No.: US 2006/0228361 A1
(43) Pub. Date: Oct. 12, 2006

(54) Dicer interacting proteins and uses therefor

(75) Inventor: Craig C. Mello, Shrewsbury, MA (US)

Correspondence Address: LAHIVE & COCKFIELD, 28 STATE STREET, BOSTON, MA, 02109, US

(73) Assignee: UNIVERSITY OF MASSACHUSETTS, Boston, MA (US)

(21) Appl. No.: 11/107,336

(22) Filed: Apr. 14, 2005

Related U.S. Application Data
(60) Provisional application No. 60/562,420, filed on Apr. 14, 2004.

(51) Int. Cl. A61K 48/00; G01N 33/53; C12Q 1/37; A61K 39/395; C07K 16/40

(52) U.S. Cl. 424/146.1; 435/7.1; 435/23; 514/44; 530/388.26

(57) ABSTRACT

Dicer (e.g., DCR-1) interactors are disclosed as are methods to positively or negatively modulate Dicer activity. Uses of Dicer interactors as drug targets are featured. Also featured are uses of Dicer interactors and modulators of same to modulate various Dicer functions in vitro, in cell cultures, and in vivo.

### RELATED APPLICATIONS

This application claims the benefit of prior-filed provisional patent application Ser. No. 60/562,420, filed Apr. 14, 2004, entitled “DICER INTERACTING PROTEINS AND USES THEREFOR.” The contents of any patents, patent applications, references, and appendices cited throughout this specification are hereby incorporated by reference in their entireties.

### STATEMENT AS TO SPONSORED RESEARCH

Funding for the work described herein was, at least in part, supported by grants from the National Institutes of Health (R01 GM058800; R21 ES012021-02).

### BACKGROUND OF THE INVENTION

RNA-mediated gene silencing phenomena, known as post-transcriptional gene silencing in plants, quelling in fungi, and RNA interference (RNAi) in animals, are mediated by double-stranded RNA (dsRNA) and mechanistically intersect at the ribonuclease Dicer. Dicer is an RNase III-family enzyme characterized by its ribonuclease activity and dsRNA-binding properties. The enzyme generates nucleotide products from dsRNA of approximately 21-23. Processing of microRNAs, for example the let-7 precursor, by Dicer has also been observed. Dicer includes a dsRNA-binding domain located at the C-terminus of the enzyme.

Given the important role of Dicer in the generation of RNA-mediated gene silencing agents, the identification of proteins that interact with and/or regulate Dicer will help improve our understanding of RNA silencing and other Dicer-related processes. Moreover, Dicer-interacting and/or Dicer-regulating proteins are useful for the identification of a variety of modulatory agents for use in regulating RNA-mediated gene silencing.

### SUMMARY OF THE INVENTION

Important in the RNAi pathway of most organisms is the ribonuclease III enzyme Dicer. In particular, Dicer has been shown to play a key role in the processing of RNA precursors triggering the activation of both endogenous and exogenous pathogen responses (i.e., RNAi) and of small RNAs active as developmental regulators called microRNAs. The enzyme and its ancillary components have been poorly characterized to date. The instant invention is based, at least in part, on the identification of numerous interacting components of the enzyme Dicer, in particular, proteins previously unknown to interact with this critical protein. Moreover, the invention provides an assay for the identification of other components of this and related enzymes. Importantly, the invention demonstrates that the identified interactors of Dicer are capable of modulating its function in, for example RNAi. Still further, the identified C. elegans proteins have related homologs in vertebrates, for example, the mouse and humans, and therefore have application in the development of human diagnostic and therapeutic agents.

Accordingly, the invention has several advantages, which include, but are not limited to, the following:

providing interacting proteins of Dicer and there use in modulating Dicer function;

methods for identifying further interactors of Dicer and their structural and functional characteristics;

method for regulating Dicer activity though the use of Dicer interactors;

methods for improving the in vitro or in vivo processing of Dicer proteins or for use as targets for pharmaceutical intervention in order to modulate the properties of Dicer in vivo for improved RNAi; and

methods for stabilizing RNAi agents/compositions comprising Dicer by the addition of stabilizing interactor proteins or the same for use in purifying Dicer and other Dicer components.

Other features and advantages of the invention will be apparent from the following detailed description and claims.

### BRIEF DESCRIPTION OF THE DRAWINGS

FIG. 1 depicts a schematic of major components of the RNAi pathway, the role of Dicer, and Dicer interacting proteins, which have roles in microRNA maturation, RNAi initiation, and as enhancers of RNAi.

FIGS. 2A-C depicts biochemical fractionation and immunoprecipitations of DCR-1 from C. elegans embryos, and adults using the coupled HA monoclonal method. dcr−/− 8×HA rescue fractions and IP were realized using a complex array rescued strain of dcr-1 (ok247) with a transgene driving a 8×HA fusion.

FIGS. 3A-C depict the molecular architecture of the eri genes.

FIGS. 4A-B depict results regarding RNAi sensitivity, enhancement, and developmental defects of the eri genes. A. N2(WT) or eri mutants were fed on unc-73 (rnai) feeding strain for a generation and F1 broods of animals were scored for their exhibition of the associated phenotype: uncoordination, twisted morphology and limited movement (see lower panel). In the upper panel, results are shown for n=15, depicted error bars are shown for a confidence interval p=0.05. B. Brood sizes of the eri mutants at 15° C. (blue) and 25° C. (purple) are shown (upper panel). WT brood size is restored at 25° C. for all the eri mutations when crossing in with N2(wt) males (see lower panel). For all the broods, n=10, depicted error bars are shown for a confidence interval p=0.05.

FIGS. 5A-E depict small RNA defects in depletions for the DCR-1 interactors. In addition to dcr-1 and drh-3, the k02e2.6 locus also required the eri genes for accumulation, and the siRNAs were also absent from the eri genes RNA preparations from animals grown at 15° C. (5A-D). The lack of small RNAs in k02e2.6 in the eri mutants correlated with an upregulation of its mRNA, as quantified by real time PCR (5E). See the Materials and Methods for further detail.

FIG. 6 depicts a schematic showing that multiple silencing pathways are initiated by DCR-1, the eri gene products, and DRH-3. Distinct subsets of DCR-1 interactions are responsible for initiation of multiple small RNA silencing pathways. Shown here are the ‘classical’ RNAi pathway involving the RDE-1, RDE-4 and the DRH-1/2 proteins, the eri ‘endo’ RNAi pathway relying on the eri gene products, and the broader drh-3 dependent endo siRNA pathway.

### DETAILED DESCRIPTION OF THE INVENTION

The present invention is based, at least in part, on the discovery of previously unrecognized activity of several proteins as Dicer-interacting proteins (i.e., Dicer-interactors) and/or Dicer modulatory proteins (e.g., positive and/or negative regulatory proteins), see Tables 1 and 3. The invention features the defining of Dicer (DCR-1) interactions with an array of proteins involved in a variety of functions in C. elegans or other species, and the usage and alteration of these interactors and/or interactions to modulate or modify the different functions or activities of Dicer. The invention also features methods for efficient Dicer purification and identification of further interactors and/or interactions. This invention features methods for more efficient in vitro Dicer processing and materials for use in said methods, e.g., by the addition of a Dicer interacting protein that enhances Dicer activity. Knowledge of these Dicer interactors and/or interactions allows for the development of drug screening and/or targeting strategies or rationales, e.g., screening and/or targeting of Dicer and/or Dicer interactors in C. elegans, as well as in other species having homologous genes, to activate or antagonize Dicer's different functions and activities or to modulate its specificity toward its different proteins.

Accordingly, the present invention features Dicer interactors and methods of use of said interactors. In certain aspects, the invention provides methods for identifying a Dicer modulator, RNAi modulator and/or gene silencing modulator, including contacting a composition comprising, or a cell or organism that expresses Dicer or a bioactive fragment thereof and a Dicer interactor or a bioactive fragment thereof with a test compound and determining the ability of the test compound to modulate interaction (e.g., binding) of Dicer or the Dicer bioactive fragment to the Dicer interactor or the Dicer interactor bioactive fragment, such that the Dicer modulator, RNAi modulator and/or gene silencing modulator is identified.

In other aspects, the present invention provides methods for identifying a Dicer modulator, RNAi modulator and/or gene silencing modulator, including contacting a composition comprising, or a cell or organism that expresses Dicer or a bioactive fragment thereof and a Dicer interactor or a bioactive fragment thereof with a test compound and determining the ability of the test compound to modulate an activity of Dicer or the Dicer bioactive fragment, such that an the modulator is identified.

In certain embodiments, the activity of Dicer or the bioactive fragment thereof may be selected from the group consisting of: (1) processing of miRNA precursors; (2) processing of siRNA precursors; (3) mediating mRNA cleavage; (4) mediating assembly of RISC (e.g., via siRNAs); (5) directing translation repression (e.g., via miRNAs); (6) a ribonuclease activity (e.g., cleavage of dsRNA); and (7) initiation of RNAi.

In other aspects, the invention provides methods for identifying a Dicer modulator, RNAi modulator and/or gene silencing modulator, including contacting a composition comprising, or a cell or organism that expresses Dicer or a bioactive fragment thereof and a Dicer interactor or a bioactive fragment thereof with a test compound and determining the ability of the test compound to modulate an activity of the protein or the protein bioactive fragment, such that the Dicer modulator, RNAi modulator and/or gene silencing modulator is identified. In various embodiments of the preceding aspects the modulator identified may be a positive modulator or a negative modulator.

In various embodiments of the preceding aspects of the invention, the Dicer interactor may be selected from the proteins described in subsections IIIA-IIIMM, infra. In other embodiments, the Dicer is either Dicer1 or Dicer2. A Dicer bioactive fragment is any fragment of Dicer having sufficient size and structure to carry out at least one activity (e.g., biological activity) of the corresponding full-length Dicer protein. Similarly, a Dicer interactor bioactive fragment is any fragment of the Dicer interactor having sufficient size and structure to carry out at least one activity (e.g., biological activity) of the corresponding full-length Dicer interactor protein. Exemplary bioactive fragments include, but are not limited to, enzymatic domains, protein binding and/or interaction domains, and nucleic acid binding domains. Preferred bioactive fragments include regions or domains as described in detail in subsections IIIA-IIIMM, infra. The Dicer, Dicer bioactive fragment, Dicer interactor or the interactor bioactive fragment may be detectably labeled, radioactively labeled, or fluorescently labeled. Furthermore, in other embodiments, the interaction or activity may be compared to an appropriate control. In addition, at least one of the Dicer, Dicer bioactive fragment, Dicer interactor or protein bioactive fragment may be immobilized.

In various embodiments, the activity of the Dicer interactor or protein bioactive fragment is an activity set forth in subsections IIIA-IIIMM, infra. Bioactive fragments and/or fragment activities (and accordingly, Dicer interactor activities) are further described in detail in the references cited throughout subsections IIIA-IIIMM, infra. The entire content of these references is incorporated herein by reference.

In the aspects of the present invention, where the method involves a cell or organism, the cell or organism may overexpress the Dicer interactor or the bioactive fragment thereof, Dicer or the bioactive fragment thereof, or both the Dicer interactor (or protein bioactive fragment) and Dicer (or Dicer bioactive fragment).

In another aspect, the invention provides a modulator as identified by any of the preceding claims. The invention also provides for a pharmaceutical composition including the modulator.

In one aspect, the invention provides a method for identifying a Dicer:Dicer interactor modulator, including contacting a cell or organism expressing, or a composition comprising, Dicer or a bioactive fragment thereof and a Dicer interactor or a bioactive fragment thereof with a test compound and determining the ability of the test compound to affect interaction (e.g., binding) of the Dicer or the bioactive fragment thereof to the Dicer interactor or the bioactive fragment thereof, such that the modulator is identified.

In another aspect, the invention provides a method for identifying a Dicer:Dicer interactor modulator, including contacting a cell or organism expressing, or a composition comprising, Dicer or a bioactive fragment thereof and a Dicer interactor or a bioactive fragment thereof with a test compound and determining the ability of the test compound to affect activity of the Dicer or the bioactive fragment thereof, such that the modulator is identified.

In another aspect, the invention provides a method for identifying a Dicer:Dicer interactor modulator, including contacting a cell or organism expressing, or a composition comprising, Dicer or a bioactive fragment thereof and a Dicer interactor or a bioactive fragment thereof with a test compound and determining the ability of the test compound to affect activity of the Dicer interactor protein or the bioactive fragment thereof, such that the modulator is identified.

In yet another aspect, the invention provides a method for identifying a Dicer:Dicer interactor modulator, including contacting a cell or organism expressing, or a composition comprising, Dicer or a bioactive fragment thereof and a Dicer interactor or a bioactive fragment thereof with a test compound and determining the ability of the test compound to affect the phosphorylation state of the Dicer interactor or the bioactive fragment thereof, such that the modulator is identified.

In certain embodiments of the preceding aspects, the ability of the test compound to affect, for example, an interaction or activity includes the ability of the test compound to either enhance or inhibit such an interaction or activity. The Dicer may be Dicer1 or Dicer2.

In certain embodiments, the present invention provides methods of modulating Dicer, RNAi or gene silencing in a subject including administering to the subject a Dicer modulator, RNAi modulator and/or gene silencing modulator identified according to any of the above methods.

In another aspect, the invention provides an antibody that specifically binds to Dicer, a Dicer-interacting protein, or fragment thereof, wherein the antibody is capable of identifying, altering, or interfering with a Dicer:Dicer interactor interaction. In a related embodiment, the invention provides an antibody capable of binding an epitope within amino acid residue positions 1145 to 1347 of Dicer (DCR-1), or corresponding residues of a homolog thereof. The invention also provides polypeptides comprising Dicer epitopes suitable for raising such antibodies, e.g., for use as immunogens or screening polypeptides. In one embodiment, the epitope is within amino acid residue positions 1145 to 1347 of Dicer (DCR-1), or corresponding residues of a homolog thereof. The invention further provides for a pharmaceutical composition including the antibody.

In another aspect, the present invention provides a pharmaceutical composition including a Dicer-interacting protein. In yet another aspect, the present invention provides a pharmaceutical composition including a Dicer interacting protein domain of a Dicer protein or a Dicer interacting domain of a Dicer interacting protein, wherein either or both domains are capable of interfering with a Dicer:Dicer interacting protein interaction.

In yet another aspect, the invention provides a modulator of Dicer activity suitable for enhancing an RNAi therapy, and pharmaceutical compositions comprising such a modulator.

In other aspects, the present invention provides methods for treating an disease or disorder including administering any of the pharmaceutical compositions described above.

Various aspects of the invention are described in further detail in the following subsections:

I. Definitions

So that the invention may be more readily understood, certain terms are first defined.

As used herein, a “Dicer interacting protein” or “Dicer interactor” includes polypeptides having the amino acid sequences set forth in subsections IV, infra, as well as homologs, paralogs, and/or orthologs of such polypeptides, i.e. polypeptides having sufficient sequence identity to function in the same manner as the described polypeptides. Such polypeptides can interact directly, for example, physically bind with Dicer or a bioactive fragment thereof, and/or interact indirectly, for example, as measured by affecting a change in Dicer activity either in vitro or in vivo.

The term “Dicer” includes polypeptides having the amino acid sequences set forth in subsections III, infra, as well as homologs, paralogs, and/or orthologs of such polypeptides, i.e. polypeptides having sufficient sequence identity to function in the same manner as the described polypeptides.

The term “Dicer activity” includes any of the following properties or functions that can be ascribed to a Dicer protein such as: protein:protein binding activity (e.g., direct association with a Dicer interacting protein), miRNA maturation activity, RNAi initiation activity, RNAi enhancer activity, helicase activity, RISC activity, target recognition activity, and/or target gene cleavage activity.

The term “modulator of Dicer activity” includes agents capable of affecting a change in Dicer activity. Modulator agents include small molecules, nucleic acids (e.g., RNAi agents, siRNAs, shRNAs), peptides, and polypeptides. Dicer interacting proteins can be modulators of Dicer either directly or indirectly, for example, by physically interacting with Dicer or by affecting a change in Dicer activity. Thus, a modulator of a Dicer interacting protein which results in a change in Dicer activity can be considered a modulator of Dicer activity, albeit indirectly.

The term “derived from” includes partial, synthetic, recombinant, or genetically engineered nucleic acids or polypeptides that encode or represent a gene product substantially similar to a gene product from a particular source, for example, a nucleic acid source, a cell, or organismal source, from, for example, a nematode, fruit fly, rat, mouse, primate, or human.

The terms “homolog,” “paralog,” “ortholog,” includes their art recognized meaning. Typically, a homolog of a given gene product is one of functional similarity as well as sequence similarity. If the homolog is derived from a different organism, the homolog may be referred to as the ortholog. If several homologs exist in a given organism, the homolog may be referred to as a paralog. Typically, the sequence similarity/identity between homologs is at least about 40%, 50%, 60%, 70%, 80%, 90%, or more (or a percentage falling within any interval or range of the foregoing). Methods for determining such similarity/identity are described, infra. Motifs conserved between homologs can have a sequence similarity/identity of at least about 70%, 80%, 90%, or more. It is understood that when comparing gene product sequence between diverse organisms, for example, nematodes and humans, sequence similarity between given homologs across the entire protein sequence may be low. However, if functional complementarity exists, and in addition, if conserved motifs exist, e.g., protein; protein interaction motifs, e.g., motifs involved in Dicer activity or Dicer:Dicer interacting protein interactions, then the gene products being compared can be considered homologs and thus selected as compositions for use in the methods of the invention, as described herein.

The phrase “introducing into the cell or organism” includes any art recognized method for introducing genetic information into an cell extract, cell, or organism. Typical modes of such transfer of genetic information include the contacting, transfection, microinjection and/or feeding of nucleic acid agents or expression vectors to an extract, cell, or organism. Other methods include cell fusion, pronuclear injection, genetic crosses/mutagenesis, and the like.

The term “bioactive fragment” includes any portion (e.g., a segment of contiguous amino acids) of a Dicer interactor or Dicer protein sufficient to exhibit or exert at least one Dicer protein- or Dicer-associated activity including, for example, the ability to bind to Dicer or Dicer interactor protein, respectively.

The phrase “encodes a gene product” includes the generation of a RNA molecule from a DNA molecule (i.e., a complementary RNA molecule generated from the DNA molecule by the process of transcription) and/or the generation of a polypeptide or protein molecule from an RNA (i.e., by the processes of transcription and translation).

The term “kit” is any manufacture (e.g. a package or container) comprising at least one reagent or component, e.g. a construct, molecule, and/or compound, the manufacture being promoted, distributed, or sold as a unit for performing the methods of the invention.

The term “target gene” includes a gene intended for downregulation via RNA interference (“RNAi”). The term “target gene product” or “target protein” refers to a gene product, e.g., a nucleic acid or protein, intended for downregulation via RNAi. The term “target RNA” refers to an RNA molecule intended for degradation by RNAi, e.g., by nucleic acid cleavage. An exemplary “target RNA” is a coding RNA molecule (e.g., an RNA molecule encoding a gene product, e.g., an mRNA and protein so encoded therefrom).

The term “expression” of a gene or nucleic acid encompasses not only cellular gene expression, but also the transcription and translation of nucleic acid(s) in cloning systems and in any other context.

The term “RNA interference” or “RNAi”, as used herein, refers generally to a sequence-specific or selective process by which a target molecule (e.g., a target gene, protein, or RNA) is downregulated. In specific embodiments, the process of “RNA interference” or “RNAi” features degradation of RNA molecules, e.g., RNA molecules within a cell, the degradation being triggered by an RNAi agent. Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

The term “RNAi agent”, includes an RNA (or analog thereof), comprising a sequence having sufficient complementarity to a target RNA (i.e., the RNA being degraded) to direct RNAi. A sequence having a “sufficiently complementary to a target RNA sequence to direct RNAi” means that the RNAi agent has a sequence sufficient to trigger the destruction of the target RNA by the RNAI machinery (e.g., the RISC complex) or process. The term RNA agent or RNAi agent includes small interfering RNA (siRNA) (also referred to in the art as short interfering RNAs) as well as small hairpin RNA or shRNA.

The term “small interfering RNA,” “siRNA,” or “short interfering RNAs” includes a double-stranded RNA agent, which is capable of directing or mediating RNA interference. Naturally occurring siRNAs are generated from longer dsRNA molecules (e.g., >25 nucleotides in length) by a cell's RNAi machinery (e.g., the RISC complex).

The term “small hairpin RNA” or “shRNA” (also referred to in the art as “short hairpin RNA”), includes an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

The term “subject”, as used herein, includes living organisms at risk for or having a cellular, neurological, e.g. neurodegenerative disease, or disorder. Examples of subjects include humans, monkeys, cows, sheep, goats, dogs, cats, mice, rats, and transgenic species thereof. Administration of the compositions of the present invention to a subject to be treated can be carried out using known procedures, at dosages and for periods of time effective to modulate RNAi in the subject as further described herein.

The term “treatment”, as used herein, is defined as the application or administration of a therapeutic agent to a subject, or application or administration of a therapeutic agent to an isolated tissue or cell line from a subject, who has a disease or disorder, a symptom of a disease or disorder, or a predisposition toward a disease or disorder, with the purpose to cure, heal, alleviate, relieve, alter, remedy, ameliorate, improve or affect the disease or disorder, the symptoms of the disease or disorder, or the predisposition toward a disease or disorder. A therapeutic agent includes, but is not limited to, small molecules, peptides, antibodies, ribozymes, antisense oligonucleotides, RNAi agents, chemotherapeutic agents, and radiation.

The term “effective amount”, as used here in, is defined as that amount necessary or sufficient to treat or prevent a disorder, e.g. a neurological or a neurodegenerative disease or disorder. The effective amount can vary depending on such factors as the size and weight of the subject, the type of illness, or the particular agent being administered. One of ordinary skill in the art would be able to study the aforementioned factors and make the determination regarding the effective amount of the agent without undue experimentation.

The term “pharmaceutical composition” as used herein, refers to an agent formulated with one or more compatible solid or liquid filler diluents or encapsulating substances, which are suitable for administration to a human or lower animal.

The phrase “a gene involved” in a disorder includes a gene, the normal or aberrant expression or function of which effects or causes a disease or disorder or at least one symptom of said disease or disorder.

The phrase “examining the function of a gene in a cell or organism” refers to examining or studying the expression, activity, function, or phenotype arising therefrom.

A “suitable control” or “appropriate control” refers to any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a Dicer activity, a RISC level of activity or amount, target gene level or target gene degradation level, a transcription rate, mRNA level, translation rate, protein level, biological activity, cellular characteristic or property, genotype, phenotype, etc. can be determined prior to introducing a nucleic acid or test compound of the invention into a cell extract, cell, or organism.

The term “cell” refers to any eukaryotic cell which exhibits RNAi activity and includes, e.g., animal cells (e.g., mammalian cells, e.g., human or murine cells), nematode cells, plant cells, and yeast. The term includes cell lines, e.g., mammalian cell lines such as HeLa cells as well as embryonic cells, e.g., embryonic stem cells and collections of cells in the form of, e.g., a tissue.

The term “cell extract” refers to a lysate or acellular preparation of a cell as defined above and can be a crude extract or partially purified as well as comprise additional agents such as recombinant polypeptides, nucleic acids, and/or buffers or stabilizers.

The term “organism” refers to multicellular organisms such as, e.g., C. elegans, Drosophila, mouse, and human.

The term “vector” refers to a nucleic acid molecule (either DNA or RNA) capable of conferring the expression of a gene product when introduced into a host cell or host cell extract. In one embodiment, the vector allows for temporal or conditional expression of one or more nucleic acids of the invention, e.g., a single strand, RNA agent, siRNA, or shRNA. The vector may be episomal or chromosomally (e.g., transgenically) integrated into a host cell genome.

The terms used herein are not intended to be limiting of the invention.

II. Overview

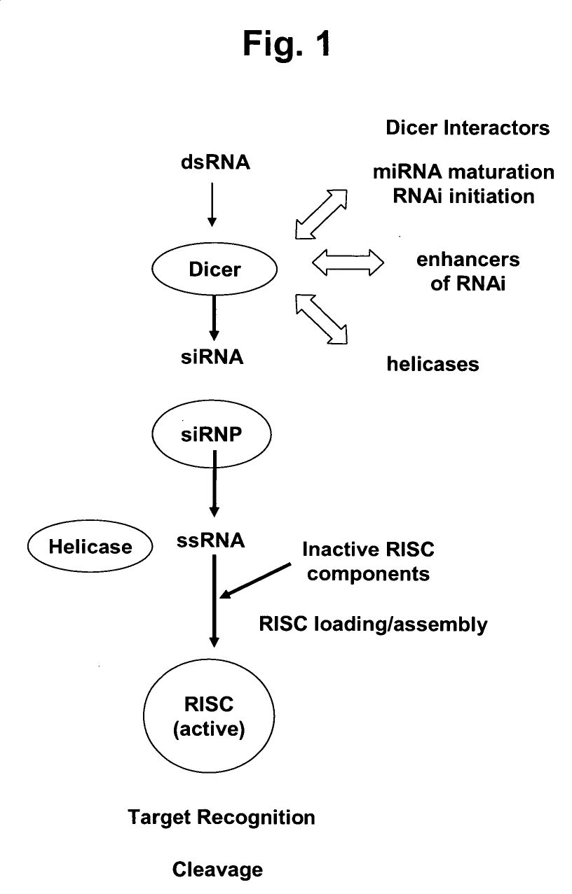

Dicer, a ribonuclease III/DExH-box helicase (DCR-1 in C. elegans) plays a central role in a variety of small RNA-directed gene silencing mechanisms for a large range of organisms (see FIGS. 1 & 6).

Its best characterized activity is the processing of double-stranded RNAs into smaller RNA hybrid species of 21 to 25 nucleotides (nt) in length with staggered 2 nucleotides overhangs at the 3′ ends of the duplex, and a 5′ phosphate group; both of which determinants have been shown to be required for efficient silencing.

This Dicer activity was first shown to act in the initiation phase of two modes of post-transcriptional gene silencing. In RNA interference (RNAi) and in the microRNA-dependent silencing, Dicer recognizes a double-stranded RNA (dsRNA) trigger to direct a potent, and sequence-specific gene silencing response. This process requires the assembly of the small RNA product in a downstream complex called RISC, for which Argonaute proteins are a central component. This complex is responsible for a cognate mRNA search, and for the subsequent silencing of the complementary transcript.

Dicer is responsible for the integration of a variety of RNA signals with distinct biological outcomes. Dicer also initiates other RNA-dependent silencing pathways such as chromosome folding and the like. Therefore a key problem to address is how some specific classes of dsRNAs are recognized and recruited to be processed by Dicer, and how RNA triggers of distinct origins potentiate different silencing responses.

The present invention provides methods and compositions for conducting in vitro and in vivo assays for identifying Dicer interacting proteins, in particular, Dicer interacting proteins that can affect Dicer activity, and modulators thereof.

III. Dicer Interacting Proteins or Dicer Interactors

According to the invention, several proteins have been identified as interacting with and/or regulating Dicer, e.g., Dicer activity. These Dicer interactors are described in detail below under subsections IIIA through IIIMM. Using methods described in the present disclosure, use of any one of these proteins, or cognate orthologs or paralogs, in appropriate screening assays would provide for the identification of Dicer modulators and/or RNAi-modulators, and/or gene silencing modulators.

IIIA. RDE-4

| | |
|---|---|
| LOCUS | NP_499265 385 aa linear INV 21- |
| NOVEMBER | |
| 2003 | |
| DEFINITION | RNAi Defective RDE-4, RNA interference |
| | promoting factor with double-stranded |
| | RNA binding motif (43.4 kD) (rde-4) |
| | [Caenorhabditis elegans]. |
| ACCESSION | NP_499265 |
| VERSION | NP_499265.1 GI: 17555186 |
| DBSOURCE | REFSEQ: accession NM 066864.2 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; |
| | Rhabditoidea; Rhabditidae; |
| | Peloderinae; Caenorhabditis. |

| | |
|---|---|
| REFERENCE | 1 (residues 1 to 385) |

| | |
|---|---|
| AUTHORS | Walhout,A. J., Reboul,J., Shtanko,O., |
| | Bertin,N., Vaglio,P., Ge,H., Lee,H., |
| | Doucette-Stamm,L., Gunsalus,K. C., |
| | Schetter,A. J., Morton,D. G., |
| | Kemphues,K. J., Reinke,V., Kim,S. K., |
| | Piano,F. and Vidal, M. |
| TITLE | Integrating interactome, phenome, and |
| | transcriptome mapping data for the C. |
| | elegans germline |
| JOURNAL | Curr. Biol. 12 (22), 1952-1958 (2002) |
| MEDLINE | 22335532 |
| PUBMED | 12445390 |

| | |
|---|---|
| REFERENCE | 2 (residues 1 to 385) |

| | |
|---|---|
| AUTHORS | Tabara,H., Yigit,E., Siomi,H. and |
| | Mello,C. C. |
| TITLE | The dsRNA binding protein RDE-4 inter- |
| | acts with RDE-1, DCR-1, and a DExH-box |
| | helicase to direct RNAi in C. elegans |
| JOURNAL | Cell 109 (7), 861-871 (2002) |
| MEDLINE | 22105477 |
| PUBMED | 12110183 |

| | |
|---|---|
| REFERENCE | 3 (residues 1 to 385) |

| | |
|---|---|
| AUTHORS | Tijsterman,M., Ketting,R. F., |
| | Okihara,K. L., Sijen,T. and |
| | Plasterk,R. H. |
| TITLE | RNA helicase MUT-14-dependent gene |
| | silencing triggered in C. elegans by |
| | short antisense RNAs |
| JOURNAL | Science 295 (5555), 694-697 (2002) |
| MEDLINE | 21669321 |
| PUBMED | 11809977 |

| | |
|---|---|
| REFERENCE | 4 (residues 1 to 385) |

| | |
|---|---|
| AUTHORS | Parrish,S. and Fire,A. |
| TITLE | Distinct roles for RDE-1 and RDE-4 |
| | during RNA interference in |
| | Caenorhabditis elegans |
| JOURNAL | RNA 7 (10), 1397-1402 (2001) |
| MEDLINE | 21535543 |
| PUBMED | 11680844 |

| | |
|---|---|
| REFERENCE | 5 (residues 1 to 385) |

| | |
|---|---|
| AUTHORS | Grishok,A., Tabara,H. and Mello,C. C. |
| TITLE | Genetic requirements for inheritance of |
| | RNAi in C. elegans |
| JOURNAL | Science 287 (5462), 2494-2497 (2000) |
| MEDLINE | 20207007 |
| PUBMED | 10741970 |

| | |
|---|---|
| REFERENCE | 6 (residues 1 to 385) |

| | |
|---|---|
| AUTHORS | Tabara,H., Sarkissian,M., Kelly,W. G., |
| | Fleenor,J., Grishok,A., Timmons,L., |
| | Fire,A. and Mello,C. C. |
| TITLE | The rde-1 gene, RNA interference, and |
| | transposon silencing in C. elegans |
| JOURNAL | Cell 99 (2), 123-132 (1999) |
| MEDLINE | 20004389 |
| PUBMED | 10535731 |

| | |
|---|---|
| COMMENT | REVIEWED REFSEQ: This record has been |
| | curated by NCBI staff. This record is |
| | derived from an annotated genomic se- |
| | quence (NC_003281). The reference se- |
| | quence was derived from AY071926.1. |
| | Summary: This gene rde-4, also known as |
| | T20G5.11, 3L306 or YK5801, maps at |
| | (III; +1.89). Its phenotype is rnai |
| | defective. It encodes a RNA interfer- |
| | ence promoting factor with double- |
| | stranded RNA binding motif. From Pfam |
| | homology, the product would have |
| | double-stranded RNA binding activity |
| | and would localize in intracellular. |
| | According to the Worm Transcriptome |
| | Project, it is well expressed at all |
| | stages of development [Kohara cDNAs]. |
| | Its sequence is defined by 10 cDNA |
| | clones. |
| | Phenotype |
| | WM49 rde-4 (ne301) III [Craig Mello, |
| | Tabara/Mello, mut-6] RNAi deficient. |
| | RNA interference results: |
| | [T. Hyman 2000] No obvious phenotype |
| | (by injecting genomic PCR product |
| | TH: T20G5.11). |
| | [J. Ahringer 2003] No obvious phenotype |
| | (by feeding genomic PCR product |
| | JA: T20G5.11). |
| | [F. Piano 2002] No P0 sterility de- |
| | tected. No postembryonic phenotypes |
| | observed among progeny. No obvious |
| | phenotype. |
| | Expression |
| | The expression profile for the gene, |
| | derived from the proportion of animals |
| | at each stage in each Kohara library |
| | is: embryos 47%, L1 or L2 larvae 29%, |
| | L3 to adult 25%. |
| | In situ hybridisation pictures to all |
| | stages of development are available |
| | from Kohara NextDB. |
| | Pattern germline enriched [Piano, |
| | 2002]. |
| | This complete mRNA is 1747 bp long. Its |
| | sequence exactly matches the genome. |
| | The premessenger has 4 exons. It covers |
| | 1.89 kb on the WS97 genome. It is tran- |
| | spliced to SL1. It has a very long 3′ |
| | UTR. The protein (385 aa, 43.4 kDa, pI |
| | 5.2) contains 2 Double-stranded RNA |
| | binding (DsRBD) domain motifs. It also |
| | contains a coil coil stretch [(Psort2]. |
| | Taxblast (threshold 10\hat{ }-3) tracks an- |
| | cestors down to caenorhabditis elegans. |
| | COMPLETENESS: full length. |
| | Method: conceptual translation. |

| | |
|---|---|
| FEATURES | Location/Qualifiers |

| | |
|---|---|
| source | 1 . . . 385 |
| | /organism = “Caenorhabditis |
| | elegans” |
| | /db_xref = “taxon: 6239” |
| | /chromosome = “III” |
| | /map = “III; +1.89 cM (interpolated |
| | genetic position)” |
| | /map = “III; covering 1888 bp, from |
| | base 10218484 to 10216597 on genome |
| | release WS97” |
| | /clone = “Primers to amplify the |
| | CDS (3468 bp, Stop included): |
| | ATGGATTTAACCAAACTAACGTTTGAA (T = |
| | 55.9), TCAATCCGTGAAATCATAGGTGT |
| | (T = 56.6). Complete CDS clones: |
| | AY071926, yk832c2. Recommended |
| | clone (from the Kohara collection): |
| | yk832c2. Other clone(s): yk627d6, |
| | yk333g4, yk596c11, yk565d11, |
| | yk469h7, yk1429h2, yk1706h7, |
| | yk1726d4. |
| | for edited clone sequences see |
| | www.wormgenes.org” |
| | /clone_lib = “Kohara embryonic |
| | lambda gt11 library: yk627d6, |
| | yk333g4, yk596c11, yk565d11, |
| | yk469h7; Kohara Sugano L1 larvae |
| | cap-selected library: yk832c2; |
| | Kohara Sugano L2 larvae cap- |
| | selected library: yk1706h7, |
| | yk1726d4; Kohara Sugano L4 larvae |
| | cap-selected library: yk1429h2; gb: |
| | AY071926” |
| Protein | 1 . . . 385 |
| | /product = “RNAi DEfective RDE-4, |
| | RNA interference promoting factor |
| | with double-stranded RNA binding |
| | motif (43.4 kD) (rde-4)” |
| Region | 41 . . . 104 |
| | /region_name = “[Pfam/InterPro |
| | description] double-stranded RNA |
| | binding (DsRBD) domain” |
| | /db_xref = “CDD: pfam00035” |
| Region | 116 . . . 149 |
| | /region_name = “[PSORT] coil coil |
| | domain: |
| | PGTTKEEALSNIDQISDKAEELKRSTSDAVQDND” |
| Region | 170 . . . 232 |
| | /region_name = “[Pfam/InterPro |
| | description] double-stranded RNA |
| | binding (DsRBD) domain” |
| | /db_xref = “CDD: pfam00035” |
| CDS | 1 . . . 385 |
| | /gene = “rde-4” |
| | /locus_tag = “3L306” |
| | /coded_by = |
| | “NM_066864.2: 1 . . . 1158” |
| | /db_xref = “AceView/WormGenes: |
| | rde-4” |
| | /db_xref = “GeneID: 176438” |
| | /db_xref = “LocusID: 176438” |
| | /db_xref = “WormBase: T20G5.11” |

| | |
|---|---|
| ORIGIN | |
| 1 | mdltkltfes vfggsdvpmk psrsednktp |
| | rnrtdlemfl kktplmvlee aakavyqktp |
| 61 | twgtvelpeg femtlilnei tvkgqatskk |
| | aarqkaavey lrkvvekgkh eiffipgttk |
| 121 | eealsnidqi sdkaeelkrs tsdavqdndn |
| | ddsiptsaef ppgisptenw vgklqeksqk |
| 181 | sklqapiyed sknerterfl victmcnqkt |
| | rgirskkkda knlaawlmwk aledgiesle |
| 241 | sydmvdvien leeaehllei qdqaskikdk |
| | hsalidilsd kkrfsdysmd fnvlsvstmg |
| 301 | ihqvlleisf rrlvspdpdd lemgaehtqt |
| | eeimkataek eklrkknmpd sqplvfaghg |
| 361 | ssaeeakqca cksaiihfnt ydftd |
| // | |

IIIB. ALG-1

ALG-1 is a homolog of rde-1 that is involved in RNA interference and affects developmental timing along with alg-2 and dcr-1 by regulating expression of the lin-4 and let-7 small temporal RNAs. The ALG-1 protein contains regions of similarity to Pfam domains PF02170 (PAZ domain, Residues 377-514), PF02171 (Piwi domain, Residues 660-961). The protein has been implicated in embryonic development, inferred from mutant phenotype Grishok, A. et al., Cell 2001 106:23-34. Homologs include H. sapiens eukaryotic translation initiation factor 2C 4, C. elegans gene T07D3.7a, M. musculus Argonaute 1 protein (Fragment), R. norvegicus eukaryotic translation initiation factor 2C 2 (eIF2C 2) (eIF-2C 2)s(Golgi ER protein 95 kDa) (GERp95) and D. melanogaster AGO1.

| | |
|---|---|
| LOCUS | NP_510322 1002 aa linear INV 21- |
| NOVEMBER | |
| 2003 | |
| DEFINITION | argonaute (plant)-Like Gene (110.9 kD) |
| | (alg-1) [Caenorhabditis elegans]. |
| ACCESSION | NP_510322 |
| VERSION | NP_510322.2 GI: 25148113 |
| DBSOURCE | REFSEQ: accession NM 077921.2 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 1002) |

| | |
|---|---|
| AUTHORS | Kamath,R. S., Fraser,A. G., Dong,Y., |
| | Poulin,G., Durbin,R., Gotta,M., |
| | Kanapin,A., Le Bot,N., Moreno,S., |
| | Sohrmann,M., Welchman,D. P., |
| | Zipperlen,P. and Ahringer,J. |
| TITLE | Systematic functional analysis of the |
| | Caenorhabditis elegans genome using |
| | RNAi |
| JOURNAL | Nature 421 (6920), 231-237 (2003) |
| MEDLINE | 22417569 |
| PUBMED | 12529635 |

| | |
|---|---|
| REFERENCE | 2 (residues 1 to 1002) |

| | |
|---|---|
| AUTHORS | Morel,J. B., Godon,C., Mourrain,P., |
| | Beclin,C., Boutet,S., Feuerbach,F., |
| | Proux,F. and Vaucheret,H. |
| TITLE | Fertile hypomorphic ARGONAUTE (agol) |
| | mutants impaired in post-transcription- |
| | al gene silencing and virus resistance |
| JOURNAL | Plant Cell 14 (3), 629-639 (2002) |
| MEDLINE | 21907852 |
| PUBMED | 11910010 |

| | |
|---|---|
| REFERENCE | 3 (residues 1 to 1002) |

| | |
|---|---|
| AUTHORS | Grishok,A., Pasquinelli,A. E., |
| | Conte,D., Li,N., Parrish,S., Ha, I., |
| | Baillie,D. L., Fire,A., Ruvkun,G. and |
| | Mello,C. C. |
| TITLE | Genes and mechanisms related to RNA |
| | interference regulate expression of the |
| | small temporal RNAs that control C. |
| | elegans developmental timing |
| JOURNAL | Cell 106 (1), 23-34 (2001) |
| MEDLINE | 21354308 |
| PUBMED | 11461699 |

| | |
|---|---|
| COMMENT | VALIDATED REFSEQ: This record has un- |
| | dergone preliminary review of the se- |
| | quence, but has not yet been subject to |
| | final review. This record is derived |
| | from an annotated genomic sequence |
| | (NC_003284). The reference sequence was |
| | derived from WormBase CDS: F48F7.1. On |
| | Nov. 21, 2002 this sequence version re- |
| | placed gi: 17549901. |
| | Summary: This gene alg-1, also known as |
| | F48F7.1, XO573 or YK3586, maps at (X; |
| | +14.45). Its phenotype is clear, trans- |
| | lucent appearance, uncoordinated loco- |
| | motion, protruding vulva. It encodes an |
| | argonaute (plant)-Like Gene. |
| | According to the Worm Transcriptome |
| | Project, it is well expressed at all |
| | stages of development [Kohara cDNAs]. |
| | Its sequence is fully supported by 17 |
| | cDNA clones. |
| | RNA interference results |
| | [J. Ahringer 2003] Clear, uncoordina- |
| | ted, protruding vulva (by feeding geno- |
| | mic PCR product JA: F48F7.1). |
| | Expression |
| | The expression profile for the gene, |
| | derived from the proportion of animals |
| | at each stage in each Kohara library |
| | is: embryos 26%, L1 or L2 larvae 27%, |
| | L3 to adult 48%. |
| | In situ hybridisation pictures to all |
| | stages of development are available |
| | from Kohara NextDB. |
| | The CDS has 6 exons. It covers 3.42 kb |
| | on the WS97 genome. The protein (1002 |
| | aa, 110.9 kDa, pI 9.3) contains one |
| | Argonaute and Dicer protein, PAZ motif, |
| | one stem cell self-renewal protein Piwi |
| | motif. It also contains a 2nd peroximal |
| | domain Psort2]. Taxblast (threshold |
| | 10\hat{ }-3) tracks ancestors down to |
| | eukaryota. |
| | COMPLETENESS: full length. |
| | Method: conceptual translation. |

| | |
|---|---|
| FEATURES | Location/Qualifiers |

| | |
|---|---|
| source | 1 . . . 1002 |
| | /organism = “Caenorhabditis |
| | elegans” |
| | /db_xref = “taxon: 6239” |
| | /chromosome = “X” |
| | /map = “X; +14.45 cM (interpolated |
| | genetic position)” |
| | /map = “X; covering 4989 bp, from |
| | base 13941769 to 13946759 on genome |
| | release WS97” |
| | /clone_lib = “Kohara embryonic |
| | lambda gt11 library: yk60e5, |
| | yk403g7, yk142f4, yk375c8, |
| | yk481b11, yk245e6; Kohara Sugano L1 |
| | larvae cap-selected library: |
| | yk759f4, yk889c6, yk1013a7, |
| | yk1108b4, yk1164h8; Kohara Sugano |
| | L2 larvae cap-selected library: |
| | yk1609d8; Kohara Sugano |
| | L4 larvae cap-selected |
| | library: yk1427e7; Kohara mixed |
| | stage library, from him-8 strain, |
| | containing 15-30% males: yk100d5, |
| | yk545h7, yk369b2; Kohara mixed |
| | stage library, from him-8 strain, |
| | containing 15-30% males: yk286h2” |
| Protein | 1 . . . 1002 |
| | /product = “argonaute (plant)-Like |
| | Gene (110.9 kD) (alg-1)” |
| Region | 377 . . . 514 |
| | /region_name = “[Pfam/InterPro |
| | description] argonaute and Dicer |
| | protein, PAZ” |
| | /db_xref = “CDD: pfam02170” |
| Region | 460 . . . 468 |
| | /region name = “[PSORT] 2nd perox- |
| | imal domain: RIQLKYPHL” |
| Region | 660 . . . 961 |
| | /region name = “[Pfam/InterPro |
| | description] stem cell self-renewal |
| | protein Piwi” |
| | /db_xref = “CDD: pfam02171” |
| CDS | 1 . . . 1002 |
| | /gene = “alg-1” |
| | /locus_tag = “XO573” |
| | /coded_by = |
| | “NM_077921.2: 1 . . . 3009” |
| | /db_xref = “AceView/WormGenes: |
| | alg-1” |
| | /db_xref = “GeneID: 181504” |
| | /db_xref = “LocusID: 181504” |
| | /db_xref = “WormBase: F48F7.1” |

| | |
|---|---|
| ORIGIN | |
| 1 | msggpqylpg vmnstiqqqp qsatssflps |
| | qpisststss qvvptsgatq qppfpsaqaa |
| 61 | astalqndle eifnspptqp qtfsdvpqrq |
| | agslapgvpi gntsvsiqep antlqqglpg |
| 121 | gapgqlpggn qsgiqfqcpr rpnhgvegrs |
| | illranhfav ripggtiqhy qvdvtpdkcp |
| 181 | rrvnreiisc lisafskyft nirpvydgkr |
| | nmytreplpi grermdfdvt lpgdsaverq |
| 241 | fsvslkwvgq vslstledam egrvrqvpfe |
| | avqamdvilr hlpslkytpv grsffsppvp |
| 301 | nasgvmagsc ppqasgavag gahsagqyha |
| | esklgggrev wfgfhqsvrp sqwkmmlnid |
| 361 | vsatafyrsm pviefiaevl elpvqalaer |
| | ralsdaqrvk ftkeirglki eithcgqmrr |
| 421 | kyrvcnvtrr paqtqtfplq letgqtiect |
| | vakyfydkyr iqlkyphlpc lqvgqeqkht |
| 481 | ylppevcniv pqqrcikklt dvqtstmika |
| | tarsaperer eisnlvrkae fsadpfahef |
| 541 | gitinpamte vkgrvlsapk llyggrtrat |
| | alpnqgvwdm rgkqfhtgid vrvwaiacfa |
| 601 | qqqhvkendl rmftnqlqri sndaqmpivg |
| | npcfckyavg veqvepmfky lkqnysgiql |
| 661 | vvvvlpgktp vyaevkrvgd tvlgiatqcv |
| | qaknairttp qtlsnlclkm nvklggvnsi |
| 721 | llpnvrprif nepviffgcd ithppagdsr |
| | kpsiaavvgs mdahpsryaa tvrvqqhrqe |
| 781 | iisdltymvr ellvqfyrnt rfkparivvy |
| | rdgvsegqff nvlqyelrai reacmmlerg |
| 841 | yqpgitfiav qkrhhtrlfa vdkkdqvgka |
| | ynippgttvd vgithptefd fylcshagiq |
| 901 | gtsrpshyhv lwddnnltad elqqltyqmc |
| | htyvrctrsv sipapayyah ivafraryhl |
| 961 | vdrehdsgeg sqpsgtsedt tlsnmaravq |
| | vhpdannvmy fa |
| // | |

IIIC. ALG-2

| | |
|---|---|
| LOCUS | NP_871992 910 aa linear INV 21- |
| NOVEMBER | |
| 2003 | |
| DEFINITION | argonaute (plant)-Like Gene (101.6 kD) |
| | (alg-2) [Caenorhabditis elegans]. |
| ACCESSION | NP_871992 |
| VERSION | NP_871992.1 GI: 32564644 |
| DBSOURCE | REFSEQ: accession NM 182192.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; |
| | Rhabditoidea; Rhabditidae; |
| | Peloderinae; Caenorhabditis. |

| | |
|---|---|
| REFERENCE | 1 (residues 1 to 910) |

| | |
|---|---|
| AUTHORS | Morel,J. B., Godon,C., Mourrain,P., |
| | Beclin,C., Boutet,S., Feuerbach, F., |
| | Proux, F. and Vaucheret,H. |
| TITLE | Fertile hypomorphic ARGONAUTE (agol) |
| | mutants impaired in post-transcription- |
| | al gene silencing and virus resistance |
| JOURNAL | Plant Cell 14 (3), 629-639 (2002) |
| MEDLINE | 21907852 |
| PUBMED | 11910010 |

| | |
|---|---|
| REFERENCE | 2 (residues 1 to 910) |

| | |
|---|---|
| AUTHORS | Grishok,A., Pasquinelli,A. E., |
| | Conte,D., Li,N., Parrish,S., Ha,I., |
| | Baillie,D. L., Fire,A., Ruvkun,G. and |
| | Mello,C. C. |
| TITLE | Genes and mechanisms related to RNA in- |
| | terference regulate expression of the |
| | small temporal RNAs that control C. |
| | elegans developmental timing |
| JOURNAL | Cell 106 (1), 23-34 (2001) |
| MEDLINE | 21354308 |
| PUBMED | 11461699 |

| | |
|---|---|
| REFERENCE | 3 (residues 1 to 910) |

| | |
|---|---|
| AUTHORS | Missotten,M., Nichols,A., Rieger,K. and |
| | Sadoul,R. |
| TITLE | Alix, a novel mouse protein undergoing |
| | calcium-dependent interaction with the |
| | apoptosis-linked-gene 2 (ALG-2) protein |
| JOURNAL | Cell Death Differ. 6 (2), 124-129 |
| | (1999) |
| MEDLINE | 99218669 |
| PUBMED | 10200558 |

| | |
|---|---|
| COMMENT | VALIDATED REFSEQ: This record has un- |
| | dergone preliminary review of the se- |
| | quence, but has not yet been subject to |
| | final review. This record is derived |
| | from an annotated genomic sequence |
| | (NC_003280). The reference sequence was |
| | derived from WormBase CDS: T07D3.7a. |
| | Summary: This gene alg-2, also known as |
| | T07D3.7, 2B167 or YK2467, maps at (II; |
| | −13.80). It encodes an argonaute |
| | (plant)-Like Gene. According to the |
| | Worm Transcriptome Project, it is ex- |
| | pressed at high level mainly in embryos |
| | and some in L1 larvae [Kohara cDNAs]. |
| | Its sequence is fully supported by 29 |
| | cDNA clones and produces, by alterna- |
| | tive splicing, 2 different transcripts |
| | a, b altogether encoding 2 different |
| | protein isoforms. |
| | Phenotype |
| | Knock-out allele, deletion obtained by |
| | the Gene Knockout Consortium ok215, |
| | ok304 (strain RB574) [R Barstead, |
| | Oklahoma MRF, USA]. Selected strain |
| | available from the CGC. |
| | RB574 alg-2 (ok304) II [Robert |
| | Barstead, OMRF Knockout Group/Barstead, |
| | UV/TMP] [Craig Mello description] |
| | Homozygous viable, contains an out of |
| | frame deletion removing nucleotides |
| | encoding amino acids 34-374. [R |
| | Barstead] Homozygous. Outer Left Se- |
| | quence: tctgagtttggctcgatgtg. Outer |
| | Right Sequence: atgttccttggataccagcg. |
| | Inner Left Sequence: |
| | agcccagaactgggaaactt. Inner Right Se- |
| | quence: aagtcgaattccgttggatg. Inner |
| | Primer PCR Product: 3297. Deletion |
| | length: 1378 bp. Deletion breakpoints: |
| | Flanking positions are T07D3 coordi- |
| | nates 2397/3776. Sequence read at break |
| | from ok304 internal left primer: |
| | TCTAATTTTCCAATTTTCAG/ |
| | GATATTGTTCCAGGACAGCG. Breakpoint data |
| | provided by the Vancouver Gene Knockout |
| | Lab (URL: www.zoology.ubc.ca/kogeno- |
| | mics/kowebpge.html).www.mutantfac- |
| | tory.ouhsc.edu/. |
| | RNA interference results: |
| | [J. Ahringer 2003] No obvious phenotype |
| | (by feeding genomic PCR product |
| | JA: T07D3.7). |
| | Expression |
| | The expression profile for the gene, |
| | derived from the proportion of animals |
| | at each stage in each Kohara library |
| | is: embryos 75%, L1 or L2 larvae 16%, |
| | L3 to adult 9%. |
| | In situ hybridisation pictures to all |
| | stages of development are available |
| | from Kohara NextDB. |
| | The CDS has 7 exons. It covers 5.26 kb |
| | on the WS97 genome. The protein (910 |
| | aa, 101.6 kDa, pI 9.2) contains one |
| | Argonaute and Dicer protein, PAZ motif, |
| | one stem cell self-renewal protein Piwi |
| | motif. It also contains a 2nd peroximal |
| | domain [Psort2]. It is predicted to |
| | localise in the cytoplasm [Psort2]. |
| | Taxblast (threshold 10\hat{ }-3) tracks an- |
| | cestors down to eukaryota. |
| | COMPLETENESS: full length. |
| | Method: conceptual translation. |

| | |
|---|---|
| FEATURES | Location/Qualifiers |

| | |
|---|---|
| source | 1 . . . 910 |
| | /organism = “Caenorhabditis elegans” |
| | /db_xref = “taxon: 6239” |
| | /chromosome = “II” |
| | /map = “II; −13.80 cM (interpolated |
| | genetic position)” |
| | /map = “II; covering 6702 bp, from |
| | base 873182 to 879885 on genome |
| | release WS97” |
| Protein | 1 . . . 910 |
| | /product = “argonaute (plant)-Like |
| | Gene (101.6 kD) (alg-2)” |
| Region | 282 . . . 419 |
| | /region name = “[Pfam/InterPro |
| | description] argonaute and Dicer |
| | protein, PAZ” |
| | /db_xref = “CDD: pfam02170” |
| Region | 365 . . . 373 |
| | /region_name = “[PSORT] 2nd |
| | peroximal domain: RIQLKYPHL” |
| Region | 566 . . . 867 |
| | /region_name = “[Pfam/InterPro |
| | description] stem cell self-renewal |
| | protein Piwi” |
| | /db_xref = “CDD: pfam02171” |
| CDS | 1 . . . 910 |
| | /gene = “alg-2” |
| | /locus_tag = “2B167” |
| | /coded_by = “NM_182192.1: |
| | 1 . . . 2733” |
| | /db_xref = “AceView/WormGenes: |
| | alg-2” |
| | /db_xref = “GeneID: 173468” |
| | /db_xref = “LocusID: 173468” |
| | /db_xref = “WormBase: T07D3.7a” |

| | |
|---|---|
| ORIGIN | |
| 1 | mfplpvhngp rlgklsifem pgdsltsssf |
| | mpdggaetss ssqlggsahg aigtkpdagv |
| 61 | qfqcpvrpnh gvegrsillr anhfavripg |
| | gsvqhyqidv fpdkcprrvn revigcliss |
| 121 | fskyftnirp vydgkrnmyt replpigtep |
| | mnfevtlpgd saverkfsvt mkwigqvcls |
| 181 | alddamegrv rqvpheavqs idvilrhlps |
| | lkytpvgrsf ftppgvmkpg mqmhqesklg |
| 241 | ggrevwfgfh qsvrpsqwkm mlnidvsata |
| | fyrampvief vaevlelpvq alaerralsd |
| 301 | aqrvkftkei rglkieithc qavrrkyrvc |
| | nvtrrpaqtq tfplqletgq tiectvakyf |
| 361 | fdkyriqlky phlpclqvgq eqkhtylppe |
| | vcdivpqqrc lkkltdvqts tmikatarsa |
| 421 | perereickl vskaelsadp fahefqitin |
| | pamtevkqrv lsapkllygg rhrattalpn |
| 481 | qgvwdmrgkq fhtgmevrtw aiacfaqqsh |
| | vkendlrmft tqlqristda gmpiigtpmf |
| 541 | ckyasgveqv epmfkylkqt ysaiqlivvv |
| | lpgktpiyae vkrvqdtvlg iatqcvqakn |
| 601 | airttpqtls nlclkmnvkl qqvnsillpn |
| | vrprifnepv iflgcdithp aagdtrkpsi |
| 661 | aavvgsmdah psryaatvrv qqhrqeiitd |
| | ltymvrellv qfyrntrfkp arivvyrdgv |
| 721 | segqlfnvlq yelraireac vmlesgyqpg |
| | itfiavqkrh htrlfaadka dqvgkafnip |
| 781 | pqttvdvgit hptefdfflc shagiqgtsr |
| | pshyhvlwdd ndltadelqq ltyqmchtyv |
| 841 | rctrsvsipa payyahlvaf raryhlvdrd |
| | hgsgeegsqp sgtssedttl ssmakavqvh |
| 901 | pdsnnvmyfa |
| // | |

IIID. DRH-1

| | |
|---|---|
| LOCUS | NP_501018 1037 aa linear INV 21- |
| NOVEMBER | |
| 2003 | |
| DEFINITION | Dicer-Related Helicase, a DExH-box |
| | helicase (119.2 kD) (drh-1) |
| | [Caenorhabditis elegans]. |
| ACCESSION | NP_501018 |
| VERSION | NP_501018.1 GI: 17539846 |
| DBSOURCE | REFSEQ: accession NM 068617.2 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 1037) |

| | |
|---|---|
| AUTHORS | Tabara,H., Yigit,E., Siomi,H. and |
| | Mello,C. C. |
| TITLE | The dsRNA binding protein RDE-4 inter- |
| | acts with RDE-1, DCR-1, and a DExH-box |
| | helicase to direct RNAi in C. elegans |
| JOURNAL | Cell 109 (7), 861-871 (2002) |
| MEDLINE | 22105477 |
| PUBMED | 12110183 |

| | |
|---|---|
| REFERENCE | 2 (residues 1 to 1037) |

| | |
|---|---|
| AUTHORS | Marcotte,E. M., Xenarios,I., van Der |
| | Bliek,A. M. and Eisenberg,D. |
| TITLE | Localizing proteins in the cell from |
| | their phylogenetic profiles |
| JOURNAL | Proc. Natl. Acad. Sci. U.S.A. 97 (22), |
| | 12115-12120 (2000) |
| MEDLINE | 20504472 |
| PUBMED | 11035803 |

| | |
|---|---|
| COMMENT | REVIEWED REFSEQ: This record has been |
| | curated by NCBI staff. This record is |
| | derived from an annotated genomic se- |
| | quence (NC_003282). The reference se- |
| | quence was derived from AU205212, |
| | AF480439.1 and AU217173. |
| | Summary: This gene drh-1, also known as |
| | F15B10.2, 4H372 or YK7673, maps at (IV; |
| | +3.32). It encodes a Dicer-Related |
| | Helicase, a DExH-box helicase. From |
| | Pfam homology, the product would have |
| | ATP binding, nucleic acid binding, ATP |
| | dependent helicase, helicase |
| | activities. |
| | According to the Worm Transcriptome |
| | Project, it is well expressed at all |
| | stages of development [Kohara cDNAs]. |
| | Its sequence is defined by 19 cDNA |
| | clones. |
| | RNA interference results: |
| | [A. Sugimoto 2000] No obvious phenotype |
| | (by injecting cDNA clone SA: yk317d8). |
| | [J. Ahringer 2003] No obvious phenotype |
| | (by feeding genomic PCR product JA: |
| | F15B10.2). |
| | Expression |
| | The expression profile for the gene, |
| | derived from the proportion of animals |
| | at each stage in each Kohara library |
| | is: embryos 6%, L1 or L2 larvae 19%, L3 |
| | to adult 74%. |
| | In situ hybridisation pictures to all |
| | stages of development are available |
| | from Kohara NextDB. |
| | Pattern [pm11035803] predicted |
| | mitochondrial. |
| | This complete mRNA is 3298 bp long. Its |
| | sequence exactly matches the genome. |
| | The premessenger has 20 exons. It |
| | covers 5.98 kb on the WS97 genome. It |
| | is transpliced to SL1 or SL2. The |
| | protein (1037 aa, 119.2 kDa, pI 6.3) |
| | contains one DEAD/DEAH box helicase |
| | motif, one helicase, C-terminal motif. |
| | Taxblast (threshold 10\hat{ }-3) tracks an- |
| | cestors down to archaea and bacteria |
| | and eukaryota. |
| | COMPLETENESS: full length. |
| | Method: conceptual translation. |

| | |
|---|---|
| FEATURES | Location/Qualifiers |

| | |
|---|---|
| source | 1 . . . 1037 |
| | /organism = “Caenorhabditis |
| | elegans” |
| | /db_xref = “taxon: 6239” |
| | /chromosome= “IV” |
| | /map = “IV; +3.32 cM (interpolated |
| | genetic position)” |
| | /map = “IV; covering 5976 bp, from |
| | base 6613343 to 6607368 on genome |
| | release WS97” |
| | /clone = “Primers to amplify the |
| | CDS (9336 bp, Stop included): |
| | ATGAGGAAAAAGCAGTGTTCTTCAATA (T = |
| | 57.4), TTATGCTTCTCTGATTAAATTGACTAC |
| | (T = 55.9). Complete CDS clones: |
| | AF480439, yk850g8, yk1388a5, |
| | yk1414c1, yk1627h8. Recommended |
| | clone (from the Kohara collection): |
| | yk850g8. Other clone(s): yk1716a1, |
| | yk447b12, yk296e5, yk6g7, yk317d10, |
| | yk354h4, yk240c5, yk134d4, |
| | yk606h12, yk317d8, yk225b1, |
| | yk207d7, yk219b1, yk1752c2. for |
| | edited clone sequences see |
| | www.wormgenes.org” |
| | /clone_lib = “Kohara embryonic |
| | lambda gt11 library: yk447b12, |
| | yk606h12; Kohara Sugano L2 larvae |
| | cap-selected library: yk1716a1, |
| | yk1388a5, yk1414c1, yk1627h8, |
| | yk1752c2; Kohara Sugano L4 larvae |
| | cap-selected library: yk850g8; |
| | Kohara mixed stage library, from |
| | him-8 strain, containing 15-30% |
| | males: yk207d7; Kohara mixed stage |
| | library, from him-8 strain, con- |
| | taining 15-30% males: yk296e5, |
| | yk6g7, yk317d10, yk354h4, yk240c5, |
| | yk134d4, yk317d8, yk225b1, yk219b1; |
| | gb: AP480439” |
| Protein | 1 . . . 1037 |
| | /product = “Dicer-Related Helicase, |
| | a DExH-box helicase (119.2 kD) |
| | (drh-1)” |
| Region | 283 . . . 510 |
| | /region_name = “[Pfam/InterPro |
| | description] DEAD/DEAN box |
| | helicase” |
| | /db_xref = “CDD: pfam00270” |
| Region | 723 . . . 810 |
| | /region_name = “[Pfam/InterPro |
| | description] helicase, C-terminal” |
| | /db_xref = “CDD: pfam00271” |
| CDS | 1 . . . 1037 |
| | /gene = “drh-1” |
| | /locus_tag = “4H372” |
| | /coded_by = “NM_068617.2: |
| | 6 . . . 3119” |
| | /db_xref = “AceView/WormGenes: |
| | drh-1” |
| | /db_xref = “GeneID: 177425” |
| | /db_xref = “LocusID: 177425” |
| | /db_xref = “WormBase: F15B10.2” |

| | |
|---|---|
| ORIGIN | |
| 1 | mrkkqcssil slydkeiilc lepiyrdpek |
| | gdgfsellpl gridelkiqs enaqefskql |
| 61 | yhdlknsils nadderlykd imtylqtylp |
| | kctvhkllnc snrevklsdf hyildhfegf |
| 121 | lrfiepkvvl ayldsypqyi davavlrkei |
| | erneednqds dfikklilrt vpllgeqavy |
| 181 | dimytiseks snnldveakq fiakvlrlkn |
| | dgflrfyqii nasrrqlngr iyicpvhesa |
| 241 | temmvylgta alntnryrmi nirvdnivqe |
| | nstprlvies vrqrihrqrq lclrnyqeel |
| 301 | cqvalqgknt ivtaptgsgk tviaaniike |
| | hfesrssegk rfkalfmtpn smilnqqaas |
| 361 | issyldhvyh tqiiqgsdnv ptrnviqskd |
| | livatpqmiv nlcnehrnsl ddesrldqff |
| 421 | lstftiiffd echntvknsp ysnimreyhy |
| | lknmgnmpeg hslpqiiglt aslgtgdknd |
| 481 | clqvrnyiag lcasmdvkdl sivkdnleel |
| | rgyspivpdk vllcerstdg pigmftnrlt |
| 541 | lmmqevegli rtalrnehig ieqrrqiett |
| | erdfrpdssf ldppadkeha gyqnwvcnqm |
| 601 | nlvsgtsfre tgtrtiinea ldvlkecfct |
| | lsyninfhpe valnylkdem eyrtpnftvn |
| 661 | miriweryhn qlvgtgsaen pmisktvqyi |
| | veqnlqrads rtiifvrtry eatilnkvln |
| 721 | sneellmlgi ksewmsglnk stassadisa |
| | skqkqmeklk mfadgeiril vstsvaeegl |
| 781 | dvpecslvik ynyatneiah vqrrgrgral |
| | nsecvlitns ialrdqesnn rdkeslmset |
| 841 | isliqnspae frkcvdeesn kiwprilred |
| | tdkaqkieeq inrnivykii ckkceailct |
| 901 | skdirsrntq ylvcdpgfws lvrktrltde |
| | qqalikynat gsincrrenc glklgqliev |
| 961 | ntvdlpclsa lsivllvegt dkriivkkwk |
| | nildkyftpt eirqldvqtm rdadqartpm |
| 1021 | vfehhangev vnlirea |
| // | |

IIIE. DRH-2

| | |
|---|---|
| LOCUS | NP_501019 620 aa linear INV 21- |
| NOVEMBER | |
| 2003 | |
| DEFINITION | Dicer-Related Helicase (71.3 kD) |
| | (drh-2) [Caenorhabditis elegans]. |
| ACCESSION | NP_501019 |
| VERSION | NP_501019.2 GI: 25145329 |
| DBSOURCE | REFSEQ: accession NM 068618.2 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE 1 | (residues 1 to 620) |

| | |
|---|---|
| AUTHORS | Tabara,H., Yigit,E., Siomi,H. and |
| | Mello,C. C. |
| TITLE | The dsRNA binding protein RDE-4 |
| | interacts with RDE-1, DCR-1, and a |
| | DExH-box helicase to direct RNAi in C. |
| | elegans |
| JOURNAL | Cell 109 (7), 861-871 (2002) |
| MEDLINE | 22105477 |
| PUBMED | 12110183 |

| | |
|---|---|
| COMMENT | REVIEWED REFSEQ: This record has been |
| | curated by NCBI staff. This record is |
| | derived from an annotated genomic se- |
| | quence (NC_003282). The reference se- |
| | quence was derived from AF480440.1 and |
| | D33924.1. On Nov. 21, 2002 this sequence |
| | version replaced gi: 17538344. |
| | Summary: This gene drh-2, also known as |
| | C01B10.1, 4H380 or YK1203, maps at (IV; |
| | +3.33). It encodes a Dicer-Related |
| | Helicase. From Pfam homology, the pro- |
| | duct would have ATP binding, nucleic |
| | acid binding, helicase activities. |
| | According to the Worm Transcriptome |
| | Project, it is well expressed mostly |
| | from L1 larvae to adult [Kohara cDNAs]. |
| | Its sequence is defined by 10 cDNA |
| | clones. |
| | RNA interference results: |
| | [A. Sugimoto 2000] No obvious phenotype |
| | (by injecting cDNA clone SA: yk272f7). |
| | [J. Ahringer 2003] No obvious phenotype |
| | (by feeding genomic PCR product JA: |
| | C01B10.1). |
| | Expression |
| | The expression profile for the gene, |
| | derived from the proportion of animals |
| | at each stage in each Kohara library |
| | is: embryos 2%, L1 or L2 larvae 27%, L3 |
| | to adult 70%. |
| | In situ hybridisation pictures to all |
| | stages of development are available |
| | from Kohara NextDB. |
| | This complete CDS mRNA is 3277 bp long. |
| | Its sequence exactly matches the |
| | genome. The premessenger has 19 exons. |
| | It covers 4.76 kb on the WS97 genome. |
| | It has a very long 5′ UTR. The protein |
| | (620 aa, 71.3 kDa, pI 6.2) contains one |
| | helicase, C-terminal motif. Taxblast |
| | (threshold 10\hat{ }-3) tracks ancestors down |
| | to archaea and viruses and bacteria and |
| | eukaryota. |
| | COMPLETENESS: full length. |
| | Method: conceptual translation. |

| | |
|---|---|
| FEATURES | Location/Qualifiers |

| | |
|---|---|
| source | 1 . . . 620 |
| | /organism = “Caenorhabditis |
| | elegans” |
| | /db_xref = “taxon: 6239” |
| | /chromosome = “IV” |
| | /map = “IV; +3.33 cM (interpolated |
| | genetic position)” |
| | /map = “IV; covering 4758 bp, from |
| | base 6618488 to 6613731 on genome |
| | release WS97” |
| | /clone = “Primers to amplify the |
| | CDS (5583 bp, Stop included): |
| | ATGATTGTAAATCTTTGCAATGAGCAC (T = |
| | 57.4), |
| | TTATGCTTGTCTAATTACATTGATTACTT (T = |
| | 55.0). Complete CDS clones: |
| | AF480440, yk38c3, yk226c6, |
| | yk1564a4, yk1605b6. Recommended |
| | clone (from the Kohara collection): |
| | yk226c6. Other clone(s): yk315f1, |
| | yk1017f9, yk1007f1, yk1080b12, |
| | yk272f7. Anomalous clones: |
| | yk1080b12 (Suspected internal dele- |
| | tion) for edited clone sequences |
| | see www.wormgenes.org” |
| | /clone_lib = “Kohara Sugano L1 |
| | larvae cap-selected library: |
| | yk1017f9, yk1007f1, yk1080b12; |
| | Kohara Sugano L2 larvae cap- |
| | selected library: yk1605b6; Kohara |
| | Sugano L4 larvae cap-selected |
| | library: yk1564a4; Kohara mixed |
| | stage library, from him-8 strain, |
| | containing 15-30% males: yk226c6, |
| | yk38c3, yk315f1, yk272f7; gb: |
| | AF480440” |
| Protein | 1 . . . 620 |
| | /product = “Dicer-Related Helicase |
| | (71.3 kD) (drh-2)” |
| Region | 305 . . . 392 |
| | /region_name = “[Pfam/InterPro de- |
| | scription] helicase, C-terminal” |
| | /db_xref = “CDD: pfam00271” |
| CDS | 1 . . . 620 |
| | /gene = “drh-2” |
| | /locus_tag = “4H380” |
| | /coded_by = “NM_068618.2: |
| | 1238 . . . 3100” |
| | /db_xref = “AceView/WormGenes: |
| | drh-2” |
| | /db_xref = “GeneID: 177426” |
| | /db_xref = “LocusID: 177426” |
| | /db_xref = “WormBase: C01B10.1” |

| | |
|---|---|
| ORIGIN | |
| 1 | mivnlcnehr dplddeyppe qfflstftii |
| | ffdechntvk nspysnvmre yhylknmqnm |
| 61 | peghsfpqii gltaslgtgd kkncmqvrsy |
| | iaglcanmdv kelsivkdnl eelldhnpfv |
| 121 | tdqvsfcers ndgpiemftk rlkqmmqeve |
| | dlirttlkne ptvkyeippt dkehnryenw |
| 181 | isnqrncvsl agsrnktlii evldvlkdcf |
| | yalsyninfn pevalkkyle kelgperirn |
| 241 | ftdnmnliwd nchrelvgig saenpmiart |
| | vqfildqneq tsdfraiifv rtkkeadfln |
| 301 | yvlndrlhel giksdwmsgq kkstassadi |
| | saskqkqmek lkmfadgenq ilvstsvaee |
| 361 | qldipecslv ikynyatnet ahvqrrgrar |
| | arnskcvlit nsialhvqes nnlakenlmt |
| 421 | etisliqnsp gefrqcvdee snkvwpriqr |
| | edtdkaqrik eqinrnivyk ivcmkcdtvl |
| 481 | ctnkdirskn tqyivcnpgf wslvrriplp |
| | leqrasnkfn stgsieclge rcgsklgqli |
| 541 | dvntvnlpcl kvksilllie stnerilvkq |
| | wknildehft pttlkqrdvq tmkdadygra |
| 601 | piefehhtan gevinvirga |
| // | |

IIIF. Helicase Homologous to DCR-2 (DRH-3)

DCR-2 has been officially renamed DRH-3 and is a paralog of DRH-1 and DRH-2 which are essential for RNAi. Importantly, the human ortholog for DRH-3 is melanoma differentiation associated protein-5.

MQPTAIRLEDYDKSKLRLPFESPYFPAYFRLLKWKFLDVCVESTRNNDIG
YFKLFESLFPPGKLEEIARMIIDEPTPVSHDPDMIKIRNADLDVKIRKQA
ETYVTLRHAHQQKVQRRRFSECFLNTVLFDEKGLRIADEVMFNYDKELYG
YSHWEDLPDGWLTAETFKNKFYDEEEVTNNPEGYQKLDRVAGAARGMIIM
KHLKSNPRCVSETTILAFEVFNKGNHQLSTDLVEDLLTEGPAFELKIENG
EEKKYAVKKWSLHKTLTMFLAIIGFKSNDKKEKNEHEEWYYGFIDAMKND
PANRAALYFLDKNWPEELEEREKERDRIRLTLLKSQRTNEEAVGEDVCTT
IRPQPKDSGYNPDAVVTELVLRTYQEELVQPALEGKNCVIVAPTGSGKTE
VAIYAALKHIEERTSQGKPSRVVLLVPKIPLVGQQKDRFLKYCNGMYEVN
GFHGSESSVSGTGRRDEVIATHVSVMTPQILINMLQSVRQNERLYVSDFS
MMIFDEVHKAAKNHPYVLINQMVQEWKYEKPQIIGLTASLSVKVDGQKDE
NQMLNDIYNMLALINAPHLSTITRQSSIDELNEHVGKPDDSVELCLPAKE
NILRDYIERYLNHAHGKFLEELASMSKSTGRNNTIPPNMINTFKKNQPKN
YEYYDSLLQGIIQELNKLNVPEKWNSQTWAKYMKVYLEARGIVDLMPAMV
AFKYMEKAIGKLNESHSETVEYSTFIKDHDTLKQTIQSVEPEIVLRLKKY
THQSVPHQFGNYGEQMVGYVLGTNKQGAVQQTSQEQQLTLDKFNNGRLKV
IVATSVVEEGLDVTACNLIIKYNCSSGSAIQLVQQRGRARAKNSRSVLLS
VKSSINETETNALISEKYMRLCVKKITENGEKQLAAEVKRVAELNAAERK
RNLEEQLNLRLRHENKIYKLMCSNCSKEFCKSIYIKKVFSNYMVFDPSVW
RFLHVESVETFIKCLKITWKCRIADYQIAEFPNFAFRQLTFRLFLCNFQM
FQKRKVSKYLSEDNQPLSDIKCFHCKLDVGRAYKIRGTYLPQLSVKALTF
VQESDYSSMTKAKWSDVEQDLFYISEAIEDDFRIMLNALSDTEENIEKKI
VLDLDSRQHNKQLEMKRFHIQQEPPTKGVAPEAQ

Also see the C. elegans Protein Database: Wormpep at http://www.sanger.ac.uk/Projects/C_{—}elegans/wormpep/; Accesion No. ce09069.

IIIG. Double Helicase

MADELARIQQYEYRQNSNLVLSVDYNLTDRRGREEPTGEVLPITDKEMRK
MKMGDRAIKGKAPVQDQKKKRKKKDDEKAQQFGRNVLVDNNELMGAYKPR
TQETKQTYEVILSFILDALGDVPREVLCGAADEVLLTLKNDKFRDKEKKK
EVEALLGPLTDDRIAVLINLSKKISDFSIEEENKPEGDGDIYENEGVNVQ
FDSDEEEDDGGMVNEIKGDSEEESEEEEGVDTDYTATLKGDGHLTEDEQK
ARGILHPRDIDAHWIQRSLAKYFKDPLIAQQKQTEVIGILKNAADDRDAE
NQLVLLLGFDQFEFIKCLRQNRLMILYCTLLRQANEKERLQIEDDMRSRP
ELHPILALLQETDEGSVVQVEKSKRDAEKSKKAATAANEAISAGQWQAGR
KMLDLNDLTFSQGSHLMSNKRCELPDGSYRRQKKSYEEIHVPALKPRPFA
EGEKLVSVSELPKWAQPAFDGYKSLNRIQSRLCDSALRSKEHLLLCAPTG
AGKTNVALLTMLQEIGNHLAEDGSVKLDEFKIVYIAPMKSLVQEMVGSFS
KRLAPFGITVGEMTGDAQMSKEQFMATQVIVCTPEKYDVVTRKGGERAYN
QMVRLLIIDEIHLLHDDRGPVLESIVVRTIRQMEQNHDECRLVGLSATLP
NYQDVATFLRVKPEHLHFPDNSYRPVPLEQQYIGVTEKKALKRFQAMNEV
VYDKIMEHAGKSQVLVFVHSRKETAKTAKAIRDACLEKDTLSAFMREGSA
STEILRTEAEQAKNLDLKDLLPYGFAIHHAGMNRVDRTLVEDLFADRHIQ
VLFSTATLAWGVNLPAHTVIIKGTQIYNPEKGRWTELGALDIMQMLGRAG
RPQYDDRGEGILITNHSELQYYLSLMNQQLPVESQMVSRLTDMLNAEVVL
GTVSSVSEATNWLGYTFLFVRMLKNPTLYGITHEQARADPLLEQRRADLI
HTACVLLDKAGLIKYDKRSGIIQATELGRIASHFYCTYESMQTYNKLLVE
TCSDIDLFRIFSMSSEFKLLSVRDEEKLELQKMAEHAPIPIKENLDEASA
KTNVLLQAYISQLKLEGFALQADMVFVAQSAGRLFRALFEIVLWRGWAGL
AQKVLTLCKMVTQRQWGSLNPLHQFKKIPSEVVRSIDKKNYSFDRLYDLD
QHQLGDLIKMPKMGKPLFKFIRQFPKLEMTTLIQPITRTTMRIELTITPD
FKWDEKVHGSAEGFWIFIEDTDGEKILHHEFFLLKQKFCSDEHVVKMIVP
MFDPMPPLYYVRIVSDRWIGAETVLPISFRHLILPEKYPPPTELLDLQPL
PISAVTNKEFQTVFAESGFKVFNPIQTQVFRTVFESNENVIVCAPNGSGK
TAIAELAVLRHFENTPEAKAVYITPMEDMATKVYADWKRRLEPAIGHTIV
LLTGEQTMDLKLAQRGQLIISTPERWDNISRRWKQRKSVQNVKLFIADDL
HMIGASNGAVFEVVCSRTRYISSQLESAVRVVALSSSLTNARDLGMWLGC
SASATFNFMPSTRPVPLDLEIKSFNLSHNASRFAAMERPVYQAICRHAGK
LEPKPALVFVPVRRQTRPVAVALLTMALADGAPKRFLRLAEHDDTFQALL
ADIEDESLRESVSCGVGFLHEGTAPKDVHIVQQLFESNAIQVCVVPRGMC
YQIEMSAYLVVVMDTQFYNGKYHVYEDYPIADMLHMVGLANRPILDSDAK
CVVMCQTSKRAYYKKFLCDPLPVESHLDHCLHDHFNAEIVTKTIENKQDA
IDYLTWTLLYRRMTQNPNYYNLQGTTHRHLSDALSELVELTLKDLENSKC
IAVKDEMDTVSLNLGMIASYYYISYQTIELFSMSLKEKTKTRALIEIISA
SSEFGNVPMRHKEDVILRQLAERLPGQLKNQKFTDPHVKVNLLIHAHLSR
VKLTAELNKDTELIVLRACRLVQACVDVLSSNGWLSPAIHAMELSQMLTQ
AMYSNEPYLKQLPHCSAALLERAKAKEVTSVFELLELENDDRSDILQMEG
AELADVARFCNHYPSIEVATELENDVVTSNDNLMLAVSLERDNDIDGLAP
PVVAPLFPQKRKEEGWWLVIGDSESNALLTIKRLVINEKSSVQLDFAAPR
PGHHKFKLFFISDSYLGADQEFDVAFKVEEPGRSNRKRKHEKEED

Also see the C. elegans Protein Database: Wormpep at http://www.sanger.ac.uk/Projects/C_{—}elegans/wormpep/; Accesion No. ce21971.

IIIH. EFT-2, EF-Tu Family GTP Binding Protein

| | |
|---|---|
| LOCUS | NP_492457 852 aa linear INV 21- |
| NOVEMBER | |
| 2003 | |
| DEFINITION | translation Elongation FacTor (94.8 kD) |
| | (eft-2) [Caenorhabditis elegans]. |
| ACCESSION | NP_492457 |
| VERSION | NP_492457.1 GI: 17506493 |
| DBSOURCE | REFSEQ: accession NM 060056.2 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 852) |

| | |
|---|---|
| AUTHORS | Fraser,A. G., Kamath,R. S., |
| | Zipperlen,P., Martinez-Campos,M., |
| | Sohrmann,M. and Ahringer,J. |
| TITLE | Functional genomic analysis of C. |
| | elegans chromosome I by systematic RNA |
| | interference |
| JOURNAL | Nature 408 (6810), 325-330 (2000) |
| MEDLINE | 20548709 |
| PUBMED | 11099033 |

| | |
|---|---|
| REFERENCE | 2 (residues 1 to 852) |

| | |
|---|---|
| AUTHORS | Ofulue,E. N. and Candido,E. P. |
| TITLE | Isolation and characterization of |
| | eft-1, an elongation factor 2-like gene |
| | on chromosome III of Caenorhabditis |
| | elegans |
| JOURNAL | DNA Cell Biol. 11 (1), 71-82 (1992) |
| MEDLINE | 92153310 |
| PUBMED | 1739435 |

| | |
|---|---|
| REFERENCE | 3 (residues 1 to 852) |

| | |
|---|---|
| AUTHORS | Ofulue,E. N. and Candido,E. P. |
| TITLE | Molecular cloning and characterization |
| | of the Caenorhabditis elegans elonga- |
| | tion factor 2 gene (eft-2) |
| JOURNAL | DNA Cell Biol. 10 (8), 603-611 (1991) |
| MEDLINE | 92029622 |
| PUBMED | 1930695 |

| | |
|---|---|
| COMMENT | REVIEWED REFSEQ: This record has been |
| | curated by NCBI staff. This record is |
| | derived from an annotated genomic se- |
| | quence (NC_003279). The reference se- |
| | quence was derived from BJ105642.1, |
| | AU205829, M86959 and AU218565. |
| | Summary: This essential gene eft-2, |
| | also known as F25H5.4, 1J741 or YK6, |
| | maps at (I; +3.37). Its phenotype is |
| | embryonic lethal, protruding vulva. It |
| | encodes a translation Elongation |
| | FacTor. From Pfam homology, the product |
| | would have GTP binding, translation |
| | elongation factor activities, would be |
| | involved in translational elongation. |
| | According to the Worm Transcriptome |
| | Project, it is expressed at very high |
| | level at all stages of development |
| | except in embryos [Kohara cDNAs]. Its |
| | sequence is defined by 1015 cDNA |
| | clones. |
| | RNA interference results |
| | [J. Ahringer 2000] embryonic lethal |
| | (100%), protruding vulva (by feeding |
| | genomic PCR product JA: F25H5.4). |
| | Function |
| | Protein properties: [C.elegansII] NMK. |
| | Encodes protein with >80% similarity to |
| | elongation factor EF-2 from yeast, |
| | Drosophila, human. [Ofolue and Candido |
| | 1992]. |
| | Expression |
| | The expression profile for the gene, |
| | derived from the proportion of animals |
| | at each stage in each Kohara library |
| | is: embryos 3%, L1 or L2 larvae 13%, |
| | L3 to adult 34%. |
| | In situ hybridisation pictures to all |
| | stages of development are available |
| | from Kohara NextDB. |
| | For a detailed expression pattern de- |
| | scription, see Wormbase Expr1390. |
| | This complete mRNA is 2819 bp long. Its |
| | sequence exactly matches the genome. |
| | The premessenger has 6 exons. It covers |
| | 3.23 kb on the WS97 genome. It is |
| | transpliced to SL1. The protein (852 |
| | aa, 94.8 kDa, pI 6.1) contains one |
| | Elongation factor, GTP-binding motif, |
| | one Elongation factor Tu, domain 2 |
| | motif, one Elongation factor G, domain |
| | IV motif, one Elongation factor G, C- |
| | terminal motif. It also contains a coil |
| | coil stretch, an ER membrane domain |
| | [Psort2]. Taxblast (threshold 10\hat{ }-3) |
| | tracks ancestors down to archaea and |
| | bacteria and eukaryota. |
| | COMPLETENESS: full length. |
| | Method: conceptual translation. |

| | |
|---|---|
| FEATURES | Location/Qualifiers |

| | |
|---|---|
| source | 1 . . . 852 |
| | /organism = “Caenorhabditis |
| | elegans” |
| | /db_xref = “taxon: 6239” |
| | /chromosome = “I” |
| | /map = “I; +3.37 cM (interpolated |
| | genetic position)” |
| | /map = “I; covering 3303 bp, from |
| | base 9171586 to 9174890 on genome |
| | release WS97” |
| Protein | 1 . . . 852 |
| | /product = “translation Elongation |
| | FacTor (94.8 kD) (eft-2)” |
| Region | 17 . . . 356 |
| | /region_name = “[Pfam/InterPro de- |
| | scription] elongation factor, GTP- |
| | binding” |
| | /db_xref = “CDD: pfam00009” |
| Region | 176 . . . 177 |
| | /region_name = “[PSORT] dileucine |
| | domain: LL” |
| Region | 298 . . . 325 |
| | /region_name = “[PSORT] coil coil |
| | domain: |
| | VMNIKKDKTAALVEKLGIKLANDEKDLE” |
| Region | 401 . . . 480 |
| | /region_name = “[Pfam/InterPro |
| | description] elongation factor Tu, |
| | domain 2” |
| | /db_xrefr = “CDD: pfam03144” |
| Region | 614 . . . 731 |
| | /region_name = “[Pfam/InterPro de- |
| | scription] elongation factor G, |
| | domain IV” |
| | /db_xref = “CDD: pfam03764” |
| Region | 655 . . . 656 |
| | /region_name = “[PSORT] dileucine |
| | domain: LL” |
| Region | 733 . . . 821 |
| | /region_name = “[Pfam/InterPro de- |
| | scription] elongation factor G, |
| | C-terminal” |
| | /db_xref = “CDD: pfam00679” |
| Region | 734 . . . 735 |
| | /region_name = “[PSORT] dileucine |
| | domain: LL” |
| Region | 833 . . . 836 |
| | /region_name = “[PSORT] nuclear |
| | localization domain: RKRK” |
| Region | 848 . . . 851 |
| | /region_name = “[PSORT] ER membrane |
| | domain: YLDK” |
| CDS | 1 . . . 852 |
| | /gene = “eft-2” |
| | /locus_tag = “1J741” |
| | /coded_by = “NM_060056.2: |
| | 124 . . . 2682” |
| | /db_xref = “AceView/WormGenes: |
| | eft-2” |
| | /db_xref = “GeneID: 172743” |
| | /db_xref = “LocusID: 172743” |
| | /db_xref = “WormBase: F25H5.4” |

| | |
|---|---|
| ORIGIN | |
| 1 | mvnftvdeir almdrkrnir nmsviahvdh |
| | gkstltdslv skagiiaqsk agetrftdtr |
| 61 | kdeqerciti kstaislffe lekkdlefvk |
| | genqfetvev dgkkekyngf linlidspgh |
| 121 | vdfssevtaa lrvtdgalvv vdcvsgvcvq |
| | tetvlrqaia erikpvlfmn kmdrallelq |
| 181 | lgaeelfqtf qriveninvi iatygdddgp |
| | mqpimvdpsi gnvgfgsqlh gwaftlkqfa |
| 241 | emyagkfgvq vdklmknlwg drffdlktkk |
| | wsstqtdesk rgfcqfvldp ifmvfdavmn |
| 301 | ikkdktaalv eklgikland ekdlegkplm |
| | kvfmrkwlpa gdtmlqmiaf hlpspvtaqk |
| 361 | yrmemlyegp hddeaavaik tcdpngplmm |
| | yiskmvptsd kgrfyafgrv fsgkvatgmk |
| 421 | ariqgpnyvp gkkedlyekt iqrtilmmgr |
| | fiepiedips gniaglvgvd qylvkggtit |
| 481 | tykdahnmrv mkfsvspvvr vaveaknpad |
| | lpklveglkr laksdpmvqc ifeesgehii |
| 541 | agagelhlei clkdleedha ciplkksdpv |
| | vsyretvqse snqiclsksp nkhnrlhcta |
| 601 | qpmpdgladd ieggtvnard efkarakila |
| | ekyeydvtea rkiwcfgpdg tgpnllmdvt |
| 661 | kgvqylneik dsvvagfqwa tregvlsden |
| | mrgvrfnvhd vtlhadaihr gggqiiptar |
| 721 | rvfyasvlta eprllepvyl veiqcpeaav |
| | ggiygvlnrr rghvfeesqv tgtpmfvvka |
| 781 | ylpvnesfgf tadlrsntgg qafpqcvfdh |
| | wqvlpgdple agtkpnqivl dtrkrkglke |
| 841 | gvpaldnyld km |
| // | |

III. EFT-4 (eIF1 alpha)

| | |
|---|---|
| LOCUS | NP_509323 463 aa linear INV 21- |
| NOVEMBER | |
| 2003 | |
| DEFINITION | translation Elongation FacTor (50.7 kD) |
| | (eft-4) [Caenorhabditis elegans]. |
| ACCESSION | NP_509323 |
| VERSION | NP_509323.1 GI: 17569207 |
| DBSOURCE | REFSEQ: accession NM 076922.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 463) |

| | |
|---|---|
| AUTHORS | Kamath,R. S., Fraser,A. G., Dong,Y., |
| | Poulin,G., Durbin,R., Gotta,M., |
| | Kanapin,A., Le Bot,N., Moreno,S., |
| | Sohrmann,M., Welchman,D. P., |
| | Zipperlen,P. and Ahringer,J. |
| TITLE | Systematic functional analysis of the |
| | Caenorhabditis elegans genome using |
| | RNAi |
| JOURNAL | Nature 421 (6920), 231-237 (2003) |
| MEDLINE | 22417569 |
| PUBMED | 12529635 |

| | |
|---|---|
| COMMENT | VALIDATED REFSEQ: This record has un- |
| | dergone preliminary review of the se- |
| | quence, but has not yet been subject to |
| | final review. This record is derived |
| | from an annotated genomic sequence |
| | (NC_003284). The reference sequence was |
| | derived from WormBase CDS: R03G5.1a. |
| | Summary: This essential gene eft-4, |
| | also known as eln-2, R03G5.1, XI443 or |
| | YK211, maps at (X; −0.81). Its pheno- |
| | type is embryonic lethal, partial, slow |
| | growth. It encodes a translation Elon- |
| | gation FacTor. From Pfam homology, the |
| | products would have GTP binding, trans- |
| | lation elongation factor activities, |
| | would be involved in translational |
| | elongation. |
| | According to the Worm Transcriptome |
| | Project, it is expressed at very high |
| | level at all stages of development |
| | [Kohara cDNAs]. Its sequence is fully |
| | supported by 406 cDNA clones and pro- |
| | duces, by alternative splicing, 4 dif- |
| | ferent transcripts a, b, c, d alto- |
| | gether encoding 4 different protein |
| | isoforms. |
| | RNA interference results |
| | [J. Ahringer 2003] Embryonic lethal |
| | (40%), slow growth (by feeding genomic |
| | PCR product JA: R03G5.1). |
| | Function |
| | Protein properties: [C.elegansII] NMK. |
| | Encodes EF1 alpha protein, aa sequence |
| | identical to eft-3. [FK]. |
| | Expression |
| | The expression profile for the gene, |
| | derived from the proportion of animals |
| | at each stage in each Kohara library |
| | is: embryos 6%, L1 or L2 larvae 58%, |
| | L3 to adult 37%. |
| | In situ hybridisation pictures to all |
| | stages of development are available |
| | from Kohara NextDB. |
| | The CDS has 3 exons. It covers 1.59 kb |
| | on the WS97 genome. The protein (463 |
| | aa, 50.7 kDa, pI 9.1) contains one |
| | Elongation factor, GTP-binding motif, |
| | one Elongation factor Tu, domain 2 |
| | motif, one Elongation factor Tu, C- |
| | terminal motif. It also contains an ER |
| | membrane domain [Psort2]. Taxblast |
| | (threshold 10\hat{ }-3) tracks ancestors down |
| | to eukaryota. |
| | COMPLETENESS: full length. |
| | Method: conceptual translation. |

| | |
|---|---|
| FEATURES | Location/Qualifiers |

| | |
|---|---|
| source | 1 . . . 463 |
| | /organism = “Caenorhabditis |
| | elegans” |
| | /db_xref = “taxon: 6239” |
| | /chromosome = “X” |
| | /map = “X; −0.81 cM (interpolated |
| | genetic position)” |
| | /map = “X; covering 2129 bp, from |
| | base 7814176 to 7816306 on genome |
| | release WS97” |
| Protein | 1 . . . 463 |
| | /product = “translation Elongation |
| | FacTor (50.7 kD) (eft-4)” |
| Region | 5 . . . 239 |
| | /region_name = “[Pfam/InterPro de- |
| | scription] elongation factor, GTP- |
| | binding” |
| | /db_xref = “CDD: pfam00009” |
| Region | 258 . . . 327 |
| | /region_name = “[Pfam/InterPro de- |
| | scription] elongation factor Tu, |
| | domain 2” |
| | /db_xref = “CDD: pfam03144” |
| Region | 333 . . . 442 |
| | /region_name = “[Pfam/InterPro de- |
| | scription] elongation factor Tu, C- |
| | terminal” |
| | /db_xref = “CDD: pfam03143” |
| Region | 459 . . . 462 |
| | /region name = “[PSORT] ER membrane |
| | domain: APKK” |
| Region | 460 . . . 463 |
| | /region_name = “[PSORT] nuclear lo- |
| | calization domain: PKKK” |
| CDS | 1 . . . 463 |
| | /gene = “eft-4” |
| | /locus_tag = “XI443” |
| | /coded_by = “NM_076922.1: |
| | 1 . . . 1392” |
| | /db_xref = “AceView/WormGenes: |
| | eft-4” |
| | /db_xref = “GeneID: 181044” |
| | /db_xref = “LocusID: 181044” |

| | |
|---|---|
| ORIGIN | |
| 1 | mgkekvhini vvighvdsgk stttghliyk |
| | cggidkrtie kfekeaqemg kgsfkyawvl |
| 61 | dklkaererg itidialwkf etakyyitii |
| | dapghrdfik nmitgtsQad cavlvvacgt |
| 121 | gefeagiskn gqtrehalla qtlgvkqliv |
| | acnkmdstep pfsearftei tnevsgfikk |
| 181 | igynpkavpf vpisgfngdn mlevssnmpw |
| | fkgwaverke gnasgktlle aldsiippqr |
| 241 | ptdrplrlpl qdvykiggig tvpvgrvetg |
| | iikpgmvvtf apqnvttevk svemhheslp |
| 301 | eavpgdnvgf nvknvsvkdi rrgsvcsdsk |
| | qdpakeartf haqviimnhp gQisngytpv |
| 361 | ldchtahiac kfnelkekvd rrtgkkvedf |
| | pkflksgdag iveliptkpl cvesftdyap |
| 421 | lgrfavrdmr qtvavgviks veksdgssgk |
| | vtksaqkaap kkk |
| // | |

IIIJ. GAP/RAN-GAP Family

SWSGDKLAWLQTWRRVISLVDPYTNSSAHVAIDCMSLTIENLLLVNLHPL
AHWLACRLVTVPPILLPRCVPALSAILNESTIRRPPPLLSANILLCFIRL
MQSKEQLVVPAICGLSAHELSIVAPRALEHLPKMLQAAKSSKDTKVSSNS
LKLFSMLASSYPGAEQILLDQLVNTDVSENAVVIVNSLAILIVQKAQIDL
VLTALKTIETHQFAMRLIPLFCSSIASLAQFSSTTLLQALLPAASLLRDE
RTRTEIEWQMVKLCMQWPQPQMPLVIRGILADRHMVLHGELVTLGGQYPV
RGFEVQRWSSAGAPPLQGEDKTVYINRQSAIISVSRKDFHAKSPCEITSR
TVVGRHIWDLDTHEDVRKPATNVTNWLRKEALKGKRPGRESQGILGAMDD
PFDDLPDYPPSRGSPSPVDGAAQFTSMIETSRRQPQPLGTSSAAHDHLPA
FTPNAKLLEWRSLSASLGFVPLVSQVHANFPRDLKHLDQTSSREVHKVAV
IYVGESQEDRASILSNTTASASAQFDSFTSELGWEVKVGRGHDGYTGGLP
VETRAPYFADAEAEVIFHVSTMLNGDVQQKWKHIGNDEVHVVWTENTRKV
YTRETIATKFCDVLIVLEQVGDKMVRVRVDTASALEFGPLFDGALVTMSE
LSQLVRLTVINASRAYRLARVEHSRPLRHREEVFCNEALAHMKPMPLAQS
INHLYVPTI

Also see the C. elegans Protein Database: Wormpep at http://www.sanger.ac.uk/Projects/C_{—}elegans/wormpep/; Accesion No. ce21437.

IIIK. HMG-I/Y DNA Binding Protein

MVEGDVDESASGTSGTNKKILFTKKPSVWKDFDNWINDEPENRYDLFQVV
KSAMLLQSGYTTILMDQVTDNGADELRISLEYSNFIKIVNSTKLVVGKEQ
CPPSNVFTLLAEIFANTPGNTSEVGRISTWLTSHLGALLHNDVIWKIHFF
DPDLFRSVYWQLIFTLKLAPGDTENLEEDENYAKLLFSCFITAVMVALWH
DHEMSFNSICPDYLKPETASEYMVMLISSPPFRSLSQFFLFGLHLLGKYQ
SEGGCVVVREEAYIAEIRQNDEEKRQSIETRTNLISDDMVYDDGEDLLEQ
IDRVQQLHEAHCIVLLKKGFLKAPDGFKIVQKGGRPRKYPASATKKRKKK
TPRSSPKKKMSKESPINHQKEPIDEQKPSTSLPIYSVATLKPRRKVVKTA
DEVGLGAPIFVMQSELLKKFREEVQRRYAEGSSASDQERVRNMVYEAYDN
IYHINRLSANEGPRILTSDQKLVMQQYKTTFRQGPTFAEETESDVEEEEE
KKVVEVVTAKVIKGSAKSSKKFKRRY

Also see the C. elegans Protein Database: Wormpep at http://www.sanger.ac.uk/Projects/C_{—}elegans/wormpep/; Accesion No. ce08872.

IIIL. HMG-I/Y DNA Binding PB1 Domain

Also see the C. elegans Protein Database: Wormpep at http://www.sanger.ac.uk/Projects/C_{—}elegans/wormpep/; Accesion No. ce20336.

IIIM. SNR-2 SM Protein

Member of the Small Nuclear Ribonucleoprotein gene class.

MTISKNNKNM AHLNYRMKII LQDGRTFIGF FKAFDKHMNI
LLAECEEHRQ IKPKAGKKTD
GEEKRILGLV LVRGEHIVSM TVDGPPPRDD DSVRLAKAGG
AGGVGQAKPG GRGMPAMPGM
PGMPPGGAPG GLSGAMRGHG GPGMAANQPG
YGGPPGGRPF

Also see the C. elegans Protein Database: Wormpep at http://www.sanger.ac.uk/Projects/C_{—}elegans/wormpep/; Accesion No. ce14704.

Homologs include Swiss-Prot. TrEMBL Accession No. Q15182 H. sapiens and TrEMBL Accession No. 070499, M. musculus Small nuclear ribonucleoprotein N.

IIIN. SNR-3 SM Protein

The SNR-3 SM protein is a member of the Small Nuclear Ribonucleoprotein SMD1 gene class. A homolog for this gene product is human SMD1.

_{—————————————.}
MKLVRFLMKL SHETVNIELK NGTQVSGTIM GVDVAMNTHL
RAVSMTVKNK EPVKLDTLSI
RGNNIRYIIL PDPLALDTLL IDDEPRKKAR AARAGASRGR
GRGGMRGGRG GRGRGRGGPR GGGPRR

Also see the C. elegans Protein Database: Wormpep at http://www.sanger.ac.uk/Projects/C_{—}elegans/wormpep/; Accesion No. ce02065.

IIIO. Dual Specificity Phosphatase

MPEPRCTAIV NFLNLSHSIL ISIFSVSVMS NYHHNHNYQH
RPRGYERLPG KRLPDRWNIY
DNVGRDIDGT RFVPEKTPLD SSFFDGKNMP VELQFGVKTL
ISLAQQANKQ IGLVIDLTNT
DRYYKKTEWA DHGVKYLKLN CPGHEVNERE DLVQDFINAV
KEEVNDKEND GKLIGVHCTH
GLNRTGYLIC RYMIDVDNYS ASDAISMFEY YRGHPMEREH
YKKSLYEAER KKKYGKSSGK
SSGNSADSTI SSEQLHRNNS Q

Homologs include, for example, Swiss Prot. Accession No. 075319, H. sapiens Dual specificity protein phosphatase 11 and TrEMLB Accession No. Q8BTR4, similar to dual specificity protein phosphatase 11.

Also see the C. elegans Protein Database: Wormpep at http://www.sanger.ac.uk/Projects/C_{—}elegans/wormpep/; Accesion No. ce03706.

IIIP. LIN-41

A homolog of LIN-41 is the human tripartite motif protein 2 (RING finger protein 86)

| LOCUS | | NP_492487 1143 aa linear INV 21- |
|---|---|---|
| NOVEMBER | | |
| 2003 | | |
| DEFINITION | | abnormal cell LINeage LIN-41, heterochronic gene; Drosophila |
| | | dappled/vertebrate TRipartite Motif protein related; B-box |
| | | zinc finger, Filamin and NHL repeat containing protein |
| | | (123.8 kD) (lin-41) [Caenorhabditis elegans]. |
| ACCESSION | | NP_492487 |
| VERSION | | NP_492487.2 GI: 25149908 |
| DBSOURCE | | REFSEQ: accession NM 060086.2 |
| KEYWORDS | | . |
| SOURCE | | Caenorhabditis elegans |
| | ORGANISM | Caenorhabditis elegans |
| | | Eukaryota; Metazoa; Nematoda; Chromadorea; Rhabditida; |
| | | Rhabditoidea; Rhabditidae; Peloderinae; Caenorhabditis. |
| REFERENCE | | 1 (residues 1 to 1143) |
| | AUTHORS | Lin,S. Y., Johnson,S. M., Abraham,M., Vella,M. C., |
| | | Pasquinelli, A., Gamberi,C., Gottlieb,E. and Slack,F. J. |
| | TITLE | The C elegans hunchback homolog, hbl-1, controls temporal |
| | | patterning and is a probable microRNA target |
| | JOURNAL | Dev. Cell 4 (5), 639-650 (2003) |
| | MEDLINE | 22623382 |
| | PUBMED | 12737800 |
| REFERENCE | | 2 (residues 1 to 1143) |
| | AUTHORS | Grosshans,H. and Slack,F. J. |
| | TITLE | Micro-RNAs: small is plentiful |
| | JOURNAL | J. Cell Biol. 156 (1), 17-21 (2002) |
| | MEDLINE | 21640444 |
| | PUBMED | 11781331 |
| REFERENCE | | 3 (residues 1 to 1143) |
| | AUTHORS | Ketting,R. F., Fischer,S. E., Bernstein,E., Sijen,T., |
| | | Hannon,G. J. and Plasterk, R. H. |
| | TITLE | Dicer functions in RNA interference and in synthesis of small |
| | | RNA involved in developmental timing in C. elegans |
| | JOURNAL | Genes Dev. 15 (20), 2654-2659 (2001) |
| | MEDLINE | 21521222 |
| | PUBMED | 11641272 |
| REFERENCE | | 4 (residues 1 to 1143) |
| | AUTHORS | Sonoda,J. and Wharton,R. P. |
| | TITLE | Drosophila Brain Tumor is a translational repressor |
| | JOURNAL | Genes Dev. 15 (6), 762-773 (2001) |
| | MEDLINE | 21172744 |
| | PUBMED | 11274060 |
| REFERENCE | | 5 (residues 1 to 1143) |
| | AUTHORS | Slack,F. J., Basson,M., Liu,Z., Ambros,V., Horvitz,H. R. and |
| | | Ruvkun, G. |
| | TITLE | The lin-41 RBCC gene acts in the C. elegans heterochronic |
| | | pathway between the let-7 regulatory RNA and the LIN-29 |
| | | transcription factor |
| | JOURNAL | Mol. Cell 5 (4), 659-669 (2000) |
| | MEDLINE | 20337950 |
| | PUBMED | 10882102 |
| REFERENCE | | 6 (residues 1 to 1143) |
| | AUTHORS | Reinhart,B. J., Slack,F. J., Basson,M., Pasquinelli,A. E., |
| | | Bettinger,J. C., Rougvie,A. E., Horvitz,H. R. and Ruvkun,G. |
| | TITLE | The 21-nucleotide let-7 RNA regulates developmental timing in |
| | | Caenorhabditis elegans |
| | JOURNAL | Nature 403 (6772), 901-906 (2000) |
| | MEDLINE | 20168806 |
| | PUBMED | 10706289 |

| | |
|---|---|
| COMMENT | REVIEWED REFSEQ: This record has been curated by NCBI staff. |
| | This record is derived from an annotated genomic sequence |
| | (NC_003279). The reference sequence was derived from AF195610. |
| | On Nov. 21, 2002 this sequence version replaced gi: 17508265. |
| | Summary: This gene lin-41, also known as C12C8.3, 1J912 or |
| | YK872, maps at (I; +3.53). Its phenotype is abnormal cell |
| | lineage, heterochronic. It encodes a heterochronic gene; |
| | Drosophila dappled/vertebrate TRipartite Motif protein related; |
| | B-box zinc finger, Filamin and NHL repeat containing protein. |
| | From Pfam homology, the products would have zinc binding |
| | activity and would localize in intracellular. |
| | According to the Worm Transcriptome Project, it is well |
| | expressed mostly from L1 larvae to adult [Kohara cDNAs]. Its |
| | sequence is defined by 11 cDNA clones and produces, by alterna- |
| | tive splicing, at least 2 different transcripts b, a altogether |
| | encoding 2 different protein isoforms. The transcripts appear to |
| | differ by common exons with different boundaries. |
| | Phenotype |
| | [from C. elegans II book] Allele ma104: heterochronic defect |
| | in L4 larvae to adult switch. [Victor Ambros]. |
| | Selected strains available from the CGC. |
| | CT8 lin-41 (ma104) I [Frank Slack, V. Ambros, mutator TR679] |
| | Dpy. Precocious heterochronic. Reduced brood size. There may be |
| | a linked Dpy mutation in this strain. |
| | MT7897 lin-41 (n2914)/unc-29 (e1072) lin-11 (n1281) I [Bob |
| | Horvitz, M. Basson, EMS] Heterozygotes are WT and segregate WT, |
| | UncVul and lin-41 (Dpy, Scrawny and Sterile). |
| | RNA interference results: |
| | [J. Ahringer 2000] No obvious phenotype (by feeding genomic PCR |
| | product JA: C12C8.3). |
| | Expression |
| | The expression profile for the gene, derived from the |
| | proportion of animals at each stage in each Kohara library is: |
| | embryos 3%, L1 or L2 larvae 50%, L3 to adult 47%. |
| | In situ hybridisation pictures to all stages of development |
| | are available from Kohara NextDB. |
| | The report below describes variant a. |
| | This complete mRNA is 4797 bp long. It is supported by 2 cDNA |
| | clones. Its sequence exactly matches the genome. The |
| | premessenger has 16 exons. It covers 7.70 kb on the WS97 genome. |
| | It is transpliced to SL1. It has a very long 3′ UTR. The protein |
| | (1143 aa, 123.8 kDa, pI 6.1) contains 2 Zn-finger, B-box motifs, |
| | one Filamin/ABP280 repeat motif, 6 NHL repeat motifs. It also |
| | contains a coil coil stretch [Psort2]. Taxblast (threshold 10\hat{ }-3) |
| | tracks ancestors down to archaea and bacteria and eukaryota. |
| | COMPLETENESS: full length. |
| | Method: conceptual translation. |

| FEATURES | | Location/Qualifiers |
|---|---|---|
| | source | 1 . . . 1143 |
| | | /organism = “Caenorhabditis elegans” |
| | | /db_xref = “taxon: 6239” |
| | | /chromosome = “I” |
| | | /map = “I; +3.53 cM (interpolated genetic position)” |
| | | /map = “I; +3.75 cM (measured genetic position)” |
| | | /map = “I; covering 7702 bp, from base 9350549 to |
| | | 9342848 on genome release WS97” |
| | | /clone = “Primers to amplify the CDS (10290 bp, Stop |
| | | included): ATGGCGACCATCGTGCCATGCT (T = 63.8), |
| | | CTAGAAGACACGGATGCAATTGTTTCCGAA (T = 63.4). Clone |
| | | specific of this variant is AF195610. Complete CDS clones: |
| | | yk1728d7. Recommended clone (from the Kohara collection): |
| | | yk1728d7. for edited clone sequences see www.wormgenes.org” |
| | | /clone_lib = “Kohara Sugano L2 larvae cap-selected |
| | | library: yk1728d7; gb: AF195610” |
| | Protein | 1 . . . 1143 |
| | | /product = “abnormal cell LINeage LIN-41, heterochronic |
| | | gene; Drosophila dappled/vertebrate TRipartite Motif |
| | | protein related; B-box zinc finger, Filamin and NHL |
| | | repeat containing protein (123.8 kD) (lin-41)” |
| | Region | 366 . . . 412 |
| | | /region name = “[Pfam/InterPro description] zn-finger, |
| | | B-box” |
| | | /db_xref = “CDD: pfam00643” |
| | Region | 470 . . . 512 |
| | | region_name = “[Pfam/InterPro description] zn-finger, |
| | | B-box” |
| | | /db_xref = “CDD: pfam00643” |
| | Region | 553 . . . 617 |
| | | /region_name = “[PSORT] coil coil domain: |
| | | TAENEIRAAFDTHVNALEERRKELLKRVETVKNLKLSVLISQAESLQSKQIDLQQAIQ |
| | | TATKLMD” |
| | Region | 688 . . . 810 |
| | | /region_name = “[Pfam/Interpro description] |
| | | Filamin/ABP280 repeat” |
| | | /db_xref = “CDD: pfam00630” |
| | Region | 841 . . . 868 |
| | | /region_name = “[Pfam/InterPro description] NHL repeat” |
| | | /db_xref = “CDD: pfam01436” |
| | Region | 888 . . . 915 |
| | | /region_name = “[Pfam/InterPro description] NHL repeat” |
| | | /db_xref = “CDD: pfam01436” |
| | Region | 935 . . . 962 |
| | | /region_name = “[Pfam/InterPro description] NHL repeat” |
| | | /db_xref = “CDD: pfam01436” |
| | Region | 983 . . . 1010 |
| | | /region_name = “[Pfam/Interpro description] NHL repeat” |
| | | /db_xref = “CDD: pfam01436” |
| | Region | 1031 . . . 1058 |
| | | /region_name = “[Pfam/InterPro description] NHL repeat” |
| | | /db_xref = “CDD: pfam01436” |
| | Region | 1116 . . . 1143 |
| | | /region_name = “[Pfam/Interpro description] NHL repeat” |
| | | /db_xref = “CDD: pfam01436” |
| | CDS | 1 . . . 1143 |
| | | /gene = “lin-41” |
| | | locus_tag = “1J912” |
| | | /coded_by = “NM_060086.2: 196 . . . 3627” |
| | | /db_xref = “AceView/WormGenes: lin-41” |
| | | /db_xref = “GeneID: 172760” |
| | | /db_xref = “LocusID: 172760” |
| | | /db_xref = “WormBase: C12C8.3a” |

| ORIGIN | | |
|---|---|---|
| 1 | | mativpcsle keegapsgpr rlqteidvda ndsgnelsmg gsssegdsms |
| | | hhrgehspnh |
| 61 | | hhqdnhlgsg ppppqftgsl fdtppsmiqs pqqqpqfqfn tgfglglpqd |
| | | sfrcsvcsks |
| 121 | | stigvlpfvc ahktcqscyq mtpssydrra cklcgavsta tanftsqmyl |
| | | sptlpspprg |
| 181 | | almsdcstpt mnnhinsstp lhqprafsfs isqmpgspsp vmqarmpssa |
| | | gglmrnrpigf |
| 241 | | pdsdssltsw splqqpsqls innlssiggh qqqspmlmqn vfdslavndd |
| | | tpvfsplspt |
| 301 | | ntsmhmppsl maspdvpkhs atiapprnsm cstprlqlat pmssqsqqtf |
| | | pipsplqsqp |
| 361 | | qqqqpmgpiq cqgceskisf aycmqcqeal cihcvqahqr vratkqhafv |
| | | elqqlmatlm |
| 421 | | sravqpqqaq qytqnvqgsv rqalgsvgsg dghvsgvend sigsgesspr |
| | | sssvcgthds |
| 481 | | viigicencp hsvllcaicv aqhpgkhrvq plgdirvavg evvnesqllq |
| | | wqcektgdti |
| 541 | | kqiidgivtn attaeneira afdthvnale errkellkrv etvknlklsv |
| | | lisqaeslqs |
| 601 | | kqidlqqaiq tatklmdssd cdemvlrqvf eklascqingn eqtepnnnil |
| | | nvlmlacqvn |
| 661 | | eddrlkftap qdgillnkar qfgniesgpc aknssivgds fkkairerqt |
| | | viyvqlrdac |
| 721 | | gdllsssiaa tqptsqallp hqephshleq amptsdvqaf vispdgstve |
| | | vtmtprengi |
| 781 | | valsyypsie gsytlnilvk qtpisgcptt mdirrgrnyd eiaakgpilt |
| | | fgkegsgdge |
| 841 | | lcrpwgicvd qrgrvivadr snnrvqifdk dgnfiskfgt sgnrpgqfdr |
| | | pagittnsln |
| 901 | | nivvadkdnh rvqvfdengm fllkfgdrgr avgyfnypwg vatnshnaia |
| | | vsdtrnhrvq |
| 961 | | iftpqgqfvr kcgfdsayff knldsprglc ylpdgqllit dfnnhrlavl |
| | | sprnmsemkv |
| 1021 | | ygsegdgdgm fvrpqgvvid peghilvcds rnnrvqvfas ddmrfigsfg |
| | | lgpvpnsgfq |
| 1081 | | mpqelpapys slggpfgapa fssaptpltp sprqlldrpt dlavgpdgri |
| | | yvvdfgnnci |
| 1141 | | rvf |
| // | | |
| LOCUS | | NP_492488 1147 aa linear INV 21- |
| NOVEMBER | | |
| 2003 | | |
| DEFINITION | | abnormal cell LINeage LIN-41, heterochronic gene; Drosophila |
| | | dappled/vertebrate TRipartite Motif protein related; B-box |
| | | zinc finger, Filamin and NHL repeat containing protein |
| | | (124.2 kD) (lin-41) [Caenorhabditis elegans]. |
| ACCESSION | | NP_492488 |
| VERSION | | NP_492488.2 GI: 25149913 |
| DESOURCE | | REFSEQ: accession NM 060087.2 |
| KEYWORDS | | . |
| SOURCE | | Caenorhabditis elegans |
| | ORGANISM | Caenorhabditis elegans |
| | | Eukaryota; Metazoa; Nematoda; Chromadorea; Rhabditida; |
| | | Rhabditoidea; Rhabditidae; Peloderinae; Caenorhabditis. |
| REFERENCE | | 1 (residues 1 to 1147) |
| | AUTHORS | Lin,S. Y., Johnson,S. M., Abraham,M., Vella,M. C., |
| | | Pasquinelli, A. Gamberi,C., Gottlieb,E. and Slack,F. J. |
| | TITLE | The C elegans hunchback homolog, hbl-1, controls temporal |
| | | patterning and is a probable microRNA target |
| | JOURNAL | Dev. Cell 4 (5), 639-650 (2003) |
| | MEDLINE | 22623382 |
| | PUBMED | 12737800 |
| REFERENCE | | 2 (residues 1 to 1147) |
| | AUTHORS | Grosshans,H. and Slack,F. J. |
| | TITLE | Micro-RNAs: small is plentiful |
| | JOURNAL | J. Cell Biol. 156 (1), 17-21 (2002) |
| | MEDLINE | 21640444 |
| | PUBMED | 11781331 |
| REFERENCE | | 3 (residues 1 to 1147) |
| | AUTHORS | Ketting,R. F., Fischer,S. E., Bernstein,E., Sijen,T., |
| | | Hannon,G. J. and Plasterk, R. H. |
| | TITLE | Dicer functions in RNA interference and in synthesis of small |
| | | RNA involved in developmental timing in C. elegans |
| | JOURNAL | Genes Dev. 15 (20), 2654-2659 (2001) |
| | MEDLINE | 21521222 |
| | PUBMED | 11641272 |
| REFERENCE | | 4 (residues 1 to 1147) |
| | AUTHORS | Sonoda,J. and Wharton,R. P. |
| | TITLE | Drosophila Brain Tumor is a translational repressor |
| | JOURNAL | Genes Dev. 15 (6), 762-773 (2001) |
| | MEDLINE | 21172744 |
| | PUBMED | 11274060 |
| REFERENCE | | 5 (residues 1 to 1147) |
| | AUTHORS | Slack,F. J., Basson,M., Liu,Z., Arnbros,V., Horvitz,H. R. and |
| | | Ruvkun, G. |
| | TITLE | The lin-41 RBCC gene acts in the C. elegans heterochronic |
| | | pathway between the let-7 regulatory RNA and the LIN-29 |
| | | transcription factor |
| | JOURNAL | Mol. Cell 5 (4), 659-669 (2000) |
| | MEDLINE | 20337950 |
| | PUBMED | 10882102 |
| REFERENCE | | 6 (residues 1 to 1147) |
| | AUTHORS | Reinhart,B. J., Slack,F. J., Basson,M., Pasquinelli,A. E., |
| | | Bettinger,J. C., Rougvie,A. E., Horvitz,H. R. and Ruvkun,G. |
| | TITLE | The 21-nucleotide let-7 RNA regulates developmental timing in |
| | | Caenorhabditis elegans |
| | JOURNAL | Nature 403 (6772), 901-906 (2000) |
| | MEDLINE | 20168806 |
| | PUBMED | 10706289 |

| | |
|---|---|
| COMMENT | REVIEWED REFSEQ: This record has been curated by NCBI staff. |
| | This record is derived from an annotated genomic sequence |
| | (NC_003279). The reference sequence was derived from AF195611. |
| | On Nov. 21, 2002 this sequence version replaced gi: 17508263. |
| | Summary: This gene lin-41, also known as C12C8.3, 1J912 or |
| | YK872, maps at (I; +3.53). Its phenotype is abnormal cell |
| | lineage, heterochronic. It encodes a heterochronic gene; |
| | Drosophila dappled/vertebrate TRipartite Motif protein related; |
| | B-box zinc finger, Filamin and NHL repeat containing protein. |
| | From Pfam homology, the products would have zinc binding activity |
| | and would localize in intracellular. |
| | According to the Worm Transcriptome Project, it is well |
| | expressed mostly from L1 larvae to adult [Kohara cDNAs]. Its |
| | sequence is defined by 11 cDNA clones and produces, by alternative |
| | splicing, at least 2 different transcripts b, a altogether encoding |
| | 2 different exons protein isoforms. The transcripts appear to differ |
| | by common with different boundaries. |
| | Phenotype |
| | [from C. elegans II book] Allele ma104: heterochronic defect |
| | in L4 larvae to adult switch. [Victor Ambros]. |
| | Selected strains available from the CGC. |
| | CT8 lin-41 (ma104) I [Frank Slack, V. Ambros, mutator TR679] |
| | Dpy. Precocious heterochronic. Reduced brood size. There may be a |
| | linked Dpy mutation in this strain. |
| | MT7897 lin-41 (n2914)/unc-29 (e1072) lin-11 (n1281) I [Bob |
| | Horvitz, M. Basson, EMS] Heterozygotes are WT and segregate WT, |
| | UncVul and lin-41 (Dpy, Scrawny and Sterile). |
| | RNA interference results: |
| | [J. Ahringer 2000] No obvious phenotype (by feeding genomic PCR |
| | product JA: C12C8.3). |
| | Expression |
| | The expression profile for the gene, derived from the |
| | proportion of animals at each stage in each Kohara library is: |
| | embryos 3%, L1 or L2 larvae 50%, L3 to adult 47%. The expression |
| | profile for the gene, derived from the proportion of animals at each |
| | stage in each Kohara library is: embryos 3%, L1 or L2 larvae 45%, L3 |
| | to adult 51%. |
| | In situ hybridisation pictures to all stages of development |
| | are available from Kohara NextDB. |
| | The report below describes variant b. |
| | This complete mRNA is 4809 bp long. It is supported by 8 cDNA |
| | clones, 7 of which match only this alternative variant. Its |
| | sequence exactly matches the genome. The premessenger has 16 |
| | exons. It covers 7.70 kb on the WS97 genome. It is transpliced to |
| | SL1. It has a very long 3′ UTR. The protein (1147 aa, 124.2 kDa, pI |
| | 6.1) contains 2 Zn-finger, B-box motifs, one Filamin/ABP280 repeat |
| | motif, 6 NHL repeat motifs. It also contains a coil coil |
| | stretch [Psort2]. Taxblast (threshold 10\hat{ }-3) tracks ancestors down |
| | to archaea and bacteria and eukaryota. |
| | COMPLETENESS: full length. |
| | Method: conceptual translation. |

| FEATURES | | Location/Qualifiers |
|---|---|---|
| | source | 1 . . . 1147 |
| | | /organism = “Caenorhabditis elegans” |
| | | /db_xref = “taxon: 6239” |
| | | /chromosome = “I” |
| | | /map = “I; +3.53 cM (interpolated genetic position)” |
| | | /map = “I; +3.75 cM (measured genetic position)” |
| | | /map = “I; covering 7702 bp, from base 9350549 to |
| | | 9342848 on genome release WS97” |
| | | /clone = “Primers to amplify the CDS (10326 bp, Stop |
| | | included): ATGGCGACCATCGTGCCATGCT (T = 63.8), |
| | | CTAGAAGACACGGATGCAATTGTTTCCGAA (T = 63.4). Clones |
| | | specific of this variant are AF195611, yk20b11, |
| | | yk307c10, yk1100f6, yk1102h6, yk1111g2, yk1223b8. |
| | | Complete CDS clones: yk1728d7. Recommended clone |
| | | (from the Kohara collection): yk1728d7. for edited |
| | | clone sequences see www.wormgenes.org” |
| | | /clone_lib = “Kohara Sugano L1 larvae cap-selected |
| | | library: yk1111g2, yk1100f6, yk1102h6, yk1223b8; |
| | | Kohara Sugano L2 larvae cap-selected library: |
| | | yk1728d7; Kohara mixed stage library, from him-8 |
| | | strain, containing 15-30% males: yk307c10; Kohara |
| | | mixed stage library, from him-8 strain, containing |
| | | 15-30% males: yk20b11; gb: AF195611” |
| | Protein | 1 . . . 1147 |
| | | /product = “abnormal cell LINeage LIN-41, heterochronic |
| | | gene; Drosophila dappled/vertebrate TRipartite Motif |
| | | protein related; B-box zinc finger, Filamin and NHL |
| | | repeat containing protein (124.2 kD) (lin-41)” |
| | Region | 366 . . . 412 |
| | | /region_name = “[Pfam/InterPro description] zn-finger, |
| | | B-box” |
| | | /db_xref = “CDD: pfam00643” |
| | Region | 474 . . . 516 |
| | | /region_name = “[Pfam/Interpro description] zn-finger, |
| | | B-box” |
| | | /db_xref = “CDD: pfam00643” |
| | Region | 557 . . . 621 |
| | | /region_name = “[PSORT] coil coil domain: |
| | | TAENEIRAAFDTHVNALEERRKELLKRVETVKNLKLSVLISQAESLQSKQIDLQQAIQ |
| | | TATKLMD” |
| | Region | 692 . . . 814 |
| | | /region_name = “[Pfam/InterPro description] |
| | | Filamin/ABP280 repeat” |
| | | /db_xref = “CDD: pfam00630” |
| | Region | 845 . . . 872 |
| | | /region_name = “[Pfam/InterPro description] NHL repeat” |
| | | /db_xref = “CDD: pfam01436” |
| | Region | 892 . . . 919 |
| | | /region_name = “[Pfam/InterPro description] NHL repeat” |
| | | /db_xref = “CDD: pfam01436” |
| | Region | 939 . . . 966 |
| | | /region_name = “[Pfam/Interpro description] NHL repeat” |
| | | /db_xref = “CDD: pfam01436” |
| | Region | 987 . . . 1014 |
| | | /region_name = “[Pfam/InterPro description] NHL repeat” |
| | | /db_xref = “CDD: pfam01436” |
| | Region | 1035 . . . 1062 |
| | | /region_name = “[Pfam/InterPro description] NHL repeat” |
| | | /db_xref = “CDD: pfam01436” |
| | Region | 1120 . . . 1147 |
| | | /region_name = “[Pfam/InterPro description] NHL repeat” |
| | | /db_xref = “CDD: pfam01436” |
| | CDS | 1 . . . 1147 |
| | | /gene = “lin-41” |
| | | /locus_tag = “1J912” |
| | | /coded_by = “NM_060087.2: 196 . . . 3639” |
| | | /db_xref = “AceView/WormGenes: lin-41” |
| | | /db_xref = “GeneID: 172760” |
| | | /db_xref = “LocusID: 172760” |

| | |
|---|---|
| ORIGIN | |
| 1 | mativpcsle keegapsgpr rlqteidvda ndsgnelsmg gsssegdsms |
| | hhrgehspnh |
| 61 | hhqdnhlgsg ppppqftgsl fdtppsmiqs pqqqpqfqfn tgfglglpqd |
| | sfrcsvcsks |
| 121 | stigvlpfvc ahktcqscyq mtpssydrra cklcgavsta tanftsqmyl |
| | sptlpspprg |
| 181 | almsdcstpt mnnhinsstp lhqprafsfs lsgmpgspsp vmgarmpssa |
| | gglmmrpigf |
| 241 | pdsdssltsw splqqpsqls innlssiggh qqqspmlmqn vfdslavndd |
| | tpvfsplspt |
| 301 | ntsmhmppsl maspdvpkhs atiapprnsm cstprlqlat pmssqsqqtf |
| | pipsplgsqp |
| 361 | qqqqpmgpiq cqgceskisf aycmqcqeal cihcvqahqr vratkqhafv |
| | elqqlmatlm |
| 421 | sravqpqqaq qytqnvggsv rqalgsvgsg dvffsghvsg vendsigsge |
| | ssprsssvcg |
| 481 | thdsviigic encphsvllc aicvaqhpgk hrvqplgdir vavgevvnes |
| | qllqwqcekt |
| 541 | gdtikqiidg ivtnattaen eiraafdthv naleerrkel lkrvetvknl |
| | klsvlisqae |
| 601 | slqskqidlq qaiqtatklm dssdcdemvl rqvfeklasc qmgnegtepn |
| | nnilnvlmla |
| 661 | cqvneddrlk ftapqdgill nkarqfgnie sgpcaknssi vgdsfkkair |
| | erqtviyvql |
| 721 | rdacqdllss siaatqptsq allphqephs hleqamptsd vqafvispdg |
| | stvevtmtpr |
| 781 | engivalsyy psiegsytln ilvkqtpisg cpttmdirrg rnydeiaakg |
| | piltfgkegs |
| 841 | gdgelcrpwg icvdqrgrvi vadrsnnrvq ifdkdgnfis kfgtsgnrpg |
| | qfdrpaqitt |
| 901 | nslnnivvad kdnhrvqvfd engmfllkfg drgravgyfn ypwgvatnsh |
| | naiavsdtrn |
| 961 | hrvqiftpqg qfvrkcgfds ayffknldsp rglcylpdgq llitdfnnhr |
| | lavlsprnms |
| 1021 | emkvyqsegd gdgmfvrpqg vvidpeghil vcdsrnnrvq vfasddmrfi |
| | gsfglgpvpn |
| 1081 | sgfqmpqelp apysslggpf gapafssapt pltpsprqll drptdlavgp |
| | dgriyvvdfg |
| 1141 | nncirvf |
| // | |

LIN-41 homologs include H. sapiens gi|37550026|ref|XP_{—}067369.3|[37550026], M. musculus gi|38090144|ref|XP_{—}356199.1|[38090144] and R. norvegicus gi|34866457|ref|XP_{—}236676.2|[34866457].

IIIQ. Low Homology MADS Box Protein, Novel

Also see the C. elegans Protein Database: Wormpep at http://www.sanger.ac.uk/Projects/C_{—}elegans/wormpep/; Accesion No. ce01506.

IIIR. RPN-9 Proteasome Subunit

MTAQDYLNGK LAAANGPLAD DWKNLKELWE KKLWHQLTVL
TRSLVKKPQF VASTDMHEFY
RLFVAEWELR VNPLQLVEIC ISIAQNIATK DKQKSMEFLS
KIGNVINKDK IAVARLHTGE
IEARLENKDK NGQIIDLKSI RTQIDSTQHE VDSLVGVTEV
HAPFYRVSSL YLREVGDFAG
YYREALRYLG VEDANNLTTE QKQVHAVLLG FAALLGENVH
NFGELLAHPI LKSLEGTRER
WIVDVLLAFN SGDLTRFFSL EGDWGGWDDL KKQKDFLTAK
IRLMAVMELA VSRPTKARSV
SFKEIATKCQ IPFDEVEFLV MKALSKDLIR GDINQVEQVV
YVTWVQPRVL DNPQIMQMAT
RISAWRNDVN SMEGIVSKEA REILTQN

Homologs include, for example, Swiss-Prot. Accession No. Q9WVJ2, M. musculus 26S proteasome non-ATPase regulatory subunit 13 (26S proteasomesregulatory subunit S11) (26S proteasome regulatory subunit p40.5). Swiss-Prot. Q9UNM6, H. sapiens 26S proteasome non-ATPase regulatory subunit and Swiss Prot. Accession No. Q04062, S. cerevisiae Regulatory Particle Non-ATPase.

IIIS. TAF 6.1

The TAF 6.1 is part of an operon with w09b6.3 (an enhancer of RNAi) and expressed as a polypeptide fusion. This protein is well conserved and the human ortholog is Transcription initiation factor TFIID subunit 6.

| | |
|---|---|
| LOCUS | NP_493919 470 aa linear INV 21- |
| NOVEMBER | |
| 2003 | |
| DEFINITION | TBP-Associated transcription Factor |
| | family member (52.7 kD) (taf-6.1) |
| | [Caenorhabditis eleqans]. |
| ACCESSION | NP_493919 |
| VERSION | NP_493919.1 GI: 17536589 |
| DBSOURCE | REFSEQ: accession NM 061518.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| COMMENT | VALIDATED REFSEQ: This record has un- |
| | dergone preliminary review of the se- |
| | quence, but has not yet been subject to |
| | final review. This record is derived |
| | from an annotated genomic sequence |
| | (NC_003280). The reference sequence was |
| | derived from WormBase CDS: W09B6.2. |
| | Summary: This gene taf-6.1, also known |
| | as W09B6.2, 2B421 or YK5540, maps at |
| | (II; −12.83). It encodes a TBP-Associ- |
| | ated transcription Factor family |
| | member. |
| | According to the Worm Transcriptome |
| | Project, it is well expressed mostly |
| | in embryos, and some at all stages of |
| | development [Kohara cDNAs]. Its se- |
| | quence is fully supported by 8 cDNA |
| | clones. |
| | RNA interference results: |
| | [J. Ahringer 2003] No obvious phenotype |
| | (by feeding genomic PCR product JA: |
| | W09B6.2). Warning: this double stranded |
| | RNA may also interfere with gene 2B417. |
| | Function |
| | Protein properties: used to be called |
| | taf-3. |
| | Expression |
| | The expression profile for the gene, |
| | derived from the proportion of animals |
| | at each stage in each Kohara library |
| | is: embryos 56%, L1 or L2 larvae 21%, |
| | L3 to adult 24%. |
| | In situ hybridisation pictures to all |
| | stages of development are available |
| | from Kohara NextDB. |
| | The CDS has 8 exons. It covers 4.31 kb |
| | on the WS97 genome. The protein (470 |
| | aa, 52.7 kDa, pI 8.7) contains no Pfam |
| | motif. It is predicted to localise in |
| | the mitochondria [Psort2]. Taxblast |
| | (threshold 10\hat{ }-3) tracks ancestors down |
| | to eukaryota. |
| | COMPLETENESS: full length. |
| | Method: conceptual translation. |

| | |
|---|---|
| FEATURES | Location/Qualifiers |

| | |
|---|---|
| source | 1 . . . 470 |
| | /organism = “Caenorhabditis |
| | elegans” |
| | /db_xref = “taxon: 6239” |
| | /chromosome = “II” |
| | /map = “II; −12.83 cM (inter- |
| | polated genetic position)” |
| | /map = “II; covering 4373 bp, from |
| | base 1127981 to 1132355 on genome |
| | release WS97” |
| | /clone_lib = “Kohara embryonic |
| | lambda gt11 library: yk314a6, |
| | yk502e6, yk649h1, yk650b11, |
| | yk670h10; Kohara Sugano L1 larvae |
| | cap-selected library: yk1035e11, |
| | yk1330h4; Kohara Sugano L4 larvae |
| | cap-selected library: yk850e10” |
| Protein | 1 . . . 470 |
| | /product = “TBP-Associated trans- |
| | cription Factor family member (52.7 |
| | kD) (taf-6.1)” |
| Region | 247 . . . 250 |
| | /region_name = “[PSORT] nuclear |
| | localization domain: KKRH” |
| CDS | 1 . . . 470 |
| | /gene = “taf-6.1” |
| | locus_tag = “2B421” |
| | /coded_by = “NM_061518.1: |
| | 1 . . . 1413” |
| | /db_xref = “AceView/WormGenes: |
| | taf-6.1” |
| | /db_xref = “GeneID: 173498” |
| | /db_xref = “LocusID: 173498” |
| | /db_xref = “WormBase: W09B6.2” |

| | |
|---|---|
| ORIGIN | |
| 1 | msktvtirrp sptktseepa ahqtpiftqt |
| | aaemlgitsl nteaaellef lsreklkeiv |
| 61 | rlsakwtqks arrrmavadv ehairstrqc |
| | gglnissvdt lnlgiqqlqp iqgtstgiys |
| 121 | flkssadvdv dkedtetfik iprdlrvisy |
| | plvnegqpvq seytvnvded dgnffekivp |
| 181 | evmtmipekn tpsssttssl qmfrdavkta |
| | kidqkvglkp stieiltveq qifmkdiitv |
| 241 | cmgqddkkrh ealytletda glqvflphlt |
| | ericksisan isqrclslii yagrvlrsls |
| 301 | hnkacdmtvt lhhvlpalls ccvqrnmclr |
| | petdnhwalr dfsaktlvgl vrdqvdkhda |
| 361 | grtarrlfdf shrifrdtgs sfsmiygtvh |
| | ilqefvagpk kaawlltelg etnarckshi |
| 421 | esgsrigasq lsiqeaqkln qqilkcensi |
| | rnrynlqqqa pgvpinrrfh |
| // | |

Also see the C. elegans Protein Database: Wormpep at http://www.sanger.ac.uk/Projects/C_{—}elegans/wormpep/; Accesion No. ce05915.

IIIU. RRM Protein

MATSFYTGGGEDGDGFNPRVHARIAEREGFQLASGSEDPRTLFVANLDPA
ITDEFLATLFNQIGAVMKAKIIFEGLNDPYAFVEFSDHNQATLALQSHNG
RELLEKEMHVTWAFEPREPGENRSKPETSRHFHVFVGDLCSEIDSTKLRE
AFVKFGEVSEAKIIRDNNTNKGKGYGFVSYPRREDAERAIDEMNGAWLGR
RTIRTNWATRKPDEDGERGGDRGDRRGGGGGGRDRYHNQSEKTYDEIFNQ
AAADNTSVYVGNIANLGEDEIRRAFDRFGPINEVRTFKIQGYAFVKEETK
ESAARAIVQMNNADIGGQIVRCSWGKSGDSGKPSERGSGGGGGSGNYGYG
YGNSGGGGGSGGPGNSQFSNFNQRPPPSGNGGSGGGSGGQNNQYWQYYSQ
YYNNPHLMQQWNNYWQKDGPPPPPAAAASSTGGN

Also see the C. elegans Protein Database: Wormpep at http://www.sanger.ac.uk/Projects/C_{—}elegans/wormpep/; Accesion No. ce21988.

IIIV. Worm Unique/Novel

MQPVLVNSRPLRVKSHESESKLNLIEQEDQFEGANYSSSSGVIICYSNGT
GEVITQEAFDDSGIHFIFSKATCIQYPSNFDPIGVGSVVQIFWSRSFERV
VRGNHIIVQIEKMEVYKCCAMLREQVFVTFNSPSTAGVAIGVTERNITVA
FHPNGSPVIRYETLKAHSIGRTEFEIKDVREFEFSNGKNRHRENTNRMVD
VILAAVPFRVEIHGNVDKIPFFVIEKCRNSPGRSGAAVITKIMKNHFMEA
NFLQNSESIYFDSTSCHSNILEKVSIGSLINVLADPTFATSSYKWYGYDV
TLCNNYLAHASTQRSFVLENNEILQNCKKLEKSPEEAETTTKNDLRFVPP
QPEKGEVKKNELPEREAKSIINSYFIDRLAEGIKIEKIDKNWRTFGEILP
KTPKKYSESLKKSIQNVLEPFGLNKPEKAAETPKIVEYFPKNPKKRVEIV
EKPTVDEIRELFGALMDAEGFALNQRVKPHFVLPDTRWKPTERRYIGIYD
DVQWTFMSTFCPKIEENSENRPLAGGWWYRRTVPRDHPVEIVQKMETRRN
IIKDCTESPFIE

Also see the C. elegans Protein Database: Wormpep at http://www.sanger.ac.uk/Projects/C_{—}elegans/wormpep/; Accesion No. ce27223.

IIIW. TBB-4

| | |
|---|---|
| LOCUS | NP_509585 444 aa linear INV 21- |
| NOVEMBER | |
| 2003 | |
| DEFINITION | tubulin, Beta (49.8 kD) (tbb-4) |
| | [Caenorhabditis elegans] |
| ACCESSION | NP_509585 |
| VERSION | NP_509585.1 GI: 17549915 |
| DBSOURCE | REFSEQ: accession NM 077184.1 |
| KEYWORDS | . |
| SOURCE | Caenorhabditis elegans |

| | |
|---|---|
| ORGANISM | Caenorhabditis elegans. |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 444) |

| | |
|---|---|
| AUTHORS | Maeda,I., Kohara,Y., Yamamoto,M. and |
| | Sugimoto,A. |
| TITLE | Large-scale analysis of gene function |
| | in Caenorhabditis elegans by high- |
| | throughput RNAi |
| JOURNAL | Curr. Biol. 11 (3), 171-176 (2001) |
| MEDLINE | 21154836 |
| PUBMED | 11231151 |

| | |
|---|---|
| COMMENT | VALIDATED REFSEQ: This record has un- |
| | dergone preliminary review of the se- |
| | quence, but has not yet been subject to |
| | final review. This record is derived |
| | from an annotated genomic sequence |
| | (NC_003284). The reference sequence was |
| | derived from WormBase CDS: B0272.1. |
| | Summary: This essential gene tbb-4, |
| | also known as B0272.1, XK54 or YK4801, |
| | maps at (X; +1.30). Its phenotype is |
| | embryonic lethal, partial. It encodes a |
| | tubulin, Beta. |
| | According to the Worm Transcriptome |
| | Project, it is well expressed mainly in |
| | embryos and some in L1 and L2 larvae |
| | [Kohara cDNAs]. Its sequence is fully |
| | supported by 7 cDNA clones. |
| | RNA interference results: |
| | [J. Ahringer 2003] No obvious phenotype |
| | (by feeding genomic PCR product JA: |
| | B0272.1). [A. Sugimoto 2002] Embryonic |
| | lethal (20%) (by injecting cDNA clone |
| | SA: yk313f12). |
| | Expression |
| | The expression profile for the gene, |
| | derived from the proportion of animals |
| | at each stage in each Kohara library |
| | is: embryos 82%, L1 or L2 larvae 14%, |
| | L3 to adult 4%. |
| | In situ hybridisation pictures to all |
| | stages of development are available |
| | from Kohara NextDB. |
| | The CDS has 7 exons. It covers 2.21 kb |
| | on the WS97 genome. The protein (444 |
| | aa, 49.8 kDa, pI 4.8) contains one |
| | Tubulin/FtsZ protein motif, one |
| | Tubulin/FtsZ protein motif. It also |
| | contains a coil coil stretch [Psort2]. |
| | It is predicted to localise in the |
| | cytoskeleton [Psort2]. Taxblast |
| | (threshold 10\hat{ }-3) tracks ancestors |
| | down to eukaryota. |
| | COMPLETENESS: full length. |
| | Method: conceptual translation. |
| FEATURES | Location/Qualifiers |

| | |
|---|---|
| source | 1 . . . 444 |
| | /organism = “Caenorhabditis |
| | elegans” |
| | /db_xref = “taxon: 6239” |
| | /chromosome = “X” |
| | /map = “X; +1.30 cM (interpolated |
| | genetic position)” |
| | /map = “X; covering 2444 bp, from |
| | base 9427407 to 9424964 on genome |
| | release WS97” |
| | /clone_lib = “Kohara embryonic |
| | lambda gt11 library: yk230e11, |
| | yk313f12, yk646g2, yk671e8, |
| | yk674c9, yk80b7; Kohara Sugano L2 |
| | larvae cap-selected library: |
| | yk1730e2” |
| Protein | 1 . . . 444 |
| | /product = “tubulin, Beta (49.8 kD) |
| | (tbb-4)” |
| Region | 45 . . . 244 |
| | /region_name = “[Pfam/InterPro de- |
| | scription] Tubulin/FtsZ protein” |
| | /db_xref = “CDD: pfam00091” |
| Region | 246 . . . 383 |
| | /region_name = “[Pfam/InterPro de- |
| | scription] Tubulin/FtsZ protein” |
| | /db_xref = “CDD: pfam03953” |
| Region | 402 . . . 438 |
| | /region_name = “[PSORT] coil coil |
| | domain: |
| | GMDEMEFTEAESNMNDLVSEYQQYQEATADDEGEFDE” |
| CDS | 1 . . . 444 |
| | /gene = “tbb-4” |
| | /locus_tag = “XK54” |
| | /coded_by = “NM_077184.1: |
| | 1 . . . 1335” |
| | /db_xref = “AceView/WormGenes: tbb-4” |
| | /db_xref = “GeneID: 181170” |
| | /db_xref = “LocusID: 181170“ |
| | /db_xref = “WormBase: B0272.1” |

| | |
|---|---|
| ORIGIN | |
| 1 | mreivhiqag qcgnqigakf wevisdehgi |
| | dptgayngds dlqlerinvy yneasggkyv |
| 61 | praclvdlep qtmdsvragp fgqlfrpdnf |
| | vfgqsgagnn wakghytega elvdnvldvv |
| 121 | rkeaescdcl qgfqmthslg ggtgsgmgtl |
| | liskireeyp drimmtfsvv pspkvsdtvv |
| 181 | epynatlsvh qlventdetf cidnealydi |
| | cfrtlklttp tygdlnhlvs mtmsgvttcl |
| 241 | rfpgqlnadl rklavnmvpf prlhffmpgf |
| | apltsrgsqq yrsltvpelt qqmfdaknmm |
| 301 | aacdprhgry ltvaamfrgr msmkevdegm |
| | lnvqnknssy fvewipnnvk tavcdipprg |
| 361 | vkmaatfvgn staiqelfkr iseqftamfr |
| | rkaflhwytg egmdemefte aesnmndlvs |
| 421 | eyqqyqeata ddegefdehd qdve |
| // | |

IIIX. RPS-14

| | |
|---|---|
| LOCUS | NP_498572 152 aa linear INV 21- |
| NOVEMBER | |
| 2003 | |
| DEFINITION | ribosomal Protein, Small subunit (16.2 |
| | kD) (rps-14) [Caenorhabditis elegans]. |
| ACCESSION | NP_498572 |
| VERSION | NP_498572.1 GI: 17554776 |
| DBSOURCE | REFSEQ: accession NM 066171.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 152) |

| | |
|---|---|
| AUTHORS | Kamath,R. S., Fraser,A. G., Dong,Y., |
| | Poulin,G., Durbin,R., Gotta, M. |
| | Kanapin,A., Le Bot,N., Moreno,S., |
| | Sohrmann,M., Welchman,D. P., |
| | Zipperlen,P. and Ahringer,J. |
| TITLE | Systematic functional analysis of the |
| | Caenorhabditis elegans genome using |
| | RNAi |
| JOURNAL | Nature 421 (6920), 231-237 (2003) |
| MEDLINE | 22417569 |
| PUBMED | 12529635 |

| | |
|---|---|
| REFERENCE | 2 (residues 1 to 152) |

| | |
|---|---|
| AUTHORS | Gonczy,P., Echeverri,C., Oegema,K., |
| | Coulson,A., Jones,S. J., Copley,R. R., |
| | Duperon,J., Oegema,J., Brehm,M., |
| | Cassin,E., Hannak,E., Kirkham,M., |
| | Pichler,S., Flohrs,K., Goessen,A., |
| | Leidel,S., Alleaume,A. M., Martin,C., |
| | Ozlu,N., Bork,P. and Hyman,A. A. |
| TITLE | Functional genomic analysis of cell |
| | division in C. elegans using RNAi of |
| | genes on chromosome III |
| JOURNAL | Nature 408 (6810), 331-336 (2000) |
| MEDLINE | 20548710 |
| PUBMED | 11099034 |

| | |
|---|---|
| COMMENT | VALIDATED REFSEQ: This record has un- |
| | dergone preliminary review of the se- |
| | quence, but has not yet been subject to |
| | final review. This record is derived |
| | from an annotated genomic sequence |
| | (NC_003281). The reference sequence was |
| | derived from WormBase CDS: F37C12.9. |
| | Summary: This essential gene rps-14, |
| | also known as F37C12.9, 3I268 or |
| | YK9313, maps at (III; −0.77). Pheno- |
| | types and affected processes are ab- |
| | normal cytoplasmic appearance, em- |
| | bryonic lethal, sterile adult, un- |
| | healthy, abnormal pseudocleavage. It |
| | encodes a ribosomal Protein, Small sub- |
| | unit. The product would be involved in |
| | pseudocleavage (sensu Nematoda). Ac- |
| | cording to the Worm Transcriptome |
| | Project, it is expressed at very high |
| | level at all stages of development |
| | [Kohara cDNAs]. Its sequence is fully |
| | supported by 144 cDNA clones. |
| | RNA interference results |
| | [T. Hyman; 2000] All embryos dead. DIC |
| | phenotype -- Multiple female pronuclei; |
| | irregular cytoplasmic appearance; |
| | karyomeres in daughter blastomeres; |
| | nuclei in AB are off-center for a |
| | while, nuclei in P1 stay close to |
| | posterior cortex for a while. Phenotype |
| | comment -- Semi-sterile. Phenotype con- |
| | firmed with independent dsRNA |
| | (F37C12.9-RNA2; similar phenotype) (by |
| | injecting genomic PCR product TH: |
| | 330a9). |
| | Same description as TH: 330a9 (by in- |
| | jecting genomic PCR product TH: 340d4). |
| | [J. Ahringer 2003] Sterile, sick (by |
| | feeding genomic PCR product JA: |
| | F37C12.9). |
| | Function |
| | Protein properties: Orthologous to |
| | yeast (S.cerevisiae) ribosomal protein |
| | rps14 using blastP. |
| | Expression |
| | The expression profile for the gene, |
| | derived from the proportion of animals |
| | at each stage in each Kohara library |
| | is: embryos 23%, L1 or L2 larvae 49%, |
| | L3 to adult 27%. |
| | In situ hybridisation pictures to all |
| | stages of development are available |
| | from Kohara NextDB. |
| | The CDS has 3 exons. It covers 0.55 kb |
| | on the WS97 genome. The protein (152 |
| | aa, 16.2 kDa, pI 10.4) contains one |
| | ribosomal protein S11 motif. It is pre- |
| | dicted to localise in the cytoplasm |
| | [Psort2]. Taxblast (threshold 10\hat{ }-3) |
| | tracks ancestors down to archaea and |
| | bacteria and eukaryota. |
| | COMPLETENESS: full length. |
| | Method: conceptual translation. |

| | |
|---|---|
| FEATURES | Location/Qualifiers |

| | |
|---|---|
| source | 1 . . . 152 |
| | /organism = “Caenorhabditis elegans” |
| | /db_xref = “taxon: 6239” |
| | /chromosome = “III” |
| | /map = “III; −0.77 cM (interpolated |
| | genetic position)” |
| | /map = “III; covering 615 bp, from |
| | base 7179511 to 7178897 on genome |
| | release WS97” |
| Protein | 1 . . . 152 |
| | /product = “ribosomal Protein, Small |
| | subunit (16.2 kD) (rps-14)” |
| Region | 3 . . . 9 |
| | /region_name = “[PSORT] nuclear lo- |
| | calization domain: PARKGKA” |
| Region | 30 . . . 148 |
| | /region_name = “[Pfam/Interpro de- |
| | scription] ribosomal protein S11” |
| | /db_xref = “CDD: pfam00411” |
| CDS | 1 . . . 152 |
| | /gene = “rps-14” |
| | /locus_tag = “31268” |
| | /coded_by = “NM_066171.1: |
| | 1 . . . 459” |
| | /db_xref = “AceView/WormGenes: |
| | rps-14” |
| | /db_xref = “GeneID: 176006” |
| | /db_xref = “LocusID: 176006” |
| | /db_xref = “WormBase: F37C12.9” |

| | |
|---|---|
| ORIGIN | |
| 1 | maparkgkak eeqavvslgp qakegelifg |
| | vahifasfnd tfvhitdisg retivrvtgg |
| 61 | mkvkadrdes spyaamlaaq dvadrckqlg |
| | inalhiklra tggtrtktpg pgaqsalral |
| 121 | aragmkigri edvtpipsdc trrkggrrgr |
| | rl |
| // | |

IIIZ. RPS-13

| | |
|---|---|
| LOCUS | NP_498393 151 aa linear INV 21- |
| NOVEMBER | |
| 2003 | |
| DEFINITION | ribosomal Protein, Small subunit (17.3 |
| | kD) (rps-13) [Caenorhabditis elegans]. |
| ACCESSION | NP_498393 |
| VERSION | NP_498393.1 GI: 17554774 |
| DBSOURCE | REFSEQ: accession NM 065992.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 151) |

| | |
|---|---|
| AUTHORS | Kamath,R. S., Fraser,A. G., Dong,Y., |
| | Poulin,G., Durbin,R., Gotta,M., |
| | Kanapin,A., Le Bot,N., Moreno,S., |
| | Sohrmann,M., Welchman,D. P., |
| | Zipperlen,P. and Ahringer,J. |
| TITLE | Systematic functional analysis of the |
| | Caenorhabditis elegans genome using |
| | RNAi |
| JOURNAL | Nature 421 (6920), 231-237 (2003) |
| MEDLINE | 22417569 |
| PUBMED | 12529635 |

| | |
|---|---|
| REFERENCE | 2 (residues 1 to 151) |

| | |
|---|---|
| AUTHORS | Gonczy,P., Echeverri,C., Oegema,K., |
| | Coulson,A., Jones,S. J., Copley,R. R., |
| | Duperon,J., Oegema,J., Brehm,M., |
| | Cassin,E., Hannak, E., Kirkham,M., |
| | Pichler,S., Flohrs,K., Goessen,A., |
| | Leidel,S., Alleaume,A. M., Martin,C., |
| | Ozlu,N., Bork,P. and Hyman,A. A. |
| TITLE | Functional genomic analysis of cell |
| | division in C. elegans using RNAi of |
| | genes on chromosome III |
| JOURNAL | Nature 408 (6810), 331-336 (2000) |
| MEDLINE | 20548710 |
| PUBMED | 11099034 |

| | |
|---|---|
| COMMENT | VALIDATED REFSEQ: This record has un- |
| | dergone preliminary review of the se- |
| | quence, but has not yet been subject to |
| | final review. This record is derived |
| | from an annotated genomic sequence |
| | (NC_003281). The reference sequence was |
| | derived from WormBase CDS: C16A3.9. |
| | Summary: This essential gene rps-13, |
| | also known as C16A3.9, 3H464 or YK2267, |
| | maps at (III; −1.25). Its phenotype is |
| | embryonic lethal, sterile adult, abnor- |
| | mal pseudocleavage. It encodes a ribo- |
| | somal Protein, Small subunit. The pro- |
| | duct would be involved in pseudo- |
| | cleavage (sensu Nematoda). According to |
| | the Worm Transcriptome Project, it is |
| | expressed at high level in L3, L4, |
| | adult and culminating in embryos |
| | [Kohara cDNAs]. Its sequence is fully |
| | supported by 34 cDNA clones. |
| | RNA interference results |
| | [T. Hyman; 2000] All embryos dead. DIC |
| | phenotype -- Multiple female pronuclei; |
| | irregular cytoplasmic appearance; |
| | aberrant pseudocleavage stage; karyo- |
| | meres in daughter blastomeres; nuclei |
| | in AB are off-center for a while, |
| | nuclei in P1 stay close to posterior |
| | cortex for a while (by injecting geno- |
| | mic PCR product TH: 309g1). Movies are |
| | available on Hyman's site. |
| | [J. Ahringer 2003] Sterile (by feeding |
| | genomic PCR product JA: C16A3.9). |
| | Function |
| | Protein properties: Orthologous to |
| | yeast (S.cerevisiae) ribosomal protein |
| | rps13 using blastP. |
| | Expression |
| | The expression profile for the gene, |
| | derived from the proportion of animals |
| | at each stage in each Kohara library |
| | is: embryos 81%, L1 or L2 larvae 1%, L3 |
| | to adult 17%. |
| | In situ hybridisation pictures to all |
| | stages of development are available |
| | from Kohara NextDB. |
| | The CDS has 3 exons. It covers 0.85 kb |
| | on the WS97 genome. The protein (151 |
| | aa, 17.3 kDa, pI 10.7) contains one |
| | Ribosomal protein S15 motif. It is pre- |
| | dicted to localise in the cytoplasm |
| | [Psort2]. Taxblast (threshold 10\hat{ }-3) |
| | tracks ancestors down to archaea and |
| | eukaryota. |
| | COMPLETENESS: full length. |
| | Method: conceptual translation. |

| | |
|---|---|
| FEATURES | Location/Qualifiers |

| | |
|---|---|
| source | 1 . . . 151 |
| | /organism = “Caenorhabditis elegans” |
| | /db_xref = “taxon: 6239” |
| | /chromosome = “III” |
| | /map = “III; −1.25 cM (interpolated |
| | genetic position)” |
| | /map = “III; covering 909 bp, from |
| | base 6374934 to 6374026 on genome |
| | release WS97” |
| | /clone_lib = “Kohara embryonic |
| | lambda gt11 library: yk74d8, yk96e4, |
| | yk139e1, yk141e12, yk196b12, |
| | yk269e10, yk319c12, yk329h11, |
| | yk332c6, yk390g10, yk418c7, yk418e3, |
| | yk432h1, yk433b12, yk436g3, yk467c6, |
| | yk474h7, yk479b1, yk502h5, yk508h12, |
| | yk533f12, yk538g7, yk572a6, yk623c2, |
| | yk628b6, yk631e5, yk641b4, yk641h7, |
| | yk666c1, yk668a5, yk627h1; mixed |
| | stage, Stratagene library |
| | [PMID1302005]: CEMSA36, CEMSH68; |
| | Kohara mixed stage library, from |
| | him-8 strain, containing 15-30% |
| | males: yk304b4” |
| Protein | 1 . . . 151 |
| | /product = “ribosomal Protein, Small |
| | subunit (17.3 kD) (rps-13)” |
| Region | 61 . . . 151 |
| | /region name = “[Pfam/InterPro de- |
| | scription] ribosomal protein S15” |
| | /db_xref = “CDD: pfam00312” |
| CDS | 1 . . . 151 |
| | /gene = “rps-13” |
| | /locus_tag = “3H464” |
| | /coded_by = “NM_065992.1: |
| | 1 . . . 456” |
| | /db_xref = “AceView/WormGenes: |
| | rps-13” |
| | /db_xref = “GeneID: 175901” |
| | /db_xref = “LocusID: 175901” |
| | /db_xref = “WormBase: C16A3.9” |

| | |
|---|---|
| ORIGIN | |
| 1 | mgrmhnpqkg maksaipyrr svpswqkmta |
| | eevqdqivkm akkglrpsqi gvilrdshgv |
| 61 | gqvrrlagnk ifrilkskgm apelpedlyh |
| | lvkkavairk hlersrkdid skyrlilves |
| 121 | rihrlaryyk tkrqlpptwk yesgtaaslv |
| | s |
| // | |

IIIAA. RPL-24

| | |
|---|---|
| LOCUS | NP_491399 159 aa linear INV 21- |
| NOVEMBER | |
| 2003 | |
| DEFINITION | ribosomal Protein, Large subunit (17.8 |
| | kD) (rpl-24.1) [Caenorhabditis elegans]. |
| ACCESSION | NP_491399 |
| VERSION | NP_491399.1 GI: 17506331 |
| DESOURCE | REFSEQ: accession NM 058998.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 159) |

| | |
|---|---|
| AUTHORS | Fraser,A. G., Kamath,R. S., |
| | Zipperlen,P., Martinez-Campos,M., |
| | Sohrmann,M. and Ahringer,J. |
| TITLE | Functional genomic analysis of C. |
| | elegans chromosome I by systematic RNA |
| | interference |
| JOURNAL | Nature 408 (6810), 325-330 (2000) |
| MEDLINE | 20548709 |
| PUBMED | 11099033 |

| | |
|---|---|
| REFERENCE | 2 (residues 1 to 159) |

| | |
|---|---|
| AUTHORS | Walhout,A. J., Sordella,R., Lu,X., |
| | Hartley,J. L., Temple,G. F., |
| | Brasch,M. A., Thierry-Mieg,N. and |
| | Vidal,M. |
| TITLE | Protein interaction mapping in C. |
| | elegans using proteins involved in |
| | vulval development |
| JOURNAL | Science 287 (5450), 116-122 (2000) |
| MEDLINE | 20082953 |
| PUBMED | 10615043 |

| | |
|---|---|
| COMMENT | VALIDATED REFSEQ: This record has un- |
| | dergone preliminary review of the se- |
| | quence, but has not yet been subject to |
| | final review. This record is derived |
| | from an annotated genomic sequence |
| | (NC_003279). The reference sequence was |
| | derived from WormBase CDS: D1007.12. |
| | Summary: This essential gene rpl-24.1, |
| | also known as D1007.12, 1F153 or |
| | YK1971, maps at (I; −1.08). Its pheno- |
| | type is embryonic lethal, sterile |
| | adult. It encodes a ribosomal Protein, |
| | Large subunit. From Pfam homology, the |
| | product would be involved in protein |
| | biosynthesis and would localize in |
| | intracellular, ribosome. |
| | According to the Worm Transcriptome |
| | Project, it is expressed at very high |
| | level at all stages of development |
| | [Kohara cDNAs]. Its sequence is fully |
| | supported by 124 cDNA clones. |
| | RNA interference results |
| | [J. Ahringer 2000] embryonic lethal |
| | (100%), larval arrest, sterile (ma- |
| | ternal brood size 1 to 5) (by feeding |
| | genomic PCR product JA: D1007.12). |
| | Expression |
| | The expression profile for the gene, |
| | derived from the proportion of animals |
| | at each stage in each Kohara library |
| | is: embryos 24%, L1 or L2 larvae 44%, |
| | L3 to adult 31%. |
| | In situ hybridisation pictures to all |
| | stages of development are available |
| | from Kohara NextDB. |
| | Interactions |
| | The protein encoded by this gene inter- |
| | acts with: protein LIN-15A: [Vidal M, |
| | pm10615043] interaction seen in a 2- |
| | hybrid screen, with lin-15a as bait. |
| | The CDS has 4 exons. It covers 1.00 kb |
| | on the WS97 genome. The protein (159 |
| | aa, 17.8 kDa, pI 11.3) contains one |
| | Ribosomal protein L24E motif. It also |
| | contains a coil coil stretch, an ER |
| | membrane domain [Psort2]. It is pre- |
| | dicted to localise in the nucleus |
| | [Psort2]. Taxblast (threshold 10\hat{ }-3) |
| | tracks ancestors down to archaea and |
| | eukaryota. |
| | COMPLETENESS: full length. |
| | Method: conceptual translation. |

| | |
|---|---|
| FEATURES | Location/Qualifiers |

| | |
|---|---|
| source | 1 . . . 159 |
| | /organism = “Caenorhabditis elegans” |
| | /db_xref = “taxon: 6239” |
| | /chromosome-“I” |
| | /map = “I; −1.08 cM (interpolated |
| | genetic position)” |
| | /map = “I; covering 1100 bp, from |
| | base 4585116 to 4586217 on genome |
| | release WS97” |
| | /clone_lib = “Kohara embryonic |
| | lambda gt11 library: yk62c11, |
| | yk63a1, yk74c6, yk78g2, yk79a4, |
| | yk79g4, yk81c9, yk83e5, yk89b7, |
| | yk96d9, yk103d8, yk138g11, yk172a2, |
| | yk210c12, yk216g11, yk325h1, |
| | yk375g5, yk401h10, yk424d6, yk449h5, |
| | yk458b1, yk469h3, yk473a2, yk476f10, |
| | yk479b3, yk483f11, yk486g10, |
| | yk489g9, yk502b6, yk533d12, |
| | yk602a11, yk606e6, yk667g3, yk505c9, |
| | yk175d3, yk460a5; Kohara Sugano L1 |
| | larvae cap-selected library: |
| | yk771e9, yk796g4, yk831f10, |
| | yk1104c7, yk1310a12, yk874a12, |
| | yk877b6, yk878b6, yk1006c9, |
| | yk1072h3, yk1087d2, yk1098e12, |
| | yk1129g2, yk1149e2, yk1165b8, |
| | yk1166d2, yk1168d9, yk1181f2, |
| | yk1193a10, yk1204f1, yk1219a9, |
| | yk1235a12, yk1272d7, yk1298e3, |
| | yk1320g11, yk1352a8, yk890c5, |
| | yk1081e12, yk1067g3; Kohara Sugano |
| | L2 larvae cap-selected library: |
| | yk818c3, yk775h12, yk810g8, |
| | yk816b10, yk1377d10, yk1386h10, |
| | yk1407b1, yk1418d5, yk1579d7, |
| | yk1583a8, yk1590g3, yk1600a2, |
| | yk1608h5, yk1670f3, yk1365h1, |
| | yk1381b7, yk1386b10, yk1390d12, |
| | yk1401g11, yk1420a1, yk1510f8, |
| | yk1517c6, yk1518f1, yk1578b5, |
| | yk1581a9, yk1587g3, yk1592d5, |
| | yk1610e10, yk1638c2, yk1667a11, |
| | yk1699g11, yk1719h1, yk1720e12, |
| | yk1722a8, yk1742e12, yk1756f2, |
| | yk1493f7, yk1360e6; Kohara Sugano L4 |
| | larvae cap-selected library: |
| | yk1437a7, yk1541g9, yk1685h11; |
| | Kohara Sugano mixed stage cap- |
| | selected library: yk732a10; mixed |
| | stage, Stratagene library |
| | [PMID1302005]: CEMSC16; Kohara mixed |
| | stage library, from him-8 strain, |
| | containing 15-30% males: yk70e3, |
| | yk71b8, yk71g7, yk99e8, yk170h5, |
| | yk206h1, yk361a7, yk379c9, yk545f5, |
| | yk547e1, yk557f9, yk545f11; Marc |
| | Vidal 2 hybrid library: mv508, |
| | mv1325, mv1525, mv1326” |
| Protein | 1 . . . 159 |
| | /product = “ribosomal Protein, Large |
| | subunit (17.8 kD) (rpl-24.1)” |
| Region | 1 . . . 71 |
| | /region_name = “[Pfam/InterPro de- |
| | scription] ribosomal protein L24E” |
| | /db_xref = “CDD: pfam01246” |
| Region | 60 . . . 76 |
| | /region_name = “[PSORT] nuclear |
| | localization domain: |
| | KKGTHGQEQVTRKKTKK” |
| Region | 104 . . . 136 |
| | /region_name = “[PSORT] coil coil |
| | domain: |
| | RRQQREQAAKIAKDANKAVRAAKAAANKEKKAS" |
| Region | 155 . . . 158 |
| | /region_name = “[PSORT] ER membrane |
| | domain: VGGK” |
| CDS | 1 . . . 159 |
| | /gene = “rpl-24.1” |
| | /locus_tag = “1F153” |
| | /coded_by = “NM_058998.1: |
| | 1 . . . 480” |
| | /db_xref = “AceView/WormGenes: |
| | rpl-24.1” |
| | /db_xref = “GeneID: 172062” |
| | /db_xref = “LocusID: 172062” |
| | /db_xref = “WormBase: D1007.12” |

| | |
|---|---|
| ORIGIN | |
| 1 | mkvetcvysg ykihpghgkr lvrtdgkvqi |
| | flsgkalkga klrrnprdir wtvlyriknk |
| 61 | kgthgqeqvt rkktkksvqv vnravaglsl |
| | dailakrnqt edfrrqqreq aakiakdank |
| 121 | avraakaaan kekkasqpkt qqktaknvkt |
| | aaprvggkr |
| // | |
| LOCUS | NP_492572 162 aa linear INV 21- |
| NOVEMBER | |
| 2003 | |
| DEFINITION | ribosomal Protein, Large subunit (18.8 |
| | kD) (rpl-24.2) [Caenorhabditis |
| | elegans]. |
| ACCESSION | NP_492572 |
| VERSION | NP_492572.1 GI: 17505458 |
| DBSOURCE | REFSEQ: accession NM 060171.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 162) |

| | |
|---|---|
| AUTHORS | Fraser,A. G., Kamath,R. S., |
| | Zipperlen,P., Martinez-Campos,M., |
| | Sohrmann,M. and Ahringer,J. |
| TITLE | Functional genomic analysis of C. |
| | elegans chromosome I by systematic RNA |
| | interference |
| JOURNAL | Nature 408 (6810), 325-330 (2000) |
| MEDLINE | 20548709 |
| PUBMED | 11099033 |

| | |
|---|---|
| COMMENT | VALIDATED REFSEQ: This record has un- |
| | dergone preliminary review of the se- |
| | quence, but has not yet been subject to |
| | final review. This record is derived |
| | from an annotated genomic sequence |
| | (NC_003279). |
| | The reference sequence was derived from |
| | WormBase CDS: C03D6.8. |
| | Summary: This gene rpl-24.2, also known |
| | as C03D6.8, 1K245 or YK5780, maps at |
| | (I; +3.90). It encodes a ribosomal |
| | Protein, Large subunit. From Pfam |
| | homology, the product would be involved |
| | in protein biosynthesis and would lo- |
| | calize in intracellular, ribosome. |
| | According to the Worm Transcriptome |
| | Project, it is expressed at high level |
| | at all stages of development [Kohara |
| | cDNAs]. Its sequence is fully supported |
| | by 22 cDNA clones. |
| | RNA interference results |
| | [J. Ahringer 2000] slow growth (by |
| | feeding genomic PCR product JA: |
| | C03D6.1). Warning: this double stranded |
| | RNA may also interfere with gene 1K244. |
| | [J. Ahringer 2000] slow growth (by |
| | feeding genomic PCR product JA: |
| | C03D6.8). Warning: this double stranded |
| | RNA may also interfere with gene 1K244. |
| | Expression |
| | The expression profile for the gene, |
| | derived from the proportion of animals |
| | at each stage in each Kohara library |
| | is: embryos 18%, L1 or L2 larvae 61%, |
| | L3 to adult 22%. |
| | In situ hybridisation pictures to all |
| | stages of development are available |
| | from Kohara NextDB. |
| | The CDS has 3 exons. It covers 0.59 kb |
| | on the WS97 genome. The protein (162 |
| | aa, 18.8 kDa, pI 10.6) contains one |
| | Ribosomal protein L24E motif. It is |
| | predicted to localise in the nucleus |
| | [Psort2]. Taxblast (threshold 10\hat{ }-3) |
| | tracks ancestors down to archaea and |
| | eukaryota. |
| | COMPLETENESS: full length. |
| | Method: conceptual translation. |

| | |
|---|---|
| FEATURES | Location/Qualifiers |

| | |
|---|---|
| source | 1 . . . 162 |
| | /organism = “Caenorhabditis elegans” |
| | /db_xrefr = “taxon: 6239” |
| | /chromosome = “I” |
| | /map = “I; +3.90 cM (interpolated |
| | genetic position)” |
| | /map = “I; covering 698 bp, from |
| | base 9677465 to 9678164 on genome |
| | release WS97” |
| | /clone_lib = “Kohara embryonic |
| | lambda gt11 library: yk331a1, |
| | yk512c8, yk663g11, yk176h7; Kohara |
| | Sugano L1 larvae cap-selected |
| | library: yk753c12, yk772h12, |
| | yk900d1, yk1127c1, yk1299f7, |
| | yk1304b7, yk1057e1, yk1255f6, |
| | yk1214c9, yk1159g10, yk1291g4, |
| | yk871c5; Kohara Sugano L2 larvae |
| | cap-selected library: yk1527g1, |
| | yk1569d5, yk1605a7, yk1668g2; Kohara |
| | mixed stage library, from him-8 |
| | strain, containing 15-30% males: |
| | yk361d3, yk582d11” |
| Protein | 1 . . . 162 |
| | /product = “ribosomal Protein, Large |
| | subunit (18.8 kD) |
| | (rpl-24.2)” |
| Region | 1 . . . 71 |
| | /region_name = “[Pfam/InterPro de- |
| | scription] ribosomal protein L24E” |
| | /db_xref = “CDD: pfam01246” |
| Region | 41 . . . 57 |
| | /region_name = “[PSORT] nuclear lo- |
| | calization domain: |
| | KKKKNPRKLRFTKAARR” |
| Region | 43 . . . 59 |
| | /region_name = “[PSORT] nuclear lo- |
| | calization domain: |
| | KKNPRKLRFTKAARRAR” |
| CDS | 1 . . . 162 |
| | /gene = “rpl-24.2” |
| | /locus_tag = “1K245” |
| | /coded_by = “NM_060171.1: |
| | 1 . . . 489” |
| | /db_xref = “AceView/WormGenes: |
| | rpl-24.2” |
| | /db_xref = “GeneID: 172815” |
| | /db_xref = “LocusID: 172815” |
| | /db_xref = “WormBase: C03D6.8” |

| | |
|---|---|
| ORIGIN | |
| 1 | mriekcyfcs spiypghgiq fvrndstvfk |
| | fcrsrcnklf kkkknprklr ftkaarrarg |
| 61 | kelindatql leqrrdepvk yeramfqkti |
| | eaaktisalk tkrygnhlrk rlqpgkivqk |
| 121 | kgllakvdkk mhlirapvan rdgvktraaa |
| | kekktaesme tn |
| // | |

IIIBB. RPS-11

| | |
|---|---|
| LOCUS | NP_502186 155 aa linear INV 21- |
| NOVEMBER | |
| 2003 | |
| DEFINITION | ribosomal Protein, Small subunit (17.7 |
| | kD) (rps-11) [Caenorhabditis elegans]. |
| ACCESSION | NP_502186 |
| VERSION | NP_502186.1 GI: 17542016 |
| DBSOURCE | REFSEQ: accession NM 069785.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 155) |

| | |
|---|---|
| AUTHORS | Kamath,R. S., Fraser,A. G., Dong,Y., |
| | Poulin,G., Durbin,R., Gotta,M., |
| | Kanapin,A., Le Bot,N., Moreno,S., |
| | Sohrmann,M., Welchman,D. P., |
| | Zipperlen,P. and Ahringer,J. |
| TITLE | Systematic functional analysis of the |
| | Caenorhabditis elegans genome using |
| | RNAi |
| JOURNAL | Nature 421 (6920), 231-237 (2003) |
| MEDLINE | 22417569 |
| PUBMED | 12529635 |

| | |
|---|---|
| REFERENCE | 2 (residues 1 to 155) |

| | |
|---|---|
| AUTHORS | Piano,F., Schetter,A. J., Mangone,M., |
| | Stein,L. and Kemphues, K. J. |
| TITLE | RNAi analysis of genes expressed in the |
| | ovary of Caenorhabditis elegans |
| JOURNAL | Curr. Biol. 10 (24), 1619-1622 (2000) |
| MEDLINE | 21065924 |
| PUBMED | 11137018 |

| | |
|---|---|
| COMMENT | VALIDATED REFSEQ: This record has un- |
| | dergone preliminary review of the se- |
| | quence, but has not yet been subject to |
| | final review. This record is derived |
| | from an annotated genomic sequence |
| | (NC_003282). The reference sequence was |
| | derived from WormBase CDS: F40F11.1. |
| | Summary: This essential gene rps-11, |
| | also known as F40F11.1, 4M367 or |
| | YK2226, maps at (IV; +5.45). Its pheno- |
| | type is sterile adult, unhealthy, |
| | catastrophic one cell arrest. It |
| | encodes a ribosomal Protein, Small sub- |
| | unit. From Pfam homology, the product |
| | would be involved in protein biosyn- |
| | thesis and would localize in intra- |
| | cellular, ribosome. |
| | According to the Worm Transcriptome |
| | Project, it is expressed at very high |
| | level at all stages of development |
| | [Kohara cDNAs]. Its sequence is |
| | fully supported by 87 cDNA clones. |
| | RNA interference results |
| | [F. Piano 2000] Embryonic lethal; egg |
| | production ceases in injected animal; |
| | catastrophic one-cell arrest (by in- |
| | jecting cDNA clone FP: SP13H3). |
| | [J. Ahringer 2003] Sterile, sick (by |
| | feeding genomic PCR product JA: |
| | F40F11.1). |
| | Function |
| | Protein properties: Orthologous to |
| | yeast (S.cerevisiae) ribosomal protein |
| | rps11 using blastP. |
| | Expression |
| | The expression profile for the gene, |
| | derived from the proportion of animals |
| | at each stage in each Kohara library |
| | is: embryos 27%, L1 or L2 larvae 44%, |
| | L3 to adult 29%. |
| | In situ hybridisation pictures to all |
| | stages of development are available |
| | from Kohara NextDB. |
| | Pattern in ovary [F Piano, 2000]. |
| | The CDS has 3 exons. It covers 0.57 kb |
| | on the WS97 genome. The protein (155 |
| | aa, 17.7 kDa, pI 10.5) contains one |
| | Ribosomal protein S17 motif. It also |
| | contains a peroxisomal domain, an ER |
| | membrane domain [Psort2]. It is pre- |
| | dicted to localise in the cytoplasm |
| | [Psort2]. Taxblast (threshold 10\hat{12}-3) |
| | tracks ancestors down to archaea and |
| | bacteria and eukaryota. |
| | COMPLETENESS: full length. |
| | Method: conceptual translation. |

| | |
|---|---|
| FEATURES | Location/Qualifiers |

| | |
|---|---|
| source | 1 . . . 155 |
| | /organism = “Caenorhabditis elegans” |
| | /db_xref = “taxon: 6239” |
| | /chromosome = “IV” |
| | /map = “IV; +5.45 cM (interpolated |
| | genetic position)” |
| | /map = “IV; covering 651 bp, from |
| | base 11602617 to 11603269 on genome |
| | release WS97” |
| | /clone_lib = “Kohara embryonic |
| | lambda gt11 library: yk67b1, yk89c6, |
| | yk106b9, yk138a8, yk172e3, yk173e4, |
| | yk258c6, yk258d2, yk273e4, yk290h1, |
| | yk327b12, yk400d7, yk468h9, yk471b5, |
| | yk477h6, yk485b4, yk500e2, yk521e9, |
| | yk572f9, yk616b11, yk629c3, |
| | yk639h12, yk644g7, yk646g7, yk647f7, |
| | yk681e7, yk325e1, yk678h4; Kohara |
| | Sugano L1 larvae cap-selected li- |
| | brary: yk752e4, yk759c3, yk1292a10, |
| | yk753e4, yk1019c9, yk883b2, yk892b7, |
| | yk898a5, yk1011f1, yk1028c1, |
| | yk1106c9, yk1304c12, yk1326e8, |
| | yk1356h8, yk1207c9, yk871f6, |
| | yk1169b3, yk1298a10, yk1246f10; |
| | Kohara Sugano L2 larvae cap-selected |
| | library: yk778e12, yk1636d2, |
| | yk1593e1, yk1639a9, yk1576e5, |
| | yk1674h10, yk1691b5, yk1359c2, |
| | yk1414d7, yk1417e8, yk1417f11, |
| | yk1418f7, yk1489g4, yk1520d6, |
| | yk1521a3, yk1531b12, yk1567h6, |
| | yk1572a9, yk1577g10, yk1639h8, |
| | yk1650d4, yk1660f1, yk1671f12, |
| | yk1718h2, yk1727a4, yk1741b8, |
| | yk1706a1, yk1750b7; Kohara Sugano L4 |
| | larvae cap-selected library: |
| | yk785e9, yk834a6, yk1439e1, |
| | yk1545a1, yk1555g5, yk1442b4, |
| | yk1552h12; Kohara mixed stage li- |
| | brary, from him-8 strain, con- |
| | taining 15-30% males: yk145g6, |
| | yk205e11, yk361b5, yk380g2; Piano |
| | ovary library: BE228125” |
| Protein | 1 . . . 155 |
| | /product = “ribosomal Protein, Small |
| | subunit (17.7 kD) (rps-11)” |
| Region | 72 . . . 142 |
| | /region_name = “[Pfam/InterPro de- |
| | scription] ribosomal protein S17” |
| | /db_xref = “CDD: pfam00366” |
| Region | 86 . . . 102 |
| | /region_name = “[PSORT] nuclear lo- |
| | calization domain: |
| | RRDYLHYIKKYRRYEKR” |
| Region | 101 . . . 104 |
| | /region_name = “[PSORT] nuclear lo- |
| | calization domain: KRHK” |
| Region | 151 . . . 154 |
| | /region_name = “[PSORT] ER membrane |
| | domain: GFSK” |
| Region | 153 . . . 155 |
| | /region_name = “[PSORT] peroxisomal |
| | domain: SKF” |
| CDS | 1 . . . 155 |
| | /gene = “rps-11” |
| | /locus_tag = “4M367” |
| | /coded_by = “NM_069785.1: |
| | 1 . . . 468” |
| | /db_xref = “AceView/WormGenes: |
| | rps-11” |
| | /db_xref = “GeneID: 178083” |
| | /db_xref = “LocusID: 178083” |
| | /db_xref = “WormBase: P40F11.1” |

| | |
|---|---|
| ORIGIN | |
| 1 | mseqterafl kqptvnlnnk arilagskkt |
| | pryirevglg fkaprdaveg tyidkkcpwa |
| 61 | gnvpirgmil tgvvlknkmt rtivvrrdyl |
| | hyikkyrrye krhknvpahc spafrdihpg |
| 121 | dlvtiqecrp lsktvrfnvl kvnksgtskk |
| | gfskf |
| // | |

IIICC. Agglutinin

MTTVRKTYRFCVFSSCLSVSCALVTQVHSSSLPIYSSPFVEKVFLHSSIY
VRLCGDMYEQWPTLEFSDLNSSILDLFTKATSQSVASSLLYELTRSDADE
NGGSIRLNNEEHLKWCMQVLNHSLTLSFATSREYETLKGAVRIYLHWLRA
LCDTPDNNIPTPLLATPEKYFRNIIDALRWIFCRREDDFDTTVGGQVPRG
LAIERQSIEIDMVLDSLKYLTRNSSRKYQDEVWARSISFLLNSSDILLSE
PNATEEMGTRTCVRVADTLFDMWLNAVLNEHIPSLTYWSSLATLARRWRH
NVPIIECWAKKILGLSTLVCRKMYGDDFLKIDIVDESVLPFENVPMTAEE
DENEVHLLYRTWFNMLCLFDSPAKILNHDATRNLCLNGNSPRRTTSSISM
SNFELASSSAAQGVSFFLAAVTLQRMVDLFYGDSRVKIDLRNYPVPDGKT
APNTRTASVLTDNHSHHTNRTSSTTGDSSRYVSLGGAVGQIIVDDHQVSM
SSGSTASGKTSTATGTSSTHTISSEIRRDQRIMSVNDRSRDPSHRTVSVT
DSVNISNQSRYSEQTSSTLTYKSAPIPETANENGHGESISQLVSNSTVSA
PVGGAGNDLTLKAGVHPSEMKIGRSSGVIGSAQHNNFYADTTSPYRSAQR
FVTNFLTANQATMPYVGGKRPKTDRMLNLVGDWLFAIVNSPTNSPRVTGN
DHSGHHKKNNDGVSDVSFISHHFVFTLLSAITTEVISIYICVSMISLTGL
NKHHLRIGIIDDETVCTSECPFSPFFAKFTITDGVDFLNNEADSKTTPTS
FDFDDFDSFHKFRFQHIYTSK

Also see the C. elegans Protein Database: Wormpep at http://www.sanger.ac.uk/Projects/C_{—}elegans/wormpep/; Accesion No. ce03050.

IIIDD. SIP-1 (hsp20)

Member of the Stress Induced Protein gene class.
MSSLCPYTGR PTGLFRDFED MMPYWAQRHS MLNNFNNIVP
QQLNEVENTA QKFCVKLDVA
AFKPEELKVN LEGHVLTIEG HHEVKTEHGF SKRSFTRQFT
LPKDVDLAHI HTVINKEGQM
TIDAPKTGSN TTVRALPIHT SAGHAVTQKP SSTTTTGKH

Homologs include, for example, Swiss-Prot. Accession No. P02511 , H. sapiens Alpha crystallin B chain.

IIIEE. CCT-6 (chaperonin)

| | |
|---|---|
| LOCUS | NP_741153 539 aa linear INV 21- |
| NOVEMBER | |
| 2003 | |
| DEFINITION | chaperonin Containing TCP-1 (58.9 kD) |
| | (cct-6) [Caenorhabditis elegans]. |
| ACCESSION | NP_741153 |
| VERSION | NP_741153.1 GI: 25144678 |
| DBSOURCE | REFSEQ: accession NM 171135.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 539) |

| | |
|---|---|
| AUTHORS | Kamath,R. S., Fraser,A. G., Dong,Y., |
| | Poulin,G., Durbin,R., Gotta,M., |
| | Kanapin,A., Le Bot,N., Moreno,S., |
| | Sohrmann,M., Welchman,D. P., |
| | Zipperlen,P. and Ahringer,J. |
| TITLE | Systematic functional analysis of the |
| | Caenorhabditis elegans genome using |
| | RNAi |
| JOURNAL | Nature 421 (6920), 231-237 (2003) |
| MEDLINE | 22417569 |
| PUBMED | 12529635 |

| | |
|---|---|
| REFERENCE | 2 (residues 1 to 539) |

| | |
|---|---|
| AUTHORS | Gonczy,P., Echeverri,C., Oegema,K., |
| | Coulson,A., Jones,S. J., Copley,R. R., |
| | Duperon,J., Oegema,J., Brehm,M., |
| | Cassin,E., Hannak, E., Kirkham,M., |
| | Pichler,S., Flohrs,K., Goessen,A., |
| | Leidel,S., Alleaume,A. M., Martin,C., |
| | Ozlu,N., Bork,P. and Hyman,A. A. |
| TITLE | Functional genomic analysis of cell di- |
| | vision in C. elegans using RNAi of |
| | genes on chromosome III |
| JOURNAL | Nature 408 (6810), 331-336 (2000) |
| MEDLINE | 20548710 |
| PUBMED | 11099034 |

| | |
|---|---|
| REFERENCE | 3 (residues 1 to 539) |

| | |
|---|---|
| AUTHORS | Leroux,M. R. and Candido,E. P. |
| TITLE | Characterization of four new tcp-1- |
| | related cct genes from the nematode |
| | Caenorhabditis elegans |
| JOURNAL | DNA Cell Biol. 14 (11), 951-960 (1995) |
| MEDLINE | 96069542 |
| PUBMED | 7576182 |

| | |
|---|---|
| COMMENT | VALIDATED REFSEQ: This record has un- |
| | dergone preliminary review of the se- |
| | quence, but has not yet been subject to |
| | final review. This record is derived |
| | from an annotated genomic sequence |
| | (NC_003281). The reference sequence was |
| | derived from WormBase CDS: F01F1.8a. |
| | Summary: This essential gene cct-6, |
| | also known as F01F1.8, 3G944 or YK828, |
| | maps at (III; −1.53). Phenotypes and |
| | affected processes are embryonic |
| | lethal, sterile adult, unhealthy, |
| | clear, translucent appearance, pro- |
| | truding vulva, small embryos, slow |
| | embryonic cell division, cytokinesis |
| | defect, abnormal cytoplasmic appear- |
| | ance. It encodes a chaperonin Contain- |
| | ing TCP-1. |
| | According to the Worm Transcriptome |
| | Project, it is expressed at very high |
| | level at all stages of development |
| | [Kohara cDNAs]. Its sequence is fully |
| | supported by 122 cDNA clones and pro- |
| | duces, by alternative splicing, 2 dif- |
| | ferent transcripts a, b altogether en- |
| | coding 2 different protein isoforms. |
| | RNA interference results |
| | [T. Hyman; 2000] All embryos dead. DIC |
| | phenotype -- Semi-sterile; complex DIC |
| | phenotype; many embryos loose struc- |
| | tural integrity upon dissection; areas |
| | lacking yolk granules; failure in dif- |
| | ferent microtubule-based processes |
| | (centration/rotation, spindle assembly, |
| | chromosome segregation). DIC phenotype |
| | comment -- see also results from |
| | C07G2.3. Phenotype comment -- Confirmed |
| | with independent dsRNA (F01F1.8-RNA2; |
| | similar phenotype) (by injecting |
| | genomic PCR product TH: 304C1). Movies |
| | are available on Hyman's site. |
| | Same description as TH: 304C1 (by in- |
| | jecting genomic PCR product TH: 341B5). |
| | [J. Ahringer 2003] Embryonic lethal |
| | (100%), sterile, sick, clear, pro- |
| | truding vulva (by feeding genomic PCR |
| | product JA: F01P1.8). |
| | Function |
| | Protein properties: [C.elegansII] NMK. |
| | Encodes one of 7-9 related subunits of |
| | eukaryotic cytosolic chaperonin |
| | CCT.Ortholog of mouse Cctz (67% aa se- |
| | quence identity) [PC]. |
| | Expression |
| | The expression profile for the gene, |
| | derived from the proportion of animals |
| | at each stage in each Kohara library |
| | is: embryos 5%, L1 or L2 larvae 53%, |
| | L3 to adult 41%. |
| | In situ hybridisation pictures to all |
| | stages of development are available |
| | from Kohara NextDB. |
| | For a detailed expression pattern de- |
| | scription, see Wormbase Expr2045. |
| | The CDS has 6 exons. It covers 1.87 kb |
| | on the WS97 genome. The protein (539 |
| | aa, 58.9 kDa, pI 5.9) contains one |
| | chaperonin Cpn60/TCP-1 motif. It is |
| | predicted to localise in the cytoplasm |
| | [Psort2]. Taxblast (threshold 10\hat{ }-3) |
| | tracks ancestors down to archaea and |
| | eukaryota. |
| | COMPLETENESS: full length. |
| | Method: conceptual translation. |

| | |
|---|---|
| FEATURES | Location/Qualifiers |

| | |
|---|---|
| source | 1 . . . 539 |
| | /organism = “Caenorhabditis elegans” |
| | /db_xref = “taxon: 6239” |
| | /chromosome = “III” |
| | /map = “III; −1.53 cM (interpolated |
| | genetic position)” |
| | /map = “III; covering 2163 bp, from |
| | base 5855637 to 5853475 on genome |
| | release WS97” |
| Protein | 1 . . . 539 |
| | /product = “chaperonin Containing |
| | TCP-1 (58.9 kD) (cct-6)” |
| Region | 30 . . . 530 |
| | /region_name = “[Pfam/InterPro de- |
| | scription] chaperonin Cpn60/TCP-1” |
| | /db_xref = “CDD: pfam00118” |
| CDS | 1 . . . 539 |
| | /gene = “cct-6” |
| | /locus_tag = “3G944” |
| | /coded_by = “NM_171135.1: |
| | 1 . . . 1620” |
| | /db_xref = “AceView/WormGenes: |
| | cct-6” |
| | /db_xref = “GeneID: 175819” |
| | /db_xref = “LocusID: 175819” |
| | /db_xref = “WormBase: F01F1.8a” |

| | |
|---|---|
| ORIGIN | |
| 1 | mssiqclnpk aelarhaaal elnisgargl |
| | qdvmrsnlgp kgtlkmlvsg agdikltkdg |
| 61 | nvllhemaiq hptasmiaka staqddvtgd |
| | gttstvllig ellkqaeslv leglhprivt |
| 121 | egfewantkt lellekfkke apverdllve |
| | vcrtalrtkl hqkladhite cvvdavlair |
| 181 | rdgeepdlhm vekmemhhds dmdttlvrgl |
| | vldhgarhpd mprhvkdayi ltcnvsleye |
| 241 | ktevnsglfy ktakereall aaerefitrr |
| | vhkiielkkk vidnspdgkn kgfvvinqkg |
| 301 | idppsldlla segilalrra krrnmerlql |
| | avggeavnsv ddltpedlgw aglvyehslg |
| 361 | eekytfieec rapksvtlli kgpnkhtitq |
| | ikdaihdglr avfntivdka vlpgaaafei |
| 421 | aayvmlkkdv enlkgraklg aeafaqallv |
| | ipktlavngg ydaqetlvkl ieektaagpd |
| 481 | iavgldletg gavepqgiwd nvtvkknsis |
| | satvlacnll lvdevmragm tnlkqpqpe |
| // | |
| LOCUS | NP_741154 429 aa linear INV 21- |
| NOVEMBER | |
| 2003 | |
| DEFINITION | chaperonin Containing TCP-1 (cct-6) |
| | [Caenorhabditis elegans]. |
| ACCESSION | NP_741154 |
| VERSION | NP_741154.1 GI: 25144680 |
| DBSOURCE | REFSEQ: accession NM 171136.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 429) |

| | |
|---|---|
| AUTHORS | Kamath,R. S., Fraser,A. G., Dong,Y., |
| | Poulin,G., Durbin,R., Gotta,M., |
| | Kanapin,A., Le Bot,N., Moreno,S., |
| | Sohrmann,M., Welchman,D. P., |
| | Zipperlen,P. and Ahringer,J. |
| TITLE | Systematic functional analysis of the |
| | Caenorhabditis elegans genome using |
| | RNAi |
| JOURNAL | Nature 421 (6920), 231-237 (2003) |
| MEDLINE | 22417569 |
| PUBMED | 12529635 |

| | |
|---|---|
| REFERENCE | 2 (residues 1 to 429) |

| | |
|---|---|
| AUTHORS | Gonczy,P., Echeverri,C., Oegema,K., |
| | Coulson,A., Jones,S. J., Copley,R. R., |
| | Duperon,J., Oegema,J., Brehm,M., |
| | Cassin,E., Hannak, E., Kirkham,M., |
| | Pichler,S., Flohrs,K., Goessen,A., |
| | Leidel,S., Alleaume,A. M., Martin,C., |
| | Ozlu,N., Bork,P. and Hyman,A. A. |
| TITLE | Functional genomic analysis of cell di- |
| | vision in C. elegans using RNAi of |
| | genes on chromosome III |
| JOURNAL | Nature 408 (6810), 331-336 (2000) |
| MEDLINE | 20548710 |
| PUBMED | 11099034 |

| | |
|---|---|
| REFERENCE | 3 (residues 1 to 429) |

| | |
|---|---|
| AUTHORS | Leroux,M. R. and Candido,E. P. |
| TITLE | Characterization of four new tcp-1- |
| | related cct genes from the nematode |
| | Caenorhabditis elegans |
| JOURNAL | DNA Cell Biol. 14 (11), 951-960 (1995) |
| MEDLINE | 96069542 |
| PUBMED | 7576182 |

| | |
|---|---|
| COMMENT | PROVISIONAL REFSEQ: This record has not |
| | yet been subject to final NCBI review. |
| | This record is derived from an an- |
| | notated genomic sequence (NC_003281). |
| | The reference sequence was derived from |
| | WormBase CDS: F01F1.8b. |
| | Summary: This essential gene cct-6, |
| | also known as F01F1.8, 3G944 or YK828, |
| | maps at (III; −1.53). Phenotypes and |
| | affected processes are embryonic |
| | lethal, sterile adult, unhealthy, |
| | clear, translucent appearance, pro- |
| | truding vulva, small embryos, slow |
| | embryonic cell division, cytokinesis |
| | defect, abnormal cytoplasmic appear- |
| | ance. It encodes a chaperonin |
| | Containing TCP-1. |
| | According to the Worm Transcriptome |
| | Project, it is expressed at very high |
| | level at all stages of development |
| | [Kohara cDNAs]. Its existence, but not |
| | its exact sequence, derived here from |
| | the genome sequencing consortium an- |
| | notation, is supported by 122 cDNA |
| | clones. It would produce, by alterna- |
| | tive splicing, 2 different transcripts |
| | a, b altogether encoding 2 different |
| | protein isoforms. |
| | RNA interference results |
| | [T. Hyman; 2000] All embryos dead. DIC |
| | phenotype -- Semi-sterile; complex DIC |
| | phenotype; many embryos loose struc- |
| | tural integrity upon dissection; areas |
| | lacking yolk granules; failure in dif- |
| | ferent microtubule-based processes |
| | (centration/rotation, spindle assembly, |
| | chromosome segregation). DIC phenotype |
| | comment -- see also results from |
| | C07G2.3. Phenotype comment -- Confirmed |
| | with independent dsRNA (F01F1.8-RNA2; |
| | similar phenotype) (by injecting geno- |
| | mic PCR product TH: 304C1). Movies are |
| | available on Hyman's site. |
| | Same description as TH: 304C1 (by in- |
| | jecting genomic PCR product TH: 341B5). |
| | [J. Ahringer 2003] Embryonic lethal |
| | (100%), sterile, sick, clear, pro- |
| | truding vulva (by feeding genomic PCR |
| | product JA: F01F1.8). |
| | Function |
| | Protein properties: [C.elegansII] NMK. |
| | Encodes one of 7-9 related subunits of |
| | eukaryotic cytosolic chaperonin |
| | CCT.Ortholog of mouse Cctz (67% aa |
| | sequence identity) [PC]. |
| | Expression |
| | The expression profile for the gene, |
| | derived from the proportion of animals |
| | at each stage in each Kohara library |
| | is: embryos 5%, L1 or L2 larvae 53%, |
| | L3 to adult 41%. |
| | In situ hybridisation pictures to all |
| | stages of development are available |
| | from Kohara NextDB. |
| | For a detailed expression pattern de- |
| | scription, see Wormbase Expr2045. |
| | The predicted CDS has 6 exons. It |
| | covers 1.54 kb on the WS97 genome. The |
| | protein (429 aa, 47.6 kDa, pI 6.3) |
| | contains one chaperonin Cpn60/TCP-1 |
| | motif. It also contains an ER membrane |
| | domain [Psort2]. It is predicted to |
| | localise in the cytoplasm [Psort2]. |
| | Taxblast (threshold 10\hat{ }-3) tracks |
| | ancestors down to archaea and |
| | eukaryota. |
| | Method: conceptual translation. |

| | |
|---|---|
| FEATURES | Location/Qualifiers |

| | |
|---|---|
| source | 1 . . . 429 |
| | /organism = “Caenorhabditis elegans” |
| | /db_xref = “taxon: 6239” |
| | /chromosome = “III” |
| | /map = “III; −1.53 cM (interpolated |
| | genetic position)” |
| | /map = “III; covering 2163 bp, from |
| | base 5855637 to 5853475 on genome |
| | release WS97” |
| Protein | 1 . . . 429 |
| | /product = “chaperonin Containing |
| | TCP-1 (cct-6)” |
| Region | 30 . . . 428 |
| | /region_name = “[Pfam/InterPro de- |
| | scription] chaperonin Cpn60/TCP-1” |
| | /db_xref = “CDD: pfam00118” |
| Region | 425 . . . 428 |
| | /region_name = “[PSORT] ER membrane |
| | domain: VEKR” |
| CDS | 1 . . . 429 |
| | /gene = “cct-6” |
| | /locus_tag = “3G944” |
| | /coded_by = “NM_171136.1: |
| | 1 . . . 1290” |
| | /db_xref = “AceView/WormGenes: |
| | cct-6” |
| | /db_xref = “GeneID: 175819” |
| | /db_xref = “LocusID: 175819” |

| | |
|---|---|
| ORIGIN | |
| 1 | mssiqclnpk aelarhaaal elnisqargl |
| | qdvmrsnlgp kgtlkmlvsg agdikltkdg |
| 61 | nvllhemaiq hptasmiaka staqddvtgd |
| | gttstvllig ellkqaeslv leqlhprivt |
| 121 | egfewantkt lellekfkke apverdllve |
| | vcrtalrtkl hqkladhite cvvdavlair |
| 181 | rdgeepdlhm vekmemhhds dmdttlvrgl |
| | vldhgarhpd mprhvkdayi ltcnvsleye |
| 241 | ktevnsqlfy ktakereall aaerefitrr |
| | vhkiielkkk vidnspdgkn kgfvvinqkg |
| 301 | idppsldlla segilalrra krrnmerlql |
| | avggeavnsv ddltpedlgw aglvyehslg |
| 361 | eekytfieec rapksvtlli kgpnkhtitq |
| | ikdaihdglr avfntivdsc spwsccfrnc |
| 421 | clrdvekrc |

IIIFF. RDE-4

The RDE-4 protein is structurally related to drosophila R2D2 and the human TAR binding protein with conservation in the dsRBDs motifs.

| | |
|---|---|
| LOCUS | CAA83012 385 aa linear INV 23- |
| FEBRUARY | |
| 2005 | |
| DEFINITION | Hypothetical protein T20G5.11 |
| | [Caenorhabditis elegans]. |
| ACCESSION | CAA83012 |
| VERSION | CAA83012.1 GI: 458490 |
| DESOURCE | embl locus CET20G5, accession Z30423.2 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 385) |

| | |
|---|---|
| AUTHORS | . |
| CONSRTM | WormBase Consortium |
| TITLE | Genome sequence of the nematode C. |
| | elegans: a platform for investigating |
| | biology |
| JOURNAL | Science 282 (5396), 2012-2018 (1998) |
| PUBMED | 9851916 |

| | |
|---|---|
| REFERENCE | 2 (residues 1 to 385) |

| | |
|---|---|
| AUTHORS | Berks,M., Lloyd,C. R. and Smith,A. |
| TITLE | Direct Submission |
| JOURNAL | Submitted (07-MAR-1994) Nematode Se- |
| | quencing Project, Sanger Institute, |
| | Hinxton, Cambridge CB10 1SA, England |
| | and Department of Genetics, Washington |
| | University, St. Louis, MO 63110, USA. |
| | E-mail: worm@sanger.ac.uk |

| | |
|---|---|
| COMMENT | Coding sequences below are predicted |
| | from computer analysis, using predic- |
| | tions from Genefinder (P. Green, U. |
| | Washington), and other available in- |
| | formation. |
| | Current sequence finishing criteria for |
| | the C. elegans genome sequencing con- |
| | sortium are that all bases are either |
| | sequenced unambiguously on both |
| | strands, or on a single strand with |
| | both a dye primer and dye terminator |
| | reaction, from distinct subclones. |
| | Exceptions are indicated by an explicit |
| | note. |
| | IMPORTANT: This sequence is NOT neces- |
| | sarily the entire insert of the spec- |
| | ified clone. It may be shorter because |
| | we only sequence overlapping sections |
| | once, or longer because we arrange for |
| | a small overlap between neighbouring |
| | submissions. |
| | This sequence is the entire insert of |
| | clone T20G5. The start of this sequence |
| | (1 . . . 100) overlaps with the end of |
| | sequence Z30974. The end of this se- |
| | quence (47996 . . . 48095) overlaps |
| | with the start of sequence AL032660. |
| | For a graphical representation of this |
| | sequence and its analysis see:- |
| | http://www.wormbase.org/perl/ace/ |
| | elegans/seq/sequence? |
| name = ZK1321; class = Sequence. | |

| | |
|---|---|
| FEATURES | Location/Qualifiers |

| | |
|---|---|
| source | 1 . . . 385 |
| | /organism = “Caenorhabditis elegans” |
| | /strain = “Bristol N2” |
| | /db_xref = “taxon: 6239” |
| | /chromosome = “III” |
| | /clone = “T20G5” |
| Protein | 1 . . . 385 |
| | /product = “Hypothetical protein |
| | T20G5.11” |
| CDS | 1 . . . 385 |
| | /gene = “rde-4” |
| | /locus_tag = “T20G5.11” |
| | /standard_name = “T20G5.11” |
| | /coded_by = “complement(join |
| | (Z30423.2: 45951 . . . 46375, |
| | Z30423.2: 46424 . . . 46544, |
| | Z30423.2: 46589 . . . 46820, |
| | Z30423.2: 46870 . . . 47249))” |
| | /note = “C. elegans RDE-4 protein; |
| | contains similarity to Pfam domain |
| | PF00035 (Double-stranded RNA binding |
| | motif)” |
| | /db_xref = “GOA: Q22617” |
| | /db_xref = “InterPro: IPR001159” |
| | /db_xref = “UniProt/TrEMBL: Q22617” |

| | |
|---|---|
| ORIGIN | |
| 1 | mdltkltfes vfggsdvpmk psrsednktp |
| | rnrtdlemfl kktplmvlee aakavyqktp |
| 61 | twgtvelpeg femtlilnei tvkgqatskk |
| | aarqkaavey lrkvvekgkh eiffipgttk |
| 121 | eealsnidqi sdkaeelkrs tsdavqdndn |
| | ddsiptsaef ppgisptenw vgklqeksqk |
| 181 | sklqapiyed sknerterfl victmcnqkt |
| | rgirskkkda knlaawlmwk aledgiesle |
| 241 | sydmvdvien leeaehllei qdqaskikdk |
| | hsalidilsd kkrfsdysmd fnvlsvstmg |
| 301 | ihqvlleisf rrlvspdpdd lemgaehtqt |
| | eeimkataek eklrkknmpd sgplvfaghg |
| 361 | ssaeeakqca cksaiihfnt ydftd |

IIIGG. DRH-3 (D2005.5)

The DRH-3 protein now has been officially renamed DRH-3, this protein is a paralog of DRH-1 and DRH-2 which are essential for RNAi and have a human ortholog: melanoma differentiation associated protein-5.

| | |
|---|---|
| LOCUS | CAB02082 1119 aa linear INV 23- |
| FEBRUARY | |
| 2005 | |
| DEFINITION | Hypothetical protein D2005.5 |
| | [Caenorhabditis elegans]. |
| ACCESSION | CAB02082 |
| VERSION | CAB02082.3 GI: 38422755 |
| DBSOURCE | embl locus CED2005, accession Z79752.2 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 1119) |

| | |
|---|---|
| AUTHORS | . |
| CONSRTM | WormBase Consortium |
| TITLE | Genome sequence of the nematode C. |
| | elegans: a platform for investigating |
| | biology |
| JOURNAL | Science 282 (5396), 2012-2018 (1998) |
| PUBMED | 9851916 |

| | |
|---|---|
| REFERENCE | 2 (residues 1 to 1119) |

| | |
|---|---|
| AUTHORS | Wilkinson, J. |
| TITLE | Direct Submission |
| JOURNAL | Submitted (04-SEP-1996) Nematode Se- |
| | quencing Project, Sanger Institute, |
| | Hinxton, Cambridge CB10 1SA, England |
| | and Department of Genetics, Washington |
| | University, St. Louis, MO 63110, USA. |
| | E-mail: worm@sanger.ac.uk |

| | |
|---|---|
| COMMENT | On Nov. 18, 2003 this sequence version |
| | replaced gi: 21615449. Coding sequences |
| | below are predicted from computer |
| | analysis, using predictions from Gene- |
| | finder (P. Green, U. Washington), and |
| | other available information. |
| | Current sequence finishing criteria for |
| | the C. elegans genome sequencing con- |
| | sortium are that all bases are either |
| | sequenced unambiguously on both |
| | strands, or on a single strand with |
| | both a dye primer and dye terminator |
| | reaction, from distinct subclones. |
| | Exceptions are indicated by an |
| | explicit note. |
| | IMPORTANT: This sequence is NOT neces- |
| | sarily the entire insert of the spec- |
| | ified clone. It may be shorter because |
| | we only sequence overlapping sections |
| | once, or longer because we arrange for |
| | a small overlap between neighbouring |
| | submissions. |
| | IMPORTANT: This sequence is not the |
| | entire insert of clone D2005. It may |
| | be shorter because we only sequence |
| | overlapping sections once, or longer |
| | because we arrange for a small overlap |
| | between neighbouring submissions. |
| | The true left end of clone D2005 is at |
| | 1 in this sequence. The true right end |
| | of clone D2005 is at 104 in sequence |
| | Z81073. The true left end of clone |
| | F30F8 is at 43337 in this sequence. |
| | The start of this sequence |
| | (1 . . . 104) overlaps with the end of |
| | sequence AL033124. The end of this |
| | sequence (43337 . . . 43440) overlaps |
| | with the start of sequence Z81073. |
| | For a graphical representation of this |
| | sequence and its analysis see:- |
| | http://www.wormbase.org/perl/ace/ |
| | elegans/seq/sequence? |
| | name = ZK1321; class = Sequence. |

| | |
|---|---|
| FEATURES | Location/Qualifiers |

| | |
|---|---|
| source | 1 . . . 1119 |
| | /organism = “Caenorhabditis elegans” |
| | /strain = “Bristol N2” |
| | /db_xref = “taxon: 6239” |
| | /chromosome = “I” |
| | /clone = “D2005” |
| Protein | 1 . . . 1119 |
| | /product = “Hypothetical protein |
| | D2005.5” |
| CDS | 1 . . . 1119 |
| | /locus_tag = “D2005.5” |
| | /standard_name = “D2005.5” |
| | /coded_by = “join(Z79752.2: |
| | 37322 . . . 37419, Z79752.2: |
| | 37479 . . . 37652, Z79752.2: |
| | 37906 . . . 37981, Z79752.2: |
| | 38029 . . . 38110, Z79752.2: |
| | 38156 . . . 38680, Z79752.2: |
| | 38868 . . . 38993, Z79752.2: |
| | 39040 . . . 39221, Z79752.2: |
| | 39303 . . . 39484, Z79752.2: |
| | 39682 . . . 40007, Z79752.2: |
| | 40444 . . . 40724, Z79752.2: |
| | 40768 . . . 41676, Z79752.2: |
| | 42116 . . . 42216, Z79752.2: |
| | 42273 . . . 42411, Z79752.2: |
| | 42459 . . . 42565, Z79752.2: |
| | 42668 . . .42719)” |
| | /note = “contains similarity to Pfam |
| | domains PF00270 (DEAD and DEAH box |
| | helicases), PF00271 (Helicases con- |
| | served C-terminal domain)” |
| | /db_xref = “GOA: Q93413” |
| | /db_xref = “InterPro: IPR001410” |
| | /db_xref = “InterPro: IPR001650” |
| | /db_xref = “InterPro: IPR011545” |
| | /db_xref = “UniProt/TrEMBL: Q93413” |

| | |
|---|---|
| ORIGIN | |
| 1 | mqptairled ydksklrlpf espyfpayfr |
| | llkwkfldvc vestrnndig yfklfeslfp |
| 61 | pgkleeiarm iideptpvsh dpdmikirna |
| | dldvkirkqa etyvtlrhah qqkvqrrrfs |
| 121 | ecflntvlfd ekglriadev mfnydkelyg |
| | yshwedlpdg wltaetfknk fydeeevtnn |
| 181 | pfgyqkldrv agaargmiim khlksnprcv |
| | settilafev fnkgnhqlst dlvedllteg |
| 241 | pafelkieng eekkyavkkw slhktltmfl |
| | aiigfksndk kekneheewy ygfidamknd |
| 301 | panraalyfl dknwpeelee rekerdrirl |
| | tllksqrtne eavgedvctt irpqpkdsgy |
| 361 | npdavvtelv lrtyqeelvq palegkncvi |
| | vaptgsgkte vaiyaalkhi eertsqgkps |
| 421 | rvvllvpkip lvgqqkdrfl kycngmyevn |
| | gfhgsessvs gtgrrdevia thvsvmtpqi |
| 481 | linmlqsvrq nerlyvsdfs mmifdevhka |
| | aknhpyvlin qmvqewkyek pqiigltasl |
| 541 | svkvdgqkde nqmlndiynm lalinaphls |
| | titrqsside lnehvgkpdd svelclpake |
| 601 | nilrdyiery lnhahgkfle elasmskstg |
| | rnntippnmi ntfkknqpkn yeyydsllqg |
| 661 | iiqelnklnv pekwnsqtwa kymkvylear |
| | givdlmpamv afkymekaig klneshsetv |
| 721 | eystfikdhd tlkqtiqsve peivlrlknt |
| | ltnqfhvape srviifvtqr staqrvsdfl |
| 781 | neskvldqfg nygeqmvgyv lgtnkqgavq |
| | qtsqeqqltl dkfnngrlkv ivatsvveeg |
| 841 | ldvtacnlii kyncssgsai qlvqqrgrar |
| | aknsrsvlls vkssinetet nalisekymr |
| 901 | lcvkkiteng ekqlaaevkr vaelnaaerk |
| | rnleeqlnlr lrhenkiykl mcsncskefc |
| 961 | ksiyikkvfs nymvfdpsvw rflhveskrk |
| | vskylsednq plsdikcfhc kldvgrayki |
| 1021 | rgtylpqlsv kaltfvqesd yssmtkakws |
| | dveqdlfyis eaieddfrim lnalsdteen |
| 1081 | iekkivldld srqhnkqlem krfhiqqepp |
| | tkgvapeaq |

IIIHH. ERI-1

The ERI-1 protein is conserved and enhances RNAi and has a human homolog: AAH35279.

| | |
|---|---|
| LOCUS | T32581 562 aa linear INV 18- |
| NOVEMBER | |
| 2002 | |
| DEFINITION | hypothetical protein T07A9.10 - |
| | Caenorhabditis elegans. |
| ACCESSION | T32581 |
| VERSION | T32581 GI: 7507339 |
| DBSOURCE | pir: locus T32581; |
| | summary: #length 562 #molecular-weight |
| | 64656 #checksum 867; |
| | genetic: #gene CESP: T07A9.10 |
| | #map_position 4 #introns 9/1; 54/1; |
| | 218/1; 258/3; 349/1; 432/3; 516/1; |
| | superfamily: vacuolar protein sorting |
| | protein VPS45; |
| | PIR dates: 29-Oct-1999 #sequence_{—} |
| | revision 29-Oct-1999 #text_change |
| | 18-Nov-2 002. |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 562) |

| | |
|---|---|
| AUTHORS | Scheet,P. and Maggi,L. |
| TITLE | Direct Submission |
| JOURNAL | Submitted (??-DEC-1997) to the EMBL |
| | Data Library |

| | |
|---|---|
| FEATURES | Location/Qualifiers |

| | |
|---|---|
| source | 1 . . . 562 |
| | /organism = “Caenorhabditis elegans” |
| | /db_xref = “taxon: 6239” |
| Protein | 1 . . . 562 |
| | /product = “hypothetical protein |
| | T07A9.10” |

| | |
|---|---|
| ORIGIN | |
| 1 | mlrelvkkqi ienilrpqny dsklghrkfs |
| | vlvldksamv vvnsclslne vfeegvtlve |
| 61 | dltrnrepmp smdaiyiisp vaesidilin |
| | dfsrktkfnp gnsyrsahif fldpccdelf |
| 121 | eklskspavk wiktlkelnl nlkpvesqif |
| | tvnsqfrgdm tktadgivsl catlnihptl |
| 181 | rfqsdfaqss eicqrveqkl kefgnegmgt |
| | daelvvldrs fdlvspllhe vtlqamvvdv |
| 241 | tafkdgvyry teagdskeiv ldekdqnwld |
| | lrhkllpevm ksvnkmvkdf kntnktepen |
| 301 | iknqsskdfs ttvrtlqpyl kmkakmaayi |
| | slteecrsky fdslekiial eqdmavehtp |
| 361 | ehvritdsqa vgrlstfilp aiptetrlrl |
| | ilifmltigk dkdeqyfnrl lhhtdipese |
| 421 | fqiikrmliw rdktqksqfq hrrpppeder |
| | fiasrwdpki knlieeiyer rlderefkva |
| 481 | gkkstsdfrp aasarygsgl agkprekrki |
| | iifvvggity semrvayels kktnttvilg |
| 541 | sdeiltpssf leslrdrntv nc |

III. RRF-3

This protein is also conserved in S. pombe and many plants.

| | |
|---|---|
| LOCUS | CAA88315 1780 aa linear INV 22- |
| MARCH 2005 | |
| DEFINITION | Hypothetical protein F10B5.7 |
| | [Caenorhabditis elegans]. |
| ACCESSION | CAA88315 |
| VERSION | CAA88315.1 GI: 3875716 |
| DESOURCE | embl locus CEF10B5, accession Z48334.1 |
| | embl locus CET05C12, accession Z66500.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

REFERENCE 1 (residues 1 to 1780)

| | |
|---|---|
| AUTHORS | . |
| CONSRTM | C. elegans Sequencing Consortium |
| TITLE | Genome sequence of the nematode C. |
| | elegans: a platform for investigating biology |
| JOURNAL | Science 282 (5396), 2012-2018 (1998) |
| PUBMED | 9851916 |

| | |
|---|---|
| REFERENCE | 2 (residues 1 to 1780) |

| | |
|---|---|
| AUTHORS | Sims,M. A. |
| TITLE | Direct Submission |
| JOURNAL | Submitted (16-FEB-1995) Nematode Se- |
| | quencing Project, Sanger Institute, |
| | Hinxton, Cambridge CB10 1SA, England |
| | and Department of Genetics, Washington |
| | University, St. Louis, MO 63110, USA. |
| | E-mail: worm@sanger.ac.uk |

| | |
|---|---|
| COMMENT | Coding sequences below are predicted |
| | from computer analysis, using predic- |
| | tions from Genefinder (P. Green, U. |
| | Washington), and other available |
| | information. |
| | Current sequence finishing criteria for |
| | the C. elegans genome sequencing con- |
| | sortium are that all bases are either |
| | sequenced unambiguously on both |
| | strands, or on a single strand with |
| | both a dye primer and dye terminator |
| | reaction, from distinct subclones. |
| | Exceptions are indicated by an |
| | explicit note. |
| | IMPORTANT: This sequence is NOT neces- |
| | sarily the entire insert of the speci- |
| | fied clone. It may be shorter because |
| | we only sequence overlapping sections |
| | once, or longer because we arrange for |
| | a small overlap between neighbouring |
| | submissions. |
| | IMPORTANT: This sequence is not the |
| | entire insert of clone F10B5. It may be |
| | shorter because we only sequence over- |
| | lapping sections once, or longer be- |
| | cause we arrange for a small overlap |
| | between neighbouring submissions. |
| | The true left end of clone F10B5 is at |
| | 1 in this sequence. The true right end |
| | of clone F10B5 is at 15182 in sequence |
| | Z66500. The true left end of clone |
| | T05C12 is at 29032 in this sequence. |
| | The true right end of clone C41C4 is |
| | at 2219 in this sequence. The start of |
| | this sequence (1 . . . 99) overlaps |
| | with the end of sequence Z48045. |
| | The end of this sequence |
| | (29032 . . . 29132) overlaps with the |
| | start of sequence Z66500. For a |
| | graphical representation of this |
| | sequence and its analysis see:- |
| | http:.//www.wormbase.org/perl/ace/ |
| | elegans/seq/sequence? |
| | name = ZK1321; class = Sequence. |

| | |
|---|---|
| FEATURES | Location/Qualifiers |

| | |
|---|---|
| source | 1 . . . 1780 |
| | /organism = “Caenorhabditis elegans” |
| | /strain = “Bristol N2” |
| | /db_xref = “taxon: 6239” |
| | /chromosome = “II” |
| | /clone = “F10B5” |
| Protein | 1 . . . 1780 |
| | /product = “Hypothetical protein |
| | F10B5.7” |
| CDS | 1 . . . 1780 |
| | /gene = “rrf-3” |
| | /locus_tag = “F10B5.7” |
| | /standard_name = “F10B5.7” |
| | /coded_by = “join(Z48334.1: |
| | 23435 . . . 23502, Z48334.1: |
| | 23558 . . . 24167, Z48334.1: |
| | 24214 . . . 24449, Z48334.1: |
| | 24497 . . . 24610, Z48334.1: |
| | 24661 . . . 25018, Z48334.1: |
| | 25064 . . . 25883, Z48334.1: |
| | 25931 . . . 26489, Z48334.1: |
| | 26532 . . . 26743, Z48334.1: |
| | 26790 . . . 27477, Z48334.1: |
| | 27526 . . . 28249, Z48334.1: |
| | 28294 . . . 28751, Z48334.1: |
| | 28797 . . . 28902, Z48334.1: |
| | 28954 . . . 29132, Z66500.1: |
| | 102 . . . 114, Z66500.1: |
| | 161 . . . 358)” |
| | /note = “C. elegans RRF-3 protein; |
| | contains similarity to Pfam domain |
| | PF05183 (RNA dependent RNA |
| | polymerase)” |
| | /db_xref = “InterPro: IPR007855” |
| | /db_xref = “UniProt/TrEMBL: Q19285” |

| | |
|---|---|
| ORIGIN | |
| 1 | mlpfdnddss ddattsvrpk hprgvpqsqs |
| | tfprgrsnfs sgtlpnrkte ctpvntltig |
| 61 | hsnkmllttf rmdrnsksks evdvqeqpvh |
| | ssssafpgnh lnnfsypvnr gylrdyllqs |
| 121 | qrpstskpvd csvlkrhslp sthilyektk |
| | hrggvnieeq eklvrmlwaa aeesetvakt |
| 181 | rqfskkqaie lnfdakligs mnndcfgycr |
| | ahmenikdvl kthlklskvd evnwikvgmv |
| 241 | praayedksy vidahlvltp ngevedenel |
| | fsefassfts ritgmlhdqv flevpkmhtl |
| 301 | ftkitpqhmd inisaiaign cpnsglflvr |
| | gdfisqentv csvklqshhn adasrenssf |
| 361 | kvagsnkyls yarfehdkrl avvyfgvrla |
| | efaddgldha gfrlnlyynl fvrivvdmsh |
| 421 | ettnsiyiqm knpphlwegi pkntifhpsk |
| | skvlnmetct ewtrvlswpg daegrgvgct |
| 481 | seafsqsswi rltmrkdddn dsvsstqlmd |
| | ivtrlsarsk akvmfgsifs irrklapspa |
| 541 | fhslgsfran yalqalitrg svftdqlfda |
| | tdenipssdn dndedddddv ddtkkpmelv |
| 601 | heplflklvr rgmkecsqat eetleqllna |
| | fderrqidvv tafttmyqsr kiqyerllkg |
| 661 | eslqdvglak plpkncvsva kvivtpsril |
| | lmapevmmvn rvvrrfgpdy alrcvfrddn |
| 721 | lgrlairdfs innidhmsni vtegiyltlk |
| | nqiqvadrvy sflgwsnsqm rdqqcylyap |
| 781 | rvnaltgevt gtvedirvwm gdfrdaisvp |
| | kmmsrmgqcf tqaqptvyss vknihiveni |
| 841 | qvrlerhhwi vepdieggve nkycfsdgcg |
| | risiklathi skilqlkevp acfqvrfkgf |
| 901 | kgilvidpti ddiinmpkvi frksqqkfge |
| | qggelqdeyi evvkyampsp vclnrpfiti |
| 961 | ldqvsekqsa sshrritnrv hyylerelcs |
| | lsnmlinenq aaeelvnrtn laidwnaask |
| 1021 | ragfelsvdp lirdmlfsiy ryniihhisk |
| | akiflppslq rsmyqvvdet gllqyqqvfi |
| 1081 | qyspsirqts nrpilktgkv litknpchvp |
| | gdvrvfdavw qpalahlvdv vvfpqhgprp |
| 1141 | hpdemagsdl dqdeysiiwd qemlldynee |
| | amvfpsssaa eedkepttdd mvefflrylq |
| 1201 | qdsigrmsha hlayadlhgl fhenchaial |
| | kcavavdfpk sgvpaeplss feqcemtpdy |
| 1261 | mmsggkpmyy strlngqlhr karkveevle |
| | efetrgsvfe reydklicpe dvdvffgnei |
| 1321 | klvqtltlrd eyvdrmqqll deygiedeas |
| | vvsghaasik rlagmerddy sfyhtdkvve |
| 1381 | lryeklyavf rakffeefgg eeiniendgk |
| | ntrlkctkam hekirqwyfv ayvqpkinka |
| 1441 | grcigqslpw vawdalcdlr rqlmldknda |
| | vlrgkypiaa rleeeiensi erqfdkflkl |
| 1501 | kdlieshkda lflrryvyfy gdqiikmlfi |
| | lkvwlerenv lpssvlsiwq lgrllirlgl |
| 1561 | gdllgnptid yeksllmptt mfqqwiskke |
| | dadeaplirn fdmgtmmlef lrylasqsfa |
| 1621 | saesislrvf yekdivepil tksaqwmplh |
| | liayrtfhsi avsgrfdalh lddedavdqi |
| 1681 | teskdpilvn eslfssrnyn ddypisrsri |
| | lqslkdwsgv keiipreitg trksdmiyvt |
| 1741 | svgtvlarqr larlilisge tirdaiannv |
| | vpnevrdefl |

IIIJJ. ERI-3 (W09B6.3)

This protein is expressed as an operon with TAF-6.1 and expressed as a fusion protein and enhances RNAi when mutated.

| | |
|---|---|
| LOCUS | NP_493918 578 aa linear INV 26- |
| JANUARY | |
| 2005 | |
| DEFINITION | putative protein (66.4 kD) (2B417) |
| | [Caenorhabditis elegans]. |
| ACCESSION | NP_493918 |
| VERSION | NP_493918.2 GI: 32565182 |
| DBSOURCE | REFSEQ: accession NM 061517.2 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| COMMENT | VALIDATED REFSEQ: This record has un- |
| | dergone preliminary review of the se- |
| | quence, but has not yet been subject |
| | to final review. This record is derived |
| | from an annotated genomic sequence |
| | (NC_003280). The reference sequence was |
| | derived from WormBase CDS: W09B6.3. On |
| | Jul. 12, 2003 this sequence version re- |
| | placed gi: 17536803. |
| | Summary: This gene 2B417, also known as |
| | W09B6.3 or YK7122, maps at (II; |
| | −12.85). It encodes a putative protein. |
| | According to the Worm Transcriptome |
| | Project, it is well expressed at all |
| | stages of development [Kohara cDNAs]. |
| | Its sequence is fully supported by 7 |
| | cDNA clones. |
| | RNA interference results: |
| | [J. Ahringer 2003] No obvious phenotype |
| | (by feeding genomic PCR product JA: |
| | W09B6.3). |
| | [J. Ahringer 2003] No obvious phenotype |
| | (by feeding genomic PCR product JA: |
| | W09B6.2). Warning: this double stranded |
| | RNA may also interfere with gene |
| | taf-6.1. |
| | Expression |
| | The expression profile for the gene, |
| | derived from the proportion of animals |
| | at each stage in each Kohara library |
| | is: embryos 21%, L1 or L2 larvae 31%, |
| | L3 to adult (including dauer) 48%. |
| | In situ hybridisation pictures to all |
| | stages of development are available |
| | from Kohara NextDB. |
| | The CDS has 11 exons. It covers 4.20 |
| | kb on the WS97 genome. The protein |
| | (578 aa, 66.4 kDa, pI 8.5) contains no |
| | Pfam motif. Taxblast (threshold 10\hat{ }-3) |
| | tracks ancestors down to caenorhabditis |
| | elegans. |
| | COMPLETENESS: full length. |
| | Method: conceptual translation. |

| | |
|---|---|
| FEATURES | Location/Qualifiers |

| | |
|---|---|
| source | 1 . . . 578 |
| | /organism = “Caenorhabditis elegans” |
| | /db_xref = “taxon: 6239” |
| | /chromosome = “II” |
| | /map = “II; −12.85 cM (interpolated |
| | genetic position)” |
| | /map = “II; covering 4252 bp, from |
| | base 1123539 to 1127792 on genome |
| | release WS97” |
| | /clone_lib = “Kohara embryonic |
| | lambda gt11 library: yk516c2, |
| | yk590b10; Kohara Sugano L1 larvae |
| | cap-selected library: yk1341c6, |
| | yk1341d5; Kohara Sugano L2 larvae |
| | cap-selected library: yk1378a11; |
| | Kohara Sugano mixed stage cap- |
| | selected library: yk724f12; Kohara |
| | mixed stage library, from him-8 |
| | strain, containing 15-30% males: |
| | yk379e7” |
| Protein | 1 . . . 578 |
| | /product = “putative protein (66.4 |
| | kD) (2B417)” |
| Region | 347 . . . 350 |
| | /region_name = “[PSORT] nuclear |
| | localization domain: KKKK” |
| Region | 414 . . . 417 |
| | /region_name = “[PSORT] vacuolar |
| | domain: ILPK” |
| Region | 456 . . . 462 |
| | /region_name = “[PSORT] nuclear |
| | localization domain: PKNPKKR” |
| Region | 459 . . . 465 |
| | /region_name = “[PSORT] nuclear |
| | localization domain: PKKRVEI” |
| CDS | 1 . . . 578 |
| | /gene = “2B417” |
| | /locus_tag = “2B417” |
| | /coded_by = “NM_061517.2: |
| | 1 . . . 1737” |
| | /db_xref = “AceView/WormGenes: |
| | 2B417” |
| | /db_xref = “GeneID: 173497” |
| | /db_xref = “WormBase: W09B6.3” |

| | |
|---|---|
| ORIGIN | |
| 1 | mqpvlvnsrp lrvksheses klnlieqedq |
| | feganyssss gviicysngt gevitqeafd |
| 61 | dsgihfifsk atciqypsnf dpigvgsvvq |
| | ifwsrsferv vrgnhiivqi ekmevykcca |
| 121 | mlreqvfvtf nspstagvai gvternitva |
| | fhpncspvir yetlkahsig rtefeikdrh |
| 181 | rentnrmvdv ilaavpfrve ihgnvdkipf |
| | fviekcrnsp grsgaavitk imknhfmean |
| 241 | flqnsesiyf dstschsnil ekvsigslin |
| | vladptfats sykwygydvt lcnnylahas |
| 301 | tqrsfvlenn eilqnckkle kspeeaettt |
| | kndlrfvppq pekgevkkkk mtnclkfnsk |
| 361 | saqfklrhli ldrcfselpe reaksiinsy |
| | fidrlaegik iekidknwrt fgeilpktpk |
| 421 | kyseslkksi qnvlepfgln kpekaaetpk |
| | iveyfpknpk krveivekpt vdeirelfga |
| 481 | lmdaegfaln qrvkphfvlp dtrwkpterr |
| | yigiyddvqw tfmstfcpki eensenrpla |
| 541 | ggwwyrrtvp rdhpveivqk metrrniikd |
| | ctespfie |

IIIKK. ERI-5 (Y38F2AR.1)

This protein has homologs in multiple species, with conservation found in the TUDOR domain. The paralog f22d6.6 plays a role in other small RNA silencing pathways in C. elegans.

| | |
|---|---|
| LOCUS | NP_500199 458 aa linear INV 26- |
| JANUARY | |
| 2005 | |
| DEFINITION | maternal tudor protein (4D159) |
| | [Caenorhabditis elegans]. |
| ACCESSION | NP_500199 |
| VERSION | NP_500199.1 GI: 17543178 |
| DBSOURCE | REFSEQ: accession NM 067798.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| COMMENT | PROVISIONAL REFSEQ: This record has not |
| | yet been subject to final NCBI review. |
| | This record is derived from an an- |
| | notated genomic sequence (NC_003282). |
| | The reference sequence was derived |
| | from WormBase CDS: Y38F2AR.1. |
| | Summary: This gene 4D159, also known as |
| | Y38F2AR.1 or YK7605, maps at (IV; |
| | −9.66). It encodes a maternal tudor |
| | protein. |
| | According to the Worm Transcriptome |
| | Project, it is moderately expressed in |
| | embryos, L1, L2 and L3 larvae [Kohara |
| | cDNAs]. Its existence, but not its |
| | exact sequence, derived here from the |
| | genome sequencing consortium annota- |
| | tion, is supported by 5 cDNA clones. |
| | RNA interference results: |
| | [J. Ahringer 2003] No obvious phenotype |
| | (by feeding genomic PCR product JA: |
| | Y38F2A_6126.j). |
| | Expression |
| | The expression profile for the gene, |
| | derived from the proportion of animals |
| | at each stage in each Kohara library |
| | is: embryos 16%, L1 or L2 larvae 66%, |
| | L3 to adult 18%. |
| | In situ hybridisation pictures to all |
| | stages of development are available |
| | from Kohara NextDB. |
| | The predicted CDS has 6 exons. It |
| | covers 5.00 kb on the WS97 genome. The |
| | protein (458 aa, 53.0 kDa, pI 4.7) |
| | contains one maternal tudor protein |
| | motif. It also contains an ER membrane |
| | domain [Psort2]. Taxblast (threshold |
| | 10\hat{ }-3) tracks ancestors down to |
| | caenorhabditis elegans. |
| | Method: conceptual translation. |

| | |
|---|---|
| FEATURES | Location/Qualifiers |

| | |
|---|---|
| source | 1 . . . 458 |
| | /organism = “Caenorhabditis elegans” |
| | /db_xref = “taxon: 6239” |
| | /chromosome = “IV” |
| | /map = “IV; −9.66 cM (interpolated |
| | genetic position)” |
| | /map = “IV; covering 5438 bp, from |
| | base 2390825 to 2396264 on genome |
| | release WS97” |
| | /clone_lib = “Kohara embryonic |
| | lambda gt11 library: yk592e2; |
| | Kohara Sugano L2 larvae cap-selected |
| | library: yk818d10, yk1502b5, |
| | yk1498b6, yk1503h5” |
| Protein | 1 . . . 458 |
| | /product = “maternal tudor protein |
| | (4D159)” |
| Region | 13 . . . 65 |
| | /region_name = “[Pfam/InterPro de- |
| | scription] maternal tudor protein” |
| | /db_xref = “CDD: pfam00567” |
| Region | 454 . . . 457 |
| | /region_name = “[PSORT] ER membrane |
| | domain: DKDS” |
| CDS | 1 . . . 458 |
| | /gene = “4D159” |
| | /locus_tag = “4D159” |
| | /coded_by = “NM_067798.1: |
| | 1 . . . 1377” |
| | /db_xref = “AceView/WormGenes: |
| | 4D159” |
| | /db_xref = “GeneID: 177029” |
| | /db_xref = “WormBase: Y38F2AR.1” |

| | |
|---|---|
| ORIGIN | |
| 1 | mamaplrprv farclilknl elieaariff |
| | idsavtanvs wkclfqiden lkfhpwqamh |
| 61 | ctlgrlvhls dswtdtqcte frnivskfak |
| | fqitanqcdv dfrsdrpsll vnlyglpngt |
| 121 | eidkkvaiee icavsmqnvm vsqfptnfmv |
| | npkleeldke qdhldville efrrdlpadw |
| 181 | aheppadyre ddadwdilqc hvaewndtal |
| | eqfrradgsf wamlepsctv spwemhvtpi |
| 241 | lapekmsdne hwifeqlvkn senqqkiddf |
| | ysnlknqrpl emeeikfalq tgrtyvmati |
| 301 | knrqkssaqw lrceiidflp nanvalryvd |
| | lgtrgilklk nlhrmhieht kiapacieig |
| 361 | rfldddlsma dsemewnthf wreivpydvp |
| | ivvgpdmefl etgklqfsqi rvagdedden |
| 421 | lldkipspsp fftersddlr tqkeddddgn |
| | vsddkdsg |

IIILL. PIR-1 (T23G7.5)

This gene is an ortholog of the well conserved PIR-1 from human and mouse and required for RNAi in C. elegans. An ortholog is the human dual specificity phosphatase 11 (DUSP11).

| | |
|---|---|
| LOCUS | CAA92703 261 aa linear INV 23- |
| FEBRUARY | |
| 2005 | |
| DEFINITION | Hypothetical protein T23G7.5 |
| | [Caenorhabditis elegans]. |
| ACCESSION | CAA92703 |
| VERSION | CAA92703.1 GI: 3880145 |
| DBSOURCE | embl locus CET23G7, accession Z68319.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 261) |

| | |
|---|---|
| AUTHORS | . |
| CONSRTM | WormBase Consortium |
| TITLE | Genome sequence of the nematode C. |
| | elegans: a platform for investigating |
| | biology |
| JOURNAL | Science 282 (5396), 2012-2018 (1998) |
| PUBMED | 9851916 |

| | |
|---|---|
| REFERENCE | 2 (residues 1 to 261) |

| | |
|---|---|
| AUTHORS | Barlow,K. |
| TITLE | Direct Submission |
| JOURNAL | Submitted (22-DEC-1995) Nematode Se- |
| | quencing Project, Sanger Institute, |
| | Hinxton, Cambridge CB10 1SA, England |
| | and Department of Genetics, Washington |
| | University, St. Louis, MO 63110, USA. |
| | E-mail: worm@sanger.ac.uk |

| | |
|---|---|
| COMMENT | Coding sequences below are predicted |
| | from computer analysis, using predic- |
| | tions from Genefinder (P. Green, U. |
| | Washington), and other available |
| | information. |
| | Current sequence finishing criteria for |
| | the C. elegans genome sequencing con- |
| | sortium are that all bases are either |
| | sequenced unambiguously on both |
| | strands, or on a single strand with |
| | both a dye primer and dye terminator |
| | reaction, from distinct subclones. |
| | Exceptions are indicated by an explicit |
| | note. |
| | IMPORTANT: This sequence is NOT neces- |
| | sarily the entire insert of the speci- |
| | fied clone. It may be shorter because |
| | we only sequence overlapping sections |
| | once, or longer because we arrange for |
| | a small overlap between neighbouring |
| | submissions. |
| | 951009: yk82b3.3 delimits the 3′ end of |
| | ZK1067.6 960305: T23G7 deleted in union |
| | with ZK1067.6 |
| | IMPORTANT: This sequence is not the |
| | entire insert of clone T23G7. It may be |
| | shorter because we only sequence over- |
| | lapping sections once, or longer be- |
| | cause we arrange for a small overlap |
| | between neighbouring submissions. The |
| | true left end of clone T23G7 is at 1 |
| | in this sequence. The true right end |
| | of clone T23G7 is at 16033 in sequence |
| | Z70038. The true left end of clone |
| | ZK1067 is at 19833 in this sequence. |
| | The true right end of clone W07A12 |
| | is at 6609 in this sequence. The start |
| | of this sequence (1 . . . 104) overlaps |
| | with the end of sequence Z68320. The |
| | end of this sequence |
| | (19833 . . . 19934) overlaps with the |
| | start of sequence Z70038. |
| | For a graphical representation of this |
| | sequence and its analysis see:- |
| | http://www.wormbase.org/perl/ace/ |
| | elegans/seq/sequence? |
| | name = ZK1321; class = Sequence. |

| | |
|---|---|
| FEATURES | Location/Qualifiers |

| | |
|---|---|
| source | 1 . . . 261 |
| | /organism = “Caenorhabditis elegans” |
| | /strain = “Bristol N2” |
| | /db_xref = “taxon: 6239” |
| | /chromosome = “II” |
| | /clone = “T23G7” |
| Protein | 1 . . . 261 |
| | /product = “Hypotheticai protein |
| | T23G7.5” |
| CDS | 1 . . . 261 |
| | /locus_tag = “T23G7.5” |
| | /standard_name = “T23G7.5” |
| | /coded_by = “join (Z68319.1: |
| | 12488 . . . 12654, Z68319.1: |
| | 12851 . . . 13093, Z68319.1: |
| | 13144 . . . 13241, Z68319.1: |
| | 13297 . . . 13407, Z68319.1: |
| | 13455 . . .13621)” |
| | /note = “contains similarity to Pfam |
| | domain PF00782 (Dual specificity |
| | phosphatase, catalytic domain)” |
| | /db_xref = “GOA: Q22707” |
| | /db_xref = “InterPro: IPR000340” |
| | /db_xref = “InterPro: IPR000387” |
| | /dbx_ref = “UniProt/TrEMBL: Q22707” |

| | |
|---|---|
| ORIGIN | |
| 1 | mpeprctaiv nflnlshsil isifsvsvms |
| | nyhhnhnyqh rprgyerlpg krlpdrwniy |
| 61 | dnvgrdidgt rfvpfktpld ssffdgknmp |
| | velqfgvktl islaqqankq iglvidltnt |
| 121 | dryykktewa dhgvkylkln cpghevnere |
| | dlvqdfinav kefvndkend gkligvhcth |
| 181 | glnrtgylic rymidvdnys asdaismfey |
| | yrghpmereh ykkslyeaer kkkygkssgk |
| 241 | ssgnsadsti sseqlhrnns q |

IIIMM. C32A3.2

| | |
|---|---|
| LOCUS | CAA88285 346 aa linear INV 23- |
| FEBRUARY | |
| 2005 | |
| DEFINITION | Hypothetical protein C32A3.2 |
| | [Caenorhabditis elegans]. |
| ACCESSION | CAA88285 |
| VERSION | CAA88285.1 GI: 3874617 |
| DBSOURCE | embl locus CEC32A3, accession Z48241.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 346) |

| | |
|---|---|
| AUTHORS | . |
| CONSRTM | WormBase Consortium |
| TITLE | Genome sequence of the nematode C. |
| | elegans: a platform for investigating |
| | biology |
| JOURNAL | Science 282 (5396), 2012-2018 (1998) |
| PUBMED | 9851916 |

| | |
|---|---|
| REFERENCE | 2 (residues 1 to 346) |

| | |
|---|---|
| AUTHORS | Thomas,K. |
| TITLE | Direct Submission |
| JOURNAL | Submitted (14-FEB-1995) Nematode Se- |
| | quencing Project, Sanger Institute, |
| | Hinxton, Cambridge CB10 1SA, England |
| | and Department of Genetics, Washington |
| | University, St. Louis, MO 63110, USA. |
| | E-mail: worm@sanger.ac.uk |

| | |
|---|---|
| COMMENT | Coding sequences below are predicted |
| | from computer analysis, using predic- |
| | tions from Genefinder (P. Green, U. |
| | Washington), and other available |
| | information. |
| | Current sequence finishing criteria for |
| | the C. elegans genome sequencing |
| | consortium are that all bases are |
| | either sequenced unambiguously on both |
| | strands, or on a single strand with |
| | both a dye primer and dye terminator |
| | reaction, from distinct subclones. |
| | Exceptions are indicated by an explicit |
| | note. |
| | IMPORTANT: This sequence is NOT neces- |
| | sarily the entire insert of the speci- |
| | fied clone. It may be shorter because |
| | we only sequence overlapping sections |
| | once, or longer because we arrange for |
| | a small overlap between neighbouring |
| | submissions. |
| | IMPORTANT: This sequence is not the |
| | entire insert of clone C32A3. It may be |
| | shorter because we only sequence over- |
| | lapping sections once, or longer be- |
| | cause we arrange for a small overlap |
| | between neighbouring submissions. |
| | The true left end of clone C32A3 is at |
| | 1 in this sequence. The true right end |
| | of clone C32A3 is at 44660 in this |
| | sequence. The true left end of clone |
| | C46F11 is at 45409 in this sequence. |
| | The true right end of clone C48D5 is |
| | at 4074 in this sequence. The start of |
| | this sequence (1 . . . 102) overlaps |
| | with the end of sequence Z36237. |
| | The end of this sequence |
| | (45409 . . . 45510) overlaps with the |
| | start of sequence Z81449. For a |
| | graphical representation of this se- |
| | quence and its analysis see:- |
| | http://www.wormbase.org/perl/ace/ |
| | elegans/seq/sequence? |
| | name = ZK1321; class = Sequence. |

| | |
|---|---|
| FEATURES | Location/Qualifiers |

| | |
|---|---|
| source | 1 . . . 346 |
| | /organism = “Caenorhabditis elegans” |
| | /strain = “Bristol N2” |
| | /db_xref = “taxon: 6239” |
| | /chromosome = “III” |
| | /clone = “C32A3” |
| Protein | 1 . . . 346 |
| | /product = “Hypothetical protein |
| | C32A3.2” |
| CDS | 1 . . . 346 |
| | /locus_tag = “C32A3.2” |
| | /standard_name = “C32A3.2” |
| | /coded_by = “complement(join |
| | (Z48241.1: 31596 . . . 31840, |
| | Z48241.1: 32812 . . . 33113, |
| | Z48241.1: 33160 . . . 33321, |
| | Z48241.1: 33366 . . . 33460, |
| | Z48241.1: 33508 . . . 33603, |
| | Z48241.1: 33657 . . . 33797))” |
| | /note = “contains similarity to Homo |
| | sapiens Kinesin-like protein KTF14; |
| | ENSEMBL: ENSP00000236917” |
| | /db_xref = “Uniprot/Swiss-Prot: |
| | Q09261” |

| | |
|---|---|
| ORIGIN | |
| 1 | mqadgekkkk ktnpersthd dtpksrtrvl |
| | fsqyfflsfs lffraifmlr slcsiavrlg |
| 61 | garqprllss aasgdgndgk gakdaidedl |
| | lnaiegvann ihpqngsekk slkntlinrl |
| 121 | vanekasfda aaasasseml ddqaliglla |
| | dvagdakvek klppksaqlr qekrglvllr |
| 181 | keifyqavqs gftteearvk setivneaqi |
| | klqeqrkall ndvrekveqe eveetersek |
| 241 | dqklftmale fmekiykddl issavqfpta |
| | hsdqqilskn ksngqqkenn gniqsimssk |
| 301 | wamnrmfhsl ityswrdiyh hwvsrnlvql |
| | lilciwfvlv yprihi |

Selected Human Homologs

Under this subsection, selected human homologs referred to above, are described in further detail.

Human Melanoma Differentiation Associated Protein-5

| | |
|---|---|
| LOCUS | NP_071451 1025 aa linear PRI 02- |
| MARCH 2005 | |
| DEFINITION | melanoma differentiation associated |
| | protein-5 [Homo sapiens]. |
| ACCESSION | NP_071451 |
| VERSION | NP_071451.2 GI: 27886568 |
| DBSOURCE | REFSEQ: accession NM 022168.2 |
| KEYWORDS | . |
| SOURCE | Homo sapiens (human) |

| | |
|---|---|
| ORGANISM | Homo sapiens |
| | Eukaryota; Metazoa; Chordata; Craniata; |
| | Vertebrata; Euteleostomi; Mammalia; |
| | Eutheria; Fuarchontoglires; Primates; |
| | Catarrhini; Hominidae; Homo. |

| | |
|---|---|
| REFERENCE | 1 (residues 1 to 1025) |

| | |
|---|---|
| AUTHORS | Andrejeva,J., Childs,K. S., |
| | Young,D. F., Carlos,T. S., Stock,N., |
| | Goodbourn,S. and Randall,R. E. |
| TITLE | The V proteins of paramyxoviruses bind |
| | the IFN-inducible RNA helicase, mda-5, |
| | and inhibit its activation of the IFN- |
| | beta promoter |
| JOURNAL | Proc. Natl. Acad. Sci. U.S.A. 101 (49), |
| | 17264-17269 (2004) |
| PUBMED | 15563593 |
| REMARK | GeneRIF: mda-5 plays a central role in |
| | an intracellular signal transduction |
| | pathway that can lead to the activation |
| | of the IFN-beta promoter, and that the |
| | V proteins of paramyxoviruses interact |
| | with mda-5 to block its activity. |

| | |
|---|---|
| REFERENCE | 2 (residues 1 to 1025) |

| | |
|---|---|
| AUTHORS | Kanq,D. C., Gopalkrishnan,R. V., |
| | Lin,L., Randolph,A., Valerie, K., |
| | Pestka,S. and Fisher,P. B. |
| TITLE | Expression analysis and genomic char- |
| | acterization of human melanoma dif- |
| | ferentiation associated gene-5, mda-5: |
| | a novel type I interferon-responsive |
| | apoptosis-inducing gene |
| JOURNAL | Oncogene 23 (9), 1789-1800 (2004) |
| PUBMED | 14676839 |
| REMARK | GeneRIF: mda-5 is a novel type I IFN- |
| | inducible gene, which may contribute to |
| | apoptosis induction during terminal |
| | differentiation and during IFN treat- |
| | ment |

| | |
|---|---|
| REFERENCE | 3 (residues 1 to 1025) |

| | |
|---|---|
| AUTHORS | Kang,D. C., Gopalkrishnan,R. V., Wu,Q., |
| | Jankowsky,E., Pyle,A. M. and Fisher, |
| | P.B. |
| TITLE | mda-5: An interferon-inducible putative |
| | RNA helicase with double-stranded RNA- |
| | dependent ATPase activity and melanoma |
| | growth-suppressive properties |
| JOURNAL | Proc. Natl. Acad. Sci. U.S.A. 99 (2), |
| | 637-642 (2002) |
| PUBMED | 11805321 |
| REMARK | GeneRIF: mda-5: An interferon-inducible |
| | putative RNA helicase with double- |
| | stranded RNA-dependent ATPase activity |
| | and melanoma growth-suppressive |
| | properties |

| | |
|---|---|
| COMMENT | REVIEWED REFSEQ: This record has been |
| | curated by NCBI staff. The reference |
| | sequence was derived from AF095844.1 |
| | and BU902097.1. On Jan. 24, 2003 this |
| | sequence version replaced gi: 11545922. |
| | Summary: DEAD box proteins, character- |
| | ized by the conserved motif Asp-Glu- |
| | Ala-Asp (DEAD), are putative RNA |
| | helicases. They are implicated in a |
| | number of cellular processes involving |
| | alteration of RNA secondary structure |
| | such as translation initiation, nu- |
| | clear and mitochondrial splicing, and |
| | ribosome and spliceosome assembly. |
| | Based on their distribution patterns, |
| | some members of this family are be- |
| | lieved to be involved in embryogenesis, |
| | spermatogenesis, and cellular growth |
| | and division. This gene encodes a DEAD |
| | box protein that is upregulated in re- |
| | sponse to treatment with beta-inter- |
| | feron (IFNB) and a protein kinase C- |
| | activating compound, mezerein (MEZ). |
| | Irreversible reprogramming of melanomas |
| | can be achieved by treatment with both |
| | these agents; treatment with either |
| | agent alone only achieves reversible |
| | differentiation. |

| | |
|---|---|
| FEATURES | Location/Qualifiers |

| | |
|---|---|
| source | 1 . . . 1025 |
| | /organism = “Homo sapiens” |
| | /db_xrefr = “taxon: 9606” |
| | /chromosome = “2” |
| | /map = “2p24.3-q24.3” |
| Protein | 1 . . . 1025 |
| | /product = “melanoma differentiation |
| | associated protein-5” |
| | /note = “DEAD/H (Asp-Glu-Ala-Asp/His) |
| | box polypeptide” |
| CDS | 1 . . . 1025 |
| | /gene = “IFIH1” |
| | /coded_by = “NM_022168.2: |
| | 223 . . . 3300” |
| | /db_xref = “CCDS: CCD52217.1” |
| | /db_xref = “GeneID: 64135” |
| | /db_xref = “MIM: 606951” |

| | |
|---|---|
| ORIGIN | |
| 1 | msngystden fryliscfra rvkmyiqvep |
| | vldyltflpa evkeqiqrtv atsgnmqave |
| 61 | lllstlekgv whlgwtrefv ealrrtgspl |
| | aarymnpelt dlpspsfena hdeylqllnl |
| 121 | lqptlvdkll vrdvldkcme eelltiedrn |
| | riaaaenngn esgvrellkr ivqkenwfsa |
| 181 | flnvlrqtgn nelvqeltgs dcsesnaeie |
| | nlsqvdgpqv eeqllsttvq pnlekevwgm |
| 241 | ennssessfa dssvvsesdt slaegsvscl |
| | deslghnsnm gsdsgtmgsd sdeenvaara |
| 301 | spepelqlrp yqmevaqpal egkniiiclp |
| | tgsgktrvav yiakdhldkk kkasepqkvi |
| 361 | vlvnkvllve qlfrkefqpf lkkwyrvigl |
| | sgdtqlkisf pevvkscdii istaqilens |
| 421 | linlengeda gvqlsdfsli iidechhtnk |
| | eavynnimrh ylmqklknnr lkkenkpvip |
| 481 | lpqilgltas pgvggatkqa kaeehilklc |
| | anldaftikt vkenldqlkn qiqepckkfa |
| 541 | iadatredpf keklleimtr iqtycgmspm |
| | sdfgtqpyeq waiqmekkaa kegnrkervc |
| 601 | aehlrkynea lqindtirmi daythletfy |
| | neekdkkfav ieddsdeggd deycdgdede |
| 661 | ddlkkplkld etdrflmtlf fennkmlkrl |
| | aenpeyenek ltklrntime qytrteesar |
| 721 | giiftktrqs ayalsqwite nekfaevgvk |
| | ahhligaghs sefkpmtqne qkeviskfrt |
| 781 | gkinlliatt vaeegldike cniviryglv |
| | tneiamvqar graradesty vlvahsgsgv |
| 841 | iehetvndfr ekmmykaihc vqnmkpeeya |
| | hkilelqmqs imekkmktkr niakhyknnp |
| 901 | slitflcknc svlacsgedi hviekmhhvn |
| | mtpefkelyi vrenkalqkk cadyqingei |
| 961 | ickcgqawgt mmvhkgldlp clkirnfvvv |
| | fknnstkkqy kkwvelpitf pnldyseccl |
| 1021 | fsded |
| // | |

Human SMD1

| | |
|---|---|
| LOCUS | NP_008869 119 aa linear PRI 26- |
| OCTOBER | |
| 2004 | |
| DEFINITION | small nuclear ribonucleoprotein D1 |
| | polypeptide 16 kDa [Homo sapiens]. |
| ACCESSION | NP_008869 |
| VERSION | NP_008869.1 GI: 5902102 |
| DBSOURCE | REFSEQ: accession NM 006938.2 |
| KEYWORDS | . |
| SOURCE | Homo sapiens (human) |

| | |
|---|---|
| ORGANISM | Homo sapiens |
| | Eukaryota; Metazoa; Chordata; Craniata; |
| | Vertebrata; Euteleostomi; Mammalia; |
| | Eutheria; Euarchontoglires; Primates; |
| | Catarrhini; Hominidae; Homo. |

| | |
|---|---|
| REFERENCE | 1 (residues 1 to 119) |

| | |
|---|---|
| AUTHORS | Fong,Y. W. and Zhou,Q. |
| TITLE | Stimulatory effect of splicing factors |
| | on transcriptional elongation |
| JOURNAL | Nature 414 (6866), 929-933 (2001) |
| PUBMED | 11780068 |

| | |
|---|---|
| REFERENCE | 2 (residues 1 to 119) |

| | |
|---|---|
| AUTHORS | Sun,D., Ou,Y. C. and Hoch,S. O. |
| TITLE | Analysis of genes for human snRNP |
| | Sm-D1 protein and identification of the |
| | promoter sequence which shows segmental |
| | homology to the promoters of Sm-E and |
| | U1 snRNA genes |
| JOURNAL | Gene 189 (2), 245-254 (1997) |
| PUBMED | 9168134 |

| | |
|---|---|
| REFERENCE | 3 (residues 1 to 119) |

| | |
|---|---|
| AUTHORS | Lehmeier,T., Raker,V., Hermann, H. and |
| | Luhrmann,R. |
| TITLE | cDNA cloning of the Sm proteins D2 and |
| | D3 from human small nuclear ribonucleo- |
| | proteins: evidence for a direct |
| | D1-D2 interaction |
| JOURNAL | Proc. Natl. Acad. Sci. U.S.A. 91 (25), |
| | 12317-12321 (1994) |
| PUBMED | 7527560 |

| | |
|---|---|
| REFERENCE | 4 (residues 1 to 119) |

| | |
|---|---|
| AUTHORS | Lehmeier,T., Foulaki,K. and Luhrmann,R. |
| TITLE | Evidence for three distinct D proteins, |
| | which react differentially with anti-Sm |
| | autoantibodies, in the cores of the |
| | major snRNPs U1, U2, U4/U6 and U5 |
| JOURNAL | Nucleic Acids Res. 18 (22), 6475-6484 |
| | (1990) |
| PUBMED | 1701240 |

| | |
|---|---|
| REFERENCE | 5 (residues 1 to 119) |

| | |
|---|---|
| AUTHORS | Rokeach,L. A., Haselby,J. A. and |
| | Hoch,S. O. |
| TITLE | Molecular cloning of a cDNA encoding |
| | the human Sm-D autoantigen |
| JOURNAL | Proc. Natl. Acad. Sci. U.S.A. 85 (13), |
| | 4832-4836 (1988) |
| PUBMED | 3260384 |

| | |
|---|---|
| COMMENT | REVIEWED REFSEQ: This record has been |
| | curated by NCBI staff. The reference |
| | sequence was derived from J03798.1. |
| | Summary: This gene encodes a small |
| | nuclear ribonucleoprotein that belongs |
| | to the SNRNP core protein family. The |
| | protein may act as a charged protein |
| | scaffold to promote SNRNP assembly or |
| | strengthen SNRNP—SNRNP interactions |
| | through nonspecific electrostatic |
| | contacts with RNA. |

| | |
|---|---|
| FEATURES | Location/Qualifiers |

| | |
|---|---|
| source | 1 . . . 119 |
| | /organism = “Homo sapiens” |
| | /db_xref = “taxon: 9606” |
| | /chromosome = “18” |
| | /map = “18q11.2” |
| Protein | 1 . . . 119 |
| | /product = “small nuclear ribonu- |
| | cleoprotein D1 polypeptide 16 kDa” |
| | /note = “snRNP core protein D1; |
| | Sm-D autoantigen; small nuclear ri- |
| | bonucleoprotein D1 polypeptide |
| | (16 kD)” |
| CDS | 1 . . . 119 |
| | /gene = “SNRPD1” |
| | /coded_by = “NM_006938.2: |
| | 132 . . . 491” |
| | /db_xref = “GeneID: 6632” |
| | /dbxref = “MIM: 601063” |

| | |
|---|---|
| ORIGIN | |
| 1 | mklvrflmkl shetvtielk ngtqvhgtit |
| | gvdvsmnthl kavkmtlknr epvqletlsi |
| 61 | rgnniryfil pdslpldtll vdvepkvksk |
| | kreavagrgr grgrgrgrgr grgrggprr |
| // | |

Human Tripartite Motif Protein 2 (RING Finger Protein 86)

| | |
|---|---|
| LOCUS | Q90040 744 aa linear PRI 01- |
| MAY 2005 | |
| DEFINITION | Tripartite motif protein 2 (RING finger |
| | protein 86). |
| ACCESSION | Q9C040 |
| VERSION | Q9C040 GI: 21363034 |
| DBSOURCE | swissprot: locus TRIM2_HUMAN, accession |
| | Q9C040; |
| | class: standard. |
| | extra accessions: O60272,Q9BSI9,Q9UFZ1, |
| | created: Feb. 28, 2003. |
| | sequence updated: Feb. 28, 2003. |
| | annotation updated: May 1, 2005. |
| | xrefs: AF220018.1, AAG53472.1, |
| | AB011089.1, BAA25443.1, BC005016.1, |
| | AAH05016.1, BC011052.1, AAH11052.1, |
| | AL110234.1, CAB53687.2, T00082 xrefs |
| | (non-sequence databases): HSSPP28990, |
| | EnsemblENSG00000109654, |
| | GenewHGNC: 15974, H-InvDBHIX0004577, |
| | GO0005737, GO0017022, GO0008270, |
| | InterProIPR01044, InterProIPR003649, |
| | InterProIPR001298, InterProIPR001258, |
| | InterProIPR000315, InterProIPR001841, |
| | PfamPF00630, PfamPF01436, PfamPF00643, |
| | PfamPF00097, PRINTSPR01406, |
| | SMARTSM00502, SMARTSM00336, |
| | SMARTSM00557, SMARTSM00184, |
| | PROSITEPS50194, PROSITEPS50119, |
| | PROSITEPS00518, PROSITEPS50089 |
| KEYWORDS | Metal-binding; Repeat; Zinc; |
| | Zinc-finger. |
| SOURCE | Homo sapiens (human) |

| | |
|---|---|
| ORGANISM | Homo sapiens |
| | Eukaryota; Metazoa; Chordata; Craniata; |
| | Vertebrata; Euteleostomi; Mammalia; |
| | Eutheria; Euarchontoglires; Primates; |
| | Catarrhini; Hominidae; Homo. |

| | |
|---|---|
| REFERENCE | 1 (residues 1 to 744) |

| | |
|---|---|
| AUTHORS | Reymond,A., Meroni,G., Fantozzi,A., |
| | Merla,G., Cairo,S., Luzi, L., |
| | Riganelli,D., Zanaria,E., Messali,S., |
| | Cainarca,S., Guffanti,A., Minucci,S., |
| | Pelicci,P. G. and Ballabio,A. |
| TITLE | The tripartite motif family identifies |
| | cell compartments |
| JOURNAL | EMBO J. 20 (9), 214014 2151 (2001) |
| PUBMED | 11331580 |
| REMARK | NUCLEOTIDE SEQUENCE. |

| | |
|---|---|
| REFERENCE | 2 (residues 1 to 744) |

| | |
|---|---|
| AUTHORS | Nagase,T., Ishikawa,K., Miyajima,N., |
| | Tanaka,A., Kotani,H., Nomura,N. and |
| | Ohara,O. |
| TITLE | Prediction of the coding sequences of |
| | unidentified human genes. IX. The com- |
| | plete sequences of 100 new cDNA clones |
| | from brain which can code for large |
| | proteins in vitro |
| JOURNAL | DNA Res. 5 (1), 31-39 (1998) |
| PUBMED | 9628581 |
| REMARK | NUCLEOTIDE SEQUENCE [LARGE SCALE MRNA]. |
| | TISSUE = Brain |

| | |
|---|---|
| REFERENCE | 3 (residues 1 to 744) |

| | |
|---|---|
| AUTHORS | Strauserg,R. L., Feingold,E. A., |
| | Grouse,L. H., Derge,J. G., Klausner,R. |
| | D., Collins,F. S., Wagner,L., |
| | Shenmen,C. M., Schuler, G. D., |
| | Altschul,S. F., Zeeberg,B., |
| | Buetow,K. H., Schaefer,C. F., |
| | Bhat, N. K., Hopkins,R. F., |
| | Jordan,H., Moore,T., Max,S. I., |
| | Wang,J., Hsieh, F., Diatchenko,L., |
| | Marusina,K., Farmer,A. A., Rubin,G. M., |
| | Hong,L., Stapleton,M., Soares,M. B., |
| | Bonaldo,M. F., Casavant,T. L., |
| | Scheetz,T. E., Brownstein,M. J., |
| | Usdin,T. B., Toshiyuki,S., Carninci,P., |
| | Prange,C., Raha,S. S., Loquellano,N. |
| | A., Peters,G. J., Abramson,R. D., |
| | Mullahy,S. J., Bosak,S. A., McEwan,P. |
| | J., McKernan,K. J., Malek,J. A., |
| | Gunaratne,P. H., Richards,S., Worley,K. |
| | C., Hale,S., Garcia,A. M., Gay,L. J., |
| | Hulyk,S. W., Villalon,D. K., Muzny,D. |
| | M., Sodergren,E. J., Lu,X., Gibbs,R. |
| | A., Fahey,J., Helton,E., Ketteman,M., |
| | Madan,A., Rodrigues,S., Sanchez,A., |
| | Whiting,M., Madan,A., Young,A. C., |
| | Shevchenko,Y., Bouffard,G. G., |
| | Blakesley,R. W., Touchman,J. W., |
| | Green,E. D., Dickson,M. C., |
| | Rodriguez,A. C., Grimwood,J., |
| | Schmutz,J., Myers,R. M., Butterfield,Y. |
| | S., Krzywinski,M. I., Skalska,U., |
| | Smailus,D. E., Schnerch,A., Schein,J. |
| | E., Jones,S. J. and Marra,M. A. |
| CONSRTM | Mammalian Gene Collection Program Team |
| TITLE | Generation and initial analysis of more |
| | than 15,000 full-length human and mouse |
| | cDNA sequences |
| JOURNAL | Proc. Natl. Acad. Sci. U.S.A. 99 (26), |
| | 16899-16903 (2002) |
| PUBMED | 12477932 |
| REMARK | NUCLEOTIDE SEQUENCE [LARGE SCALE MRNA]. |
| | TISSUE = Brain, and Placenta |

| | |
|---|---|
| REFERENCE | 4 (residues 1 to 744) |

| | |
|---|---|
| AUTHORS | . |
| CONSRTM | The German cDNA consortium |
| TITLE | Direct Submission |
| JOURNAL | Submitted (??-AUG-1999) |
| REMARK | NUCLEOTIDE SEQUENCE [LARGE SCALE MRNA] |
| | OF 515-744. |
| | TISSUE = Kidney |

| | |
|---|---|
| COMMENT | On Mar. 15, 2005 this sequence ver- |
| | sion replaced gi: 7513001. [FUNCTION] |
| | May contribute to the alteration of |
| | neural cellular mechanisms (By sim- |
| | ilarity). |
| | [SUBUNIT] Interacts with myosin V (By |
| | similarity). |
| | [SUBCELLULAR LOCATION] Cytoplasmic |
| | (By similarity). |
| | [DOMAIN] The interaction with myosin V |
| | is dependent upon its NHL repeats, |
| | which form a beta-propeller (NHL) |
| | domain containing six blades (By |
| | similarity). |
| | [SIMILARITY] Belongs to the TRIM/RBCC |
| | family. |
| | [SIMILARITY] Contains 1 B box-type zinc |
| | finger. |
| | [SIMILARITY] Contains 1 filamin repeat. |
| | [SIMILARITY] Contains 6 NHL repeats. |
| | [SIMILARITY] Contains 1 RING-type zinc finger. |

| | |
|---|---|
| FEATURES | Location/Qualifiers |

| | |
|---|---|
| source | 1 . . . 744 |
| | /organism = “Homo sapiens” |
| | /db_xref = “taxon: 9606” |
| gene | 1 . . . 744 |
| | /gene = “TRIM2” |
| | /note = “synonyms: KIAA0517, RNF86” |
| Protein | 1 . . . 744 |
| | /gene = “TRIM2” |
| | /product = “Tripartite motif |
| | protein 2” |
| Region | 23 . . . 64 |
| | /gene = “TRIM2” |
| | /region_name = “Zinc finger region” |
| | /note = “RING-type.” |
| | /evidence = experimental |
| Region | 113 . . . 154 |
| | /gene = “TRIM2” |
| | /region_name = “Zinc finger region” |
| | /note = “B box-type.” |
| | /evidence = experimental |
| Region | 320 . . . 421 |
| | /gene = “TRIM2” |
| | /region_name = “Repetitive region” |
| | /note = “Filamin.” |
| | /evidence = experimental |
| Region | 486 . . . 513 |
| | /gene = “TRIM2” |
| | /region_name = “Repetitive region” |
| | /note = “NHL 1.” |
| | /evidence = experimental |
| Region | 515 |
| | /gene = “TRIM2” |
| | /region_name = “Conflict” |
| | /note = “N −> G (in REF. 4).” |
| | /evidence = “experimental |
| Region | 533 . . . 560 |
| | /gene = “TRIM2” |
| | /region_name = “Repetitive region” |
| | /note = “NHL 2.” |
| | /evidence = experimental |
| Region | 575 . . . 602 |
| | /gene = “TRIM2” |
| | /region_name = “Repetitive region” |
| | /note = “NHL 3.” |
| | /evidence = experimental |
| Region | 622 . . . 649 |
| | /gene = “TRIM2” |
| | /region_name = “Repetitive region” |
| | /note = “NHL 4.” |
| | /evidence = experimental |
| Region | 669 . . . 696 |
| | /gene = “TRIM2” |
| | /region_name = “Repetitive region” |
| | /note = “NHL 5.” |
| | /evidence = experimental |
| Region | 713 . . . 740 |
| | /gene = “TRIM2” |
| | /region_name = “Repetitive region” |
| | /note = “NHL 6.” |
| | /evidence = experimental |
| Region | 737 . . . 744 |
| | /gene = “TRIM2” |
| | /region_name = “Conflict” |
| | /note = “FKVYRYLQ −> |
| | LILIYSRHLFFYESKC (in REF. 3; |
| | AAH05016).” |
| | /evidence = experimental |

| | |
|---|---|
| ORIGIN | |
| 1 | masegtnips pvvrqidkqf licsiclery |
| | knpkvlpclh tfcerclqny ipahsltlsc |
| 61 | pvcrqtsilp ekgvaalqnn ffitnlmdvl |
| | qrtpgsnaee ssiletvtav aagkplscpn |
| 121 | hdgnvmefyc qscetamcre ctegehaehp |
| | tvplkdvveq hkaslqvgld avnkrlpeid |
| 181 | salqfiseii hqltnqkasi vddihstfde |
| | lqktlnvrks vllmelevny glkhkvlqsq |
| 241 | ldtllqgqes ikscsnftaq alnhgtetev |
| | llvkkqmsek lneladqdfp lhprendqld |
| 301 | fiveteglkk sihnlgtilt tnavasetva |
| | tgeglrqtii gqpmsvtitt kdkdgelckt |
| 361 | gnayltaels tpdgsvadge ildnkngtye |
| | flytvqkegd ftlslrlydq hirgspfklk |
| 421 | virsadvspt tegvkrrvks pgsghvkqka |
| | vkrpasmyst gkrkenpied dlifrvgtkg |
| 481 | rnkgeftnlq gvaastngki liadsnnqcv |
| | qifsndgqfk srfgirgrsp gqlqrptgva |
| 541 | vhpsgdiiia dydnkwvsif ssdgkfktki |
| | gsgklmgpkg vsvdrnghii vvdnkaccvf |
| 601 | ifqpngkivt rfgsrgngdr qfagphfaav |
| | nsnneiiitd fhnhsvkvfn qegefmlkfg |
| 661 | sngegngqfn aptgvavdsn gniivadwgn |
| | sriqvfdgsg sflsyintsa dplygpqgla |
| 721 | ltsdghvvva dsgnhcfkvy rylq |
| // | |

Human TFIID Subunit 6

| | |
|---|---|
| LOCUS | P49848 677 aa linear PRI 01- |
| MAY 2005 | |
| DEFINITION | Transcription initiation factor TFIID |
| | subunit 6 (Transcription initiation |
| | factor TFIID 70 kDa subunit) |
| | (TAF(II)70) (TAFII-70) (TAFII-80) |
| | (TAFII80). |
| ACCESSION | P49848 |
| VERSION | P49848 GI: 1729810 |
| DBSOURCE | swissprot: locus TAF6_HUMAN, accession |
| | P49848; |
| | class: standard. |
| | created: Oct. 1, 1996. |
| | sequence updated: Oct. 1, 1996. |
| | annotation updated: May 1, 2005. |
| | xrefs: L25444.1, AAA63643.1, U31659.1, |
| | AAA84390.1, AY149894.1, AAN10295.1, |
| | BC018115.1, AAH18115.1 |
| | xrefs (non-sequence databases): |
| | HSSPP49847, TRANSFACT00783, |
| | TRANSFACT02208, GenewHGNC: 11540, |
| | H-InvDBHIX0006909, ReactomeP49848, |
| | MIM 602955, GO0005669, GO0005673, |
| | GO0016251, GO0005515, |
| | InterProIPR007124, InterProIPR009072, |
| | InterProIPR004823, PfamPF02969 |
| KEYWORDS | Direct protein sequencing; Nuclear |
| | protein; Polymorphism; Transcription; |
| | Transcription regulation. |
| SOURCE | Homo sapiens (human) |

| | |
|---|---|
| ORGANISM | Homo sapiens |
| | Eukaryota; Metazoa; Chordata; Craniata; |
| | Vertebrata; Euteleostomi; Mammalia; |
| | Eutheria; Euarchontoglires; Primates; |
| | Catarrhini; Hominidae; Homo. |

| | |
|---|---|
| REFERENCE | 1 (residues 1 to 677) |

| | |
|---|---|
| AUTHORS | Weinzierl,R. O., Ruppert,S., Dynlacht, |
| | B. D., Tanese,N. and Tjian, R. |
| TITLE | Cloning and expression of Drosophila |
| | TAFII60 and human TAFII70 reveal con- |
| | served interactions with other subunits |
| | of TFIID |
| JOURNAL | EMBO J. 12 (13), 5303-5309 (1993) |
| PUBMED | 8262073 |
| REMARK | NUCLEOTIDE SEQUENCE, AND PARTIAL |
| | PROTEIN SEQUENCE. |

| | |
|---|---|
| REFERENCE | 2 (residues 1 to 677) |

| | |
|---|---|
| AUTHORS | Hisatake,K., Ohta,T., Takada,R., |
| | Guermah,M., Horikoshi,M., Nakatani,Y. |
| | and Roeder,R. G. |
| TITLE | Evolutionary conservation of human |
| | TATA-binding-polypeptide-associated |
| | factors TAFII31 and TAFII80 and in- |
| | teractions of TAFII80 with other TAFs |
| | and with general transcription factors |
| JOURNAL | Proc. Natl. Acad. Sci. U.S.A. 92 (18), |
| | 8195-8199 (1995) |
| PUBMED | 7667268 |
| REMARK | NUCLEOTIDE SEQUENCE. |
| | TISSUE = Placenta |

| | |
|---|---|
| REFERENCE | 3 (residues 1 to 677) |

| | |
|---|---|
| AUTHORS | Rieder,M. J., Livingston,R. J., |
| | Daniels,M. R., Montoya,M. A., Chung,M. |
| | -W., Miyamoto,K. E., Nguyen,C. P., |
| | Nguyen,D. A., Poel,C.L., Robertson, |
| | P. D., Schackwitz,W. S., Sherwood, |
| | J. K., Witrak,L. A. and Nickerson, |
| | D. A. |
| TITLE | Direct Submission |
| JOURNAL | Submitted (??-SEP-2002) |
| REMARK | NUCLEOTIDE SEQUENCE, AND VARIANT |
| | SER-36. |

| | |
|---|---|
| REFERENCE | 4 (residues 1 to 677) |

| | |
|---|---|
| AUTHORS | Strausberg,R. L., Feingold,E. A., |
| | Grouse,L. H., Derge,J. G., Klausner, |
| | R. D., Collins,F. S., Wagner,L., |
| | Shenmen,C. M., Schuler,G. D., |
| | Altschul,S. F., Zeeberg,B., Buetow, |
| | K. H., Schaefer,C. F., Bhat, N. K., |
| | Hopkins,R. F., Jordan,H., Moore,T., |
| | Max,S. I., Wang,J., Hsieh, F., |
| | Diatchenko,L., Marusina,K., Farmer,A. |
| | A., Rubin,G. M., Hong,L., Stapleton,M., |
| | Soares,M. B., Bonaldo,M. F., Casavant, |
| | T. L., Scheetz,T. E., Brownstein,M. J., |
| | Usdin,T. B., Toshiyuki,S., Carninci,P., |
| | Prange,C., Raha,S. S., Loquellano,N. |
| | A., Peters, G. J., Abramson,R. D., |
| | Mullahy,S. J., Bosak,S. A., McEwan,P. |
| | J., McKernan,K. J., Malek,J. A., |
| | Gunaratne,P. H., Richards,S., Worley, |
| | K. C., Hale,S., Garcia,A. M., Gay,L. |
| | J., Hulyk,S. W., Villalon,D. K., |
| | Muzny,D. M., Sodergren,E. J., Lu,X., |
| | Gibbs,R. A., Fahey,J., Helton,E., |
| | Ketteman,M., Madan,A., Rodrigues,S., |
| | Sanchez,A., Whiting,M., Madan,A., |
| | Young,A. C., Shevchenko,Y., Bouffard, |
| | G. G., Blakesley,R. W., Touchman,J. |
| | W., Green,E. D., Dickson,M. C., |
| | Rodriguez,A. C., Grimwood,J., Schmutz, |
| | J., Myers,R. M., Butterfield,Y. S., |
| | Krzywinski,M. I., Skalska,U., Smailus, |
| | D. E., Schnerch,A., Schein,J. E., |
| | Jones,S. J. and Marra,M. A. |
| CONSRTM | Mammalian Gene Collection Program Team |
| TITLE | Generation and initial analysis of more |
| | than 15,000 full-length human and mouse |
| | cDNA sequences |
| JOURNAL | Proc. Natl. Acad. Sci. U.S.A. 99 (26), |
| | 16899-16903 (2002) |
| PUBMED | 12477932 |
| REMARK | NUCLEOTIDE SEQUENCE [LARGE SCALE MRNA]. |
| | TISSUE = Pancreas |

| | |
|---|---|
| COMMENT | [FUNCTION] TAFs are components of the |
| | transcription factor IID (TFIID) com- |
| | plex, PCAF histone acetylase complex |
| | and TBP-free TAFII complex (TFTC). |
| | TIIFD is multimeric protein complex |
| | that plays a central role in mediating |
| | promoter responses to various |
| | activators and repressors. |
| | [SUBUNIT] TFIID and PCAF are composed |
| | of TATA binding protein (TBP) and a |
| | number of TBP-associated factors |
| | (TAFs). TBP is not part of TFTC. Binds |
| | tightly to TAFII-250 and also directly |
| | interacts with TAFII-40. |
| | [SUBCELLULAR LOCATION] Nuclear. |
| | [SIMILARITY] Belongs to the TAF6 |
| | family. |

| | |
|---|---|
| FEATURES | Location/Qualifiers |

| | |
|---|---|
| source | 1 . . . 677 |
| | /organism = “Homo sapiens” |
| | /db_xref = “taxon: 9606” |
| gene | 1 . . . 677 |
| | /gene = “TAF6” |
| | /note = “synonyms: TAF2E, TAFII70” |
| Protein | 1 . . . 677 |
| | /gene = “TAF6” |
| | /product = “Transcription initiation |
| | factor TFIID subunit 6” |
| Region | 36 |
| | /gene = “TAF6” |
| | /region_name = “Variant” |
| | /note = “C −> S./FTId = |
| | VAR_0143492.” |
| | /evidence = experimental |

| | |
|---|---|
| ORIGIN | |
| 1 | maeekklkls ntvlpsesmk vvaesmgiaq |
| | iqeetcqllt devsyrikei aqdalkfmhm |
| 61 | qkrqklttsd idyalklknv eplygfhaqe |
| | fipfrfasgg grelyfyeek evdlsdiint |
| 121 | plprvpldvc lkahwlsieg cqpaipenpp |
| | papkeqqkae ateplksakp gqeedgplkg |
| 181 | kgqgattadg kgkekkappl legaplrlkp |
| | rsihelsveq qlyykeitea cvgsceakra |
| 241 | ealqsiatdp glyqmlprfs tfisegvrvn |
| | vvqnnlalli ylmrmvkalm dnptlyleky |
| 301 | vhelipavmt civsrqlclr pdvdnhwalr |
| | dfaarlvaqi ckhfstttnn iqsritktft |
| 361 | kswvdektpw ttrygsiagl aelghdvikt |
| | lilprlqqeg erirsvldgp vlsnidriga |
| 421 | dhvqslllkh capvlaklrp ppdnqdayra |
| | efgslgpllc sqvvkaraqa alqaqqvnrt |
| 481 | tltitqprpt ltlsqapqpg prtpgllkvp |
| | gsialpvqtl vsaraaappq psppptkfiv |
| 541 | msssssapst qqvlslstsa pgsgstttsp |
| | vtttvpsvqp ivklvstatt appstapsgp |
| 601 | gsvqkyivvs lpptgegkgg ptshpspvpp |
| | passpsplsg salcggkqea gdspppapgt |
| 661 | pkangsqpns gspqpap |
| // | |

Human TAR-Binding Protein

| | |
|---|---|
| LOCUS | NP_000958 403 aa linear PRI 02- |
| MARCH 2005 | |
| DEFINITION | ribosomal protein L3 [Homo sapiens]. |
| ACCESSION | NP_000958 |
| VERSION | NP_000958.1 GI: 4506649 |
| DBSOURCE | REFSEQ: accession NM 000967.2 |
| KEYWORDS | . |
| SOURCE | Homo sapiens (human) |

| | |
|---|---|
| ORGANISM | Homo sapiens |
| | Eukaryota; Metazoa; Chordata; Craniata; |
| | Vertebrata; Euteleostomi; Mammalia; |
| | Eutheria; Euarchontoglires; Primates; |
| | Catarrhini; Hominidae; Homo. |

| | |
|---|---|
| REFERENCE | 1 (residues 1 to 403) |

| | |
|---|---|
| AUTHORS | Collins,J. E., Wright,C. L., Edwards,C. |
| | A., Davis,M. P., Grinham, J. A., Cole, |
| | C. G., Goward, M.E., Aguado,B., Mallya, |
| | M., Mokrab,Y., Huckle,E. J., Beare,D. |
| | M. and Dunham,I. |
| TITLE | A genome annotation-driven approach to |
| | cloning the human ORFeome |
| JOURNAL | Genome Biol. 5 (10), R84 (2004) |
| PUBMED | 15461802 |

| | |
|---|---|
| REFERENCE | 2 (residues 1 to 403) |

| | |
|---|---|
| AUTHORS | Uechi,T., Tanaka,T. and Kenmochi,N. |
| TITLE | A complete map of the human ribosomal |
| | protein genes: assignment of 80 genes |
| | to the cytogenetic map and implications |
| | for human disorders |
| JOURNAL | Genomics 72 (3), 223-230 (2001) |
| PUBMED | 11401437 |

| | |
|---|---|
| REFERENCE | 3 (residues 1 to 403) |

| | |
|---|---|
| AUTHORS | Duga,S., Asselta,R., Malcovati,M., |
| | Tenchini,M. L., Ronchi,S. and |
| | Simonic, T. |
| TITLE | The intron-containing L3 ribosomal pro- |
| | tein gene (RPL3): sequence analysis and |
| | identification of U43 and of two novel |
| | intronic small nucleolar RNAs |
| JOURNAL | Biochim. Biophys. Acta 1490 (3), |
| | 225-236 (2000) |
| PUBMED | 10684968 |

| | |
|---|---|
| REFERENCE | 4 (residues 1 to 403) |

| | |
|---|---|
| AUTHORS | Kenmochi,N., Kawaguchi,T., Rozen,S., |
| | Davis,E., Goodman,N., Hudson,T. J., |
| | Tanaka,T. and Page,D. C. |
| TITLE | A map of 75 human ribosomal protein |
| | genes |
| JOURNAL | Genome Res. 8 (5), 509-523 (1998) |
| PUBMED | 9582194 |

| | |
|---|---|
| REFERENCE | 5 (residues 1 to 403) |

| | |
|---|---|
| AUTHORS | Wool,I. G., Chan,Y. L. and Gluck,A. |
| TITLE | Structure and evolution of mammalian |
| | ribosomal proteins |
| JOURNAL | Biochem. Cell Biol. 73 (11-12), |
| | 933-947 (1995) |
| PUBMED | 8722009 |
| REMARK | Review article |

| | |
|---|---|
| REFERENCE | 6 (residues 1 to 403) |

| | |
|---|---|
| AUTHORS | Reddy,T. R., Suhasini,M., Rappaport,J., |
| | Looney,D. J., Kraus,G. and Wong-Staal, |
| | F. |
| TITLE | Molecular cloning and characterization |
| | of a TAR-binding nuclear factor from T |
| | cells |
| JOURNAL | AIDS Res. Hum. Retroviruses 11 (6), |
| | 663-669 (1995) |
| PUBMED | 7576925 |

| | |
|---|---|
| REFERENCE | 7 (residues 1 to 403) |

| | |
|---|---|
| AUTHORS | Matoba,R., Okubo,K., Hori,N., |
| | Fukushima,A. and Matsubara,K. |
| TITLE | The addition of 5′-coding information |
| | to a 3′-directed cDNA library improves |
| | analysis of gene expression |
| JOURNAL | Gene 146 (2), 199-207 (1994) |
| PUBMED | 8076819 |

| | |
|---|---|
| COMMENT | REVIEWED REFSEQ: This record has been |
| | curated by NCBI staff. The reference |
| | sequence was derived from BC012146.1 |
| | and BC008492.1. |
| | Summary: Ribosomes, the organelles that |
| | catalyze protein synthesis, consist of |
| | a small 40S subunit and a large 60S |
| | subunit. Together these subunits are |
| | composed of 4 RNA species and approxi- |
| | mately 80 structurally distinct pro- |
| | teins. This gene encodes a ribosomal |
| | protein that is a component of the 60S |
| | subunit. The protein belongs to the L3P |
| | family of ribosomal proteins. It is |
| | located in the cytoplasm. The protein |
| | can bind to the HIV-1 TAR mRNA, and it |
| | has been suggested that the protein |
| | contributes to tat-mediated trans- |
| | activation. This gene is co-transcribed |
| | with the small nucleolar RNA genes U43, |
| | U86, U83a, and U83b, which are located |
| | in its first, third, fifth, and seventh |
| | introns, respectively. As is typical |
| | for genes encoding ribosomal proteins, |
| | there are multiple processed pseudo- |
| | genes of this gene dispersed through |
| | the genome. |

| | |
|---|---|
| FEATURES | Location/Qualifiers |

| | |
|---|---|
| source | 1 . . . 403 |
| | /organism = “Homo sapiens” |
| | /db_xref = “taxon: 9606” |
| | /chromosome = “22” |
| | /map = “22q13” |
| Protein | 1 . . . 403 |
| | /product = “ribosomal protein L3” |
| | /note = “60S ribosomal protein L3; |
| | HIV-1 TAR RNA-binding protein B“ |
| CDS | 1 . . . 403 |
| | /gene = “RPL3“ |
| | /coded_by = “NM_000967.2: |
| | 27 . . . 1238” |
| | /db_xref = “CCDS: CCDS13988.1” |
| | /db_xref = “GeneID: 6122” |
| | /db_xref = “MIM: 604163” |

| | |
|---|---|
| ORIGIN | |
| 1 | mshrkfsapr hgslgflprk rssrhrgkvk |
| | sfpkddpskp vhltaflgyk agmthivrev |
| 61 | drpgskvnkk evveavtive tppmvvvgiv |
| | gyvetprglr tfktvfaehi sdeckrrfyk |
| 121 | nwhkskkkaf tkyckkwqde dgkkqlekdf |
| | ssmkkycqvi rviahtgmrl lplrqkkahl |
| 181 | meiqvnggtv aekldwarer leqqvpvnqv |
| | fgqdemidvi gvtkgkgykg vtsrwhtkkl |
| 241 | prkthrglrk vacigawhpa rvafsvarag |
| | qkgyhhrtei nkkiykigqg ylikdgklik |
| 301 | nnastdydls dksinplggf vhygevtndf |
| | vmlkgcvvgt kkrvltlrks llvqtkrral |
| 361 | ekidlkfidt tskfghgrfq tmeekkafmg |
| | plkkdriake ega |
| // | |

Human ERI-1 (AAH35279)

| | |
|---|---|
| LOCUS | AAH35279 349 aa linear PRI 05- |
| APRIL 2005 | |
| DEFINITION | Histone mRNA 3′ end-specific |
| | exonuclease [Homo sapiens]. |
| ACCESSION | AAH35279 |
| VERSION | AAH35279.1 GI: 23271401 |
| DBSOURCE | accession BC035279.1 |
| KEYWORDS | MGC. |
| SOURCE | Homo sapiens (human) |

| | |
|---|---|
| ORGANISM | Homo sapiens |
| | Eukaryota; Metazoa; Chordata; Craniata; |
| | Vertebrata; Euteleostomi; Mammalia; |
| | Eutheria; Euarchontoglires; Primates; |
| | Catarrhini; Hominidae; Homo. |

| | |
|---|---|
| REFERENCE | 1 (residues 1 to 349) |

| | |
|---|---|
| AUTHORS | Strausberg,R. L., Feingold,E. A., |
| | Grouse,L. H., Derge,J. G., Klausner, |
| | R. D., Collins,F. S., Wagner,L., |
| | Shenmen,C. M., Schuler,G. D., |
| | Altschul,S. F., Zeeberg,B., Buetow, |
| | K. H., Schaefer,C. F., Bhat, N. K., |
| | Hopkins,R. F., Jordan,H., Moore,T., |
| | Max,S. I., Wang,J., Hsieh, F., |
| | Diatchenko,L., Marusina,K., Farmer,A. |
| | A., Rubin,G. M., Hong,L., Stapleton, |
| | M., Soares,M. B., Bonaldo,M. F., |
| | Casavant,T. L., Scheetz,T. E., |
| | Brownstein,M. J., Usdin,T. B., |
| | Toshiyuki,S., Carninci,P., Prange,C., |
| | Raha,S. S., Loquellano,N. A., Peters, |
| | G. J. Abramson,R. D., Muilahy,S. J., |
| | Bosak,S. A., McEwan,P. J., McKernan, |
| | K. J., Malek,J. A., Gunaratne,P. H., |
| | Richards,S., Worley,K. C., Hale,S., |
| | Garcia,A. M., Gay,L. J., Hulyk,S. W., |
| | Villalon,D. K., Muzny,D. M., |
| | Sodergren,E. J., Lu,X., Gibbs,R. A., |
| | Fahey,J., Helton,E., Ketteman,M., |
| | Madan,A., Rodrigues,S., Sanchez,A., |
| | Whiting,M., Madan,A., Young,A. C., |
| | Shevchenko,Y., Bouffard,G. G., |
| | Blakesley,R. W., Touchman,J. W., |
| | Green,E. D., Dickson,M. C., |
| | Rodriguez,A. C., Grimwood,J., |
| | Schmutz,J., Myers, R. M., Butterfield, |
| | Y. S., Krzywinski,M. I., Skalska,U., |
| | Smailus,D. E., Schnerch,A., Schein, |
| | J. E., Jones,S. J. and Marra,M. A. |
| CONSRTM | Mammalian Gene Collection Program |
| | Team |
| TITLE | Generation and initial analysis of |
| | more than 15,000 full-length human |
| | and mouse cDNA sequences |
| JOURNAL | Proc. Natl. Acad. Sci. U.S.A. 99 |
| | (26), 16899-16903 (2002) |
| PUBMED | 12477932 |

| | |
|---|---|
| REFERENCE | 2 (residues 1 to 349) |

| | |
|---|---|
| AUTHORS | . |
| CONSRTM | NIH MGC Project |
| TITLE | Direct Submission |
| JOURNAL | Submitted (31-JUL-2002) National |
| | Institutes of Health, Mammalian |
| | Gene Collection (MGC), Bethesda, |
| | MD 20892-2590, USA |
| REMARK | NIH-MGC Project URL: |
| | http://mgc.nci.nih.gov |

| | |
|---|---|
| COMMENT | Contact: MGC help desk |
| | Email: cgapbs-r@mail.nih.gov |
| | Tissue Procurement: Life Technologies, |
| | Inc. |
| | cDNA Library Preparation: Life |
| | Technologies, Inc. |
| | cDNA Library Arrayed by: The I.M.A.G.E. |
| | Consortium (LLNL) DNA |
| | Sequencing by: Baylor College of Med- |
| | icine Human Genome Sequencing Center |
| | Center code: BCM-HGSC |
| | Web site: |
| | http://www.hgsc.bcm.tmc.edu/cdna/ |
| | Contact: amg@bcm.tmc.edu |
| | Gunaratne, P. H., Garcia, A. M., Lu, |
| | X., Hulyk, S. W., Loulseged, H., Kowis, |
| | C. R., Sneed, A. J., Martin, R. G., |
| | Muzny, D. M., Nanavati, A. N., Gibbs, |
| | R. A. |
| | Clone distribution: MGC clone distribu- |
| | tion information can be found through |
| | the I.M.A.G.E. Consortium/LLNL at: |
| | http://image.llnl.gov |
| | Series: IRAK Plate: 50 Row: g |
| | Column: 1 |
| | This clone was selected for full length |
| | sequencing because it passed the fol- |
| | lowing selection criteria: matched |
| | mRNA gi: 31543183. |
| | Method: conceptual translation. |

| | |
|---|---|
| FEATURES | Location/Qualifiers |

| | |
|---|---|
| source | 1 . . . 349 |
| | /organism = “Homo sapiens” |
| | /db_xref = “taxon: 9606” |
| | /clone = “MGC: 35395 IMAGE: 5186320' |
| | /tissue_type = “Colon, Kidney, |
| | Stomach, adult, whole pooled” |
| | /clone_lib = “NIH_MGC_116” |
| | /lab_host = “DH10B” |
| | /note = “Vector: pCMV-SPORT6” |
| Protein | 1 . . . 349 |
| | /product = “histone mRNA 3′ end- |
| | specific exonuclease” |
| CDS | 1 . . . 349 |
| | /gene = “3′ HEXO” |
| | /coded_by = “BC035279.1: |
| | 125 . . . 1174” |
| | /db_xref = “GeneID: 90459” |

| | |
|---|---|
| ORIGIN | |
| 1 | medpqskepa geavalalle sprpeggeep |
| | prpspeetqq ckfdgqetkg skfitssasd |
| 61 | fsdpvykeia itngcinrms keelraklse |
| | fkletrgvkd vlkkrlknyy kkqklmlkes |
| 121 | nfadsyydyi ciidfeatce egnppefvhe |
| | iiefpvvlln thtleiedtf qqyvrpeint |
| 181 | qlsdfcislt gitqdqvdra dtfpqvlkkv |
| | idwmklkelg tkykyslltd gswdmskfln |
| 241 | iqcqlsrlky ppfakkwini rksygnfykv |
| | prsqtkltim leklgmdydg rphcglddsk |
| 301 | niariavrml qdgcelrine kmhagqlmsv |
| | ssslpiegtp ppqmphfrk |
| // | |

Human TUDOR Protein

| | |
|---|---|
| LOCUS | Q9BXT4 777 aa linear PRI 01- |
| MAY 2005 | |
| DEFINITION | Tudor domain containing protein 1. |
| ACCESSION | Q9BXT4 |
| VERSION | Q9BXT4 GI: 17368689 |
| DBSOURCE | swissprot: locus TDRD1_HUMAN, |
| | accession Q9BXT4; |
| | class: standard. |
| | extra accessions: Q9H7B3, created: |
| | Feb. 28, 2003. |
| | sequence updated: Feb. 28, 2003. |
| | annotation updated: May 1, 2005. |
| | xrefs: AF285606.1, AAK31985.1, |
| | AK024735.1, BAB14982.1 |
| | xrefs (non-sequence databases): |
| | GenewHGNC: 11712, MIM 605796, Inter- |
| | ProIPR008191, InterProIPR002999, |
| | PfamPF00567, SMARTSM00333, |
| | PROSITEPS50304 |
| KEYWORDS | Repeat. |
| SOURCE | Homo sapiens (human) |

| | |
|---|---|
| ORGANISM | Homo sapiens |
| | Eukaryota; Metazoa; Chordata; Craniata; |
| | Vertebrata; Euteleostomi; Mammalia; |
| | Eutheria; Euarchontoglires; Primates; |
| | Catarrhini; Hominidae; Homo. |

| | |
|---|---|
| REFERENCE | 1 (residues 1 to 777) |

| | |
|---|---|
| AUTHORS | Wang,P. J., McCarrey,J. R., Yang,F. and |
| | Page,D. C. |
| TITLE | An abundance of X-linked genes ex- |
| | pressed in spermatogonia |
| JOURNAL | Nat. Genet. 27 (4), 422-426 (2001) |
| PUBMED | 11279525 |
| REMARK | NUCLEOTIDE SEQUENCE. |
| | TISSUE = Testis |

| | |
|---|---|
| REFERENCE | 2 (residues 1 to 777) |

| | |
|---|---|
| AUTHORS | Ota,T., Suzuki,Y., Nishikawa,T., |
| | Otsuki,T., Sugiyama,T., Irie, R., |
| | Wakamatsu,A., Hayashi,K., Sato,H., |
| | Nagai,K., Kimura,K., Makita, H. |
| | Sekine,M., Obayashi,M., Nishi,T., |
| | Shibahara,T., Tanaka,T., Ishii,S., |
| | Yamamoto,J., Saito,K., Kawai,Y., |
| | Isono,Y., Nakamura, Y., Nagahari,K., |
| | Murakami,K., Yasuda,T., Iwayanagi,T., |
| | Wagatsuma,M., Shiratori,A., Sudo,H., |
| | Hosoiri,T., Kaku,Y., Kodaira,H., |
| | Kondo, H., Sugawara,M., Takahashi,M., |
| | Kanda,K., Yokoi,T., Furuya,T., Kikkawa, |
| | E., Omura,Y., Abe,K., Kamihara,K., |
| | Katsuta,N., Sato, K., Tanikawa,M., |
| | Yamazaki,M., Ninomiya,K., Ishibashi,T., |
| | Yamashita, H., Murakawa,K., Fujimori, |
| | K., Tanai,H., Kimata,M., Watanabe,M., |
| | Hiraoka,S., Chiba,Y., Ishida,S., Ono, |
| | Y., Takiguchi,S., Watanabe, S., Yosida, |
| | M., Hotuta,T., Kusano,J., Kanehori,K., |
| | Takahashi-Fujii, A. Hara,H., Tanase,T. |
| | O., Nomura,Y., Togiya,S., Komai,F., |
| | Hara,R., Takeuchi,K., Arita,M., Imose, |
| | N., Musashino,K., Yuuki,H., Oshima,A., |
| | Sasaki,N., Aotsuka,S., Yoshikawa,Y., |
| | Matsunawa,H., Ichihara,T., Shiohata,N., |
| | Sano,S., Moriya,S., Momiyama,H., |
| | Satoh,N., Takami, S., Terashima,Y., |
| | Suzuki,O., Nakagawa,S., Senoh,A., |
| | Mizoguchi,H., Goto,Y., Shimizu,F., |
| | Wakebe,H., Hishigaki,H., Watanabe,T., |
| | Sugiyama,A., Takemoto,M., Kawakami,B., |
| | Yamazaki,M., Watanabe, K., Kumagai,A., |
| | Itakura,S., Fukuzumi,Y., Fujimori,Y., |
| | Komiyama,M., Tashiro,H., Tanigami,A., |
| | Fujiwara,T., Ono,T., Yamada,K., Fujii, |
| | Y., Ozaki,K., Hirao,M., Ohmori,Y., |
| | Kawabata,A., Hikiji,T., Kobatake,N., |
| | Inagaki,H., Ikema,Y., Okamoto,S., |
| | Okitani,R., Kawakami,T., Noguchi,S., |
| | Itoh,T., Shigeta,K., Senba,T., |
| | Matsumura,K., Nakajima,Y., Mizuno,T., |
| | Morinaga,M., Sasaki,M., Togashi,T., |
| | Oyama,M., Hata,H., Watanabe,M., |
| | Komatsu,T., Mizushima-Sugano, J., |
| | Satoh,T., Shirai,Y., Takahashi,Y., |
| | Nakagawa,K., Okumura,K., Nagase,T., |
| | Nomura,N., Kikuchi,H., Masuho,Y., |
| | Yamashita,R., Nakai,K., Yada,T., |
| | Nakamura,Y., Ohara,O., Isogai,T. and |
| | Sugano, S. |
| TITLE | Complete sequencing and characteriza- |
| | tion of 21,243 full-length human cDNAs |
| JOURNAL | Nat. Genet. 36 (1), 40-45 (2004) |
| PUBMED | 14702039 |
| REMARK | NUCLEOTIDE SEQUENCE [LARGE SCALE MRNA] |
| | OF 67-777. |

| | |
|---|---|
| COMMENT | [TISSUE SPECIFICITY] Testis and ovary |
| | specific. [SIMILARITY] Contains 3 Tudor |
| | domains. |

| | |
|---|---|
| FEATURES | Location/Qualifiers |

| | |
|---|---|
| source | 1 . . . 777 |
| | /organism = “Homo sapiens” |
| | /db_xref = “taxon: 9606” |
| gene | 1 . . . 777 |
| | /gene = “TDRD1” |
| Protein | 1 . . . 777 |
| | /gene = “TDRD1” |
| | /product = “Tudor domain containing |
| | protein 1” |
| Region | 138 . . . 197 |
| | /gene = “TDRD1” |
| | /region_name = “Domain” |
| | /note = “Tudor 1.” |
| | /evidence = experimental |
| Region | 359 . . . 418 |
| | /gene = “TDRD1” |
| | /region_name = “Domain” |
| | /note = “Tudor 2.” |
| | /evidence = experimental |
| Region | 587 . . . 645 |
| | /gene = “TDRD1” |
| | /region_name = “Domain” |
| | /note = “Tudor 3.” |
| | /evidence = experimental |
| Region | 737 |
| | /gene = “TDRD1” |
| | /region_name = “Conflict” |
| | /note = “T −> M (in REF. 2).” |
| | /evidence = experimental |
| Region | 775 . . . 777 |
| | /gene = “TDRD1” |
| | /region_name = “Conflict” |
| | /note = “VKS −> KKKKK (in REF. 2).” |
| | /evidence = experimental |

| | |
|---|---|
| ORIGIN | |
| 1 | meqycsikiv dileeevvtf avevelpnsg |
| | klldhvliem gyglkpsgqd skkenadqsd |
| 61 | pedvgkmtte nnivvdksdl ipkvltlnvg |
| | defcgvvahi qtpedffcqq lqsgrklael |
| 121 | qaslskycdq lpprsdfypa igdiccaqfs |
| | eddqwyrasv layaseesvl vgyvdygnfe |
| 181 | ilslmrlcpi ipkllelpmq aikcvlagvk |
| | pslgiwtpea iclmkklvqn kiitvkvvdk |
| 241 | lensslveli dksetphvsv skvlldagfa |
| | vgeqsmvtdk psdvketsvp lgvegkvnpl |
| 301 | ewtwvelgvd qtvdvvvcvi yspgefychv |
| | lkedalkkln dlnkslaehc qqklpngfka |
| 361 | eigqpccaff agdgswyral vkeilpnghv |
| | kvhfvdygni eevtadelrm isstflnlpf |
| 421 | qgircqladi qsrnkhwsee aitrfgmcva |
| | giklqarvve vtengigvel tdlstcypri |
| 481 | isdvlidehl vlksasphkd lpndrlvnkh |
| | elqvhvqglq atssaeqwkt ielpvdktiq |
| 541 | anvleiispn ifyalpkgmp enqeklcmlt |
| | aelleycnap ksrppyrpri gdaccakyts |
| 601 | ddfwyravvl gtsdtdvevl yadygnietl |
| | plcrvqpits shlalpfqii rcsleglmel |
| 661 | ngsssqliim llknfmlnqn vmlsvkgitk |
| | nvhtvsvekc sengtvdvad klvtfglakn |
| 721 | itpqrqsaln tekmyrtncc ctelqkqvek |
| | hehillflln nstnqnkfie mkklvks |
| // | |

Human Dual Specificity Phosphatase II (DUSPII)

| | |
|---|---|
| LOCUS | NP_003575 330 aa linear PRI 02- |
| MARCH 2005 | |
| DEFINITION | dual specificity phosphatase 11 [Homo |
| | sapiens]. |
| ACCESSION | NP_003575 |
| VERSION | NP_003575.1 GI: 4503415 |
| DBSOURCE | REFSEQ: accession NM 003584.1 |
| KEYWORDS | . |
| SOURCE | Homo sapiens (human) |

| | |
|---|---|
| ORGANISM | Homo sapiens |
| | Eukaryota; Metazoa; Chordata; Craniata; |
| | Vertebrata; Euteleostomi; Mammalia; |
| | Eutheria; Euarchontoglires; Primates; |
| | Catarrhini; Hominidae; Homo. |

| | |
|---|---|
| REFERENCE | 1 (residues 1 to 330) |

| | |
|---|---|
| AUTHORS | Yuan,Y., Li,D. M. and Sun,H. |
| TITLE | PIR1, a novel phosphatase that exhibits |
| | high affinity to RNA ribonucleoprotein |
| | complexes |
| JOURNAL | J. Biol. Chem. 273 (32), 20347-20353 |
| | (1998) |
| PUBMED | 9685386 |

| | |
|---|---|
| COMMENT | REVIEWED REFSEQ: This record has been |
| | curated by NCBI staff. The reference |
| | sequence was derived from AF023917.1. |
| | Summary: The protein encoded by this |
| | gene is a member of the dual specific- |
| | ity protein phosphatase subfamily. |
| | These phosphatases inactivate their |
| | target kinases by dephosphorylating |
| | both the phosphoserine/threonine and |
| | phosphotyrosine residues. They neg- |
| | atively regulate members of the mito- |
| | gen-activated protein (MAP) kinase |
| | superfamily (MAPK/ERK, SAPK/JNK, p38), |
| | which is associated with cellular |
| | proliferation and differentiation. |
| | Different members of the family of |
| | dual specificity phosphatases show |
| | distinct substrate specificities for |
| | various MAP kinases, different tissue |
| | distribution and subcellular localiza- |
| | tion, and different modes of inducibil- |
| | ity of their expression by extracel- |
| | lular stimuli. This gene product is |
| | localized to the nucleus, and is novel |
| | in that it binds directly to RNA and |
| | splicing factors, and thus suggested |
| | to participate in nuclear mRNA meta- |
| | bolism. |

| | |
|---|---|
| FEATURES | Location/Qualifiers |

| | |
|---|---|
| source | 1 . . . 330 |
| | /organism = “Homo sapiens” |
| | /db_xref = “taxon: 9606” |
| | /chromosome = “2” |
| | /map = “2p13.1” |
| Protein | 1 . . . 330 |
| | /product = “dual specificity phos- |
| | phatase 11” |
| | /EC_number = “3.1.3.16” |
| | /EC_number = “3.1.3.48” |
| | /note = “serine/threonine specific |
| | protein phosphatase; RNA/RNP com- |
| | plex-interacting phosphatase” |
| CDS | 1 . . . 330 |
| | /gene = “DUSP11” |
| | /coded_by = “NM_003584.1: |
| | 125 . . . 1117” |
| | /note = “go_component: nucleus |
| | [goid 0005634] [evidence TAS] |
| | [pmid 9685386]; |
| | go_function: RNA binding [goid |
| | 0003723] [evidence TAS] |
| | [pmid 9685386]; |
| | go_function: hydrolase activity |
| | [goid 0016787] [evidence IEA]; |
| | go_function: protein tyrosine |
| | phosphatase activity [goid |
| | 0004725] [evidence TAS] |
| | [pmid 9685386]; |
| | go_process: RNA processing [goid |
| | 0006396] [evidence TAS] |
| | [pmid 9685386]; |
| | go_process: protein amino acid |
| | dephosphorylation [goid 0006470] |
| | [evidence IEA]” |
| | /db_xref = “CCDS: CCDS1928.1” |
| | /db_xref = “GeneID: 8446” |
| | /db_xref = “MIM: 603092” |

| | |
|---|---|
| ORIGIN | |
| 1 | msqwhhprsg wgrrrdfsgr ssakkkggnh |
| | iperwkdylp vgqrmpgtrf iafkvplqks |
| 61 | tekklapeec fspldlfnki reqneelgli |
| | idltytqryy kpedlpetvp ylkiftvghq |
| 121 | vpddetifkf khavngflke nkdndkligv |
| | hcthglnrtg ylicrylidv egvrpddaie |
| 181 | lfnrcrqhcl erqnyiedlq ngpirknwns |
| | svprssdfed sahlmqpvhn kpvkqgpryn |
| 241 | lhqiqghsap rhfhtqtqsl qqsvrkfsen |
| | phvyqrhhlp ppgppgedys hrryswnvkp |
| 301 | nasraaqdrr rwypynysrl sypacwewtq |
| // | |

IV. Dicer

Dicer proteins for use in the present invention can be from any suitable source. Preferred sources include C. elegans, H. sapeins and M. musculus, as depicted infia, although the skilled artisan will appreciate that other sources can readily be used based on the significant conservation exhibited between Dicer homologs. For example, Dicer homologs from D. melanogaster, Rattus norvegicus, and primate are useful (see, e.g., Accession Nos. gi:51316117; gi:34867687; and gi:55641327, respectively).

| | |
|---|---|
| LOCUS | NP_498761 1845 aa linear INV 21- |
| NOVEMBER | |
| 2003 | |
| DEFINITION | DiCer Related, LEThal LET-740 (dcr-1) |
| | [Caenorhabditis elegans]. |
| ACCESSION | NP_498761 |
| VERSION | NP_498761.1 GI: 17552834 |
| DBSOURCE | REFSEQ: accession NM 066360.1 |
| KEYWORDS | . |
| SOURCE | Caenorhabditis elegans |

| | |
|---|---|
| ORGANISM | Caenorhabditis elegans |
| | Eukaryota; Metazoa; Nematoda; |
| | Chromadorea; Rhabditida; Rhabditoidea; |
| | Rhabditidae; Peloderinae; |
| | Caenorhabditis. |

| | |
|---|---|
| REFERENCE | 1 (residues 1 to 1845) |

| | |
|---|---|
| AUTHORS | Dillin,A., Hsu,A. L., Arantes-Oliveira, |
| | N., Lehrer-Graiwer,J., Hsin,H., Fraser, |
| | A. G., Kamath,R. S., Ahringer,J. and |
| | Kenyon,C. |
| TITLE | Rates of behavior and aging specified |
| | by mitochondrial function during |
| | development |
| JOURNAL | Science 298 (5602), 2398-2401 (2002) |
| MEDLINE | 22382053 |
| PUBMED | 12471266 |

| | |
|---|---|
| REFERENCE | 2 (residues 1 to 1845) |

| | |
|---|---|
| AUTHORS | Piano,F., Schetter,A. J., Morton,D. G., |
| | Gunsalus,K. C., Reinke, V. Kim,S. K. |
| | and Kemphues,K. J. |
| TITLE | Gene clustering based on RNAi pheno- |
| | types of ovary-enriched genes in |
| | C. elegans |
| JOURNAL | Curr. Biol. 12 (22), 1959-1964 (2002) |
| MEDLINE | 22335533 |
| PUBMED | 12445391 |

| | |
|---|---|
| REFERENCE | 3 (residues 1 to 1845) |

| | |
|---|---|
| AUTHORS | Walhout,A. J., Reboul,J., Shtanko,O., |
| | Bertin,N., Vaglio,P., Ge, H., Lee,H., |
| | Doucette-Stamin,L., Gunsalus,K. C., |
| | Schetter,A. J., Morton,D. G., Kemphues, |
| | K. J., Reinke,V., Kim,S. K., Piano,F. |
| | and Vidal, M. |
| TITLE | Integrating interactome, phenome, and |
| | transcriptome mapping data for the |
| | C. elegans germline |
| JOURNAL | Curr. Biol. 12 (22), 1952-1958 (2002) |
| MEDLINE | 22335532 |
| PUBMED | 12445390 |

| | |
|---|---|
| REFERENCE | 4 (residues 1 to 1845) |

| | |
|---|---|
| AUTHORS | Tabara,H., Yigit,E., Siomi,H. and |
| | Mello,C.C. |
| TITLE | The dsRNA binding protein RDE-4 inter- |
| | acts with RDE-1, DCR-1, and a DExH-box |
| | helicase to direct RNAi in C. elegans |
| JOURNAL | Cell 109 (7), 861-871 (2002) |
| MEDLINE | 22105477 |
| PUBMED | 12110183 |

| | |
|---|---|
| 2REFERENCE | 5 (residues 1 to 1845) |

| | |
|---|---|
| AUTHORS | Banerjee,O. and Slack,F. |
| TITLE | Control of developmental timing by |
| | small temporal RNAs: a paradigm for |
| | RNA-mediated regulation of gene |
| | expression |
| JOURNAL | Bioessays 24 (2), 119-129 (2002) |
| MEDLINE | 21823375 |
| PUBMED | 11835276 |

| | |
|---|---|
| REFERENCE | 6 (residues 1 to 1845) |

| | |
|---|---|
| AUTHORS | Ketting,R. F., Fischer,S. E., |
| | Bernstein,E., Sijen,T., Hannon,G. J. |
| | and Plasterk, R. H. |
| TITLE | Dicer functions in RNA interference and |
| | in synthesis of small RNA involved in |
| | developmental timing in C. elegans |
| JOURNAL | Genes Dev. 15 (20), 2654-2659 (2001) |
| MEDLINE | 21521222 |
| PUBMED | 11641272 |

| | |
|---|---|
| REFERENCE | 7 (residues 1 to 1845) |

| | |
|---|---|
| AUTHORS | Knight,S. W. and Bass,B. L. |
| TITLE | A role for the RNase III enzyme DCR-1 |
| | in RNA interference and germ line de- |
| | velopment in Caenorhabditis elegans |
| JOURNAL | Science 293 (5538), 2269-2271 (2001) |
| MEDLINE | 21451181 |
| PUBMED | 11486053 |

| | |
|---|---|
| REFERENCE | 8 (residues 1 to 1845) |

| | |
|---|---|
| AUTHORS | Jones,S. J., Riddle,O. L., Pouzyrev,A. |
| | T., Velculescu,V. E., Hillier,L., Eddy, |
| | S. R., Stricklin,S. L., Baillie,D. L., |
| | Waterston, R. and Marra,M. A. |
| TITLE | Changes in gene expression associated |
| | with developmental arrest and longevity |
| | in Caenorhabditis elegans |
| JOURNAL | Genome Res. 11 (8), 1346-1352 (2001) |
| MEDLINE | 21376140 |
| PUBMED | 11483575 |

| | |
|---|---|
| REFERENCE | 9 (residues 1 to 1845) |

| | |
|---|---|
| AUTHORS | Grishok,A., Pasquinelli,A. E., Conte, |
| | D., Li,N., Parrish,S., Ha, I., Baillie, |
| | D. L., Fire,A., Ruvkun,G. and Mello, |
| | C. C. |
| TITLE | Genes and mechanisms related to RNA in- |
| | terference regulate expression of the |
| | small temporal RNAs that control C. |
| | elegans developmental timing |
| JOURNAL | Cell 106 (1), 23-34 (2001) |
| MEDLINE | 21354308 |
| PUBMED | 11461699 |

| | |
|---|---|
| REFERENCE | 10 (residues 1 to 1845) |

| | |
|---|---|
| AUTHORS | Stewart,H. I., O'Neil,N. J., Janke,D. |
| | L., Franz,N. W., Chamberlin,H. M., |
| | Howell,A. M., Gilchrist,E. J., Ha,T. |
| | T., Kuervers,L. M., Vatcher,G. P., |
| | Danielson,J. L. and Baillie,D. L. |
| TITLE | Lethal mutations defining 112 comple- |
| | mentation groups in a 4.5 Mb sequenced |
| | region of Caenorhabditis elegans |
| | chromosome III |
| JOURNAL | Mol. Gen. Genet. 260 (2-3), 280-288 |
| | (1998) |
| MEDLINE | 99077298 |
| PUBMED | 9862482 |

| | |
|---|---|
| COMMENT | PROVISIONAL REFSEQ: This record has not |
| | yet been subject to final NCBI review. |
| | This record is derived from an anno- |
| | tated genomic sequence (NC_003281). |
| | The reference sequence was derived |
| | from WormBase CDS: K12H4.8. |
| | Summary: This essential gene dcr-1, |
| | also known as let-740, K12H4.8, |
| | 3J162 or YK334, maps at (III; −0.30). |
| | Phenotypes and affected processes are |
| | required for RNA interference, required |
| | for synthesis of microrna, sterile |
| | adult, lethal. It encodes a DiCer Re- |
| | lated. From Pfam homology, the product |
| | would have ATP binding, nucleic acid |
| | binding, ATP dependent helicase, heli- |
| | case, RNA binding, double-stranded RNA |
| | binding, ribonuclease III activities, |
| | would be involved in RNA processing |
| | and would localize in intracellular. |
| | According to the Worm Transcriptome |
| | Project, it is expressed at high level |
| | at all stages of development [Kohara |
| | cDNAs], except dauers [SAGE]. Its |
| | existence, but not its exact sequence, |
| | derived here from the genome sequenc- |
| | ing consortium annotation, is sup- |
| | ported by 26 cDNA clones. |
| | Phenotype |
| | [WormBase] dcr-1 is required both for |
| | RNA interference and for synthesis of |
| | small developmental RNAs. Fertilization |
| | of dcr-1 oocytes does not occur. While |
| | this fertilization defect can be res- |
| | cued by a dcr-1(+) transgene, fertil- |
| | ized eggs fail to hatch, and mothers |
| | are defective in egg-laying. Whereas |
| | wild-type oocytes normally do not un- |
| | dergo cell division in the gonad, dcr- |
| | 1 (pk1531) oocytes undergo such divi- |
| | sion frequently. dcr-1 mutations also |
| | cause postembryonic defects: alae are |
| | absent in 60%, and a burst vulva is |
| | observed in 80%, of dcr-1 (pk1531) |
| | homozygotes. The postembryonic defects |
| | are consistent with the hypothesis that |
| | dcr-1 mutants hyperactivate lin-41 in |
| | vivo because they are unable to form |
| | active let-7 stRNA; in vitro assays of |
| | DCR-1 protein confirm that it can gen- |
| | erate let-7 stRNA from a double- |
| | stranded let-7 precursor. [Ann Rose, |
| | 1998, pm9862482] let-704 homozygous |
| | s2624 and s2795 each develop into |
| | sterile adults. Knock-out allele, de- |
| | letion obtained by the Gene Knockout |
| | Consortium ok247 (strain BB1) [R |
| | Barstead, Oklahoma MRF, USA]. Selected |
| | strains available from the CGC. BC4825 |
| | [David Baillie]. NL687 dcr-1 (pk1351)/+ |
| | III [Ronald Plasterk, Fischer/Thijssen, |
| | UV/TMP] Heterozygotes are WT and segre- |
| | gate WT and animals with protruding |
| | vulvas (dcr-1 homozygotes). PD8753 dcr-1 |
| | (ok247) III/hT2[qIs48] (I; III) [Andrew |
| | Fire, Barstead/Moulder] [Brenda Bass |
| | description] Heterozygotes are WT and |
| | segregate WT, Uncs, and Steriles. [B |
| | Barstead] dcr-1 homozygotes are com- |
| | pletely sterile. qIs48 is an insertion |
| | of ccEx9747 with markers: myo-2: :GFP |
| | expressed brightly in the pharynx |
| | throughout development, pes-10: :GFP |
| | expressed in embryos, and a gut pro- |
| | moter driving GFP in the intestine. |
| | Segregates WT glowing hets, non-glowing |
| | steriles, very rare homozygous hT2 |
| | glowing animals, and dead eggs. |
| | BB1. |
| | RNA interference results: |
| | [T. Hyman 2000] No obvious phenotype |
| | (by injecting genomic PCR product TH: |
| | K12H4.8). [J. Ahringer 2003] No obvious |
| | phenotype (by feeding genomic PCR pro- |
| | duct JA: K12H4.8). [F. Piano 2002] No |
| | P0 sterility detected. Pleiotropic |
| | phenotypes (may include abnormal trans- |
| | lucence, Dpy, Egl, Gon, Muv, Pvl, Sma) |
| | observed in <10% of progeny. No obvious |
| | phenotype. |
| | Function |
| | Protein properties: [Wormbase] biden- |
| | tate ribonuclease, contains a helicase |
| | domain, a PAZ domain, two RNAse III |
| | domains, and a double-stranded RNA- |
| | binding domain. |
| | Expression |
| | The expression profile for the gene, |
| | derived from the proportion of animals |
| | at each stage in each Kohara library |
| | is: embryos 7%, L1 or L2 larvae 19%, |
| | L3 to adult 75%. |
| | In situ hybridisation pictures to all |
| | stages of development are available |
| | from Kohara NextDB. |
| | Pattern [pm11483575] From SAGE compar- |
| | ative analysis of dauer and mixed |
| | stages, this gene is one of 533 whose |
| | expression is lowered in dauer larvae, |
| | a facultative developmentally arrested |
| | and long lived stage in C. elegans life |
| | cycle. germline enriched [Piano, 2002]. |
| | The predicted CDS has 26 exons. It |
| | covers 8.17 kb on the WS97 genome. The |
| | protein (1845 aa, 210.9 kDa, pI 5.6) |
| | contains one DEAD/DEAH box helicase |
| | motif, one helicase, C-terminal motif, |
| | one Protein of unknown function DUF283 |
| | motif, one Argonaute and Dicer protein, |
| | PAZ motif, 2 Ribonuclease III family |
| | motifs, one Double-stranded RNA binding |
| | (DsRBD) domain motif. It also contains |
| | 3 coil coil stretch [Psort2]. It is |
| | predicted to localise in the cytoplasm |
| | [Psort2]. Taxblast (threshold 10\hat{ }-3) |
| | tracks ancestors down to archaea and |
| | viruses and bacteria and eukaryota. |
| | Method: conceptual translation. |

| | |
|---|---|
| FEATURES | Location/Qualifiers |

| | |
|---|---|
| source | 1 . . . 1845 |
| | /organism = “Caenorhabditis elegans” |
| | /db_xref = “taxon: 6239” |
| | /chromosome = “III” |
| | /map = “III; −0.30 cM (interpolated |
| | genetic position)” |
| | /map = “III; covering 6084 bp, from |
| | base 8077912 to 8071829 on genome |
| | release WS97” |
| | /clone_lib = “Kohara embryonic |
| | lambda gt11 library: yk571d8, |
| | yk675c6; Kohara Sugano L1 larvae |
| | cap-selected library: yk1080g6, |
| | yk1084b3, yk1086f1, yk1249b10, |
| | yk1271d8; Kohara Sugano L2 larvae |
| | cap-selected library: yk1627e3, |
| | yk1734b12; Kohara Sugano L4 larvae |
| | cap-selected library: yk1448b2, |
| | yk1548a2, yk1554a2; Kohara mixed |
| | stage library, from him-8 strain, |
| | containing 15-30% males: yk11h10, |
| | yk18g7, yk24e10, yk86c11, yk181d7, |
| | yk192e1, yk243c2, yk249e11, yk318d2, |
| | yk355e9, yk355h8, yk419h11, |
| | yk154a11; early embryos, Stratagene |
| | library [PMID1302005]: T02268” |
| Protein | 1 . . . 1845 |
| | /product = “DiCer Related, LEThal |
| | LET-740 (dcr-1)” |
| Region | 3 . . . 218 |
| | /region_name = “[Pfam/InterPro |
| | description] DEAD/DEAH box helicase” |
| | /db_xref = “CDD: pfam00270” |
| Region | 190 . . . 218 |
| | /region_name = “[PSORT] coil coil 4: |
| | PEKLMEQLKKLESAMDSVIETASDLVSLS” |
| Region | 339 . . . 345 |
| | /region_name = “[PSORT] nuclear lo- |
| | calization domain: PEMKKIK” |
| Region | 427 . . . 498 |
| | /region_name = “[Pfam/InterPro de- |
| | scription] helicase, C-terminal” |
| | /db_xref = “CDD: pfam00271” |
| Region | 503 . . . 602 |
| | /region_name = “[Pfam/InterPro de- |
| | scription] protein of unknown |
| | function DUF283” |
| | /db_xref = “CDD: pfam03368” |
| Region | 669 . . . 675 |
| | /region_name = “[PSORT] nuclear lo- |
| | calization domain: PKRRKFE” |
| Region | 764 . . . 770 |
| | /region_name = “[PSORT] nuclear lo- |
| | calization domain: PLNKRKD” |
| Region | 782 . . . 961 |
| | /region_name = “[Pfam/InterPro de- |
| | scription] argonaute and Dicer pro- |
| | tein, PAZ” |
| | /db_xref = “CDD: pfam02170” |
| Region | 891 . . . 897 |
| | /region_name = “[PSORT] nuclear lo- |
| | calization domain: PRRSRTV” |
| Region | 1008 . . . 1036 |
| | /region_name = “[PSORT] coil coil 4: |
| | IQQLRDLNQKSIEDQERETRENDKIDDGE” |
| Region | 1179 . . . 1214 |
| | /region_name = “[PSORT] coil coil 4: |
| | PKQLTKEEEQFKKLQNDLLKQAKERLEALEMSEDME” |
| Region | 1215 . . . 1218 |
| | /region_name = “[PSORT] nuclear lo- |
| | calization domain: KPRR” |
| Region | 1348 . . . 1524 |
| | /region_name = “[Pfam/InterPro |
| | description] ribonuclease III |
| | family” |
| | /db_xref = “CDD: pfam00636” |
| Region | 1614 . . . 1740 |
| | /region_name = “[Pfam/InterPro |
| | description] ribonuclease III |
| | family” |
| | /db_xref = “CDD: pfam00636” |
| Region | 1769 . . . 1829 |
| | /region_name = “[Pfam/InterPro de- |
| | scription] double-stranded RNA |
| | binding (DsRBD) domain” |
| | /db_xref = “CDD: pfam00035” |
| CDS | 1 . . . 1845 |
| | /gene = “dcr-1” |
| | /locus_tag = “3J162” |
| | /coded_by = “NM_066360.1: |
| | 1 . . . 5538” |
| | /db_xref = “AceView/WormGenes: dcr-1” |
| | /db_xref = “GeneID: 176138” |
| | /db_xref = “LocusID: 176138” |
| | /db_xref = “WormBase: K12H4.8” |

| | |
|---|---|
| ORIGIN | |
| 1 | mvrvradlqc fnprdyqvel ldkatkknti |
| | vqlgtgsgkt fiavlllkey gvqlfapldq |
| 61 | ggkraffvve kvnlveqqai hievhtsfkv |
| | gqvhgqtssg lwdskeqcdq fmkrhhvvvi |
| 121 | taqclldlir haylkiedmc vlifdechha |
| | lgsqhpyrsi mvdykllkkd kpvprvlglt |
| 181 | aslikakvap eklmeqlkkl esamdsviet |
| | asdlvslsky gakpyevvii ckdfeigclg |
| 241 | ipnfdtviei fdetvafvnt ttefhpdldl |
| | dprrpikdsl kttravfrql gpwaawrtaq |
| 301 | vwekelgkii ksqvlpdktl rflnmaktsm |
| | itikrllepe mkkiksieal rpyvpqrvir |
| 361 | lfeiletfnp efqkermkle kaehlsaiif |
| | vdqryiaysl llmmrhiksw epkfkfvnpd |
| 421 | yvvgasgrnl assdsqglhk rqtevlrrfh |
| | rneincliat svleegvdvk qcnlvikfdr |
| 481 | pldmrsyvqs kgrarragsr yvitveekdt |
| | aaycsklpsd iftrlvphnq iipieengvt |
| 541 | kycaelllpi nspikhaivl knpmpnkkta |
| | qmavaleacr qlhlegeldd nllpkgresi |
| 601 | akllehidee pdeyapgiaa kvgsskrkql |
| | ydkkiaraln esfveadkec fiyafelerf |
| 661 | reaeltlnpk rrkfedpfny eycfgflsak |
| | eipkippfpv flrqgnmkvr livapkkttv |
| 721 | taaqlqeiql fhnylftqvl qmcktgnlef |
| | dgtsnaplnt livplnkrkd dmsytinmky |
| 781 | vsevvanmen mpripkdevr rqykfnaedy |
| | kdaivmpwyr nleqpvfyyv aeilpewrps |
| 841 | skfpdthfet fneyfikkyk leiydqnqsl |
| | ldvdftstrl nhlqpriqnq prrsrtvsns |
| 901 | stsnipqasa sdskesntsv phssqrqilv |
| | pelmdihpis atlwnviaal psifyrvnql |
| 961 | lltdelreti lvkafgkekt klddnvewns |
| | layateyeek qtiivkkiqq lrdlnqksie |
| 1021 | dqeretrend kiddgeelfn igvwdpeeav |
| | rigveissrd drmdgedqdt vgltqglhdg |
| 1081 | nisdeddelp fvmhdytarl tsnrngigaw |
| | sgsesivpsg wgdwdgpepd nspmpfqilg |
| 1141 | gpgglnvqal madvgrvfdp stassslsqt |
| | vqestvsppk qltkeeeqfk klqndllkqa |
| 1201 | kerlealems edmekprrle dtvnledygd |
| | dqenqedent ptnfpktide eieelsigar |
| 1261 | kkqeiddnaa ktdvlerenc evlpvainek |
| | srsfsfekes kaingrlirq rseeyvshid |
| 1321 | sdiglgvspc llltalttsn aadgmslerf |
| | etigdsflkf attdylyhtl ldqhegklsf |
| 1381 | arskevsncn lyrlgkklgi pqlivankfd |
| | ahdswlppcy iptcdfkapn tddaeekdne |
| 1441 | ierildgqvi eekpenktgw diggdvskst |
| | tdgietitfp kqarvgnddi splpynlltq |
| 1501 | qhisdksiad avealigvhl ltlgpnptlk |
| | vmnwmglkvi qkdqksdvps pllrfidtpt |
| 1561 | npnaslnfln nlwqqfqftq leekigyrfk |
| | eraylvqaft hasyinnrvt gcyqrleflg |
| 1621 | davldymitr ylfedsrqys pgvltdlrsa |
| | lvnntifasl avkfefqkhf iamcpglyhm |
| 1681 | iekfvklcse rnfdtnfnae mymvtteeei |
| | degqeediev pkamgdifes vagaiyldsg |
| 1741 | rnldttwqvi fhmmrgtiel ccanpprspi |
| | relmefeqsk vrfskmeril esgkvrvtve |
| 1801 | vvnnmrftgm grnyriakat aakralkylh |
| | qieqqrrqsp slttv |
| // | |
| LOCUS | NP_803187 1922 aa linear PRI 22- |
| DECEMBER | |
| 2003 | |
| DEFINITION | dicerl; helicase-mol; K12H4.8-LIKE; |
| | helicase with RNAse motif [Homo |
| | sapiens]. |
| ACCESSION | NP_803187 |
| VERSION | NP_803187.1 GI: 29294651 |
| DESOURCE | REFSEQ: accession NM 177438.1 |
| KEYWORDS | . |
| SOURCE | Homo sapiens (human) |

| | |
|---|---|
| ORGANISM | Homo sapiens |
| | Eukaryota; Metazoa; Chordata; Craniata; |
| | Vertebrata; Euteleostomi; Mammalia; |
| | Eutheria; Primates; Catarrhini; |
| | Hominidae; Homo. |

| | |
|---|---|
| REFERENCE | 1 (residues 1 to 1922) |

| | |
|---|---|
| AUTHORS | Handa,V., Saha,T. and Usdin,K. |
| TITLE | The fragile X syndrome repeats form RNA |
| | hairpins that do not activate the in- |
| | terferon-inducible protein kinase, PKR, |
| | but are cut by Dicer |
| JOURNAL | Nucleic Acids Res. 31 (21), 6243-6248 |
| | (2003) |
| PUBMED | 14576312 |
| REMARK | GeneRIF: fragile X syndrome CGG repeats |
| | readily form RNA hairpins and is di- |
| | gested by the human Dicer enzyme, a |
| | step central to the RNA interference |
| | effect on gene expression |

| | |
|---|---|
| REFERENCE | 2 (residues 1 to 1922) |

| | |
|---|---|
| AUTHORS | Kawasaki,H., Suyama,E., Iyo,M. and |
| | Taira,K. |
| TITLE | siRNAs generated by recombinant human |
| | Dicer induce specific and significant |
| | but target site-independent gene si- |
| | lencing in human cells |
| JOURNAL | Nucleic Acids Res. 31 (3), 981-987 |
| | (2003) |
| PUBMED | 12560494 |

| | |
|---|---|
| REFERENCE | 3 (residues 1 to 1922) |

| | |
|---|---|
| AUTHORS | Doi,N., Zenno,S., Ueda,R., Ohki- |
| | Hamazaki,H., Ui-Tei,K. and Saigo, K. |
| TITLE | Short-interfering-RNA-mediated gene |
| | silencing in mammalian cells requires |
| | Dicer and eIF2C translation initiation |
| | factors |
| JOURNAL | Curr. Biol. 13 (1), 41-46 (2003) |
| PUBMED | 12526743 |

| | |
|---|---|
| REFERENCE | 4 (residues 1 to 1922) |

| | |
|---|---|
| AUTHORS | Zhang,H., Kolb,F. A., Brondani,V., |
| | Billy,E. and Filipowicz,W. |
| TITLE | Human Dicer preferentially cleaves |
| | dsRNAs at their termini without a |
| | requirement for ATP |
| JOURNAL | EMBO J. 21 (21), 5875-5885 (2002) |
| PUBMED | 12411505 |
| REMARK | GeneRIF: purification and properties of |
| | a recombinant human Dicer |

| | |
|---|---|
| REFERENCE | 5 (residues 1 to 1922) |

| | |
|---|---|
| AUTHORS | Provost,P., Dishart,D., Doucet,J., |
| | Frendewey,D., Samuelsson,B. and |
| | Radinark, O. |
| TITLE | Ribonuclease activity and RNA binding |
| | of recombinant human Dicer |
| JOURNAL | EMBO J. 21 (21), 5864-5874 (2002) |
| PUBMED | 12411504 |
| REMARK | GeneRIF: cloning and expression of the |
| | 218 kDa human Dicer, and characteriza- |
| | tion of its ribonuclease activity and |
| | dsRNA-binding properties |

| | |
|---|---|
| REFERENCE | 6 (residues 1 to 1922) |

| | |
|---|---|
| AUTHORS | Matsuda,S., Ichigotani,Y., Okuda,T., |
| | Irimura,T., Nakatsugawa, S. and |
| | Hamaguchi, M. |
| TITLE | Molecular cloning and characterization |
| | of a novel human gene (HERNA) which |
| | encodes a putative RNA-helicase |
| JOURNAL | Biochim. Biophys. Acta 1490 (1-2), |
| | 163-169 (2000) |
| PUBMED | 10786632 |

| | |
|---|---|
| REFERENCE | 7 (residues 1 to 1922) |

| | |
|---|---|
| AUTHORS | Provost,P., Samuelsson,B. and |
| | Radmark,O. |
| TITLE | Interaction of 5-lipoxygenase with |
| | cellular proteins |
| JOURNAL | Proc. Natl. Acad. Sci. U.S.A. 96 (5), |
| | 1881-1885 (1999) |
| PUBMED | 10051563 |

| | |
|---|---|
| COMMENT | REVIEWED REFSEQ: This record has been |
| | curated by NCBI staff. The reference |
| | sequence was derived from AB023145.2, |
| | AB028449.1, AK091094.1, AW297296.1, |
| | BI913232.1 and BQ937506.1. |
| | Summary: This gene encodes a protein |
| | possessing an RNA helicase motif con- |
| | taining a DEXH box in its amino |
| | terminus and an RNA motif in the car- |
| | boxy terminus. The encoded protein |
| | functions as a ribonuclease and is re- |
| | quired by the RNA interference and |
| | small temporal RNA (stRNA) pathways to |
| | produce the active small RNA component |
| | that represses gene expression. Two |
| | transcript variants encoding the same |
| | protein have been identified for this |
| | gene. |
| | Transcript Variant: This variant (1) |
| | represents the longer transcript. |
| | Variants 1 and 2 encode the same |
| | isoform. |

| | |
|---|---|
| FEATURES | Location/Qualifiers |

| | |
|---|---|
| source | 1 . . . 1922 |
| | /organism = “Homo sapiens” |
| | /db_xref = “taxon: 9606” |
| | /chromosome = “14” |
| | /map = “14q32.2” |
| Protein | 1 . . . 1922 |
| | /product = “dicer1” |
| | /EC_number = “3.1.26.-” |
| | /note = “helicase-moi; K12H4.8-LIKE; |
| | helicase with RNAse motif” |
| Region | 37 . . . >208 |
| | /region_name = “ERCC4-like helicases |
| | [DNA replication, recombination, and |
| | repair]” |
| | /note = “MPH1” |
| | /db_xref = “CDD: 10833” |
| Region | 40 . . . 211 |
| | /region_name = “DEAD-like helicases |
| | superfamily” |
| | /note = “DEXDc” |
| | /db_xref = “CDD: 22813” |
| Region | 107 . . . 1899 |
| | /region_name = “dsRNA-specific |
| | nuclease Dicer and related ribo- |
| | nucleases [RNA processing and |
| | modification]” |
| | /note = “KOG0701” |
| | /db_xref = “CDD: 18495' |
| variation | 257 |
| | /replace = “*” |
| | /replace = “C” |
| | /db_xref = “dbSNP: 12432511” |
| Region | <499 . . . 553 |
| | /region_name = “Helicase conserved |
| | C-terminal domain” |
| | /note = “helicase_C” |
| | /db_xref = “CDD: 22962” |
| variation | 499 |
| | /replace = “R” |
| | /replace = “T” |
| | /db_xref = “dbSNP: 4566088” |
| Region | 625 . . . 722 |
| | /region_name = “Domain of unknown |
| | function” |
| | /note = “DUF283” |
| | /db_xref = “CDD: 5126” |
| Region | 895 . . . 1064 |
| | /region_name = “PAZ domain” |
| | /note = “PAZ” |
| | /db_xref = “CDD: 17101” |
| Region | 1296 . . . >1387 |
| | /region_name = “Ribonuclease III |
| | family” |
| | /note = “RIBOc” |
| | /db_xref = “CDD: 22830” |
| Region | 1682 . . . 1846 |
| | /region_name = “Ribonuclease III |
| | family” |
| | /note = “RIBOc” |
| | /db_xref = “CDD: 22830” |
| CDS | 1 . . . 1922 |
| | /gene = “DICER1” |
| | /coded_by = “NM_177438.1: |
| | 239 . . . 6007” |
| | /note = “go_component: intracellular |
| | [goid 0005622] [evidence NAS] |
| | [pmid 12560494]; |
| | go_function: double-stranded RNA |
| | binding [goid 0003725] [evidence |
| | IDA] [pmid 12411504]; |
| | go_function: endonuclease activity |
| | [goid 0004519] [evidence IEA]; |
| | go_function: ATP binding [goid |
| | 0005524] [evidence IEA]; |
| | go_function: ribonuclease III |
| | activity [goid 0004525] [evidence |
| | IDA] [pmid 12560494]; |
| | go_function: ATP dependent helicase |
| | activity [goid 0008026] [evidence |
| | IEA]; |
| | go_function: hydrolase activity |
| | [goid 0016787] [evidence IEA]; |
| | go_process: RNA processing [goid |
| | 0006396] [evidence IEA]; |
| | go_process: RNA interference, |
| | targeting of mRNA for destruction |
| | [goid 0030423] [evidence IEP] [pmid |
| | 12560494]” |
| | /db_xref = “GeneID: 23405” |
| | /db_xref = “LocusID: 23405” |
| | /db_xref = “MIM: 606241” |

| | |
|---|---|
| ORIGIN | |
| 1 | mkspalqpls maglqlmtpa sspmgpffgl |
| | pwqqeaihdn iytprkyqve lleaaldhnt |
| 61 | ivclntgsgk tfiavlltke lsyqirgdfs |
| | rngkrtvflv nsanqvaqqv savrthsdlk |
| 121 | vgeysnlevn aswtkerwnq eftkhqvlim |
| | tcyvalnvlk ngylslsdin llvfdechla |
| 181 | ildhpyreim klcencpscp rilgltasil |
| | ngkcdpeele ekiqklekil ksnaetatdl |
| 241 | vvldrytsqp ceivvdcgpf tdrsglyerl |
| | lmeleealnf indcnisvhs kerdstlisk |
| 301 | qilsdcravl vvlgpwcadk vagmmvrelq |
| | kyikheqeel hrkfllftdt flrkihalce |
| 361 | ehfspasldl kfvtpkvikl leilrkykpy |
| | erqqfesvew ynnrnqdnyv swsdseddde |
| 421 | deeieekekp etnfpspftn ilcgiifver |
| | rytavvlnrl ikeagkqdpe layissnfit |
| 481 | ghgigknqpr nkqmeaefrk qeevlrkfra |
| | hetnlliats iveegvdipk cnlvvrfdlp |
| 541 | teyrsyvqsk grarapisny imladtdkik |
| | sfeedlktyk aiekilrnkc sksvdtgetd |
| 601 | idpvmddddv fppyvlrpdd ggprvtinta |
| | ighinrycar lpsdpfthla pkcrtrelpd |
| 661 | gtfystlylp insplrasiv gppmscvrla |
| | ervvalicce klhkigeldd hlmpvgketv |
| 721 | kyeeeldlhd eeetsvpgrp gstkrrqcyp |
| | kaipeclrds yprpdqpcyl yvigmvlttp |
| 781 | lpdelnfrrr klyppedttr cfgiltakpi |
| | pqiphfpvyt rsgevtisie lkksgfmlsl |
| 841 | qmlelitrlh qyifshilrl ekpalefkpt |
| | dadsaycvlp lnvvndsstl didfkfmedi |
| 901 | eksearigip stkytketpf vfkledyqda |
| | viipryrnfd qphrfyvadv ytdltplskf |
| 961 | pspeyetfae yyktkynldl tnlnqplldv |
| | dhtssrlnll tprhlnqkgk alplssaekr |
| 1021 | kakweslqnk qilvpelcai hpipaslwrk |
| | avclpsilyr lhclltaeel raqtasdagv |
| 1081 | gvrslpadfr ypnldfgwkk sidsksfisi |
| | snsssaendn yckhstivpe naahqganrt |
| 1141 | sslenhdqms vncrtllses pgklhvevsa |
| | dltainglsy nqnlangsyd lanrdfcqgn |
| 1201 | qlnyykqeip vqpttsysiq nlysyenqpq |
| | psdectllsn kyldgnanks tsdgspvmav |
| 1261 | mpgttdtiqv lkgrmdseqs psigyssrtl |
| | gpnpglilqa ltlsnasdgf nlerlemlgd |
| 1321 | sflkhaitty lfctypdahe grlsymrskk |
| | vsncnlyrlg kkkglpsrmv vsifdppvnw |
| 1381 | lppgyvvnqd ksntdkwekd emtkdcmlan |
| | gkldedyeee deeeeslmwr apkeeadyed |
| 1441 | dfleydqehi rfidnmlmgs gafvkkisls |
| | pfsttdsaye wkmpkksslg smpfssdfed |
| 1501 | fdysswdamc yldpskavee ddfvvgfwnp |
| | seencgvdtg kqsisydlht eqciadksia |
| 1561 | dcveallgcy ltscgeraaq lflcslglkv |
| | lpvikrtdre kalcptrenf nsqqknlsvs |
| 1621 | caaasvassr ssvlkdseyg clkipprcmf |
| | dhpdadktln hlisgfenfe kkinyrfknk |
| 1681 | ayllqaftha syhyntitdc yqrleflgda |
| | ildylitkhl yedprqhspg vltdlrsalv |
| 1741 | nntifaslav kydyhkyfka vspelfhvid |
| | dfvqfqlekn emqgmdselr rseedeekee |
| 1801 | dievpkamgd ifeslagaiy mdsgmsletv |
| | wqvyypmmrp liekfsanvp rspvrellem |
| 1861 | epetakfspa ertydgkvrv tvevvgkgkf |
| | kgvgrsyria ksaaarralr slkanqpqvp |
| 1921 | ns |
| // | |
| LOCUS | NP_683750 1917 aa linear ROD 16- |
| MARCH 2004 | |
| DEFINITION | dicer1; endoribonuclease Dicer [Mus |
| | musculus]. |
| ACCESSION | NP_683750 |
| VERSION | NP_683750.1 GI: 22507359 |
| DBSOURCE | REFSEQ: accession NM 148948.1 |
| KEYWORDS | . |
| SOURCE | Mus musculus (house mouse) |

| | |
|---|---|
| ORGANISM | Mus musculus |
| | Eukaryota; Metazoa; Chordata; Craniata; |
| | Vertebrata; Euteleostomi; Mammalia; |
| | Eutheria; Rodentia; Sciurognathi; |
| | Muridae; Murinae; Mus. |

| | |
|---|---|
| REFERENCE | 1 (residues 1 to 1917) |

| | |
|---|---|
| AUTHORS | Bernstein,E., Kim,S. Y., Carmeli,M. A., |
| | Murchison,E. P., Alcorn, H., Li,M. Z., |
| | Mills,A. A., Elledge,S. J., Anderson, |
| | K. V. and Hannon, G. J. |
| TITLE | Dicer is essential for mouse |
| | development |
| JOURNAL | Nat. Genet. 35 (3), 215-217 (2003) |
| PUBMED | 14528307 |
| REMARK | GeneRIF: role in lethality early in |
| | development |

| | |
|---|---|
| REFERENCE | 2 (residues 1 to 1917) |

| | |
|---|---|
| AUTHORS | Okazaki,N., Kikuno,R., Ohara,R., |
| | Inamoto,S., Koseki,H., Hiraoka, S., |
| | Saga,Y., Nagase,T., Ohara,O. and |
| | Koga, H. |
| TITLE | Prediction of the coding sequences of |
| | mouse homologues of KIAA gene: III. the |
| | complete nucleotide sequences of 500 |
| | mouse KIAA-homologous cDNAs identified |
| | by screening of terminal sequences of |
| | cDNA clones randomly sampled from size- |
| | fractionated libraries |
| JOURNAL | DNA Res. 10 (4), 167-180 (2003) |
| PUBMED | 14621295 |

| | |
|---|---|
| REFERENCE | 3 (residues 1 to 1917) |

| | |
|---|---|
| AUTHORS | Doi,N., Zenno,S., Ueda,R., Ohki- |
| | Hamazaki,H., Ui-Tei,K. and Saigo, K. |
| TITLE | Short-interfering-RNA-mediated gene |
| | silencing in mammalian cells requires |
| | Dicer and eIF2C translation initiation |
| | factors |
| JOURNAL | Curr. Biol. 13 (1), 41-46 (2003) |
| PUBMED | 12526743 |

| | |
|---|---|
| REFERENCE | 4 (residues 1 to 1917) |

| | |
|---|---|
| AUTHORS | Nicholson,R. H. and Nicholson,A. W. |
| TITLE | Molecular characterization of a mouse |
| | cDNA encoding Dicer, a ribonuclease III |
| | ortholog involved in RNA interference |
| JOURNAL | Mamm. Genome 13 (2), 67-73 (2002) |
| PUBMED | 11889553 |

| | |
|---|---|
| COMMENT | PROVISIONAL REFSEQ: This record has not |
| | yet been subject to final NCBI review. |
| | The reference sequence was derived from |
| | AF430845.1. |

| | |
|---|---|
| FEATURES | Location/Qualifiers |

| | |
|---|---|
| source | 1 . . . 1917 |
| | /organism = “Mus musculus” |
| | /strain = “CZECHII” |
| | /db_xref = “taxon: 10090” |
| | /chromosome = “12” |
| | /map = “12F1” |
| Protein | 1 . . . 1917 |
| | /product = “dicer1” |
| | /note = “endoribonuclease Dicer” |
| Region | 38 . . . >226 |
| | /region_name = “ERCC4-like helicases |
| | [DNA replication, recombination, and |
| | repair]” |
| | /note = “MPH1” |
| | /db_xref = “CDD: 10833” |
| Region | 41 . . . 242 |
| | /region_name = “DEAD-like helicases |
| | superfamily” |
| | /note = “DEXDc” |
| | /db_xref = “CDD: 24291” |
| Region | 109 . . . 1894 |
| | /region_name = “dsRNA-specific |
| | nuclease Dicer and related ribo- |
| | nucleases [RNA processing and |
| | modification]” |
| | /note = “KOG0701” |
| | /db_xref = “CDD: 18495” |
| Region | <500 . . . 554 |
| | /region_name = “Helicase conserved |
| | C-terminal domain” |
| | /note = “Helicase_C” |
| | /db_xref = “CDD: 24402” |
| Region | 631 . . . 723 |
| | /region_name = “Domain of unknown |
| | function” |
| | /note = “DUF283” |
| | /db_xref = “CDD: 26059” |
| Region | <926 . . . >1039 |
| | /region_name = “Germ-line stem cell |
| | division protein Hiwi/Piwi” |
| | /note = “KOG1042” |
| | /db_xref = “CDD: 18835” |
| Region | 1297 . . . >1388 |
| | /region_name = “Ribonuclease III |
| | family” |
| | /note = “RIBOc” |
| | /db_xref = “CDD: 22830” |
| Region | 1677 . . . 1841 |
| | /region_name = “Ribonuclease III |
| | family” |
| | /note = “RIBOc” |
| | /db_xref = “CDD: 22830” |
| CDS | 1 . . . 1917 |
| | /gene = “Dicer1” |
| | /coded_by = “NM_148948.1: |
| | 255 . . . 6008” |
| | /note = “go_component: cellular_{—} |
| | component unknown [goid 0008372] |
| | [evidence ND]; |
| | go_component: intracellular [goid |
| | 0005622] [evidence ISS] [pmid |
| | 12466851]; |
| | go_function: ribonuclease III |
| | activity [goid 0004525] [evidence |
| | IDA] [pmid 14528307]; |
| | go_function: nuclease activity |
| | [goid 0004518] [evidence IEA]; |
| | go_function: RNA binding [goid |
| | 0003723] [evidence IEA]; |
| | go_function: helicase activity [goid |
| | 0004386] [evidence IEA]; |
| | go_function: endonuclease activity |
| | [goid 0004519] [evidence IEA]; |
| | go_function: ATP binding [goid |
| | 0005524] [evidence IEA]; |
| | go_function: ATP-dependent helicase |
| | activity [goid 0008026] [evidence |
| | IEA]; |
| | go_function: hydrolase activity |
| | [goid 0016787] [evidence IEA]; |
| | go_function: nucleic acid binding |
| | [goid 0003676] [evidence IEA]; |
| | go_function: double-stranded RNA |
| | binding [goid 0003725] [evidence |
| | ISS] [pmid 12466851]; |
| | go_process: biological_process un- |
| | known [goid 0000004] [evidence ND]; |
| | go_process: RNA processing [goid |
| | 0006396] [evidence IDA] [pmid |
| | 14528307]; |
| | go_process: stem cell maintenance |
| | [goid 0019827] [evidence IMP] [pmid |
| | 14528307]; |
| | go_process: RNA interference, |
| | production of guide RNAs [goid |
| | 0030422] [evidence IDA] [pmid |
| | 14528307]” |
| | /db_xref = “GeneID: 192119” |
| | /db_xref = “LocusID: 192119” |
| | /db_xref = “MGI: 2177178” |

| | |
|---|---|
| ORIGIN | |
| 1 | mnekpcfaal smaglqlmtp asspmgpffg |
| | lpwqqeaihd niytprkyqv elleaaldhn |
| 61 | tivclntgsg ktfiavlltk elahqirgdl |
| | nphakrtvfl vnsanqvcqq vsavrthsdl |
| 121 | kvgeysdlev naswtkerws qeftkhqvli |
| | mtcyvaltvl kngylslsdi nllvfdechl |
| 181 | aildhpyrei mklcescpsc prilqltasi |
| | lngkcdpeel eekiqkleri lrsdaetatd |
| 241 | lvvldrytsq pceivvdcgp ftdrsglyer |
| | llmeleaald findcnvavy skerdstlis |
| 301 | kqilsdcrav lvvlgpwcad kvagmmvrel |
| | qkyikheqee lhrkfllftd tllrkihalc |
| 361 | eeyfspasld lkyvtpkvmk lleilrkykp |
| | yerqqfesve wynnrnqdny vswsdseddd |
| 421 | ddeeieekek petnfpspft nilcgiifve |
| | rrytavvlnr likeagkqdp elayissnfi |
| 481 | tghqigknqp rskqmeaefr kqeevlrkfr |
| | ahetnlliat svveegvdip kcnlvvrfdl |
| 541 | pteyrsyvqs kgrarapisn yvmladtdki |
| | ksfeedlkty kaiekilrnk csksadgaea |
| 601 | dvhagvdded afppyvlrpd dggprvtint |
| | aighinryca rlpsdpfthl apkcrtrelp |
| 661 | dgtfystlyl pinsplrasi vgppmdsvrl |
| | aervvalicc eklhkigeld ehlmpvqket |
| 721 | vkyeeeldlh deeetsvpgr pgstkrrqcy |
| | pkaipeclrd sypkpdqpcy lyvigmvltt |
| 781 | plpdelnfrr rklyppedtt rcfgiltakp |
| | ipqiphfpvy trsgevtisi elkksgfils |
| 841 | qqmlelitrl hqyifshilr lekpalefkp |
| | tgaesaycvl plnvvndsgt ldidfkfmed |
| 901 | ieksearigi pstkysketp fvfkledyqd |
| | aviipryrnf dqphrfyvad vytdltplsk |
| 961 | fpspeyetfa eyyktkynld ltnlnqplld |
| | vdhtssrlnl ltprhlnqkg kalplssaek |
| 1021 | rkakweslqn kqilvpelca ihpipaslwr |
| | kavclpsily rlhclltaee lraqtasdag |
| 1081 | vgvrslpvdf rypnldfgwk ksidsksfis |
| | tcnsslaesd nyckhsttvv pehaahqgat |
| 1141 | rpslenhdqm svnckrlpae spaklqsevs |
| | tdltaingls ynknlangsy dlvnrdfcqg |
| 1201 | nqlnyfkqei pvqpttsypi qnlynyenqp |
| | kpsnecplls ntyldgnant stsdgspavs |
| 1261 | tmpammnavk alkdrmdseq spsvgyssrt |
| | lgpnpglilq altlsnasdg fnlerlemlg |
| 1321 | dsflkhaitt ylfctypdah egrlsymrsk |
| | kvsncnlyrl gkkkglpsrm vvsifdppvn |
| 1381 | wlppgyvvnq dksnsekwek demtkdclla |
| | ngklgeacee eedltwrapk eeaededdfl |
| 1441 | eydqehiqfi dsmlmgsgaf vrkislspfs |
| | asdsayewkm pkkaslgsmp fasqledfdy |
| 1501 | sswdamcyld pskaveeddf vvgfwnpsee |
| | ncgvdtgkqs isydlhteqc iadksiadcv |
| 1561 | eallgcylts cgeraaqlfl cslglkvlpv |
| | ikrtsrekal dpaqengssq qkslsgscaa |
| 1621 | pvgprssagk dleygclkip prcmfdhpda |
| | ektlnhlisg fetfekkiny rfknkayllq |
| 1681 | afthasyhyn titdcyqrle flgdaildyl |
| | itkhlyedpr qhspgvltdl rsalvnntif |
| 1741 | aslavkydyh kyfkavspel fhviddfvkf |
| | qleknemqgm dselrrseed eekeedievp |
| 1801 | kamgdifesl agaiymdsgm slevvwqvyy |
| | pmmqpliekf sanvprspvr ellemepeta |
| 1861 | kfspaertyd gkvrvtvevv gkgkfkgvgr |
| | syriaksaaa rralrslkan qpqvpns |

V. Screening Assays

According to the invention, the following assays may be used to identify compounds that modulate interaction (e.g., binding) of Dicer or bioactive fragments thereof with Dicer interactors or bioactive fragments thereof, or modulate a Dicer activity or Dicer interactor activity, and hence, modulators of gene silencing or RNAi. Such modulators are particularly useful in regulation of (1) processing of miRNA precursors; (2) processing of siRNA precursors; (3) mediating mRNA cleavage; (4) mediating assembly of RISC (e.g., via siRNAs); (5) directing translation repression (e.g., via miRNAs); (6) a ribonuclease activity (e.g., cleavage of dsRNA); and (7) initiation of RNAi. The assays feature identifying modulators of the activity of Dicer interactors or bioactive fragments thereof, including, but not limited to, those activities identified in supra.

The assays of the present invention are used to identify modulators of the activity of Dicer or bioactive fragments thereof or Dicer interactors or bioactive fragments thereof. The modulators of the present invention are particularly useful in modulating Dicer and/or RNAi related activities but can also affect non-RNAi related activities.

VA. Cell Free Assays

In one embodiment, an assay of the present invention is a cell-free assay in which a composition comprising assay reagents (e.g., a Dicer interactor polypeptide, Dicer polypeptide or biologically active portions thereof), is contacted with a test compound and the ability of the test compound to modulate binding of the Dicer interactor polypeptide to the Dicer polypeptide (or bioactive fragments thereof) is determined. Binding of the Dicer interactor or Dicer (or bioactive fragments thereof) can be accomplished, for example, by coupling the polypeptide or fragment with a radioisotope or enzymatic label such that binding of polypeptide reagents can be determined by detecting the labeled compound or polypeptide in a complex. For example, test compounds or polypeptides can be labeled with ¹²⁵I, ³⁵S, ³³P, ³²P, ¹⁴C, or ³H, either directly or indirectly, and the radioisotope detected by direct counting of radioemmission or by scintillation counting. Alternatively, polypeptides can be enzymatically labeled with, for example, horseradish peroxidase, alkaline phosphatase, or luciferase, and the enzymatic label detected by determination of conversion of an appropriate protein to product.

Determination of binding of reagents can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon of surface plasmon resonance (SPR) can be used as an indication of real-time reactions between biological molecules.

In a preferred embodiment, the assay includes contacting Dicer polypeptide or biologically active portion thereof with a Dicer target molecule, e.g., a Dicer interactor or a bioactive fragment thereof to form an assay mixture, contacting the assay mixture with a test compound, and determining the ability of the test compound to interact with the Dicer polypeptide, wherein determining the ability of the test compound to interact with the Dicer polypeptide comprises determining the ability of the test compound to preferentially bind to Dicer or the bioactive portion thereof as compared to the Dicer target molecule (e.g., a Dicer protein). In another embodiment, the assay includes contacting the Dicer interactor polypeptide or biologically active portion thereof with a Dicer interactor target molecule, e.g., Dicer or a bioactive fragment thereof to form an assay mixture, contacting the assay mixture with a test compound, and determining the ability of the test compound to modulate binding between the Dicer interactor polypeptide and the Dicer polypeptide.

In another embodiment, the assay is a cell-free assay in which a composition comprising a Dicer polypeptide and a Dicer interactor polypeptide (or bioactive portions thereof) is contacted with a test compound and the ability of the test compound to modulate (e.g., stimulate or inhibit) the activity of the Dicer polypeptide or Dicer interactor polypeptide (or biologically active portions thereof) is determined.

Determining the ability of the test compound to modulate the activity of a Dicer or a Dicer interactor polypeptide can be accomplished, for example, by determining the ability of the Dicer interactor polypeptide to modulate the activity of a downstream binding partner or target molecule by one of the methods described herein for cell or organism-based assays. For example, the catalytic/enzymatic activity of the target molecule on an appropriate downstream protein can be determined as previously described.

In yet another embodiment, the cell-free assay involves contacting a Dicer interactor polypeptide or biologically active portion thereof with a Dicer interactor target molecule that binds the Dicer interactor polypeptide to form an assay mixture, contacting the assay mixture with a test compound, and determining the ability of the test compound (e.g., Dicer) to preferentially modulate the activity of a Dicer interactor binding partner or target molecule, as compared to the Dicer protein.

In more than one embodiment of the above assay methods of the present invention, it may be desirable to immobilize either the Dicer interactor or Dicer (or target molecules) to facilitate separation of complexed from uncomplexed forms of one or both of the proteins, as well as to accommodate automation of the assay. The ability of a test compound to modulate Dicer interactor polypeptide activity, Dicer polypeptide activity, interaction of a Dicer interactor polypeptide with a Dicer polypeptide (or target interaction or activity) in the presence and absence of a candidate compound, can be accomplished in any vessel suitable for containing the reactants. Examples of such vessels include microtitre plates, test tubes, and micro-centrifuge tubes. In one embodiment, a fusion protein can be provided so as to add a domain that allows one or both of the proteins to be bound to a matrix. For example, glutathione-S-transferase/Dicer interactor fusion proteins, glutathione-S-transferase/Dicer fusion proteins, or target fusion proteins can be adsorbed onto glutathione sepharose beads (Sigma Chemical, St. Louis, Mo.) or glutathione derivatized microtitre plates, which are then combined with the test compound or the test compound and either the non-adsorbed Dicer polypeptide or Dicer interactor polypeptide (or target polypeptide), and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of Dicer interactor binding or activity or Dicer binding or activity (or target binding or activity) determined using standard techniques.

Additional exemplary Dicer and/or Dicer interactor fusion proteins (or target fusion proteins) include, but are not limited to, chitin binding domain (CBD) fusion proteins, hemagglutinin epitope tagged (HA)-fusion proteins, His fusion proteins (e.g., His₆ tagged proteins), FLAG tagged fusion proteins, AU1 tagged proteins, and the like.

Other techniques for immobilizing proteins on matrices can also be used in the screening assays of the invention. For example, either a Dicer polypeptide, a Dicer interactor polypeptide or target polypeptide can be immobilized utilizing conjugation of biotin and streptavidin. Biotinylated Dicer polypeptide, Dicer interactor polypeptide or target polypeptide can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with Dicer polypeptide, Dicer interactor polypeptide or target polypeptide but which do not interfere with binding of the Dicer interactor polypeptide to Dicer polypeptide (or protein to target binding) can be derivatized to the wells of the plate, and unbound Dicer or Dicer interactor polypeptide (or target) trapped in the wells by antibody conjugation. Methods for detecting such complexes, in addition to those described above for the GST-immobilized complexes, include immunodetection of complexes using antibodies reactive with the Dicer interactor polypeptide, Dicer polypeptide or target polypeptide, as well as enzyme-linked assays which rely on detecting an enzymatic activity associated with the Dicer interactor polypeptide, Dicer polypeptide or target polypeptide.

In one aspect of the invention, the Dicer interactor or Dicer polypeptides can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with Dicer interactor or Dicer (“binding proteins” or “target molecules”) and are involved in Dicer interactor or Dicer activity. Such target molecules are also likely to be involved in the regulation of cellular activities modulated by the Dicer interactor polypeptides or Dicer polypeptides.

At least one exemplary two-hybrid system is based on the modular nature of most transcription factors, which consist of separable DNA-binding and activation domains. Briefly, the assay utilizes two different DNA constructs. In one construct, the gene that codes for a first polypeptide (the “bait” polypeptide, e.g., Dicer or Dicer protein) is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene that encodes the protein that interacts with the bait polypeptide.

Another exemplary two-hybrid system, referred to in the art as the CytoTrap™ system, is based in the modular nature of molecules of the Ras signal transduction cascade. Briefly, the assay features a fusion protein comprising the “bait” protein and Son-of-Sevenless (SOS) and the cDNAs for unidentified proteins (the “prey”) in a vector that encodes myristylated target proteins. Expression of an appropriate bait-prey combination results in translocation of SOS to the cell membrane where it activates Ras. Cytoplasmic reconstitution of the Ras signaling pathway allows identification of proteins that interact with the bait protein of interest, for example, a Dicer or Dicer interactor protein. Additional mammalian two hybrid systems are also known in the art and can be utilized to identify Dicer or Dicer interactor interacting proteins. Moreover, at least one of the above-described assays can be utilized to identify Dicer-interacting domains or regions of the Dicer interactor protein or alternatively, to identify Dicer protein-interacting domain or regions of the Dicer protein.

VB. Cell or Organism Based Assays

In one embodiment, an assay is a cell or organism-based assay in which a cell or organism capable of expressing a Dicer interactor polypeptide, or biologically active portion thereof, is contacted with a test compound and the ability of the test compound to modulate the expression of the Dicer interactor polypeptide, or biologically active portion thereof, determined. In another embodiment, an assay is a cell or organism-based assay in which a cell or organism which expresses a Dicer interactor polypeptide or Dicer polypeptide (or biologically active portions thereof) is contacted with a test compound and the ability of the test compound to modulate the activity of the Dicer interactor polypeptide or Dicer polypeptide (or biologically active portions thereof) determined. The cell, for example, can be of mammalian origin or a yeast cell. The organism can be a nematode, for example, C. elegans or C. briggsae or D. melanogaster. The polypeptides, for example, can be expressed heterologously or native to the cell or organism. Determining the ability of the test compound to modulate the activity of a Dicer interactor or Dicer polypeptide (or biologically active portions thereof) can be accomplished by assaying for any of the activities of a Dicer interactor or Dicer polypeptide described herein. Determining the ability of the test compound to modulate the activity of a Dicer interactor polypeptide or Dicer polypeptide (or biologically active portions thereof) can also be accomplished by assaying for the activity of a Dicer downstream molecule. In one embodiment, determining the ability of the test compound to modulate the activity of a Dicer interactor polypeptide, or biologically active portion thereof, is accomplished by assaying for the ability to bind Dicer or a bioactive portion thereof. In another embodiment, determining the ability of the test compound to modulate the activity of a Dicer interactor polypeptide, or biologically active portion thereof, is accomplished by assaying for the activity of the Dicer interactor polypeptide. In a preferred embodiment, the cell or organism overexpresses the Dicer interactor polypeptide, or biologically active portion thereof, and optionally, overexpresses Dicer, or biologically active portion thereof. In another preferred embodiment, the cell or organism expresses Dicer, or biologically active portion thereof. In yet another preferred example, the cell or organism is contacted with a compound that stimulates a Dicer protein-associated activity or Dicer-associated activity and the ability of a test compound to modulate the Dicer protein-associated activity is determined.

As used herein, the term “bioactive” fragment includes any portion (e.g., a segment of contiguous amino acids) of a Dicer interactor or Dicer protein sufficient to exhibit or exert at least one Dicer protein- or Dicer-associated activity including, for example, the ability to bind to Dicer or Dicer protein, respectively. In various embodiments, the Dicer may be one of two isoforms, Dicer1 or Dicer2. In another embodiment, the bioactive peptide is derived from the amino acid sequence of Dicer. In another embodiment, the bioactive peptide corresponds to a fragment or domain as set forth in subsections IA-IEE, supra or a smaller bioactive fragment thereof. In another embodiment, the bioactive peptide is derived from a Dicer interactor and can include, for example, amino acid residues sufficient to effect enzymatic or nucleic acid-binding activity.

According to the cell or organism-based assays of the present invention, determining the ability of the test compound to modulate the activity of the Dicer polypeptide or biologically active portion thereof, can be determined by assaying for any of the native activities of a Dicer polypeptide as described herein. Moreover, the activity of Dicer, can be determined by assaying for an indirect activity which is coincident to the activity of Dicer. Furthermore, determining the ability of the test compound to modulate the activity of the Dicer and/or Dicer interactor polypeptide or biologically active portion thereof, can be determined by assaying for an activity which is not native to the Dicer interactor or Dicer polypeptide, but for which the cell or organism has been recombinantly engineered. For example, the cell or organism can be engineered to express a target molecule which is a recombinant protein comprising a bioactive portion of Dicer operatively linked to a non-Dicer polypeptide or a bioactive portion of a Dicer interactor operatively linked to a non-Dicer interactor polypeptide. It is also intended that in preferred embodiments, the cell or organism-based assays of the present invention comprise a final step of identifying the compound as a modulator of Dicer interactor activity or Dicer activity.

VI. Assay Reagents

VIA. Test Compounds

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In a preferred embodiment, the library is a natural product library.

VIB. Antibodies Bioactive Fragments and Fusion Proteins

Preferred aspects of the invention feature Dicer polypeptides, Dicer interactor polypeptides and biologically active portions (i.e., bioactive fragments) of Dicer polypeptides or Dicer interactor polypeptides, including polypeptide fragments suitable for use in making Dicer interactor or Dicer fusion proteins. In one embodiment, Dicer polypeptides or Dicer interactor polypeptides can be isolated from cells or tissue sources by an appropriate purification scheme using standard protein purification techniques. Dicer polypeptide or Dicer interactor polypeptides can be further derived from said isolated polypeptides using standard enzymatic techniques. In another embodiment, Dicer interactor polypeptides, Dicer polypeptides or bioactive fragments thereof are produced by recombinant DNA techniques. Alternative to recombinant expression, Dicer interactor polypeptides, Dicer polypeptides or bioactive fragments thereof can be synthesized chemically using standard peptide synthesis techniques.

Polypeptides of the invention are preferably “isolated” or “purified”. The terms “isolated” and “purified” are used interchangeably herein. “Isolated” or “purified” means that the protein or polypeptide is substantially free of cellular material or other contaminating proteins from the cell or tissue source from which the polypeptide is derived, substantially free of other protein fragments, for example, non-desired fragments in a digestion mixture, or substantially free from chemical precursors or other chemicals when chemically synthesized. The language “substantially free of cellular material” includes preparations in which the polypeptide is separated from other components of the cells from which it is isolated or recombinantly produced. In one embodiment, the language “substantially free of cellular material” includes preparations of polypeptide having less than about 30% (by dry weight) of non-Dicer interactor or non-Dicer polypeptide (also referred to herein as a “contaminating protein”), more preferably less than about 20% of non-Dicer interactor or non-Dicer polypeptide, still more preferably less than about 10% of non-Dicer interactor or non-Dicer polypeptide, and most preferably less than about 5% non-Dicer interactor or non-Dicer polypeptide. When the polypeptide or protein is recombinantly produced, it is also preferably substantially free of culture medium, i.e., culture medium represents less than about 20%, more preferably less than about 10%, and most preferably less than about 5% of the volume of the polypeptide preparation. When the polypeptide or protein is produced by, for example, chemical or enzymatic processing from isolated or purified Dicer interactor or Dicer protein, the preparation is preferably free of enzyme reaction components or chemical reaction components and is free of non-desired Dicer interactor or Dicer fragments, i.e., the desired polypeptide represents at least 75% (by dry weight) of the preparation, preferably at least 80%, more preferably at least 85%, and even more preferably at least 90%, 95%, 99% or more or the preparation.

The language “substantially free of chemical precursors or other chemicals” includes preparations of polypeptide in which the polypeptide is separated from chemical precursors or other chemicals which are involved in the synthesis of the polypeptide. In one embodiment, the language “substantially free of chemical precursors or other chemicals” includes preparations having less than about 30% (by dry weight) of chemical precursors or reagents, more preferably less than about 20% chemical precursors or reagents, still more preferably less than about 10% chemical precursors or reagents, and most preferably less than about 5% chemical precursors or reagents.

Bioactive fragments of Dicer interactor or Dicer include polypeptides comprising amino acid sequences sufficiently identical to or derived from the amino acid sequence of the Dicer interactor protein or the Dicer protein, respectively, which include less amino acids than the full length protein, and exhibit at least one biological activity of the full-length protein. Typically, biologically active portions comprise a domain or motif with at least one activity of the full-length protein. A biologically active portion of a Dicer interactor or Dicer polypeptide can be a polypeptide which is, for example, 10, 20, 30, 40, 50, 100, 150, 200, 250, 300, 350, 400, 450, 500, 550, 600, 650, 700, 750, 800, 850, 900, 950, 1000 or more amino acids in length. In a preferred embodiment, a bioactive portion of a Dicer protein comprises a portion comprising a Dicer interactor interacting domain. Moreover, other biologically active portions, in which other regions of the protein are deleted, can be prepared by recombinant techniques and evaluated for one or more of the functional activities of a native Dicer interactor or Dicer protein. Mutants of Dicer and/or Dicer interactors can also be utilized as assay reagents, for example, mutants having reduced, enhanced or otherwise altered biological properties identified according to one of the activity assays described herein.

As defined herein, a Dicer polypeptide or Dicer interactor polypeptide of the invention includes polypeptides having the amino acid sequences set forth in subsections IA-IMM or II, infra, as well as homologs an/or orthologs of said polypeptides, i.e. polypeptides having sufficient sequence identity to function in the same manner as the described polypeptides. To determine the percent identity of two amino acid sequences (or of two nucleotide or amino acid sequences), the sequences are aligned for optimal comparison purposes (e.g., gaps can be introduced in the first sequence or second sequence for optimal alignment). The amino acid residues or nucleotides at corresponding amino acid positions or nucleotide positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10. BLAST alignments can be generated and percent identity calculated using BLAST protein searches (e.g., the XBLAST program) using Dicer protein, Dicer or a portion thereof as a query, score=50, wordlength=3.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Research 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The invention also provides Dicer interactors and Dicer chimeric or fusion proteins. As used herein, a Dicer interactor or Dicer “chimeric protein” or “fusion protein” comprises a Dicer interactor or Dicer polypeptide operatively linked to a non-Dicer interactor polypeptide or non-Dicer polypeptide, respectively. A “Dicer interactor polypeptide” or “Dicer polypeptide” refers to a polypeptide having an amino acid sequence corresponding to the Dicer interactor or Dicer protein, respectively, whereas a “non-Dicer interactor polypeptide” or “non-Dicer polypeptide” refers to a polypeptide having an amino acid sequence corresponding to a protein which is not substantially identical to the Dicer interactor protein or Dicer protein. Within a fusion protein the Dicer interactor or Dicer polypeptide can correspond to all or a portion of a Dicer interactor or Dicer protein. In a preferred embodiment, a Dicer interactor or Dicer fusion protein comprises at least one biologically active portion of a Dicer interactor or Dicer protein, respectively. In another preferred embodiment, a Dicer interactor or Dicer fusion protein comprises at least two biologically active portions of a Dicer interactor or Dicer protein, respectively. In yet another preferred embodiment, a fusion protein can comprise Dicer protein, or a bioactive portion thereof, operatively linked to Dicer, or a bioactive portion thereof, such that Dicer interactor and Dicer, or their respective bioactive portions are brought into close proximity. Within the fusion protein, the term “operatively linked” is intended to indicate that the Dicer interactor or Dicer polypeptide and the non-Dicer interactor polypeptide or non-Dicer polypeptide are fused in-frame to each other. The non-Dicer interactor polypeptide or non-Dicer polypeptide can be fused to the N-terminus or C-terminus of the Dicer interactor polypeptide or Dicer polypeptide, respectively.

For example, in one embodiment, the fusion protein is a GST-fusion protein in which the Dicer interactor or Dicer sequences are fused to the C-terminus of the GST sequences. In another embodiment, the fusion protein is a chitin binding domain (CBD) fusion protein in which the Dicer interactor or Dicer sequences are fused to the N-terminus of chitin binding domain (CBD) sequences. Such fusion proteins can facilitate the purification of recombinant Dicer interactor or Dicer.

Preferably, a chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety. A Dicer protein- or Dicer-encoding nucleic acid can be cloned into such an expression vector such that the fusion moiety is linked in-frame to the Dicer interactor or Dicer polypeptide.

A Dicer interactor polypeptide or Dicer polypeptide, or a portion or fragment of Dicer interactor or Dicer, can also be used as an immunogen to generate antibodies that bind Dicer interactor or Dicer or that block Dicer protein/Dicer binding using standard techniques for polyclonal and monoclonal antibody preparation. A full-length polypeptide can be used or, alternatively, the invention provides antigenic peptide fragments for use as immunogens. Preferably, an antigenic fragment comprises at least 8 amino acid residues of the amino acid sequence of a Dicer interactor or Dicer and encompasses an epitope of Dicer interactor or Dicer such that an antibody raised against the peptide forms a specific immune complex with Dicer interactor or Dicer, respectively. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of Dicer interactor or Dicer that are located on the surface of the protein, e.g., hydrophilic regions. Antigenic determinants at the termini of Dicer interactor are preferred for the development of antibodies that do not interfere with the Dicer protein:Dicer interaction. Alternatively, interfering antibodies can be generated towards antigenic determinants located within the Dicer interacting domain of Dicer protein. The latter are preferred for therapeutic purposes.

A Dicer interactor or Dicer immunogen typically is used to prepare antibodies by immunizing a suitable subject, (e.g., rabbit, goat, mouse or other mammal) with the immunogen. An appropriate immunogenic preparation can contain, for example, recombinantly expressed Dicer interactor or Dicer polypeptide or a chemically synthesized Dicer interactor or Dicer polypeptide. The preparation can further include an adjuvant, such as Freund's complete or incomplete adjuvant, or similar immunostimulatory agent. Immunization of a suitable subject with an immunogenic Dicer interactor or Dicer preparation induces a polyclonal anti-Dicer interactor or anti-Dicer antibody response, respectively.

Accordingly, another aspect of the invention pertains to anti-Dicer interactor or anti-Dicer antibodies. The term “antibody” as used herein refers to immunoglobulin molecules and immunologically active portions of immunoglobulin molecules, i.e., molecules that contain an antigen binding site which specifically binds (immunoreacts with) an antigen, such as Dicer interactor or Dicer. Examples of immunologically active portions of immunoglobulin molecules include F(ab) and F(ab+)₂ fragments which can be generated by treating the antibody with an enzyme such as pepsin. The invention provides polyclonal and monoclonal antibodies that bind Dicer protein. The term “monoclonal antibody” or “monoclonal antibody composition”, as used herein, refers to a population of antibody molecules that contain only one species of an antigen binding site capable of immunoreacting with a particular epitope of Dicer interactor or Dicer. A monoclonal antibody composition thus typically displays a single binding affinity for a particular Dicer interactor or Dicer polypeptide with which it immunoreacts.

Polyclonal anti-Dicer interactor or anti-Dicer antibodies can be prepared as described above by immunizing a suitable subject with a Dicer interactor or Dicer immunogen, respectively. The antibody titer in the immunized subject can be monitored over time by standard techniques, such as with an enzyme linked immunosorbent assay (ELISA) using immobilized Dicer interactor or Dicer. If desired, the antibody molecules can be isolated from the mammal (e.g., from the blood) and further purified by well known techniques, such as protein A chromatography to obtain the IgG fraction. At an appropriate time after immunization, e.g., when the anti-Dicer interactor or anti-Dicer antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a Dicer interactor or Dicer immunogen as described above, and the culture supernatants of the resulting hybridoma cells are screened to identify a hybridoma producing a monoclonal antibody that binds Dicer interactor or Dicer, respectively.

Any of the many well known protocols used for fusing lymphocytes and immortalized cell lines can be applied for the purpose of generating an anti-Dicer interactor monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind Dicer interactor or Dicer, e.g., using a standard ELISA assay.

Alternative to preparing monoclonal antibody-secreting hybridomas, a monoclonal anti-Dicer interactor or anti-Dicer antibody can be identified and isolated by screening a recombinant combinatorial immunoglobulin library (e.g., an antibody phage display library) with Dicer interactor or Dicer to thereby isolate immunoglobulin library members that bind Dicer interactor or Dicer, respectively. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

An anti-Dicer interactor or anti-Dicer antibody (e.g., monoclonal antibody) can be used to isolate Dicer interactor or Dicer, bioactive portions thereof, or fusion proteins by standard techniques, such as affinity chromatography or immunoprecipitation. Anti-Dicer antibodies or anti-Dicer interactor antibodies made according to any of the above-described techniques can be used to detect protein levels in donor or acceptor fractions as part of certain assay methodologies described herein. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

VIC. Recombinant Expression Vectors and Assay Cells or Organisms

Another aspect of the invention pertains to vectors, preferably expression vectors, for producing the proteins reagents of the instant invention. As used herein, the term “vector” refers to a nucleic acid molecule capable of transporting another nucleic acid to which it has been linked. A preferred vector is a “plasmid”, which refers to a circular double stranded DNA loop into which additional DNA segments can be ligated. In the present specification, “plasmid” and “vector” can be used interchangeably as the plasmid is the most commonly used form of vector.

The recombinant expression vectors of the invention comprise a nucleic acid that encodes, for example protein or Dicer or a bioactive fragment or Dicer interactor or bioactive fragment, in a form suitable for expression of the nucleic acid in a host cell or organism, which means that the recombinant expression vectors include one or more regulatory sequences, selected on the basis of the host cells or organisms to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell or organism when the vector is introduced into the host cell or organism). The term “regulatory sequence” is intended to includes promoters, enhancers and other expression control elements (e.g., polyadenylation signals). The expression vectors can be introduced into host cell or organisms to thereby produce proteins, including fusion proteins or peptides. Alternatively, retroviral expression vectors and/or adenoviral expression vectors can be utilized to express the proteins of the present invention.

The recombinant expression vectors of the invention can be designed for expression of Dicer interactor or Dicer polypeptides in prokaryotic or eukaryotic cells. For example, Dicer interactor or Dicer polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990).

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Purified fusion proteins are particularly useful in the cell-free assay methodologies of the present invention.

In yet another embodiment, a protein or Dicer-encoding or Dicer-protein-encoding nucleic acid is expressed in mammalian cells, for example, for use in the cell or organism-based assays described herein. When used in mammalian cells, the expression vector's control functions are often provided by viral regulatory elements. In another embodiment, the recombinant mammalian expression vector is capable of directing expression of the nucleic acid preferentially in a particular cell type (e.g., tissue-specific regulatory elements are used to express the nucleic acid).

Another aspect of the invention pertains to assay cells into which a recombinant expression vector has been introduced. An assay cell can be prokaryotic or eukaryotic, but preferably is eukaryotic. Cell lines are cultured according to art-recognized techniques. Vector DNA can be introduced into prokaryotic or eukaryotic cells via conventional transformation or transfection techniques. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals. An assay cell of the invention, can be contacted with a test compound and assayed for any Dicer interactor and/or Dicer biological activity in order to identify the compound as a modulator. Biological activities that can further be assayed as part of the methodologies of the present invention include, but are not limited to, (1) processing of miRNA precursors; (2) processing of siRNA precursors; (3) mediating mRNA cleavage; (4) mediating assembly of RISC (e.g., via siRNAs); (5) directing translation repression (e.g., via miRNAs); (6) a ribonuclease activity (e.g., cleavage of dsRNA); and (7) initiation of RNAi. In addition, other biological activities which may be assayed for include those listed in Table 1 and/or subsections IA-IMM and II, supra.

VII. Pharmaceutical Compositions

This invention further pertains to modulators identified by the above-described screening assays. Modulators identified by the above-described screening assays can be tested in an appropriate animal model. For example, a Dicer modulator, RNAi modulator and/or gene silencing modulator identified as described herein can be used in an animal model to determine the efficacy, toxicity, or side effects of treatment with such a modulator. Alternatively, a modulator identified as described herein can be used in an animal model to determine the mechanism of action of such an agent. Furthermore, this invention pertains to uses of modulators identified by the above-described screening assays for therapeutic treatments as described infra.

Accordingly, the modulators of the present invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

VIII. Methods of Treatment

The present invention also features methods of treatment or therapeutic methods. In one embodiment, the invention features a method of treating a subject (e.g., a human subject in need thereof) with a modulatory compound identified according to the present invention, such that a desired therapeutic effect is achieved. In another embodiment, the method involves administering to an isolated tissue or cell line from the subject a modulatory compound identified according to the methodology described herein, such that a desired therapeutic effect is achieved. In a preferred embodiment, the invention features a method of treating a subject having a disease or disorder characterized by overexpression or aberrant expression of a particular protein. For example, positive modulators of Dicer and/or RNAi can be used to enhance RNAi of deleterious proteins. Likewise, negative modulators of Dicer and/or RNAi can be used to alleviate symptoms resulting from the RNAi pathway. Desired therapeutic effects include a modulation of any Dicer protein-, Dicer- or Dicer protein/Dicer-associated activity, as described herein. Desired therapeutic effects also include, but are not limited to curing or healing the subject, alleviating, relieving, altering or ameliorating a disease or disorder in the subject or at least one symptom of said disease or disorder in the subject, or otherwise improving or affecting the health of the subject. A preferred aspect of the invention pertains to methods of modulating Dicer protein/Dicer interactions for therapeutic purposes.

The modulators identified by the methods disclosed herein may be used in a subject to modulate (1) processing of miRNA precursors; (2) processing of siRNA precursors; (3) mediating mRNA cleavage; (4) mediating assembly of RISC (e.g., via siRNAs); (5) directing translation repression (e.g., via miRNAs); (6) a ribonuclease activity (e.g., cleavage of dsRNA); and/or (7) initiation of RNAi.

The effectiveness of treatment of a subject with a Dicer modulator, RNAi modulator and/or gene silencing modulator can be accomplished by (i) detecting the level of activity in the subject prior to treating with an appropriate modulator; (ii) detecting the level of activity in the subject post treatment with the modulator; (iii) comparing the levels pre-administration and post administration; and (iv) altering the administration of the modulator to the subject accordingly. For example, increased administration of the modulator may be desirable if the subject continues to demonstrate undesireable symptoms of the disease or disorder being treated.

IX. Diagnostic Assays

The present invention also features diagnostic assays, for determining aberrant Dicer protein:Dicer interaction, expression or activity, in the context of a biological sample (e.g., blood, serum, cells, tissue) to thereby determine whether an individual is afflicted with a disease or disorder associated with said aberrancy or is at risk of developing such a disorder. The invention also provides for prognostic (or predictive) assays for determining whether an individual is at risk of developing such a disorder (e.g., a disorder associated with aberrant Dicer interactor expression or activity). Such assays can be used for prognostic or predictive purpose to thereby prophylactically treat an individual prior to the onset of a disease or disorder. A preferred agent for detecting a Dicer interactor or Dicer protein is an antibody capable of binding to protein or Dicer, respectively, preferably an antibody with a detectable label. The term “biological sample” is intended to include tissues, cells and biological fluids isolated from a subject, as well as tissues, cells and fluids present within a subject. The invention also encompasses kits for the detection of aberrant Dicer protein:Dicer interaction, expression or activity in a biological sample. For example, the kit can comprise a labeled compound or agent capable of detecting Dicer interactor and/or Dicer in a biological sample; means for determining the amount of Dicer interactor and/or Dicer in the sample; and/or means for comparing the amount of Dicer interactor in the sample with a standard. The compound or agent can be packaged in a suitable container. The kit can further comprise instructions for using the kit.

X. Uses

The invention has several further advantageous uses which include, but are not limited to, the following: providing interacting proteins of Dicer and there use in modulating Dicer function; methods for identifying further interactors of Dicer and their structural and functional characteristics; method for regulating Dicer activity though the use of Dicer interactors; methods for improving the in vitro or in vivo processing of Dicer proteins or for as targets for pharmaceutical intervention in order to modulate the properties of Dicer in vivo for improved RNAi; and methods for stabilizing RNAi agents/compositions comprising Dicer by the addition of stabilizing interactor proteins or the same for use in purifying Dicer and other Dicer components.

This invention is further illustrated by the following examples which should not be construed as limiting. The contents of all references, patents and published patent applications cited throughout this application are hereby incorporated by reference.

Exemplification

Throughout the examples, the following materials and methods were used unless otherwise stated.

Materials and Methods

In general, the practice of the present invention employs, unless otherwise indicated, conventional techniques of nucleic acid chemistry, recombinant DNA technology, molecular biology, biochemistry, cell biology and transgenic animal biology. See, e.g., DNA Cloning, Vols. 1 and 2, (D. N. Glover, Ed. 1985); Oligonucleotide Synthesis (M. J. Gait, Ed. 1984); Oxford Handbook of Nucleic Acid Structure, Neidle, Ed., Oxford Univ Press (1999); RNA Interference: The Nuts & Bolts of siRNA Technology, by D. Engelke, DNA Press, (2003); Gene Silencing by RNA Interference: Technology and Application, by M. Sohail, CRC Press (2004); Sambrook, Fritsch and Maniatis, Molecular Cloning: Cold Spring Harbor Laboratory Press (1989); and Current Protocols in Molecular Biology, eds. Ausubel et al., John Wiley & Sons (1992), which are incorporated in their entireties by reference herein.

C. elegans Strains

Typical C. elegans strains for carrying out the invention as described herein include, for example, N2; alg-2 strain (ok304); dcr-1 for rescue; dcr-1 counterselectable; f20 counterselectable; drh-3 counterselectable; bn-2 (glp-4); rde-4 ne337; eri-1 (mg366); rrf-3 (pk); eri-3 (tm); and eri-5 (mg, tm).

Antibody Development and Purification

Antisera against C. elegans Dicer, i.e., DCR-1, were raised in rabbits as described by Capralogics services (Capralogics, Hardwick, Mass., USA). The antisera used for the somatic purifications, and for the immunoblot analyses were developed using a fragment encoded by residues 1145 to 1347 of the protein fused to the pCal-KC (Stratagene) encoded fusion. For their affinity purification, another fragment encoding residues 966 to 1347 was expressed as a pET-42a (Novagen) fusion, purified under denaturing conditions, using Guanidine HCl (Ultra grade, FLUKA) 6M/150 mM NaCl/HEPES 25 mM pH 8.0 as a lysis, binding and washing buffer. The purified fusion was eluted in Guanidine HCl 6M/150 mM NaCl/MES 25 mM pH4.8 and added directly to the Affigel 10 (Biorad) and allowed to rock O/N for covalent coupling of the fusion.

The matrix was then washed in coupling buffer (5 column volumes) in Tris (10 column volumes) and remaining active sites were blocked using triethanolamine/HCl for 2 h at 4 degrees. The matrix was then rinsed extensively in PBS and used for affinity-purification of the antisera. The sera (4 ml per batch) were diluted 1:5 in PBS, filtered sterile and loaded directly on the prepared affinity matrixes. After batch binding, the beads were washed extensively with PBS in a column and, the antibodies were eluted (8 column volumes) using glycine 0.2M pH2.2, while harvesting the fractions if 3:7 volumes of Potassium Phosphate solution at pH10, to neutralize the fractions. Consecutive purifications (3) were realized with the same serum batch with similar antibody recovery.

Fractions were then examined by SDS-PAGE, and quantified by comparison with BSA standards. The fractions containing the antibodies were dialyzed against PBS/5% glycerol, and concentrated to ˜1 microgram per microliter using the Centricon 10 centrifuge dialysis system (Millipore). The concentrated antibodies were frozen at −80 until used.

Dicer (Dcr-1) Transgenic Rescue

A fragment encoding the 3′ portion of the Dicer (dcr-1) gene was cloned into Bluescript SK (Stratagene) and a Not I site was inserted prior to its stop codon. A NotI cassette encoding 8 copies of HA, and the yeast sup4o gene embedded in an artificial C. elegans intron was then inserted in frame at the 3′ portion of the recombination cassette. This fusion was then prepared by PCR and used to transform a yeast strain bearing the YAC Y97B3. The strain was then selected on URA-/LYS- for YAC recombinants.

Confirmed recombinants, were screened by PCR and a genomic preparation of the strain was realized. A C. elegans strain bearing dcr-1 (ok247), and the dpy-13 lesions balanced by the sDp-3 free duplication was used for rescue. The genomic DNA was injected in the balanced animals germline at 200 ng per microliter with an additional 50 ng per microliter of sur-5::gfp expressing vector, as a secondary marker for transformation. Mosaics (F1) were selected on the basis of their GFP signal, singled out, and transmitting lines were identified with regard of the GFP signal of the F2s.

Genomic DNA was then prepared from 2-5 animals of such lines, and examined for the presence of the recombinant YAC DNA by PCR. 4 out of 22 independent transgenic lines had the YAC, and of such, 2 of the strains consistently produced dpy animals with fertile progeny. Single picks from such animals led to dpy populations, in which only GFP+ animals were fertile thereby indicating that the rescue was due to the recombinant YAC.

Fractionation and Immunoblot Analysis

For the somatic purifications, and the RNA analysis, the C. elegans strains were grown in standard conditions, as synchronous populations and harvested as adults with a single row of embryos, or allowed to grow 12 h after the L4 to young adult transition, in the case of sterile animals. Animals were rinsed in M9 twice and floated on sucrose if gravid adults were used. Animals were allowed to rock in M9 for 30 min at RT to allow digestion of the gut bacterial load. For embryonic preparations, gravid adults were hypochlorided as previously described, rinsed in M9 three times, and further rinsed in cold water. The animals were then pelleted using a table-top falcon centrifuge, and frozen at −80° C. as a compacted pellet after all the supernatant was drained.

Preparations where further processed using one volume of hypotonic buffer, 10 mM HEPES KOAc pH 7.5; 10 mM K(OAc); 2 mM Mg(OAC)₂; 1 mM DTT with 4× concentration complete protease inhibitors and RNase inhibitors. The suspension was then transferred to a cold Dounce homogenizer, and stroked 20-30 times, on ice. The resulting slurry was then transferred to an Eppendorf, and the recovered volume was adjusted to 110 mM KCl (yields Isotonic buffer), vortexed and allowed to sit on ice for 10 minutes.

The nuclear fraction was prepared in the following manner. The slurry was first centrifugation at 1500×g for 30 sec at 4°. The supernatant was recovered and adjusted to 10% glycerol, 0.01% Triton X-100, and vortexed and allowed to sit on ice for 10 min. The slurry was then loaded on a sucrose cushion (10 mM HEPES pH7.5; 10 mM KCl; 1M sucrose; 10% glycerol; 1 mM EDTA), and centrifuged at 20000×g for 10 min at 4°. The pellet yields the nuclear fraction.

The S100 and P100 fractions were prepared as follows. The supernatant from a short 1500×g centrifugation was further centrifuged at 10000×g for 10 min at 4°, and the supernatant was recovered (S10 fraction). This fraction was then loaded in a Beckman microfuge polyallomer tube and further spun 1 h 4° C. in a TLA100.3 to yield the S100 and P100 fractions. Equivalent volumes of each fraction was precipitated in 2 volumes acetone and resuspended in 1× SDS-PAGE buffer for the fraction analyses.

Immunoblots were realized using PBS/0.1% tween/5% milk for blocking and blotting, and PBS/0.1% tween for washings. Primary antibodies were incubated at RT for lh, and the corresponding HRP-coupled secondaries were used at 1:5000 for 1 h before 3× 5 min washes and ECL development (Pierce).

Purifications and Immunoprecipitations

Immunoprecipitation matrixes were prepared by DimethylPimeliimidate (DMP) (Sigma) covalent coupling to rProtA-agarose beads (Pierce) in sodium borate pH9.0 buffer. The beads were then stripped and blocked in 0.2M glycine pH2.2, rinsed extensively in PBS and kept until use at 4° C. with thymerosal as antibacterial agent. For typical preparations, 1 mg purified polyclonal antibodies were covalently coupled to 200 ul rProtA beads. In the case of embryonic purifications, agarose coupled matrixes from both antibody clones were used.

For the Dicer (DCR-1) purifications, the S100 fraction was further quantified and diluted to 3 mg per ml concentration in 1% Triton X-100 supplemented Isotonic buffer before the suitable buffer-equilibrated matrixes (30 ul bead volume per 2 ml IP) were added to the mixtures. Immunoprecipitations were carried out at 4° C. for 1 h, and beads were then washed 3 times in the immunoprecipitation buffer.

Immunoprecipitates were then treated with 20 ug per ml RNaseA for 30 minutes on ice in the same buffer, then washed three more times. The beads were washed one more time in cold PBS, and all the supernatant was drained. Bound proteins were eluted in 8M urea/50 mM HEPES pH7.5, and acetone-precipitated. ⅕^{th} the elution volume was kept and monitored on silver stain and/or by immunoblot for a qualitative evaluation of the immunoprecipitation process.

RNA Interferences

Feeding and microinjection RNAi was carried out as previously reported by Conte Jr. D. and Mello, C. C. 2003. RNA interference in Caenorhabditis Elegans. In Current Protocols in Molecular Biology.

Northern Blotting and Real Time PCR

Small RNAs were prepared from N2(wt), bn2(glp4), which lack a germline tissue, and mutants for rde-4(ne337), rrf-3(pk1426), eri-1(mg366), eri-3(tm1361), and eri-5(mg370), all at 25° C. Homozygous dcr-1(ok247), f20d12.1, and drh-3(tm]217) sterile adult animals were isolated using the counterselectable genetic balancer method. For alg-1 and alg-2 depleted preparations, alg-2(ok204) L1 animals were exposed to an alg-1(rnai) feeding strain, and the bursting young adults were harvested and used for small RNA preparations. The isolated small RNA preparations were typically examined by Northern blotting for a variety of endogenous small RNAs as well as miR58, tncR7, and the X chromosome locus contig of small RNAs described in the art. Real time PCR was performed with primer pairs having efficiencies validated for a multiple 10 fold dilution range around the N2(wt) level, and fold changes were calculated using the delta delta Ct method.

Imaging and Video Microscopy

DAPI staining of intact animals was done as described in the art. Endomitotic (Emo) phenotype was scored by intense and irregular DAPI staining or expression of histoneH2::gfp in germ cell nuclei. Nematode gonads were dissected as described in the art with slight modifications. Briefly, young adult worms were placed in a drop of PBS containing 0.15 mM of levamisole on a glass slide for gonad extrusion. The dissected gonads were then fixed in 4% paraformaldehyde in PBS for 5 minutes, followed by three washes of PBS before staining with DAPI for 5 minutes. Gonads were then mounted for imaging after 3 washes with PBS. DIC or fluorescence images were collected by a Hamamatsu Ocre-ER digital camera mounted on a Zeiss Axioplan 2 under the control of Openlab 3.0 software. In time-lapse video microscopy experiments, young adult animals expressing a histoneH2::gfp fusion in the germline (AZ212) were cut open in M9 solution and embryos were mounted on 2% agarose pads in M9 solution for recording by a Leica TCS SP2 confocal microscope system. Movies were processed on a Macintosh computer using the public domain Image J 10.2 program (developed at the U.S. National Institutes of Health.

Multi Dimensional Protein Identification Technology

The MudPIT assays were performed essentially as described in Graumann et al., Mol Cell Proteomics, 3(3):226-37 (2004) and Liu et al., Biotechniques. 32(4):898, 900, 902 passim (2002).

### EXAMPLE 1

### Methods for Identifying Dicer Interacting Proteins

To identify Dicer interacting proteins a protenomic approach was employed. In particular, a combined and comparative proteomic approach was designed and used to identify novel factors implicated in molecular interactions with Dicer (DCR-1) in the nematode C. elegans. The approach featured a combined transgenic and immuno-biochemical purification scheme with an innovative Mass Spectrometry technology called MudPit (Multi-dimensional protein identification technology) in order to identify proteins interacting with DCR-1 in the embryo and in the adult of the animal and compared with the interactors identified in parallel as being interactors of the RDE-4 and RDE-1 proteins. The MudPit technology has been previously described (see, e.g., Graumann et al., Mol Cell Proteomics, 3(3):226-37 (2004); Liu et al., Biotechniques. 32(4):898, 900, 902 passim (2002)).

Using this approach, several interactions were identified which have important significance as to how DCR-1 can be up- or down-regulated and how DCR-1 is implicated in different functions. Table 1 lists the DCR-1 interactors identified using the above approach. Table 2 shows the corresponding protein interaction data obtained for each interactor. Many of these interactors are widely conserved and have homologs in other species such as human or mouse. These interactors are implicated as activators or inhibitors of the DCR-1 activity, specificity, and/or stability and can be utilized for improved in vitro processing of a variety of DCR-1 proteins. The interactors can also be used as part of a rationale or also as targets for pharmaceutical intervention in order to modulate the properties of DCR-1 in vivo.

TABLE 1
List of Dicer Interacting Proteins Identified in Pilot Scale

| | Protein | | |
|---|---|---|---|
| § | CE# | Description | Phenotype |
| IIA | | RDE-4 | RDE |
| IIB | | ALG-1 | EXP |
| IIC | | ALG-2 | EXP |
| IID | | DRH-1 | RDE |
| IIE | | DRH-2 | RDE |
| IIF | ce09069 | helicase | ND |
| | | homologous to DCR-2 | |
| IIG | ce21971 | Double helicase | EMB |
| IIH | | EFT-2 EF-Tu family | EMB; Pvl; Ste; Lva |
| | | GTP binding | |
| III | | EFT-4 (elF1 alpha) | EMB; Gro; Lva; |
| | | | Unc; Ste |
| IIJ | ce21437 | GAP/RAN-GAP family | ND |
| IIK | ce08872 | HMG-I/Y DNA | WT |
| | | binding | |
| IIL | ce20336 | HMG-I/Y DNA | Dpy; EMB; Lvl; |
| | | binding PB1 domain | Ste; Unc; Lva |
| IIM | ce14704 | SNR-2 SM protein | EMB; Ste; EXP; Lva |
| IIN | ce02065 | SNR-3 SM protein | EMB; Clr; Sck; Lva |
| IIO | ce03706 | Dual specificity | EMB; EXP |
| | | phosphatase | |
| IIP | | LIN-41 | |
| IIQ | ce001506 | low homology | GRO |
| | | MADS box, novel | |
| IIR | | RPN-9 proteasome | EMB; BMD, Unc, |
| | | subunits | Gro, Lva |
| IIS | ce14736 | TAF 6.1 | WT (ND) |
| IIT | ce05915 | T54 homology | Unc Stp Gro |
| IIU | ce21988 | RRM protein (3 | ND |
| | | domains) | |
| IIV | ce27223 | Worm | WT (ND) |
| | | unique/Novel | |
| IIW | ce00850 | TBB-4 | EMB |
| IIX | | RPS-14 | |
| IIZ | | RPS-13 | |
| IIAA | | RPL-24 | |
| IIBB | | RPS-11 | |
| IICC | ce03050 | Agglutinin | |
| IIDD | | SIP-1 (hsp20) | WT |
| IIEE | | CCT-6 | |
| | | (chaperonin) | |

TABLE 2
List of Dicer: Dicer Interacting Protein Interaction Results

| | | IP | | | | | IP 1001(2) - | R4 (2) - |
|---|---|---|---|---|---|---|---|---|
| § | description | Controls | IP 1 | IP 2 | IP 3 | IP 4 | ctls | ctrl |
| IIA | RDE-4 | NP | P | P | P | P | P | P |
| IIB | ALG-1 | NP | | P | P | P | P | |
| IIC | ALG-2 | NP | | P | P | P | P | |
| IID | DRH-1 | NP | P | P | P | P | P | p |
| IIE | DRH-2 | NP | P | P | P | P | P | P |
| IIF | helicase homologous | NP | | | P | P | P | |
| | to DCR-2 | | | | | | | |
| IIG | Double helicase | NP | P | P | P | P | | |
| IIH | EFT-2 EF-Tu family | NP | | P | P | P | P | P |
| | GTP binding | | | | | | | |
| III | EFT-4 (elF1 alpha) | NP | | P | P | P | P | |
| IIJ | GAP/RAN-GAP family | NP | | P | P | P | | |
| IIK | HMG-I/Y dna binding | NP | | P | P | P | | |
| IIL | HMG-I/Y dna binding | NP | | | P | P | | |
| | PB1 domain | | | | | | | |
| IIM | SNR-2 SM protein | NP | | | P | P | | |
| IIN | SNR-3 SM protein | NP | | | P | P | P | |
| IIO | Dual specificity | NP | | P | P | P | P | |
| | phosphatase | | | | | | | |
| IIP | LIN-41 | NP | | | P | P | P | |
| IIQ | low homology MADS | NP | P | P | P | P | P | |
| | box, novel | | | | | | | |
| IIR | RPN-9 proteasome | NP | | P | P | P | | |
| | subunits | | | | | | | |
| IIS | TAF 6.1 | NP | | | P | P | P | |
| IIT | T54 homology | NP | | | P | P | | |
| IIU | RRM protein (3 | NP | | | P | P | | |
| | domains) | | | | | | | |
| IIV | Worm unique/Novel | NP | | | P | P | P | |
| IIW | TBB-4 | NP | P | P | P | P | | P |
| IIX | RPS-14 | NP | | P | P | P | | |
| IIZ | RPS-13 | NP | P | P | P | P | | |
| IIAA | RPL-24 | NP | | | P | P | | |
| IIBB | RPS-11 | NP | P | P | P | P | | |
| IICC | Agglutinin | NP | | | P | P | | |
| IIDD | SIP-1 (hsp20) | NP | | P | P | P | | |
| IIEE | CCT-6 (chaperonin) | NP | | P | P | P | | |

NP = not present;
P = present;
IP = immunoprecipitation;
1 to 4 corresponds to four independent purifications with that affinity matrix;
1001 = second antibody matrix;
R4 (2) - ctls = interactors found in two independent purifications of RDE-4, absent from the controls, and also present in the DCR-1 purifications.

### EXAMPLE 2

### Methods for Conducting a Whole Organism Search for Dicer Interactions

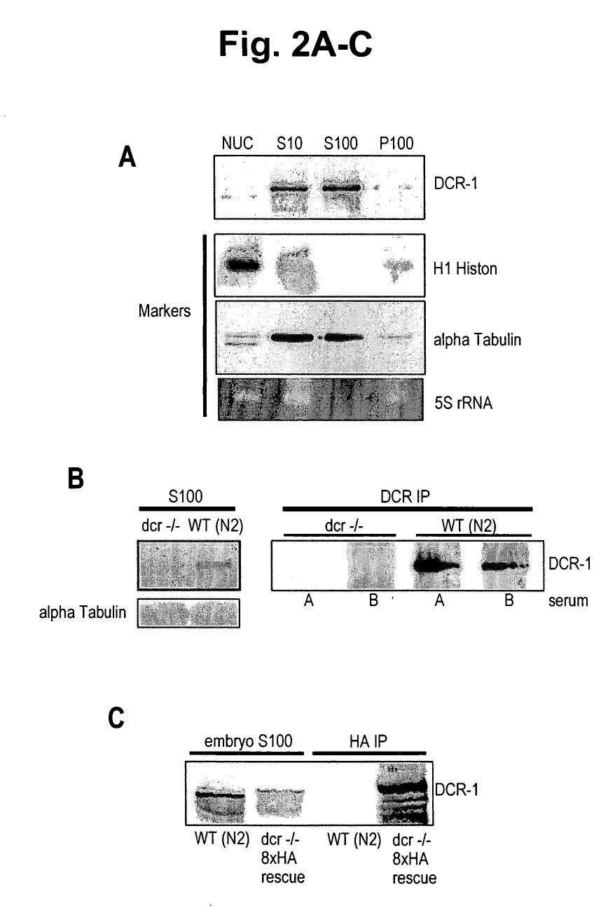

In order to identify Dicer interacting proteins in a whole organism, strategies to affinity-purify Dicer (DCR-1) by multiple independent matrixes, both from embryos and gravid adults C. elegans, were developed. Fractionation analysis showed that most, if not all the C. elegans Dicer protein can be found in the S100 fraction (FIG. 2).

For the adult purification, rabbit polyclonal sera having efficient immunoprecipitation capacity for the Dicer protein were identified. The antisera were affinity-purified against their respective antigen, coupled covalently to agarose matrixes, and used for batch immuno-affinity purifications. Controls included preparations from extracts genetically null for any Dicer expression (dcr-1 deletion allele (ok247)), or mock purification comprising neutralized affinity beads.

For the embryonic purifications, a transgenic dcr-1::8×HA genomic fusion driven by its own promoter, was used. The transgene allowed the sterility phenotype of dcr-1−/− to be rescued, and a robust expression in young embryos indicating it can support the functions of DCR-1 in the germline. Purification of DCR-1::8×HA fusion protein was carried out using two distinct monoclonal HA-directed affinity matrixes, and used the non-transgenic WT (N2) embryos as a control.

The purified proteins were eluted and analyzed using the Multi-Dimensional Protein Identification Technology (MudPIT). Interacting proteins were identified by comparison of the detected peptides with both the predicted and confirmed ORF library of the C. elegans genome. Protein candidates were not investigated further if they were also found in the depleted control, or in the mock purification (uncoupled matrix only). Chaperones, and two structural proteins, which were found in multiple non-related purifications, and known to be common non-specific interactions, were intentionally excluded. A high confidence set of interactions for proteins that could be detected in multiple purifications, with at least two independent matrixes, was defined. Using this strategy, 16 proteins were shown to interact with DCR-1. Table 3 depicts the interactions that were detected using this criteria.

TABLE 3
List of Dicer Interacting Proteins

| | gene | DCR-1 | structural | |
|---|---|---|---|---|
| | name | purification | description | Phenotype |
| 1FF | RDE-1 | E* W | Piwi/PAZ domain | Rde |
| 1A | RDE-4 | E A** W | dsRBD | Rde |
| 1D | DRH-1 | E A W | DEAH/D box | Rde |
| 1E | DRH-2 | E A W | DEAH/D box | Rde |
| 1GG | D2005.5 | E A | DEAH/D box | early |
| | (DRH-3) | | | embryonic |
| | | | | arrest, sterile |
| 1HH | ERI-1 | E | SAP domain, | ts sterile, eri |
| | | | Exonuclease | |
| 1II | RRF-3 | E | rdrp | ts sterile, eri |
| 1JJ | W09B6.3 | E A | Novel (operon | ts sterile, eri |
| | (eri-3) | | and fusion | |
| | | | with TAF-6.1) | |
| 1KK | Y38F2AR.1 | E A | TUDOR domain | ts sterile, eri |
| | (eri-5) | | | |
| 1S | TAF-6.1 | E A | TATA box binding | ND; eri |
| | | | protein associated | |
| | | | factor (operon | |
| | | | and fusion with | |
| | | | eri-3) | |
| 1B | ALG-1 | A W | Piwi/PAZ domain | heterochronic |
| 1C | ALG-2 | A W | Piwi/PAZ domain | heterochronic |
| 1P | LIN-41 | A | RBCC (NHL | heterochronic, |
| | | | family) | pleiotropic |
| 1LL | T23G7.5 | E A | Phosphatase | rde, L4 |
| | (PIR-1) | | | developmental |
| | | | | arrest |
| 1H | EFT-2 | A | EFT-2 family | lethal, |
| | | | GTPases | pleiotropic |
| 1N | SNR-3 | A | SM domain | lethal, |
| | | | | pleiotropic |
| 1MM | C32A3.2 | A | Novel | WT |

Abbreviations are as follows:
E: embryonic purification,
A: gravid adult purification,
W: western detection,
rde: required for RNAi;
eri: enhancer of RNAi;
*weak peptide coverage only,
**due to the robust interaction, weak peptide coverage of RDE-4 is also detected in the dcr counterselected allele, likely due to interaction with the maternal load.

Proteins known to be involved in the initiation step of RNAi were found in all the DCR-1 purifications. The double-stranded binding protein RDE-4 was shown to interact with DCR-1. RDE-4 was also shown to interact with the argonaute family protein RDE-1, and the helicase DRH. In addition, RDE-4, DRH-1 and DRH-2 proteins were detected as interactors when pulling down with DCR-1.

In addition to the proteins involved in initiation of RNAi, other proteins having characterized functions that relate to small RNAs, were detected. Two argonaute proteins, ALG-1 and ALG-2, were also detected in the adult DCR-1 purifications. These paralog proteins are required for the efficient processing of a variety of miRNA precursors, but were heretofore unknown to interact physically with DCR-1.

Interactions with the rdrp RRF-3, and the SAP domain ERI-1 nuclease were also detected in the embryonic purifications. Interestingly when the genes coding for these proteins were inactivated, an enhancement of the classical RNAi response is observed (eri phenotype) indicating that rrf-3 and eri-1 encode negative regulators of RNAi.

In addition, the protein D2005.5 was detected which did not have a characterized small RNA-related function, but is a paralog of the dicer-related helicases drh-1 and drh-2.

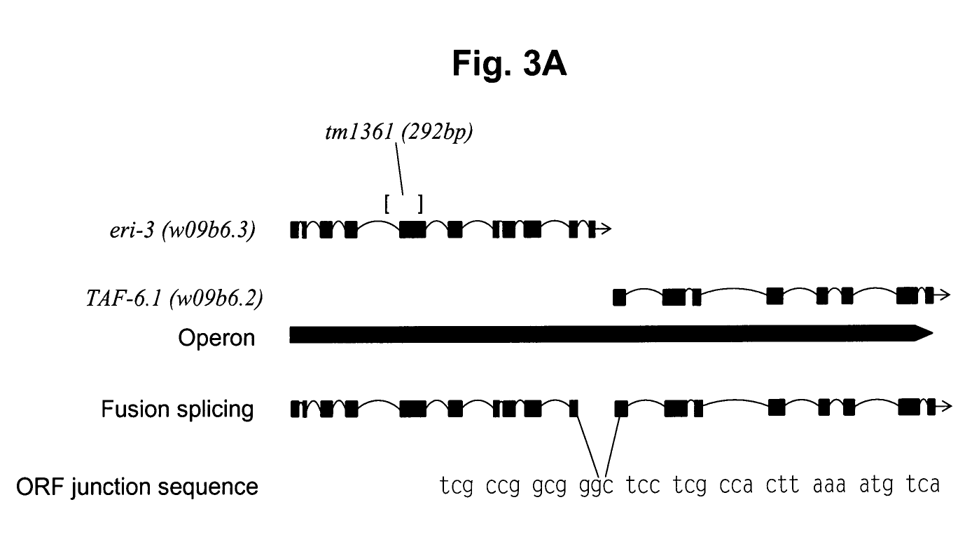

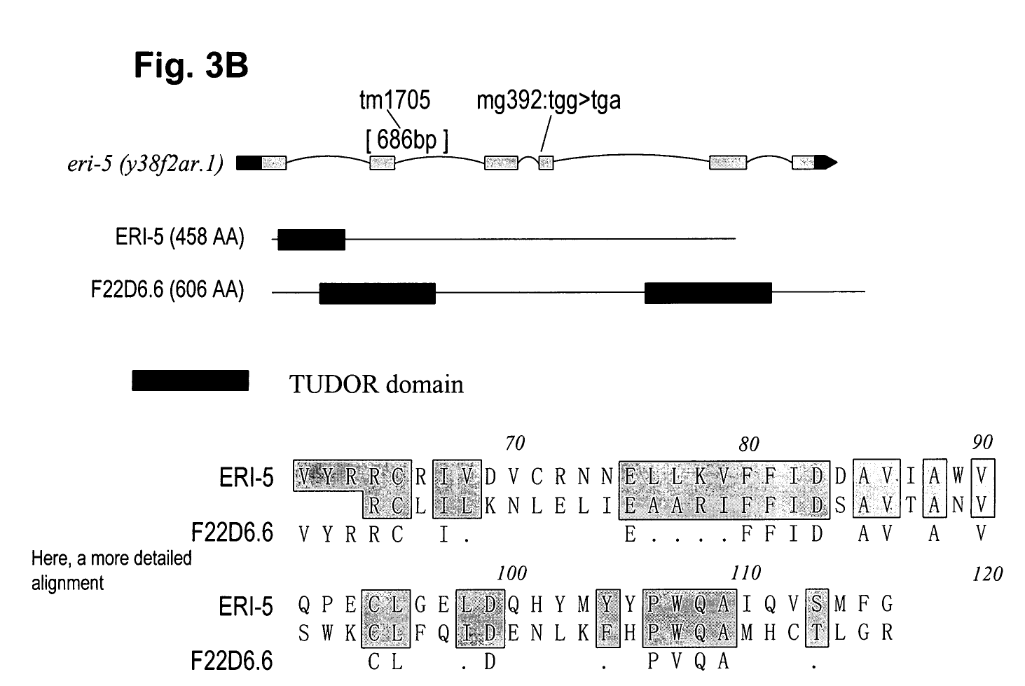

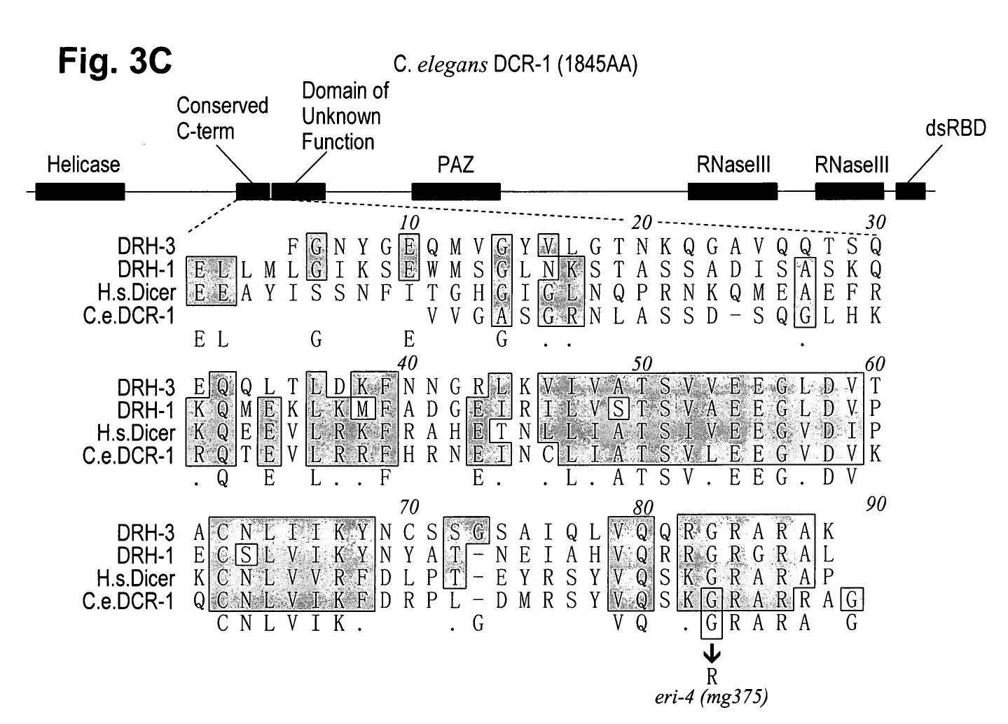

For eight other proteins, no previous link with DCR-1 functions, or with small RNA-mediated silencing was known. Four detected proteins have known functions: snRNP core protein D1 (SNR-3), the translation elongation factor 2 (EFT-2), the NHL family ring finger-B box-Coiled coil translational repressor LIN-41, and subunit TAF6 of the transcription initiation factor TFIID (TAF-6.1). Finally four others have unknown functions. This subgroup includes T23G7.1 (an ortholog of the mammalian PIR1), the novel proteins C32A3.2, W09B6.3 (expressed as an operon with TAF-6.1), and the TUDOR domain protein Y38F2AR.1 (FIG. 3)

### EXAMPLE 3

### Methods for Determining In Vivo Activity of Dicer Interacting Proteins

To address the possible functions of these proteins in DCR-1-related mechanisms, the phenotypes of the rnai knock down for their corresponding genes (Table 3) was examined. The genes snr-3 and eft-2 (rnai) demonstrate pleiotropic phenotypes and growth defects. For the remaining Dicer interacting proteins, deletion alleles for d2005.5 (tm1217), t23g7.5 (tm1496), c32a3.2(tm1314), w09b6.3(tm1361) and y38j2ar.1(tm1705) were generated.

In addition, because two interacting protein were encoded by eri genes, the location of the genes encoding the interacting proteins with the mapping intervals of alleles generated in a screen for mutants that increase sensitivity of a neuronal gfp reporter to gfp rnai, were generated. Using this strategy three genes, TAF-6.1, w09b6.3 (part of a common operon), and y38f2ar.1 were mapped within the intervals of the eri-3 and eri-5 mutations, respectively. These three gene sequences were found to comprise nonsense point mutations. In addition, another enhancer (eri-4 (mg375)) mapped in proximity to DCR-1, and a point mutation (glycine 351 to arginine) was discovered in the C-terminus extremity of the conserved C-terminus sub-domain of its helicase domain.

### EXAMPLE 3

### Methods for Determining the In Vivo RNAi Activity of Dicer Interacting Proteins

The potency of the RNAi activity in whole animals, either for enhancement or deficiency (Table 2), was examined. First, their response in a high sensitivity unc-22 (rnai) somatic (Po) assay, was determined. This assay revealed that the interactor T23G7.5 allele exhibited a drastically reduced sensitivity to rnai when assayed in the soma, both for endogenous unc-22 (rnai) silencing and for gfp (rnai) silencing of a transgenic reporter. Possibly due to the maternal load the effect on RNAi was important, but partial. The mutant on itself also presents developmental defects: the homozygous null grows normally and suddenly arrests at the L4 stage, never reaching adulthood. A generalized loss of gene expression could not be responsible for the lack of RNAi response, as the arrested animals could still transcribe and translate a reporter de novo to a WT level. This protein encodes a conserved RNA phosphatase with homology to a family of capping enzymes, and associates with RNP particles in mammalian culture cells. Its enzymatic activity was shown to have specificity toward the removal of the β- and γ-phosphate residues on the 5′ end of triphosphate RNA substrates. This interaction was consistently detected both in the adult and embryonic purifications of DCR-1, and indicating its role in RNAi mechanisms. Thus, T23G7.5 was determined to be essential for development and RNAi

### EXAMPLE 4

### Methods for Determining the In Vivo RNAi Enhancer Activity of Dicer Interacting Proteins

The Dicer interacting proteins, w09b6.3 and y38f2ar.1 were determined enhancers of RNAi. Briefly, mutants using rnai targets, which do not exhibit a phenotype, or exhibit a very low penetrance in the WT (N2) genetic background, were assayed to test the possibility that these genes encode enhancers of rnai (eri). As observed, unc-73 (rnai) does not usually exhibit a strong penetrance when wt (N2) animals are exposed (4+−4%). As previously observed, eri-1(mg366) and rrf-3(pk1426) gave a very penetrant effect when exposed to unc-73 E. coli feeding strain (98+−2%). Similarly, a drastically higher penetrance was observed in the w09b6.3 (tm1361), y38f2ar.1 (mg392), and DCR-1(mg375) alleles (100%, 82.5+−11%, and 100%, respectively). Injection assays for lin-1 (rnai), and feeding assays for dpy-13 (rnai), hmr-1(mai), or gfp(mai) also supported these observations (not shown). Thus, it was concluded that the w09b6.3, and y38f2ar.1 mutations are enhancers of rnai (eri).

### EXAMPLE 5

### Methods for Determining the In Vivo Developmental Effects of Dicer Interacting Proteins

The Dicer interacting proteins eri-3, eri-4 and eri-5 were determined to have similar developmental defects. In addition to the similar effect on rnai, the rrf-3 and eri-1 genes were previously shown to have indistinguishable developmental defects and to act in the same genetic pathway. Known developmental defects include a strong sterility phenotype at 25° C., which is rescued at 15° C., or by crossing with WT males, suggestive of sperm defects. Mutant animals also exhibit spontaneous silencing of some simple transgenic arrays in the soma and a low incidence of X chromosome non-disjunction, visible by a High Incidence of Males (HIM) phenotype.

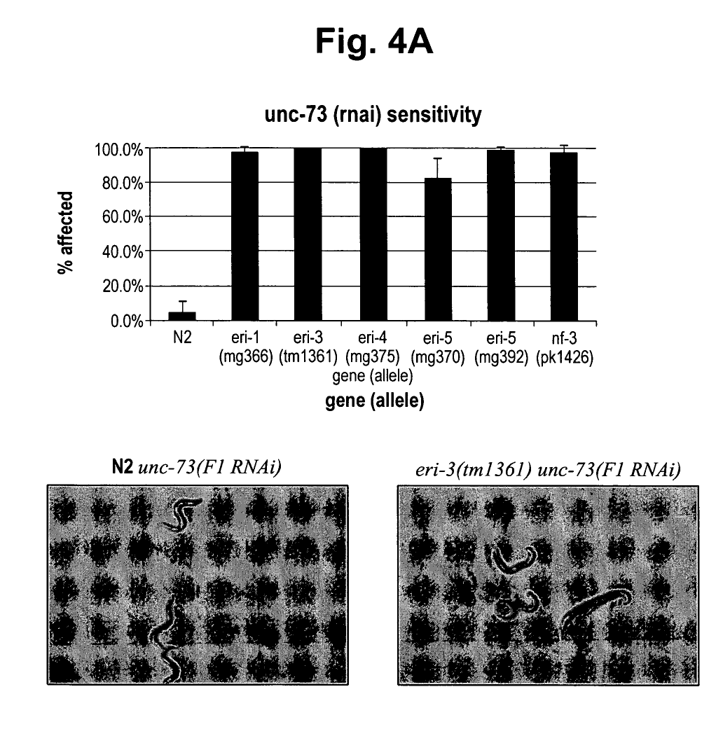

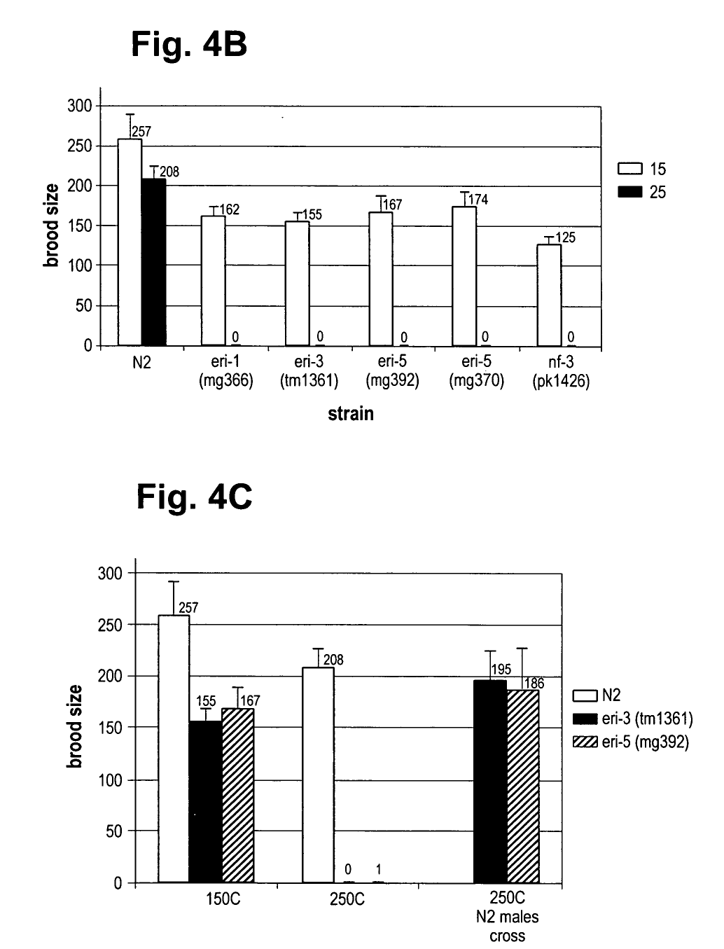

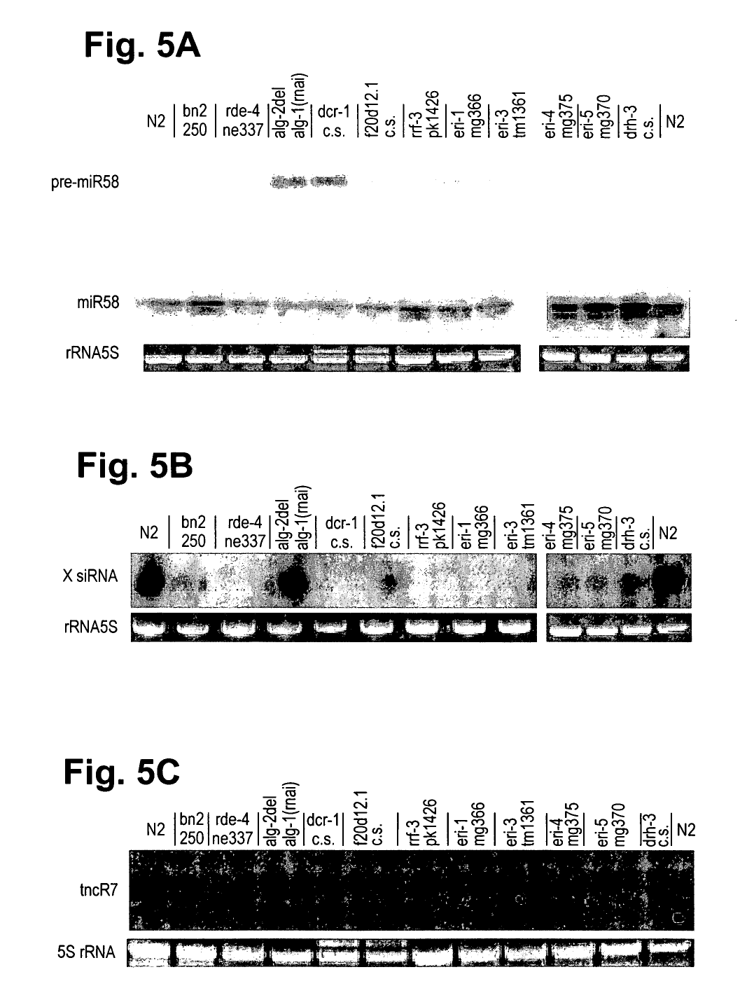

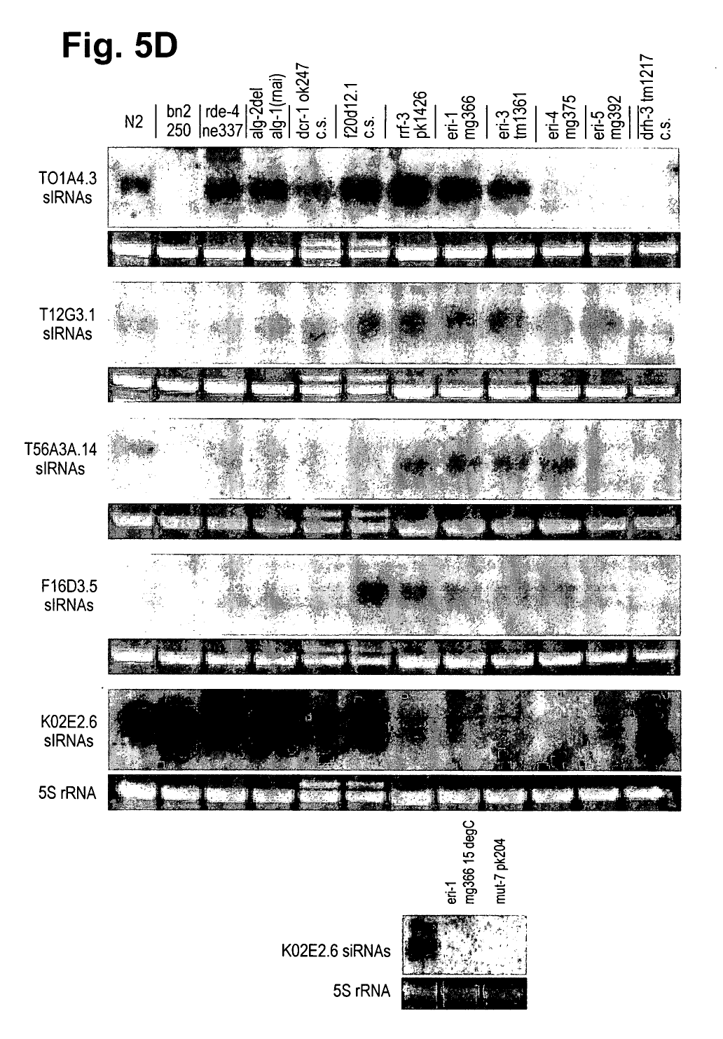

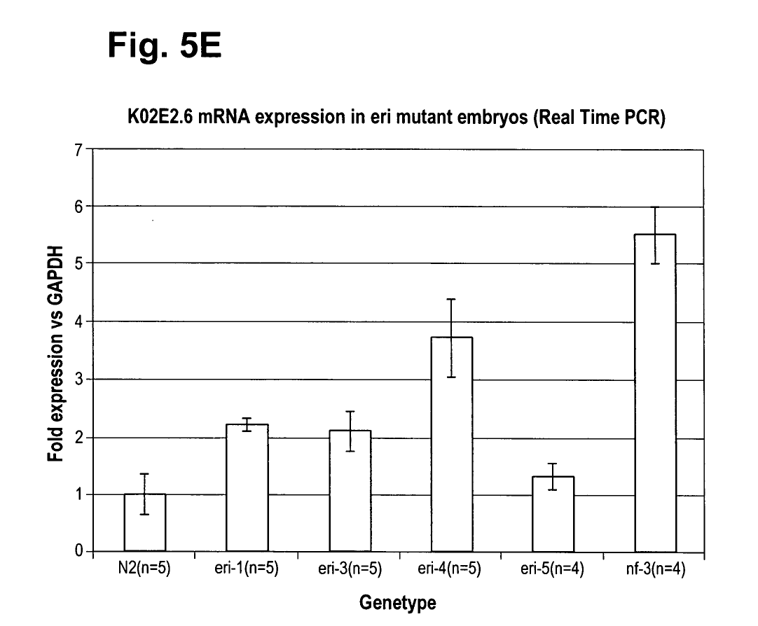

To test the idea that eri-3, eri-4 and eri-5 were acting on the same developmental mechanism, the defects associated with their corresponding alleles, were examined. Akin to alleles of rrf-3 and eri-1, mutations in these two genes led to mean brood sizes of 0±1 for w09b6.3 and of 1±1 for y38f2ar.1 −/− animals at 25° C. In contrast, at 15° C. the same alleles gave mean brood sizes of 155±12 for w09b6.3 and 167±20 for y38f2ar.1. Interest what was observed for rrf-3 and eri-1 alleles, the temperature sensitive sterility phenotype of w09b6.3 and y38f2ar.1 can be rescued by crossing with wt males, and therefore believed to be defective in sperm function. Additionally, a 3 to 5 fold increase in incidence of males was also observed in the corresponding mutants, compared to the WT(N2) spontaneous incidence (˜0.1%). Altogether, the unique, and specific combination of defects observed in the eri-3, eri-4 and eri-5 mutants indicates their involvement in a common pathway with eri-1 and rrf-3 (FIG. 4)

### EXAMPLE 6

### Methods for Determining the In Vivo Helicase/Chromosomal Effects of Dicer Interacting Proteins

The Dicer interacting protein DRH-3, when depleted, was determined to cause sterility and chromosome segregation defects. Mutations in the gene encoding the DCR-1-interacting protein D2005.5 also led to dramatic fertility defects. Because it encodes a paralog of the DEAX/D box helicase drh-1 and drh-2, this Dicer interacting protein was renamed drh-3. In contrast, despite the close homology, drh-3 is not required for initiation of the classical RNAi pathway, at least in the soma where it could be examined (see Table 3). Instead, while the drh-3(tm1217) allele animals were sterile as homozygous and examination of a pie-1::his-3::gfp transgenic strain revealed abnormally shaped oocytes with proximal mitosis, and occasional occurrence of multinucleation, rnai depletion led to a slow onset, but penetrant early embryonic arrest (Table 3). Although the terminal phenotype of this arrest was variable, the observed embryonic arrest was progressively earlier in embryos laid in periods extending two or three days after the adult injection. Interestingly and consistently with the phenotype exhibited in the deletion mutant, earlier injection of Po animals (L3 or L4 animals) also led to sterility. The earliest defects in the affected embryos using time-lapse videomicroscopy were also characterized. As observed, the first cell division was abnormal, and chromosomes lagging on the mitotic spindle could be observed at metaphase. Chromosome segregation later resulted in abnormally shaped nuclei.

Thus, it was noted that this initial developmental defect resembled the observed defects described in S. pombe for mutants in the RNAi machinery.

### EXAMPLE 7

### Methods for Determining the In Vivo Effects of Dicer Interacting Proteins on the Accumulation of Endogenous Small RNAs

The Dicer interacting protein drh-3 and the eri are required for the accumulation of classes of endogenous small RNAs.

Because divergent phenotypes were observed in many genes encoding the different DCR-1 interacting proteins, different phenotypic groups would be reflected by defects in accumulation and/or processing of different classes of small RNA. To test this hypothesis, the status of 5 classes of small RNAs known to require dcr-1 for efficient production, in the mutants generated, was examined. Sensitivity to exogenous dsRNA-triggering was used as a functional output for involvement in the classical RNAi pathway. Also examined, was the processing of miRNA precursors, the accumulation of the tncR, and small RNA-derived from an X chromosome-derived contig. Finally, accumulation of endogenous siRNAs (endo siRNAs) for a variety of loci previously shown to naturally produce these small RNAs, was examined

Because dcr-1 −/−, and drh-3−/− are sterile, a counter-selectable balancer strategy to select for nulls within large populations of animals, was employed. The maternal load of the two gene products was sufficient to lead the animals through early development and sterile adults could be studied. To look for an alg-1/alg-2 depletion effect on small RNAs, animals depleted of alg-1 by rnai in an alg-2−/− (ok304) animal background, were used.

A variety of miRNA were examined and no defects in the mature form accumulation nor in precursor processing was observed in the rde-4, the eri, nor in the drh-3 mutants. In contrast, a moderate to strong miRNA precursor accumulation was visible in alg-1/2, and dcr-1 depleted animals. These results indicate that these two proteins are crucial to most, if not all the miRNA maturation. However, the effect observed here on the precursor accumulation depends on the timing of the miRNA transcription and export relative to the depletion of ALG-1 protein by RNAi or the turnover of the maternal load of DCR-1 in the counterseleted F1 nulls.

Examination of the small RNA populations in the drh-3 counter-selectable nulls revealed that, while this protein is dispensable for normal accumulation of miRNAs, X-derived small RNAs, or the examined tncRs, it is required for the accumulation of all the examined ORF-derived endo-siRNA. Acting as controls, alg-depleted animals, and another counterselectable sterile mutant j20d12.1 did not show such defects in accumulation of these small RNAs. While most of the endo-siRNAs detected were only detected in the germline, drh-3 was also required for the production of soma-derived endo-siRNA k02e2.6. This result implicates the DCR-1-interacting protein DRH-3 specifically in the production and/or stabilization of a broad range of the ORF-derived endogenous-siRNAs.

The five eri mutants exhibited very consistent molecular defects in the accumulation of the mature small RNAs. While they enhanced the classical RNAi response (when triggered from exogenous sources of dsRNA), their mutation prevented accumulation of the examined tncR, and of the X locus-derived small RNAs. Interestingly, rde-4 was also required for the accumulation of the X locus-derived small RNAs, but dispensable for the tncRs or the ORF-derived endo-siRNAs, showing the modulatory nature of the contribution of the DCR-1 interactors for production of diverse small RNA classes.

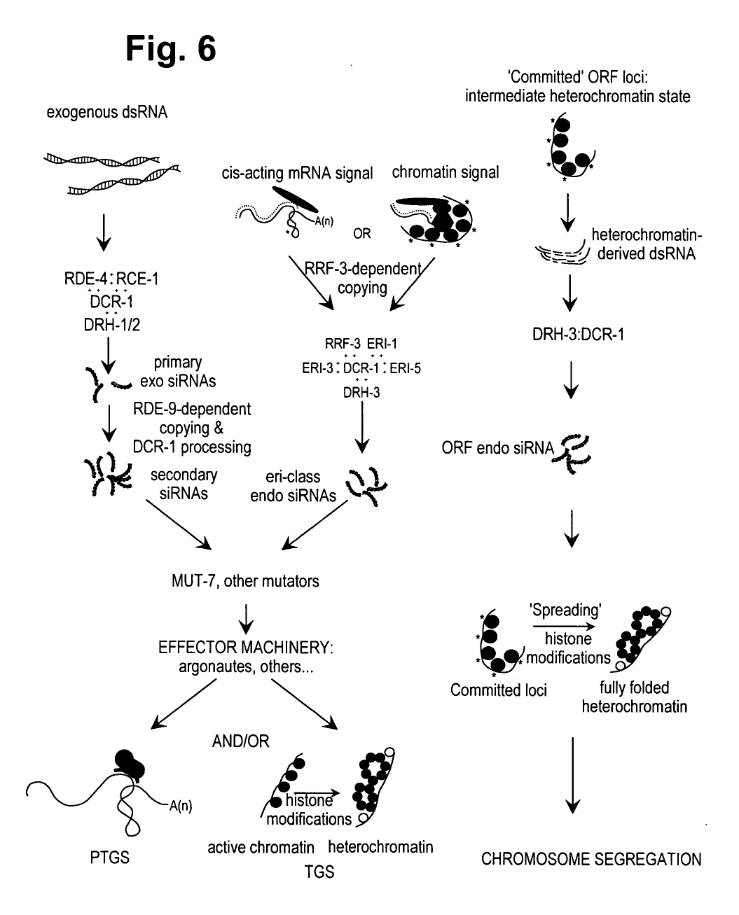

The eri mutations did not affect the accumulation of most of the endo-siRNAs. However, surprisingly, the eri mutants also failed to accumulate endo-siRNAs from a very restricted number of genes. Interestingly, the eri genes also exhibited this defect at the permissive temperature in gravid adults, showing that the developmental process involving the eri genes, and not their function in endo-siRNAs accumulation is a temperature-sensitive process. This observation, and the presence of these endo-siRNAs in germline-less animals (FIG. 6, bn2(glp-4)) rules out the idea that the eri genes fail to show these endo-siRNAs because they lack the tissue where they are produced.

These results support the idea that different combinations of DCR-1-interacting proteins are required for efficient accumulation of distinct classes of endogenous small RNAs. These results support a function for the DCR-1-interacting ERI proteins in the initiation of a variety of distinct endogenous small RNA-mediated silencing mechanisms (FIG. 6).

Equivalents

Those skilled in the art will recognize, or be able to ascertain using no more than routine experimentation, many equivalents to the specific embodiments of the invention described herein.

What is claimed:

1. A method of identifying a Dicer interacting protein comprising,
contacting a composition having a candidate Dicer interacting protein or bioactive fragment thereof with a Dicer protein or a bioactive fragment thereof, and
determining the presence of one or more proteins capable of interacting with Dicer, as compared to an appropriate control, such that a Dicer interacting protein, is identified.

2. A method of identifying a modulator of Dicer activity comprising,
contacting a composition comprising Dicer or a bioactive fragment thereof and a Dicer interacting protein or a bioactive fragment thereof with a test compound, and
determining the ability of the test compound to modulate activity between Dicer or bioactive fragment thereof and the Dicer interacting protein or bioactive fragment thereof, such that a modulator of Dicer activity, is identified.

3. The method of claims 1 or 2, wherein Dicer or bioactive fragment thereof is derived from an organism selected from the group consisting of nematode, fruit fly, mouse, rat, and human.

4. The method of claims 1 or 2, wherein the Dicer interacting protein or bioactive fragment thereof is derived from an organism selected from the group consisting of nematode, fruit fly, mouse, rat, primate, and human.

5. The method of claim 2, wherein the Dicer activity is selected from the group consisting of protein:protein binding activity, miRNA maturation activity, RNAi initiation activity, RNAi enhancer activity, helicase activity, RISC activity, target recognition activity, and target gene cleavage activity.

6. The method of claims 1 or 2, wherein the composition comprises an extract selected from the group consisting of a cellular extract, nuclear extract, cytoplasmic extract, protein extract, S100 fraction, partially purified protein extract, and purified protein extract.

7. The method of claims 1 or 2, wherein the Dicer interacting protein is selected from the group consisting of RDE-4, ALG-1, ALG-2, DRH-1, DRH-2, helicase homologous to DCR-2 (DRH-3), double helicase, EFT-2 EF-Tu family GTP binding, EFT-4 (eIF1 alpha), GAP/RAN-GAP family, HMG-I/Y DNA binding, HMG-I/Y DNA, binding PB1 domain, SNR-2 SM protein, SNR-3 SM protein, Dual specificity phosphatase, LIN-41, low homology MADS box, novel, RPN-9 proteasome subunits, TAF 6.1, T54 homology, RRM protein (3 domains), Worm unique/Novel (ce27223), TBB-4, RPS-14, RPS-13, RPL-24, RPS-11, Agglutinin, SIP-1 (hsp20), CCT-6 (chaperonin), RDE-1, DRH-3, ERI-1, RRF-3, ERI-3, ERI-5, PIR-1, C32A3.2, and orthologs, paralogs, or bioactive fragments thereof

8. The method of claims 1 or 2, wherein the Dicer interacting proteins are further subjected to a multidimensional protein interaction technology (MudPIT).

9. A method of identifying a modulator of Dicer activity, comprising contacting a cell or cell extract having Dicer or a bioactive fragment and a Dicer interacting protein or bioactive fragment thereof, with a test compound and determining the ability of the test compound to modulate an activity selected from the group consisting of protein:protein binding activity, miRNA maturation activity, RNAi initiation activity, RNAi enhancer activity, helicase activity, RISC activity, target recognition activity, and target gene cleavage activity.

10. The method of claim 9, wherein said cell or cell extract comprises recombinantly expressed Dicer.

11. The method of claim 9, wherein said cell or cell extract comprises a Dicer interacting protein selected from the group consisting of RDE-4, ALG-1, ALG-2, DRH-1, DRH-2, helicase homologous to DCR-2 (DRH-3), double helicase, EFT-2 EF-Tu family GTP binding, EFT-4 (eIF1 alpha), GAP/RAN-GAP family, HMG-I/Y DNA binding, HMG-I/Y DNA, binding PB1 domain, SNR-2 SM protein, SNR-3 SM protein, Dual specificity phosphatase, LIN-41, low homology MADS box, novel, RPN-9 proteasome subunits, TAF 6.1, T54 homology, RRM protein (3 domains), Worm unique/Novel (ce27223), TBB-4, RPS-14, RPS-13, RPL-24, RPS-11, Agglutinin, SIP-1 (hsp20), CCT-6 (chaperonin), RDE-1, DRH-3, ERI-1, RRF-3, ERI-3, ERI-5, PIR-1, and C32A3.2.

12. The method of claim 9, wherein the ability of the test compound to modulate enhancers of RNAi is determined.

13. The method of claim 9, wherein the Dicer or bioactive fragment thereof complexed with a Dicer interacting protein is isolated by immunoaffinity chromatography.

14. The method of claim 13, wherein the Dicer interacting protein or bioactive fragment thereof is subjected to MudPIT.

15. The method of claim 9, wherein a detectable label is associated with a component selected from the group consisting of Dicer, a Dicer interacting protein, and test compound.

16. A modulator identified by any one of the preceding claims.

17. A method of identifying a modulator of Dicer comprising,
contacting a S100 fraction comprising Dicer or a bioactive fragment thereof and a Dicer interacting protein with a test compound, and
determining the activity of Dicer or bioactive fragment in the presence of the test compound as compared to an appropriate control, wherein the test compound is a potential modulator of Dicer based on its ability to affect the activity of Dicer or a bioactive fragment thereof.

18. An antibody that specifically binds to a component selected from the group consisting of Dicer, Dicer interacting protein, and Dicer:Dicer interacting protein complex.

19. The antibody of claim 18, wherein the Dicer interacting protein is selected from the group consisting of RDE-4, ALG-1, ALG-2, DRH-1, DRH-2, helicase homologous to DCR-2 (DRH-3), double helicases, EFT-2 EF-Tu family GTP binding, EFT-4 (eIF1 alpha), GAP/RAN-GAP family, HMG-I/Y DNA binding, HMG-I/Y DNA, binding PB1 domain, SNR-2 SM protein, SNR-3 SM protein, Dual specificity phosphatase, LIN-41, low homology MADS box, novel, RPN-9 proteasome subunits, TAF 6.1, T54 homology, RRM protein (3 domains), Worm unique/Novel (ce27223), TBB-4, RPS-14, RPS-13, RPL-24, RPS-11, Agglutinin, SIP-1 (hsp20), CCT-6 (chaperonin), RDE-1, DRH-3, ERI-1, RRF-3, ERI-3, ERI-5, PIR-1, and C32A3.2.

20. A pharmaceutical composition comprising the modulator of claim 17.

21. A method of activating target-specific RNA interference (RNAi) in an organism comprising,
administering to the organism an RNAi agent and a modulator of Dicer activity, wherein the modulator is administered in an amount sufficient for enhancing the activity of the RNAi agent, thereby achieving degradation of a target mRNA in the organism.

22. The method of claim 21, wherein the target mRNA encodes a gene product involved or predicted to be involved in a human disease or disorder.

23. A method of treating a disease or disorder associated with the activity of a gene product encoded by a target mRNA in a subject comprising,
administering to the subject an RNAi agent, and a modulator of Dicer activity, wherein said modulator is administered in an amount sufficient for enhancing the activity of the RNAi agent, thereby treating the disease or disorder associated with a gene product encoded by the target mRNA.

24. A method for deriving information about the function of a gene in a cell or organism comprising,
introducing into the cell or organism a Dicer interacting protein or an RNAi agent specific therefore, and
maintaining the cell or organism under target-specific RNAi conditions,
determining a characteristic or property of the cell or organism, and
comparing the characteristic or property to a suitable control, the comparison yielding information about the function of the gene.

25. A method of deriving information about the function of a Dicer interacting protein in a extract, cell, or organism comprising,
exposing an extract, cell, or organism capable of expressing Dicer and a Dicer interacting protein to an RNAi agent, maintaining the lysate, cell, or organism under conditions such that target-specific RNAi can occur,
determining a characteristic or property of the extract, cell, or organism, and comparing the characteristic or property to a suitable control, the comparison yielding information about the function of the gene.

26. A method of validating a candidate Dicer interacting protein as a suitable target for drug discovery comprising,
introducing into a cell or organism a Dicer interacting protein or an RNAi agent specific therefore, and
maintaining the cell or organism under target-specific RNAi conditions,
determining a characteristic or property of the cell or organism, and
comparing the characteristic or property to a suitable control, the comparison yielding information about whether the candidate protein is a suitable target for drug discovery.

27. The method of claims 24, 25, or 26, wherein the organism is selected from the group consisting of nematode, fruit fly, mouse, rat, primate, and human.

28. A method of treating a disease or disorder associated with the activity of a gene product encoded by a target RNA in a subject comprising,
administering to the subject an agent sufficient to modulate Dicer activity in one or more cells and
administering an RNAi agent in an amount sufficient for degradation of the target RNA to occur, thereby treating the disease or disorder associated with the gene product encoded by the target gene.

29. The method of claim 28, wherein the subject is a human patient.

30. A kit comprising a reagent for activating target-specific RNA interference (RNAi) in a cell or organism, the kit comprising:
a component selected from the group consisting of a molecule encoding a Dicer interacting protein, an RNAi agent specific for a Dicer interacting protein, a modulator of Dicer activity, and a modulator of a Dicer interacting protein, and
instructions for use.


SEQUENCE LISTING (112 sequences)

SEQ ID NO: 1 - DNA, 27 bp, Artificial Sequence
  Description of Artificial Sequence Synthetic primer
atggatttaaccaaactaacgtttgaa

SEQ ID NO: 2 - DNA, 23 bp, Artificial Sequence
  Description of Artificial Sequence Synthetic primer
tcaatccgtgaaatcataggtgt

SEQ ID NO: 3 - protein, 34 aa, Caenorhabditis elegans
PGTTKEEALSNIDQISDKAEELKRSTSDAVQDND

SEQ ID NO: 4 - protein, 385 aa, Caenorhabditis elegans
MDLTKLTFESVFGGSDVPMKPSRSEDNKTPRNRTDLEMFLKKTPLMVLEEAAKAVYQKTPTWGTVELPEGFEMTLILNEITVKGQATSKKAARQKAAVEYLRKVVEKGKHEIFFIPGTTKEEALSNIDQISDKAEELKRSTSDAVQDNDNDDSIPTSAEFPPGISPTENWVGKLQEKSQKSKLQAPIYEDSKNERTERFLVICTMCNQKTRGIRSKKKDAKNLAAWLMWKALEDGIESLESYDMVDVIENLEEAEHLLEIQDQASKIKDKHSALIDILSDKKRFSDYSMDFNVLSVSTMGIHQVLLEISFRRLVSPDPDDLEMGAEHTQTEEIMKATAEKEKLRKKNMPDSGPLVFAGHGSSAEEAKQCACKSAIIHFNTYDFTD

SEQ ID NO: 5 - protein, 9 aa, Caenorhabditis elegans
RIQLKYPHL

SEQ ID NO: 6 - protein, 1002 aa, Caenorhabditis elegans
MSGGPQYLPGVMNSTIQQQPQSATSSFLPSGPISSTSTSSQVVPTSGATQQPPFPSAQAAASTALQNDLEEIFNSPPTQPQTFSDVPQRQAGSLAPGVPIGNTSVSIGEPANTLGGGLPGGAPGQLPGGNQSGIQFQCPRRPNHGVEGRSILLRANHFAVRIPGGTIQHYQVDVTPDKCPRRVNREIISCLISAFSKYFTNIRPVYDGKRNMYTREPLPIGRERMDFDVTLPGDSAVERQFSVSLKWVGQVSLSTLEDAMEGRVRQVPFEAVQAMDVILRHLPSLKYTPVGRSFFSPPVPNASGVMAGSCPPQASGAVAGGAHSAGQYHAESKLGGGREVWFGFHQSVRPSQWKMMLNIDVSATAFYRSMPVIEFIAEVLELPVQALAERRALSDAQRVKFTKEIRGLKIEITHCGQMRRKYRVCNVTRRPAQTQTFPLQLETGQTIECTVAKYFYDKYRIQLKYPHLPCLQVGQEQKHTYLPPEVCNIVPGQRCIKKLTDVQTSTMIKATARSAPEREREISNLVRKAEFSADPFAHEFGITINPAMTEVKGRVLSAPKLLYGGRTRATALPNQGVWDMRGKQFHTGIDVRVWAIACFAQQQHVKENDLRMFTNQLQRISNDAGMPIVGNPCFCKYAVGVEQVEPMFKYLKQNYSGIQLVVVVLPGKTPVYAEVKRVGDTVLGIATQCVQAKNAIRTTPQTLSNLCLKMNVKLGGVNSILLPNVRPRIFNEPVIFFGCDITHPPAGDSRKPSIAAVVGSMDAHPSRYAATVRVQQHRQEIISDLTYMVRELLVQFYRNTRFKPARIVVYRDGVSEGQFFNVLQYELRAIREACMMLERGYQPGITFIAVQKRHHTRLFAVDKKDQVGKAYNIPPGTTVDVGITHPTEFDFYLCSHAGIQGTSRPSHYHVLWDDNNLTADELQQLTYQMCHTYVRCTRSVSIPAPAYYAHLVAFRARYHLVDREHDSGEGSQPSGTSEDTTLSNMARAVQVHPDANNVMYFA

SEQ ID NO: 7 - DNA, 20 bp, Caenorhabditis elegans
tctgagtttggctcgatgtg

SEQ ID NO: 8 - DNA, 20 bp, Caenorhabditis elegans
atgttccttggataccagcg

SEQ ID NO: 9 - DNA, 20 bp, Caenorhabditis elegans
agcccagaactgggaaactt

SEQ ID NO: 10 - DNA, 20 bp, Caenorhabditis elegans
aagtcgaattccgttggatg

SEQ ID NO: 11 - DNA, 20 bp, Artificial Sequence
  Description of Artificial Sequence Synthetic primer
tctaattttccaattttcag

SEQ ID NO: 12 - DNA, 20 bp, Artificial Sequence
  Description of Artificial Sequence Synthetic primer
gatattgttccaggacagcg

SEQ ID NO: 13 - protein, 910 aa, Caenorhabditis elegans
MFPLPVHNGPRLGKLSIFEMPGDSLTSSSFMPDGGAETSSSSQLGGSAHGAIGTKPDAGVQFQCPVRPNHGVEGRSILLRANHFAVRIPGGSVQHYQIDVFPDKCPRRVNREVIGCLISSFSKYFTNIRPVYDGKRNMYTREPLPIGTEPMNFEVTLPGDSAVERKFSVTMKWIGQVCLSALDDAMEGRVRQVPHEAVQSIDVILRHLPSLKYTPVGRSFFTPPGVMKPGMQMHQESKLGGGREVWFGFHQSVRPSQWKMMLNIDVSATAFYRAMPVIEFVAEVLELPVQALAERRALSDAQRVKFTKEIRGLKIEITHCGAVRRKYRVCNVTRRPAQTQTFPLQLETGQTIECTVAKYFFDKYRIQLKYPHLPCLQVGQEQKHTYLPPEVCDIVPGQRCLKKLTDVQTSTMIKATARSAPEREREICKLVSKAELSADPFAHEFGITINPAMTEVKGRVLSAPKLLYGGRHRATTALPNQGVWDMRGKQFHTGMEVRTWAIACFAQQSHVKENDLRMFTTQLQRISTDAGMPIIGTPMFCKYASGVEQVEPMFKYLKQTYSAIQLIVVVLPGKTPIYAEVKRVGDTVLGIATQCVQAKNAIRTTPQTLSNLCLKMNVKLGGVNSILLPNVRPRIFNEPVIFLGCDITHPAAGDTRKPSIAAVVGSMDAHPSRYAATVRVQQHRQEIITDLTYMVRELLVQFYRNTRFKPARIVVYRDGVSEGQLFNVLQYELRAIREACVMLESGYQPGITFIAVQKRHHTRLFAADKADQVGKAFNIPPGTTVDVGITHPTEFDFFLCSHAGIQGTSRPSHYHVLWDDNDLTADELQQLTYQMCHTYVRCTRSVSIPAPAYYAHLVAFRARYHLVDRDHGSGEEGSQPSGTSSEDTTLSSMAKAVQVHPDSNNVMYFA

SEQ ID NO: 14 - protein, 4 aa, Artificial Sequence
  Description of Artificial Sequence Synthetic peptide motif
DEAD

SEQ ID NO: 15 - protein, 4 aa, Artificial Sequence
  Description of Artificial Sequence Synthetic peptide motif
DEAH

SEQ ID NO: 16 - DNA, 27 bp, Artificial Sequence
  Description of Artificial Sequence Synthetic primer
atgaggaaaaagcagtgttcttcaata

SEQ ID NO: 17 - DNA, 27 bp, Artificial Sequence
  Description of Artificial Sequence Synthetic primer
ttatgcttctctgattaaattgactac

SEQ ID NO: 18 - protein, 1037 aa, Caenorhabditis elegans
MRKKQCSSILSLYDKEIILCLEPIYRDPEKGDGFSELLPLGRIDELKIQSENAQEFSKQLYHDLKNSILSNADDERLYKDIMTYLQTYLPKCTVHKLLNCSNREVKLSDFHYILDHFEGFLRFIEPKVVLAYLDSYPQYIDAVAVLRKEIERNEEDNQDSDFIKKLILRTVPLLGEQAVYDIMYTISEKSSNNLDVEAKQFIAKVLRLKNDGFLRFYQIINASRRQLNGRIYICPVHESATEMMVYLGTAALNTNRYRMINIRVDNIVQENSTPRLVIESVRQRIHRQRQLCLRNYQEELCQVALQGKNTIVTAPTGSGKTVIAANIIKEHFESRSSEGKRFKALFMTPNSMILNQQAASISSYLDHVYHTQIIQGSDNVPTRNVIQSKDLIVATPQMIVNLCNEHRNSLDDESRLDQFFLSTFTIIFFDECHNTVKNSPYSNIMREYHYLKNMGNMPEGHSLPQIIGLTASLGTGDKNDCLQVRNYIAGLCASMDVKDLSIVKDNLEELRGYSPIVPDKVLLCERSTDGPIGMFTNRLTLMMQEVEGLIRTALRNEHIGIEQRRQIETTERDFRPDSSFLDPPADKEHAGYQNWVCNQMNLVSGTSFRETGTRTIINEALDVLKECFCTLSYNINFHPEVALNYLKDEMEYRTPNFTVNMIRIWERYHNQLVGTGSAENPMISKTVQYIVEQNLQRADSRTIIFVRTRYEATILNKVLNSNEELLMLGIKSEWMSGLNKSTASSADISASKQKQMEKLKMFADGEIRILVSTSVAEEGLDVPECSLVIKYNYATNEIAHVQRRGRGRALNSECVLITNSIALRDQESNNRDKESLMSETISLIQNSPAEFRKCVDEESNKIWPRILREDTDKAQKIEEQINRNIVYKIICKKCEAILCTSKDIRSRNTQYLVCDPGFWSLVRKTRLTDEQQALIKYNATGSINCRRENCGLKLGQLIEVNTVDLPCLSALSIVLLVEGTDKRIIVKKWKNILDKYFTPTEIRQLDVQTMRDADQARTPMVFEHHANGEVVNLIREA

SEQ ID NO: 19 - DNA, 27 bp, Artificial Sequence
  Description of Artificial Sequence Synthetic primer
atgattgtaaatctttgcaatgagcac

SEQ ID NO: 20 - DNA, 29 bp, Artificial Sequence
  Description of Artificial Sequence Synthetic primer
ttatgcttgtctaattacattgattactt

SEQ ID NO: 21 - protein, 620 aa, Caenorhabditis elegans
MIVNLCNEHRDPLDDEYPPEQFFLSTFTIIFFDECHNTVKNSPYSNVMREYHYLKNMGNMPEGHSFPQIIGLTASLGTGDKKNCMQVRSYIAGLCANMDVKELSIVKDNLEELLDHNPFVTDQVSFCERSNDGPIEMFTKRLKQMMQEVEDLIRTTLKNEPTVKYEIPPTDKEHNRYENWISNQRNCVSLAGSRNKTLIIEVLDVLKDCFYALSYNINFNPEVALKKYLEKELGPERIRNFTDNMNLIWDNCHRELVGIGSAENPMIARTVQFILDQNEQTSDFRAIIFVRTKKEADFLNYVLNDRLHELGIKSDWMSGQKKSTASSADISASKQKQMEKLKMFADGENQILVSTSVAEEGLDIPECSLVIKYNYATNETAHVQRRGRARARNSKCVLITNSIALHVQESNNLAKENLMTETISLIQNSPGEFRQCVDEESNKVWPRIQREDTDKAQRIKEQINRNIVYKIVCMKCDTVLCTNKDIRSKNTQYIVCNPGFWSLVRRIPLPLEQRASNKFNSTGSIECLGERCGSKLGQLIDVNTVNLPCLKVKSILLLIESTNERILVKQWKNILDEHFTPTTLKQRDVQTMKDADYGRAPIEFEHHTANGEVINVIRQA

SEQ ID NO: 22 - protein, 1134 aa, Caenorhabditis elegans
MQPTAIRLEDYDKSKLRLPFESPYFPAYFRLLKWKFLDVCVESTRNNDIGYFKLFESLFPPGKLEEIARMIIDEPTPVSHDPDMIKIRNADLDVKIRKQAETYVTLRHAHQQKVQRRRFSECFLNTVLFDEKGLRIADEVMFNYDKELYGYSHWEDLPDGWLTAETFKNKFYDEEEVTNNPFGYQKLDRVAGAARGMIIMKHLKSNPRCVSETTILAFEVFNKGNHQLSTDLVEDLLTEGPAFELKIENGEEKKYAVKKWSLHKTLTMFLAIIGFKSNDKKEKNEHEEWYYGFIDAMKNDPANRAALYFLDKNWPEELEEREKERDRIRLTLLKSQRTNEEAVGEDVCTTIRPQPKDSGYNPDAVVTELVLRTYQEELVQPALEGKNCVIVAPTGSGKTEVAIYAALKHIEERTSQGKPSRVVLLVPKIPLVGQQKDRFLKYCNGMYEVNGFHGSESSVSGTGRRDEVIATHVSVMTPQILINMLQSVRQNERLYVSDFSMMIFDEVHKAAKNHPYVLINQMVQEWKYEKPQIIGLTASLSVKVDGQKDENQMLNDIYNMLALINAPHLSTITRQSSIDELNEHVGKPDDSVELCLPAKENILRDYIERYLNHAHGKFLEELASMSKSTGRNNTIPPNMINTFKKNQPKNYEYYDSLLQGIIQELNKLNVPEKWNSQTWAKYMKVYLEARGIVDLMPAMVAFKYMEKAIGKLNESHSETVEYSTFIKDHDTLKQTIQSVEPEIVLRLKKYTHQSVPHQFGNYGEQMVGYVLGTNKQGAVQQTSQEQQLTLDKFNNGRLKVIVATSVVEEGLDVTACNLIIKYNCSSGSAIQLVQQRGRARAKNSRSVLLSVKSSINETETNALISEKYMRLCVKKITENGEKQLAAEVKRVAELNAAERKRNLEEQLNLRLRHENKIYKLMCSNCSKEFCKSIYIKKVFSNYMVFDPSVWRFLHVESVETFIKCLKITWKCRIADYQIAEFPNFAFRQLTFRLFLCNFQMFQKRKVSKYLSEDNQPLSDIKCFHCKLDVGRAYKIRGTYLPQLSVKALTFVQESDYSSMTKAKWSDVEQDLFYISEAIEDDFRIMLNALSDTEENIEKKIVLDLDSRQHNKQLEMKRFHIQQEPPTKGVAPEAQ

SEQ ID NO: 23 - protein, 2145 aa, Caenorhabditis elegans
MADELARIQQYEYRQNSNLVLSVDYNLTDRRGREEPTGEVLPITDKEMRKMKMGDRAIKGKAPVQDQKKKRKKKDDEKAQQFGRNVLVDNNELMGAYKPRTQETKQTYEVILSFILDALGDVPREVLCGAADEVLLTLKNDKFRDKEKKKEVEALLGPLTDDRIAVLINLSKKISDFSIEEENKPEGDGDIYENEGVNVQFDSDEEEDDGGMVNEIKGDSEEESEEEEGVDTDYTATLKGDGHLTEDEQKARGILHPRDIDAHWIQRSLAKYFKDPLIAQQKQTEVIGILKNAADDRDAENQLVLLLGFDQFEFIKCLRQNRLMILYCTLLRQANEKERLQIEDDMRSRPELHPILALLQETDEGSVVQVEKSKRDAEKSKKAATAANEAISAGQWQAGRKMLDLNDLTFSQGSHLMSNKRCELPDGSYRRQKKSYEEIHVPALKPRPFAEGEKLVSVSELPKWAQPAFDGYKSLNRIQSRLCDSALRSKEHLLLCAPTGAGKTNVALLTMLQEIGNHLAEDGSVKLDEFKIVYIAPMKSLVQEMVGSFSKRLAPFGITVGEMTGDAQMSKEQFMATQVIVCTPEKYDVVTRKGGERAYNQMVRLLIIDEIHLLHDDRGPVLESIVVRTIRQMEQNHDECRLVGLSATLPNYQDVATFLRVKPEHLHFFDNSYRPVPLEQQYIGVTEKKALKRFQAMNEVVYDKIMEHAGKSQVLVFVHSRKETAKTAKAIRDACLEKDTLSAFMREGSASTEILRTEAEQAKNLDLKDLLPYGFAIHHAGMNRVDRTLVEDLFADRHIQVLFSTATLAWGVNLPAHTVIIKGTQIYNPEKGRWTELGALDIMQMLGRAGRPQYDDRGEGILITNHSELQYYLSLMNQQLPVESQMVSRLTDMLNAEVVLGTVSSVSEATNWLGYTFLFVRMLKNPTLYGITHEQARADPLLEQRRADLIHTACVLLDKAGLIKYDKRSGIIQATELGRIASHFYCTYESMQTYNKLLVETCSDIDLFRIFSMSSEFKLLSVRDEEKLELQKMAEHAPIPIKENLDEASAKTNVLLQAYISQLKLEGFALQADMVFVAQSAGRLFRALFEIVLWRGWAGLAQKVLTLCKMVTQRQWGSLNPLHQFKKIPSEVVRSIDKKNYSFDRLYDLDQHQLGDLIKMPKMGKPLFKFIRQFPKLEMTTLIQPITRTTMRIELTITPDFKWDEKVHGSAEGFWIFIEDTDGEKILHHEFFLLKQKFCSDEHVVKMIVPMFDPMPPLYYVRIVSDRWIGAETVLPISFRHLILPEKYPPPTELLDLQPLPISAVTNKEFQTVFAESGFKVFNPIQTQVFRTVFESNENVIVCAPNGSGKTAIAELAVLRHFENTPEAKAVYITPMEDMATKVYADWKRRLEPAIGHTIVLLTGEQTMDLKLAQRGQLIISTPERWDNISRRWKQRKSVQNVKLFIADDLHMIGASNGAVFEVVCSRTRYISSQLESAVRVVALSSSLTNARDLGMWLGCSASATFNFMPSTRPVPLDLEIKSFNLSHNASRFAAMERPVYQAICRHAGKLEPKPALVFVPVRRQTRPVAVALLTMALADGAPKRFLRLAEHDDTFQALLADIEDESLRESVSCGVGFLHEGTAPKDVHIVQQLFESNAIQVCVVPRGMCYQIEMSAYLVVVMDTQFYNGKYHVYEDYPIADMLHMVGLANRPILDSDAKCVVMCQTSKRAYYKKFLCDPLPVESHLDHCLHDHFNAEIVTKTIENKQDAIDYLTWTLLYRRMTQNPNYYNLQGTTHRHLSDALSELVELTLKDLENSKCIAVKDEMDTVSLNLGMIASYYYISYQTIELFSMSLKEKTKTRALIEIISASSEFGNVPMRHKEDVILRQLAERLPGQLKNQKFTDPHVKVNLLIHAHLSRVKLTAELNKDTELIVLRACRLVQACVDVLSSNGWLSPAIHAMELSQMLTQAMYSNEPYLKQLPHCSAALLERAKAKEVTSVFELLELENDDRSDILQMEGAELADVARFCNHYPSIEVATELENDVVTSNDNLMLAVSLERDNDIDGLAPPVVAPLFPQKRKEEGWWLVIGDSESNALLTIKRLVINEKSSVQLDFAAPRPGHHKFKLFFISDSYLGADQEFDVAFKVEEPGRSNRKRKHEKEED

SEQ ID NO: 24 - protein, 28 aa, Caenorhabditis elegans
VMNIKKDKTAALVEKLGIKLANDEKDLE

SEQ ID NO: 25 - protein, 4 aa, Caenorhabditis elegans
RKRK

SEQ ID NO: 26 - protein, 4 aa, Caenorhabditis elegans
YLDK

SEQ ID NO: 27 - protein, 852 aa, Caenorhabditis elegans
MVNFTVDEIRALMDRKRNIRNMSVIAHVDHGKSTLTDSLVSKAGIIAGSKAGETRFTDTRKDEQERCITIKSTAISLFFELEKKDLEFVKGENQFETVEVDGKKEKYNGFLINLIDSPGHVDFSSEVTAALRVTDGALVVVDCVSGVCVQTETVLRQAIAERIKPVLFMNKMDRALLELQLGAEELFQTFQRIVENINVIIATYGDDDGPMGPIMVDPSIGNVGFGSGLHGWAFTLKQFAEMYAGKFGVQVDKLMKNLWGDRFFDLKTKKWSSTQTDESKRGFCQFVLDPIFMVFDAVMNIKKDKTAALVEKLGIKLANDEKDLEGKPLMKVFMRKWLPAGDTMLQMIAFHLPSPVTAQKYRMEMLYEGPHDDEAAVAIKTCDPNGPLMMYISKMVPTSDKGRFYAFGRVFSGKVATGMKARIQGPNYVPGKKEDLYEKTIQRTILMMGRFIEPIEDIPSGNIAGLVGVDQYLVKGGTITTYKDAHNMRVMKFSVSPVVRVAVEAKNPADLPKLVEGLKRLAKSDPMVQCIFEESGEHIIAGAGELHLEICLKDLEEDHACIPLKKSDPVVSYRETVQSESNQICLSKSPNKHNRLHCTAQPMPDGLADDIEGGTVNARDEFKARAKILAEKYEYDVTEARKIWCFGPDGTGPNLLMDVTKGVQYLNEIKDSVVAGFQWATREGVLSDENMRGVRFNVHDVTLHADAIHRGGGQIIPTARRVFYASVLTAEPRLLEPVYLVEIQCPEAAVGGIYGVLNRRRGHVFEESQVTGTPMFVVKAYLPVNESFGFTADLRSNTGGQAFPQCVFDHWQVLPGDPLEAGTKPNQIVLDTRKRKGLKEGVPALDNYLDKM

SEQ ID NO: 28 - protein, 4 aa, Caenorhabditis elegans
APKK

SEQ ID NO: 29 - protein, 4 aa, Caenorhabditis elegans
PKKK

SEQ ID NO: 30 - protein, 463 aa, Caenorhabditis elegans
MGKEKVHINIVVIGHVDSGKSTTTGHLIYKCGGIDKRTIEKFEKEAQEMGKGSFKYAWVLDKLKAERERGITIDIALWKFETAKYYITIIDAPGHRDFIKNMITGTSQADCAVLVVACGTGEFEAGISKNGQTREHALLAQTLGVKQLIVACNKMDSTEPPFSEARFTEITNEVSGFIKKIGYNPKAVPFVPISGFNGDNMLEVSSNMPWFKGWAVERKEGNASGKTLLEALDSIIPPQRPTDRPLRLPLQDVYKIGGIGTVPVGRVETGIIKPGMVVTFAPQNVTTEVKSVEMHHESLPEAVPGDNVGFNVKNVSVKDIRRGSVCSDSKQDPAKEARTFHAQVIIMNHPGQISNGYTPVLDCHTAHIACKFNELKEKVDRRTGKKVEDFPKFLKSGDAGIVELIPTKPLCVESFTDYAPLGRFAVRDMRQTVAVGVIKSVEKSDGSSGKVTKSAQKAAPKKK

SEQ ID NO: 31 - protein, 709 aa, Caenorhabditis elegans
SWSGDKLAWLQTWRRVISLVDPYTNSSAHVAIDCMSLTIENLLLVNLHPLAHWLACRLVTVPPILLPRCVPALSAILNESTIRRPPPLLSANILLCFIRLMQSKEQLVVPAICGLSAHELSIVAPRALEHLPKMLQAAKSSKDTKVSSNSLKLFSMLASSYPGAEQILLDQLVNTDVSENAVVIVNSLAILIVQKAQIDLVLTALKTIETHQFAMRLIPLFCSSIASLAQFSSTTLLQALLRAASLLRDERTRTEIEWQMVKLCMQWPQPQMPLVIRGILADRHMVLHGELVTLGGQYPVRGFEVQRWSSAGAPPLQGEDKTVYINRQSAIISVSRKDFHAKSPCEITSRTVVGRHIWDLDTHEDVRKPATNVTNWLRKEALKGKRPGRESQGILGAMDDPFDDLPDYPPSRGSPSPVDGAAQFTSMIETSRRQPQPLGTSSAAHDHLPAFTPNAKLLEWRSLSASLGFVPLVSQVHANFPRDLKHLDQTSSREVHKVAVIYVGESQEDRASILSNTTASASAQFDSFTSELGWEVKVGRGHDGYTGGLPVETRAPYFADAEAEVIFHVSTMLNGDVQQKWKHIGNDEVHVVWTENTRKVYTRETIATKFCDVLIVLEQVGDKMVRVRVDTASALEFGPLFDGALVTMSELSQLVRLTVINASRAYRLARVEHSRPLRHREEVFCNEALAHMKPMPLAQSINHLYVPTI

SEQ ID NO: 32 - protein, 526 aa, Caenorhabditis elegans
MVEGDVDESASGTSGTNKKILFTKKPSVWKDFDNWINDEPENRYDLFQVVKSAMLLQSGYTTILMDQVTDNGADELRISLEYSNFIKIVNSTKLVVGKEQCPPSNVFTLLAEIFANTPGNTSEVGRISTWLTSHLGALLHNDVIWKIHFFDPDLFRSVYWQLIFTLKLAPGDTENLEEDENYAKLLFSCFITAVMVALWHDHEMSFNSICPDYLKPETASEYMVMLISSPPFRSLSQFFLFGLHLLGKYQSEGGCVVVREEAYIAEIRQNDEEKRQSIETRTNLISDDMVYDDGEDLLEQIDRVQQLHEAHCIVLLKKGFLKAPDGFKIVQKGGRPRKYPASATKKRKKKTPRSSPKKKMSKESPINHQKEPIDEQKPSTSLPIYSVATLKPRRKVVKTADEVGLCAPIFVMQSELLKKFREEVQRRYAEGSSASDQERVRNMVYEAYDNIYHINRLSANEGPRILTSDQKLVMQQYKTTFRQGPTFAEETESDVEEEEEKKVVEVVTAKVIKGSAKSSKKFKRRY

SEQ ID NO: 33 - protein, 160 aa, Caenorhabditis elegans
MTISKNNKMMAHLNYRMKIILQDGRTFIGFFKAFDKHMNILLAECEEHRQIKPKAGKKTDGEEKRILGLVLVRGEHIVSMTVDGPPPRDDDSVRLAKAGGAGGVGQAKPGGRGMPAMPGMPGMPPGGAPGGLSGAMRGHGGPGMAAMQPGYGGPPGGRPF

SEQ ID NO: 34 - protein, 126 aa, Caenorhabditis elegans
MKLVRFLMKLSHETVNIELKNGTQVSGTIMGVDVAMNTHLRAVSMTVKNKEPVKLDTLSIRGNNIRYIILPDPLALDTLLIDDEPRKKARAARAGASRGRGRGGMRGGRGGRGRGRGGPRGGGPRR

SEQ ID NO: 35 - protein, 261 aa, Caenorhabditis elegans
MPEPRCTAIVNFLNLSHSILISIFSVSVMSNYHHNHNYQHRPRGYERLPGKRLPDRWNIYDNVGRDIDGTRFVPFKTPLDSSFFDGKNMPVELQFGVKTLISLAQQANKQIGLVIDLTNTDRYYKKTEWADHGVKYLKLNCPGHEVNEREDLVQDFINAVKEFVNDKENDGKLIGVHCTHGLNRTGYLICRYMIDVDNYSASDAISMFEYYRGHPMEREHYKKSLYEAERKKKYGKSSGKSSGNSADSTISSEQLHRNNSQ

SEQ ID NO: 36 - DNA, 22 bp, Artificial Sequence
  Description of Artificial Sequence Synthetic primer
atggcgaccatcgtgccatgct

SEQ ID NO: 37 - DNA, 30 bp, Artificial Sequence
  Description of Artificial Sequence Synthetic primer
ctagaagacacggatgcaattgtttccgaa

SEQ ID NO: 38 - protein, 65 aa, Caenorhabditis elegans
TAENEIRAAFDTHVNALEERRKELLKRVETVKNLKLSVLISQAESLQSKQIDLQQAIQTATKLMD

SEQ ID NO: 39 - protein, 1143 aa, Caenorhabditis elegans
MATIVPCSLEKEEGAPSGPRRLQTEIDVDANDSGNELSMGGSSSEGDSMSHHRGEHSPNHHHQDNHLGSGPPPPQFTGSLFDTPPSMIQSPQQQPQFQFNTGFGLGLPQDSFRCSVCSKSSTIGVLPFVCAHKTCQSCYQMTPSSYDRRACKLCGAVSTATANFTSQMYLSPTLPSPPRGALMSDCSTPTMNNHINSSTPLHQPRAFSFSLSGMPGSPSPVMGARMPSSAGGLMMRPIGFPDSDSSLTSWSPLQQPSQLSINNLSSIGGHQQQSPMLMQNVFDSLAVNDDTPVFSPLSPTNTSMHMPPSLMASPDVPKHSATIAPPRNSMCSTPRLQLATPMSSQSQQTFPIPSPLGSQPQQQQPMGPIQCQGCESKISFAYCMQCQEALCIHCVQAHQRVRATKQHAFVELQQLMATLMSRAVQPQQAQQYTQNVGGSVRQALGSVGSGDGHVSGVENDSIGSGESSPRSSSVCGTHDSVIIGICENCPHSVLLCAICVAQHPGKHRVQPLGDIRVAVGEVVNESQLLQWQCEKTGDTIKQIIDGIVTNATTAENEIRAAFDTHVNALEERRKELLKRVETVKNLKLSVLISQAESLQSKQIDLQQAIQTATKLMDSSDCDEMVLRQVFEKLASCQMGNEGTEPNNNILNVLMLACQVNEDDRLKFTAPQDGILLNKARQFGNIESGPCAKNSSIVGDSFKKAIRERQTVIYVQLRDACGDLLSSSIAATQPTSQALLPHQEPHSHLEQAMPTSDVQAFVISPDGSTVEVTMTPRENGIVALSYYPSIEGSYTLNILVKGTPISGCPTTMDIRRGRNYDEIAAKGPILTFGKEGSGDGELCRPWGICVDQRGRVIVADRSNNRVQIFDKDGNFISKFGTSGNRPGQFDRPAGITTNSLNNIVVADKDNHRVQVFDENGMFLLKFGDRGRAVGYFNYPWGVATNSHNAIAVSDTRNHRVQIFTPQGQFVRKCGFDSAYFFKNLDSPRGLCYLPDGQLLITDFNNHRLAVLSPRNMSEMKVYGSEGDGDGMFVRPQGVVIDPEGHILVCDSRNNRVQVFASDDMRFIGSFGLGPVPNSGFQMPQELPAPYSSLGGPFGAPAFSSAPTPLTPSPRQLLDRPTDLAVGPDGRIYVVDFGNNCIRVF

SEQ ID NO: 40 - DNA, 22 bp, Artificial Sequence
  Description of Artificial Sequence Synthetic primer
atggcgaccatcgtgccatgct

SEQ ID NO: 41 - DNA, 30 bp, Artificial Sequence; identical to SEQ ID NO: 37
  Description of Artificial Sequence Synthetic primer

SEQ ID NO: 42 - protein, 65 aa, Caenorhabditis elegans; identical to SEQ ID NO: 38

SEQ ID NO: 43 - protein, 1147 aa, Caenorhabditis elegans
MATIVPCSLEKEEGAPSGPRRLQTEIDVDANDSGNELSMGGSSSEGDSMSHHRGEHSPNHHHQDNHLGSGPPPPQFTGSLFDTPPSMIQSPQQQPQFQFNTGFGLGLPQDSFRCSVCSKSSTIGVLPFVCAHKTCQSCYQMTPSSYDRRACKLCGAVSTATANFTSQMYLSPTLPSPPRGALMSDCSTPTMNNHINSSTPLHQPRAFSFSLSGMPGSPSPVMGARMPSSAGGLMMRPIGFPDSDSSLTSWSPLQQPSQLSINNLSSIGGHQQQSPMLMQNVFDSLAVNDDTPVFSPLSPTNTSMHMPPSLMASPDVPKHSATIAPPRNSMCSTPRLQLATPMSSQSQQTFPIPSPLGSQPQQQQPMGPIQCQGCESKISFAYCMQCQEALCIHCVQAHQRVRATKQHAFVELQQLMATLMSRAVQPQQAQQYTQNVGGSVRQALGSVGSGDVFFSGHVSGVENDSIGSGESSPRSSSVCGTHDSVIIGICENCPHSVLLCAICVAQHPGKHRVQPLGDIRVAVGEVVNESQLLQWQCEKTGDTIKQIIDGIVTNATTAENEIRAAFDTHVNALEERRKELLKRVETVKNLKLSVLISQAESLQSKQIDLQQAIQTATKLMDSSDCDEMVLRQVFEKLASCQMGNEGTEPNNNILNVLMLACQVNEDDRLKFTAPQDGILLNKARQFGNIESGPCAKNSSIVGDSFKKAIRERQTVIYVQLRDACGDLLSSSIAATQPTSQALLPHQEPHSHLEQAMPTSDVQAFVISPDGSTVEVTMTPRENGIVALSYYPSIEGSYTLNILVKGTPISGCPTTMDIRRGRNYDEIAAKGPILTFGKEGSGDGELCRPWGICVDQRGRVIVADRSNNRVQIFDKDGNFISKFGTSGNRPGQFDRPAGITTNSLNNIVVADKDNHRVQVFDENGMFLLKFGDRGRAVGYFNYPWGVATNSHNAIAVSDTRNHRVQIFTPQGQFVRKCGFDSAYFFKNLDSPRGLCYLPDGQLLITDFNNHRLAVLSPRNMSEMKVYGSEGDGDGMFVRPQGVVIDPEGHILVCDSRNNRVQVFASDDMRFIGSFGLGPVPNSGFQMPQELPAPYSSLGGPFGAPAFSSAPTPLTPSPRQLLDRPTDLAVGPDGRIYVVDFGNNCIRVF

SEQ ID NO: 44 - protein, 387 aa, Caenorhabditis elegans
MTAQDYLNGKLAAANGPLADDWKNLKELWEKKLWHQLTVLTRSLVKKPQFVASTDMHEFYRLFVAEWELRVNPLQLVEICISIAQNIATKDKQKSMEFLSKIGNVINKDKIAVARLHTGEIEARLENKDKNGQIIDLKSIRTQIDSTQHEVDSLVGVTEVHAPFYRVSSLYLREVGDFAGYYREALRYLGVEDANNLTTEQKQVHAVLLGFAALLGENVHNFGELLAHPILKSLEGTRERWIVDVLLAFNSGDLTRFFSLEGDWGGWDDLKKQKDFLTAKIRLMAVMELAVSRPTKARSVSFKEIATKCQIPFDEVEFLVMKALSKDLIRGDINQVEQVVYVTWVQPRVLDNPQIMQMATRISAWRNDVNSMEGIVSKEAREILTQN

SEQ ID NO: 45 - protein, 4 aa, Caenorhabditis elegans
KKRH

SEQ ID NO: 46 - protein, 470 aa, Caenorhabditis elegans
MSKTVTIRRPSPTKTSEEPAAHQTPIFTQTAAEMLGITSLNTEAAELLEFLSREKLKEIVRLSAKWTQKSARRRMAVADVEHAIRSTRQCGGLNISSVDTLNLGIQQLQPIQGTSTGIYSFLKSSADVDVDKEDTETFIKIPRDLRVISYPLVNEGQPVQSEYTVNVDEDDGNFFEKIVPEVMTMIPEKNTPSSSTTSSLQMFRDAVKTAKIDQKVGLKPSTIEILTVEQQIFMKDIITVCMGQDDKKRHEALYTLETDAGLQVFLPHLTERICKSISANISQRCLSLIIYAGRVLRSLSHNKACDMTVTLHHVLPALLSCCVGRNMCLRPETDNHWALRDFSAKTLVGLVRDQVDKHDAGRTARRLFDFSHRIFRDTGSSFSMIYGTVHILQEFVAGPKKAAWLLTELGETNARCKSHIESGSRIGASQLSIQEAQKLNQQILKCENSIRNRYNLQQQAPGVPINRRFH

SEQ ID NO: 47 - protein, 298 aa, Caenorhabditis elegans
MLRRERDSKCLKFLKIQFLSSKSFFITIKFYSDKSEYDKEKDLLEIERKKLESQPPSTSTSQSSKDYKSKSSSSKHDKNSSEYERNNKMWARTDLLVRFIDEDFKRGSLYKQKVRIVDVAGYNDVTIEDRGNTHYNIRQSWLETVIPREIGEKLMIVAGKRSGQLALMLGKDKRKEKLTARLVATNDVVTAYFEDVCAVKIRHEEDSPQLYGMTQFLFKILLVCTIINEFDTLNILFYVPTLSHSHISFNTKLAQLLATSGHQVTVLLAQVDRKCIKLEKKPTILPILQLLTLSNLII

SEQ ID NO: 48 - protein, 434 aa, Caenorhabditis elegans
MATSFYTGGGEDGDGFNPRVHARIAEREGFQLASGSEDPRTLFVANLDPAITDEFLATLFNQIGAVMKAKIIFEGLNDPYAFVEFSDHNQATLALQSHNGRELLEKEMHVTWAFEPREPGENRSKPETSRHFHVFVGDLCSEIDSTKLREAFVKFGEVSEAKIIRDNNTNKGKGYGFVSYPRREDAERAIDEMNGAWLGRRTIRTNWATRKPDEDGERGGDRGDRRGGGGGGRDRYHNQSEKTYDEIFNQAAADNTSVYVGNIANLGEDEIRRAFDRFGPINEVRTFKIQGYAFVKFETKESAARAIVQMNNADIGGQIVRCSWGKSGDSGKPSERGSGGGGGSGNYGYGYGNSGGGGGSGGPGNSQFSNFNQRPPPSGNGGSGGGSGGQNNQYWQYYSQYYNNPHLMQQWNNYWQKDGPPPPPAAAASSTGGN

SEQ ID NO: 49 - protein, 562 aa, Caenorhabditis elegans
MQPVLVNSRPLRVKSHESESKLNLIEQEDQFEGANYSSSSGVIICYSNGTGEVITQEAFDDSGIHFIFSKATCIQYPSNFDPIGVGSVVQIFWSRSFERVVRGNHIIVQIEKMEVYKCCAMLREQVFVTFNSPSTAGVAIGVTERNITVAFHPNCSPVIRYETLKAHSIGRTEFEIKDVREFEFSNGKNRHRENTNRMVDVILAAVPFRVEIHGNVDKIPFFVIEKCRNSPGRSGAAVITKIMKNHFMEANFLQNSESIYFDSTSCHSNILEKVSIGSLINVLADPTFATSSYKWYGYDVTLCNNYLAHASTQRSFVLENNEILQNCKKLEKSPEEAETTTKNDLRFVPPQPEKGEVKKNELPEREAKSIINSYFIDRLAEGIKIEKIDKNWRTFGEILPKTPKKYSESLKKSIQNVLEPFGLNKPEKAAETPKIVEYFPKNPKKRVEIVEKPTVDEIRELFGALMDAEGFALNQRVKPHFVLPDTRWKPTERRYIGIYDDVQWTFMSTFCPKIEENSENRPLAGGWWYRRTVPRDHPVEIVQKMETRRNIIKDCTESPFIE

SEQ ID NO: 50 - protein, 37 aa, Caenorhabditis elegans
GMDEMEFTEAESNMNDLVSEYQQYQEATADDEGEFDE

SEQ ID NO: 51 - protein, 444 aa, Caenorhabditis elegans
MREIVHIQAGQCGNQIGAKFWEVISDEHGIDPTGAYNGDSDLQLERINVYYNEASGGKYVPRACLVDLEPGTMDSVRAGPFGQLFRPDNFVFGQSGAGNNWAKGHYTEGAELVDNVLDVVRKEAESCDCLQGFQMTHSLGGGTGSGMGTLLISKIREEYPDRIMMTFSVVPSPKVSDTVVEPYNATLSVHQLVENTDETFCIDNEALYDICFRTLKLTTPTYGDLNHLVSMTMSGVTTCLRFPGQLNADLRKLAVNMVPFPRLHFFMPGFAPLTSRGSQQYRSLTVPELTQQMFDAKNMMAACDPRHGRYLTVAAMFRGRMSMKEVDEQMLNVQNKNSSYFVEWIPNNVKTAVCDIPPRGVKMAATFVGNSTAIQELFKRISEQFTAMFRRKAFLHWYTGEGMDEMEFTEAESNMNDLVSEYQQYQEATADDEGEFDEHDQDVE

SEQ ID NO: 52 - protein, 7 aa, Caenorhabditis elegans
PARKGKA

SEQ ID NO: 53 - protein, 152 aa, Caenorhabditis elegans
MAPARKGKAKEEQAVVSLGPQAKEGELIFGVAHIFASFNDTFVHITDISGRETIVRVTGGMKVKADRDESSPYAAMLAAQDVADRCKQLGINALHIKLRATGGTRTKTPGPGAQSALRALARAGMKIGRIEDVTPIPSDCTRRKGGRRGRRL

SEQ ID NO: 54 - protein, 151 aa, Caenorhabditis elegans
MGRMHNPGKGMAKSAIPYRRSVPSWQKMTAEEVQDQIVKMAKKGLRPSQIGVILRDSHGVGQVRRLAGNKIFRILKSKGMAPELPEDLYHLVKKAVAIRKHLERSRKDIDSKYRLILVESRIHRLARYYKTKRQLPPTWKYESGTAASLVS

SEQ ID NO: 55 - protein, 17 aa, Caenorhabditis elegans
KKGTHGQEQVTRKKTKK

SEQ ID NO: 56 - protein, 33 aa, Caenorhabditis elegans
RRQQREQAAKIAKDANKAVRAAKAAANKEKKAS

SEQ ID NO: 57 - protein, 4 aa, Caenorhabditis elegans
VGGK

SEQ ID NO: 58 - protein, 159 aa, Caenorhabditis elegans
MKVETCVYSGYKIHPGHGKRLVRTDGKVQIFLSGKALKGAKLRRNPRDIRWTVLYRIKNKKGTHGQEQVTRKKTKKSVQVVNRAVAGLSLDAILAKRNQTEDFRRQQREQAAKIAKDANKAVRAAKAAANKEKKASQPKTQQKTAKNVKTAAPRVGGKR

SEQ ID NO: 59 - protein, 17 aa, Caenorhabditis elegans
KKKKNPRKLRFTKAARR

SEQ ID NO: 60 - protein, 17 aa, Caenorhabditis elegans
KKNPRKLRFTKAARRAR

SEQ ID NO: 61 - protein, 162 aa, Caenorhabditis elegans
MRIEKCYFCSSPIYPGHGIQFVRNDSTVFKFCRSRCNKLFKKKKNPRKLRFTKAARRARGKELINDATQLLEQRRDEPVKYERAMFQKTIEAAKTISALKTKRYGNLIRKRLQPGKIVQKKGLLAKVDKKMHLIRAPVANRDGVKTRAAAKEKKTAESMETN

SEQ ID NO: 62 - protein, 17 aa, Caenorhabditis elegans
RRDYLHYIKKYRRYEKR

SEQ ID NO: 63 - protein, 4 aa, Caenorhabditis elegans
KRHK

SEQ ID NO: 64 - protein, 4 aa, Caenorhabditis elegans
GFSK

SEQ ID NO: 65 - protein, 155 aa, Caenorhabditis elegans
MSEQTERAFLKQPTVNLNNKARILAGSKKTPRYIREVGLGFKAPRDAVEGTYIDKKCPWAGNVPIRGMILTGVVLKNKMTRTIVVRRDYLHYIKKYRRYEKRHKNVPAHCSPAFRDIHPGDLVTIGECRPLSKTVRFNVLKVNKSGTSKKGFSKF

SEQ ID NO: 66 - protein, 821 aa, Caenorhabditis elegans
MTTVRKTYRFCVFSSCLSVSCALVTQVHSSSLPIYSSPFVEKVFLHSSIYVRLCGDMYEQWPTLEFSDLNSSILDLFTKATSQSVASSLLYELTRSDADENGGSIRLNNEEHLKWCMQVLNHSLTLSFATSREYETLKGAVRIYLHWLRALCDTPDNNIPTPLLATPEKYFRNIIDALRWIFCRREDDFDTTVGGQVPRGLAIERQSIEIDMVLDSLKYLTRNSSRKYQDEVWARSISFLLNSSDILLSEPNATEEMGTRTCVRVADTLFDMWLNAVLNEHIPSLTYWSSLATLARRWRHNVPIIECWAKKILGLSTLVCRKMYGDDFLKIDIVDESVLPFENVPMTAEEDENEVHLLYRTWFNMLCLFDSPAKILNHDATRNLCLNGNSPRRTTSSISMSNFELASSSAAQGVSFFLAAVTLQRMVDLFYGDSRVKIDLRNYPVPDGKTAPNTRTASVLTDNHSHHTNRTSSTTGDSSRYVSLGGAVGQIIVDDHQVSMSSGSTASGKTSTATGTSSTHTISSEIRRDQRIMSVNDRSRDPSHRTVSVTDSVNISNQSRYSEQTSSTLTYKSAPIPETANENGHGESISQLVSNSTVSAPVGGAGNDLTLKAGVHPSEMKIGRSSGVIGSAQHNNFYADTTSPYRSAQRFVTNFLTANQATMPYVGGKRPKTDRMLNLVGDWLFAIVNSPTNSPRVTGNDHSGHHKKNNDGVSDVSFISHHFVFTLLSAITTEVISIYICVSMISLTGLNKHHLRIGIIDDETVCTSECPFSPFFAKFTITDGVDFLNNEADSKTTPTSFDFDDFDSFHKFRFQHIYTSK

SEQ ID NO: 67 - protein, 159 aa, Caenorhabditis elegans
MSSLCPYTGRPTGLFRDFEDMMPYWAQRHSMLNNFNNIVPQQLNEVENTAQKFCVKLDVAAFKPEELKVNLEGHVLTIEGHHEVKTEHGFSKRSFTRQFTLPKDVDLAHIHTVINKEGQMTIDAPKTGSNTTVRALPIHTSAGHAVTQKPSSTTTTGKH

SEQ ID NO: 68 - protein, 539 aa, Caenorhabditis elegans
MSSIQCLNPKAELARHAAALELNISGARGLQDVMRSNLGPKGTLKMLVSGAGDIKLTKDGNVLLHEMAIQHPTASMIAKASTAQDDVTGDGTTSTVLLIGELLKQAESLVLEGLHPRIVTEGFEWANTKTLELLEKFKKEAPVERDLLVEVCRTALRTKLHQKLADHITECVVDAVLAIRRDGEEPDLHMVEKMEMHHDSDMDTTLVRGLVLDHGARHPDMPRHVKDAYILTCNVSLEYEKTEVNSGLFYKTAKEREALLAAEREFITRRVHKIIELKKKVIDNSPDGKNKGFVVINQKGIDPPSLDLLASEGILALRRAKRRNMERLQLAVGGEAVNSVDDLTPEDLGWAGLVYEHSLGEEKYTFIEECRAPKSVTLLIKGPNKHTITQIKDAIHDGLRAVFNTIVDKAVLPGAAAFEIAAYVMLKKDVENLKGRAKLGAEAFAQALLVIPKTLAVNGGYDAQETLVKLIEEKTAAGPDIAVGLDLETGGAVEPQGIWDNVTVKKNSISSATVLACNLLLVDEVMRAGMTNLKQPQPE

SEQ ID NO: 69 - protein, 4 aa, Caenorhabditis elegans
VEKR

SEQ ID NO: 70 - protein, 429 aa, Caenorhabditis elegans
MSSIQCLNPKAELARHAAALELNISGARGLQDVMRSNLGPKGTLKMLVSGAGDIKLTKDGNVLLHEMAIQHPTASMIAKASTAQDDVTGDGTTSTVLLIGELLKQAESLVLEGLHPRIVTEGFEWANTKTLELLEKFKKEAPVERDLLVEVCRTALRTKLHQKLADHITECVVDAVLAIRRDGEEPDLHMVEKMEMHHDSDMDTTLVRGLVLDHGARHPDMPRHVKDAYILTCNVSLEYEKTEVNSGLFYKTAKEREALLAAEREFITRRVHKIIELKKKVIDNSPDGKNKGFVVINQKGIDPPSLDLLASEGILALRRAKRRNMERLQLAVGGEAVNSVDDLTPEDLGWAGLVYEHSLGEEKYTFIEECRAPKSVTLLIKGPNKHTITQIKDAIHDGLRAVFNTIVDSCSPWSCCFRNCCLRDVEKRC

SEQ ID NO: 71 - protein, 385 aa, Caenorhabditis elegans; identical to SEQ ID NO: 4

SEQ ID NO: 72 - protein, 1119 aa, Caenorhabditis elegans
MQPTAIRLEDYDKSKLRLPFESPYFPAYFRLLKWKFLDVCVESTRNNDIGYFKLFESLFPPGKLEEIARMIIDEPTPVSHDPDMIKIRNADLDVKIRKQAETYVTLRHAHQQKVQRRRFSECFLNTVLFDEKGLRIADEVMFNYDKELYGYSHWEDLPDGWLTAETFKNKFYDEEEVTNNPFGYQKLDRVAGAARGMIIMKHLKSNPRCVSETTILAFEVFNKGNHQLSTDLVEDLLTEGPAFELKIENGEEKKYAVKKWSLHKTLTMFLAIIGFKSNDKKEKNEHEEWYYGFIDAMKNDPANRAALYFLDKNWPEELEEREKERDRIRLTLLKSQRTNEEAVGEDVCTTIRPQPKDSGYNPDAVVTELVLRTYQEELVQPALEGKNCVIVAPTGSGKTEVAIYAALKHIEERTSQGKPSRVVLLVPKIPLVGQQKDRFLKYCNGMYEVNGFHGSESSVSGTGRRDEVIATHVSVMTPQILINMLQSVRQNERLYVSDFSMMIFDEVHKAAKNHPYVLINQMVQEWKYEKPQIIGLTASLSVKVDGQKDENQMLNDIYNMLALINAPHLSTITRQSSIDELNEHVGKPDDSVELCLPAKENILRDYIERYLNHAHGKFLEELASMSKSTGRNNTIPPNMINTFKKNQPKNYEYYDSLLQGIIQELNKLNVPEKWNSQTWAKYMKVYLEARGIVDLMPAMVAFKYMEKAIGKLNESHSETVEYSTFIKDHDTLKQTIQSVEPEIVLRLKNTLTNQFHVAPESRVIIFVTQRSTAQRVSDFLNESKVLDQFGNYGEQMVGYVLGTNKQGAVQQTSQEQQLTLDKFNNGRLKVIVATSVVEEGLDVTACNLIIKYNCSSGSAIQLVQQRGRARAKNSRSVLLSVKSSINETETNALISEKYMRLCVKKITENGEKQLAAEVKRVAELNAAERKRNLEEQLNLRLRHENKIYKLMCSNCSKEFCKSIYIKKVFSNYMVFDPSVWRFLHVESKRKVSKYLSEDNQPLSDIKCFHCKLDVGRAYKIRGTYLPQLSVKALTFVQESDYSSMTKAKWSDVEQDLFYISEAIEDDFRIMLNALSDTEENIEKKIVLDLDSRQHNKQLEMKRFHIQQEPPTKGVAPEAQ

SEQ ID NO: 73 - protein, 562 aa, Caenorhabditis elegans
MLRELVKKQIIENILRPQNYDSKLGHRKFSVLVLDKSAMVVVNSCLSLNEVFEEGVTLVEDLTRNREPMPSMDAIYIISPVAESIDILINDFSRKTKFNPGNSYRSAHIFFLDPCCDELFEKLSKSPAVKWIKTLKELNLNLKPVESQIFTVNSQFRGDMTKTADGIVSLCATLNIHPTLRFQSDFAQSSEICQRVEQKLKEFGNEGMGTDAELVVLDRSFDLVSPLLHEVTLQAMVVDVTAFKDGVYRYTEAGDSKEIVLDEKDQNWLDLRHKLLPEVMKSVNKMVKDFKNTNKTEPENIKNQSSKDFSTTVRTLQPYLKMKAKMAAYISLTEECRSKYFDSLEKIIALEQDMAVEHTPEHVRITDSQAVGRLSTFILPAIPTETRLRLILIFMLTIGKDKDEQYFNRLLHHTDIPESEFQIIKRMLIWRDKTQKSQFQHRRPPPEDERFIASRWDPKIKNLIEEIYERRLDEREFKVAGKKSTSDFRPAASARYGSGLAGKPREKRKIIIFVVGGITYSEMRVAYELSKKTNTTVILGSDEILTPSSFLESLRDRNTVNC

SEQ ID NO: 74 - protein, 1780 aa, Caenorhabditis elegans
MLPFDNDDSSDDATTSVRPKHPRGVPQSQSTFPRGRSNFSSGTLPNRKTECTPVNTLTIGHSNKMLLTTFRMDRNSKSKSEVDVQEQPVHSSSSAFPGNHLNNFSYPVNRGYLRDYLLQSQRPSTSKPVDCSVLKRHSLPSTHILYEKTKHRGGVNIEEQEKLVRMLWAAAEESETVAKTRQFSKKQAIELNFDAKLIGSMNNDCFGYCRAHMENIKDVLKTHLKLSKVDEVNWIKVGMVPRAAYEDKSYVIDAHLVLTPNGEVEDENELFSEFASSFTSRITGMLHDQVFLEVPKMHTLFTKITPQHMDINISAIAIGNCPNSGLFLVRGDFISQENTVCSVKLQSHHNADASRENSSFKVAGSNKYLSYARFEHDKRLAVVYFGVRLAEFADDGLDHAGFRLNLYYNLFVRIVVDMSHETTNSIYIQMKNPPHLWEGIPKNTIFHPSKSKVLNMETCTEWTRVLSWPGDAEGRGVGCTSEAFSQSSWIRLTMRKDDDNDSVSSTQLMDIVTRLSARSKAKVMFGSIFSIRRKLAPSPAFHSLGSFRANYALQALITRGSVFTDQLFDATDENIPSSDNDNDEDDDDDVDDTKKPMELVHEPLFLKLVRRGMKECSQATEETLEQLLNAFDERRQIDVVTAFTTMYQSRKIQYERLLKGESLQDVGLAKPLPKNCVSVAKVIVTPSRILLMAPEVMMVNRVVRRFGPDYALRCVFRDDNLGRLAIRDFSINNIDHMSNIVTEGIYLTLKNGIQVADRVYSFLGWSNSQMRDQGCYLYAPRVNALTGEVTGTVEDIRVWMGDFRDAISVPKMMSRMGQCFTQAQPTVYSSVKNIHIVENIQVRLERHHWIVEPDIEGGVENKYCFSDGCGRISIKLATHISKILQLKEVPACFQVRFKGFKGILVIDPTIDDIINMPKVIFRKSQQKFGEGGGELQDEYLEVVKYAMPSPVCLNRPFITILDQVSEKQSASSHRRITNRVHYYLERELCSLSNMLINENQAAEELVNRTNLAIDWNAASKRAGFELSVDPLIRDMLFSIYRYNIIHHISKAKIFLPPSLGRSMYGVVDETGLLQYGQVFIQYSPSIRQTSNRPILKTGKVLITKNPCHVPGDVRVFDAVWQPALAHLVDVVVFPQHGPRPHPDEMAGSDLDGDEYSIIWDQEMLLDYNEEAMVFPSSSAAEEDKEPTTDDMVEFFLRYLQQDSIGRMSHAHLAYADLHGLFHENCHAIALKCAVAVDFPKSGVPAEPLSSFEQCEMTPDYMMSGGKPMYYSTRLNGQLHRKARKVEEVLEEFETRGSVFEREYDKLICPEDVDVFFGNEIKLVQTLTLRDEYVDRMQQLLDEYGIEDEASVVSGHAASIKRLAGMERDDYSFYHTDKVVELRYEKLYAVFRAKFFEEFGGEEINIENDGKNTRLKCTKAMHEKIRQWYFVAYVQPKINKAGRCIGQSLPWVAWDALCDLRRQLMLDKNDAVLRGKYPIAARLEEEIENSIERQFDKFLKLKDLIESHKDALFLRRYVYFYGDQIIKMLFILKVWLERENVLPSSVLSIWQLGRLLIRLGLGDLLGNPTIDYEKSLLMPTTMFQQWISKKEDADEAPILRNFDMGTMMLEFLRYLASQSFASAESISLRVFYEKDIVEPILTKSAQWMPLHLIAYRTFHSIAVSGRFDALHLDDEDAVDQITESKDPILVNESLFSSRNYNDDYPISRSRILQSLKDWSGVKEIIPREITGTRKSDMIYVTSVGTVLARQRLARLLLLSGETIRDAIANNVVPNEVRDEFL

SEQ ID NO: 75 - protein, 4 aa, Caenorhabditis elegans
KKKK

SEQ ID NO: 76 - protein, 4 aa, Caenorhabditis elegans
ILPK

SEQ ID NO: 77 - protein, 7 aa, Caenorhabditis elegans
PKNPKKR

SEQ ID NO: 78 - protein, 7 aa, Caenorhabditis elegans
PKKRVEI

SEQ ID NO: 79 - protein, 578 aa, Caenorhabditis elegans
MQPVLVNSRPLRVKSHESESKLNLIEQEDQFEGANYSSSSGVIICYSNGTGEVITQEAFDDSGIHFIFSKATCIQYPSNFDPIGVGSVVQIFWSRSFERVVRGNHIIVQIEKMEVYKCCAMLREQVFVTFNSPSTAGVAIGVTERNITVAFHPNCSPVIRYETLKAHSIGRTEFEIKDRHRENTNRMVDVILAAVPFRVEIHGNVDKIPFFVIEKCRNSPGRSGAAVITKIMKNHFMEANFLQNSESIYFDSTSCHSNILEKVSIGSLINVLADPTFATSSYKWYGYDVTLCNNYLAHASTQRSFVLENNEILQNCKKLEKSPEEAETTTKNDLRFVPPQPEKGEVKKKKMTNCLKFNSKSAQFKLRHLILDRCFSELPEREAKSIINSYFIDRLAEGIKIEKIDKNWRTFGEILPKTPKKYSESLKKSIQNVLEPFGLNKPEKAAETPKIVEYFPKNPKKRVEIVEKPTVDEIRELFGALMDAEGFALNQRVKPHFVLPDTRWKPTERRYIGIYDDVQWTFMSTFCPKIEENSENRPLAGGWWYRRTVPRDHPVEIVQKMETRRNIIKDCTESPFIE

SEQ ID NO: 80 - protein, 4 aa, Caenorhabditis elegans
DKDS

SEQ ID NO: 81 - protein, 458 aa, Caenorhabditis elegans
MAMAPLRPRVFARCLILKNLELIEAARIFFIDSAVTANVSWKCLFQIDENLKFHPWQAMHCTLGRLVHLSDSWTDTQCTEFRNIVSKFAKFQITANQCDVDFRSDRPSLLVNLYGLPNGTEIDKKVAIEEICAVSMQNVMVSQFPTNFMVNPKLEELDKEQDHLDVILLEEFRRDLPADWAHEPPADYREDDADWDILQCHVAEWNDTALEQFRRADGSFWAMLEPSCTVSPWEMHVTPILAPEKMSDNEHWIFEQLVKNSENQQKIDDFYSNLKNQRPLEMEEIKFALQTGRTYVMATIKNRQKSSAQWLRCEIIDFLPNANVALRYVDLGTRGILKLKNLHRMHIEHTKIAPACIEIGRFLDDDLSMADSEMEWNTHFWREIVPYDVPIVVGPDMEFLETGKLQFSQIRVAGDEDDENLLDKIPSPSPFFTERSDDLRTQKEDDDDGNVSDDKDSG

SEQ ID NO: 82 - protein, 261 aa, Caenorhabditis elegans; identical to SEQ ID NO: 35

SEQ ID NO: 83 - protein, 346 aa, Caenorhabditis elegans
MQADGEKKKKKTNPERSTHDDTPKSRTRVLFSQYFFLSFSLFFRAIFMLRSLCSIAVRLGGARQPRLLSSAASGDGNDGKGAKDAIDEDLLNAIEGVANNIHPQNGSEKKSLKNTLINRLVANEKASFDAAAASASSEMLDDQALIGLLADVAGDAKVEKKLPPKSAQLRQEKRGLVLLRKEIFYQAVQSGFTTEEARVKSETIVNEAQIKLQEQRKALLNDVREKVEQEEVEETERSEKDQKLFTMALEFMEKIYKDDLISSAVQFPTAHSDQQILSKNKSNGQQKENNGNIQSIMSSKWAMNRMFHSLITYSWRDIYHHWVSRNLVQLLILCIWFVLVYPRIHI

SEQ ID NO: 84 - protein, 1025 aa, Homo sapiens
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIENLSQVDGPQVEEQLLSTTVQPNLEKEVWGMENNSSESSFADSSVVSESDTSLAEGSVSCLDESLGHNSNMGSDSGTMGSDSDEENVAARASPEPELQLRPYQMEVAQPALEGKNIIICLPTGSGKTRVAVYIAKDHLDKKKKASEPGKVIVLVNKVLLVEQLFRKEFQPFLKKWYRVIGLSGDTQLKISFPEVVKSCDIIISTAQILENSLLNLENGEDAGVQLSDFSLIIIDECHHTNKEAVYNNIMRHYLMQKLKNNRLKKENKPVIPLPQILGLTASPGVGGATKQAKAEEHILKLCANLDAFTIKTVKENLDQLKNQIQEPCKKFAIADATREDPFKEKLLEIMTRIQTYCQMSPMSDFGTQPYEQWAIQMEKKAAKEGNRKERVCAEHLRKYNEALQINDTIRMIDAYTHLETFYNEEKDKKFAVIEDDSDEGGDDEYCDGDEDEDDLKKPLKLDETDRFLMTLFFENNKMLKRLAENPEYENEKLTKLRNTIMEQYTRTEESARGIIFTKTRQSAYALSQWITENEKFAEVGVKAHHLIGAGHSSEFKPMTQNEQKEVISKFRTGKINLLIATTVAEEGLDIKECNIVIRYGLVTNEIAMVQARGRARADESTYVLVAHSGSGVIEHETVNDFREKMMYKAIHCVQNMKPEEYAHKILELQMQSIMEKKMKTKRNIAKHYKNNPSLITFLCKNCSVLACSGEDIHVIEKMHHVNMTPEFKELYIVRENKALQKKCADYQINGEIICKCGQAWGTMMVHKGLDLPCLKIRNFVVVFKNNSTKKQYKKWVELPITFPNLDYSECCLFSDED

SEQ ID NO: 85 - protein, 119 aa, Homo sapiens
MKLVRFLMKLSHETVTIELKNGTQVHGTITGVDVSMNTHLKAVKMTLKNREPVQLETLSIRGNNIRYFILPDSLPLDTLLVDVEPKVKSKKREAVAGRGRGRGRGRGRGRGRGRGGPRR

SEQ ID NO: 86 - protein, 8 aa, Homo sapiens
FKVYRYLQ

SEQ ID NO: 87 - protein, 16 aa, Homo sapiens
LILIYSRHLFFYESKC

SEQ ID NO: 88 - protein, 744 aa, Homo sapiens
MASEGTNIPSPVVRQIDKQFLICSICLERYKNPKVLPCLHTFCERCLQNYIPAHSLTLSCPVCRQTSILPEKGVAALQNNFFITNLMDVLQRTPGSNAEESSILETVTAVAAGKPLSCPNHDGNVMEFYCQSCETAMCRECTEGEHAEHPTVPLKDVVEQHKASLQVQLDAVNKRLPEIDSALQFISEIIHQLTNQKASIVDDIHSTFDELQKTLNVRKSVLLMELEVNYGLKHKVLQSQLDTLLQGQESIKSCSNFTAQALNHGTETEVLLVKKQMSEKLNELADQDFPLHPRENDQLDFIVETEGLKKSIHNLGTILTTNAVASETVATGEGLRQTIIGQPMSVTITTKDKDGELCKTGNAYLTAELSTPDGSVADGEILDNKNGTYEFLYTVQKEGDFTLSLRLYDQHIRGSPFKLKVIRSADVSPTTEGVKRRVKSPGSGHVKQKAVKRPASMYSTGKRKENPIEDDLIFRVGTKGRNKGEFTNLQGVAASTNGKILIADSNNQCVQIFSNDGQFKSRFGIRGRSPGQLQRPTGVAVHPSGDIIIADYDNKWVSIFSSDGKFKTKIGSGKLMGPKGVSVDRNGHIIVVDNKACCVFIFQPNGKIVTRFGSRGNGDRQFAGPHFAAVNSNNEIIITDFHNHSVKVFNQEGEFMLKFGSNGEGNGQFNAPTGVAVDSNGNIIVADWGNSRIQVFDGSGSFLSYINTSADPLYGPQGLALTSDGHVVVADSGNHCFKVYRYLQ

SEQ ID NO: 89 - protein, 677 aa, Homo sapiens
MAEEKKLKLSNTVLPSESMKVVAESMGIAQIQEETCQLLTDEVSYRIKEIAQDALKFMHMGKRQKLTTSDIDYALKLKNVEPLYGFHAQEFIPFRFASGGGRELYFYEEKEVDLSDIINTPLPRVPLDVCLKAHWLSIEGCQPAIPENPPPAPKEQQKAEATEPLKSAKPGQEEDGPLKGKGQGATTADGKGKEKKAPPLLEGAPLRLKPRSIHELSVEQQLYYKEITEACVGSCEAKRAEALQSIATDPGLYQMLPRFSTFISEGVRVNVVQNNLALLIYLMRMVKALMDNPTLYLEKYVHELIPAVMTCIVSRQLCLRPDVDNHWALRDFAARLVAQICKHFSTTTNNIQSRITKTFTKSWVDEKTPWTTRYGSIAGLAELGHDVIKTLILPRLQQEGERIRSVLDGPVLSNIDRIGADHVQSLLLKHCAPVLAKLRPPPDNQDAYRAEFGSLGPLLCSQVVKARAQAALQAQQVNRTTLTITQPRPTLTLSQAPQPGPRTPGLLKVPGSIALPVQTLVSARAAAPPQPSPPPTKFIVMSSSSSAPSTQQVLSLSTSAPGSGSTTTSPVTTTVPSVQPIVKLVSTATTAPPSTAPSGPGSVQKYIVVSLPPTGEGKGGPTSHPSPVPPPASSPSPLSGSALCGGKQEAGDSPPPAPGTPKANGSQPNSGSPQPAP

SEQ ID NO: 90 - protein, 403 aa, Homo sapiens
MSHRKFSAPRHGSLGFLPRKRSSRHRGKVKSFPKDDPSKPVHLTAFLGYKAGMTHIVREVDRPGSKVNKKEVVEAVTIVETPPMVVVGIVGYVETPRGLRTFKTVFAEHISDECKRRFYKNWHKSKKKAFTKYCKKWQDEDGKKQLEKDFSSMKKYCQVIRVIAHTQMRLLPLRQKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFVMLKGCVVGTKKRVLTLRKSLLVQTKRRALEKIDLKFIDTTSKFGHGRFQTMEEKKAFMGPLKKDRIAKEEGA

SEQ ID NO: 91 - protein, 349 aa, Homo sapiens
MEDPQSKEPAGEAVALALLESPRPEGGEEPPRPSPEETQQCKFDGQETKGSKFITSSASDFSDPVYKEIAITNGCINRMSKEELRAKLSEFKLETRGVKDVLKKRLKNYYKKQKLMLKESNFADSYYDYICIIDFEATCEEGNPPEFVHEIIEFPVVLLNTHTLEIEDTFQQYVRPEINTQLSDFCISLTGITQDQVDRADTFPQVLKKVIDWMKLKELGTKYKYSLLTDGSWDMSKFLNIQCQLSRLKYPPFAKKWINIRKSYGNFYKVPRSQTKLTIMLEKLGMDYDGRPHCGLDDSKNIARIAVRMLQDGCELRINEKMHAGQLMSVSSSLPIEGTPPPQMPHFRK

SEQ ID NO: 92 - protein, 5 aa, Homo sapiens
KKKKK

SEQ ID NO: 93 - protein, 777 aa, Homo sapiens
MEQYCSIKIVDILEEEVVTFAVEVELPNSGKLLDHVLIEMGYGLKPSGQDSKKENADQSDPEDVGKMTTENNIVVDKSDLIPKVLTLNVGDEFCGVVAHIQTPEDFFCQQLQSGRKLAELQASLSKYCDQLPPRSDFYPAIGDICCAQFSEDDQWYRASVLAYASEESVLVGYVDYGNFEILSLMRLCPIIPKLLELPMQAIKCVLAGVKPSLGIWTPEAICLMKKLVQNKIITVKVVDKLENSSLVELIDKSETPHVSVSKVLLDAGFAVGEQSMVTDKPSDVKETSVPLGVEGKVNPLEWTWVELGVDQTVDVVVCVIYSPGEFYCHVLKEDALKKLNDLNKSLAEHCQQKLPNGFKAEIGQPCCAFFAGDGSWYRALVKEILPNGHVKVHFVDYGNIEEVTADELRMISSTFLNLPFQGIRCQLADIQSRNKHWSEEAITRFQMCVAGIKLQARVVEVTENGIGVELTDLSTCYPRIISDVLIDEHLVLKSASPHKDLPNDRLVNKHELQVHVQGLQATSSAEQWKTIELPVDKTIQANVLEIISPNLFYALPKGMPENQEKLCMLTAELLEYCNAPKSRPPYRPRIGDACCAKYTSDDFWYRAVVLGTSDTDVEVLYADYGNIETLPLCRVQPITSSHLALPFQIIRCSLEGLMELNGSSSQLIIMLLKNFMLNQNVMLSVKGITKNVHTVSVEKCSENGTVDVADKLVTFGLAKNITPQRQSALNTEKMYRTNCCCTELQKQVEKHEHILLFLLNNSTNQNKFIEMKKLVKS

SEQ ID NO: 94 - protein, 330 aa, Homo sapiens
MSQWHHPRSGWGRRRDFSGRSSAKKKGGNHIPERWKDYLPVGQRMPGTRFIAFKVPLQKSFEKKLAPEECFSPLDLFNKIREQNEELGLIIDLTYTQRYYKPEDLPETVPYLKIFTVGHQVPDDETIFKFKHAVNGFLKENKDNDKLIGVHCTHGLNRTGYLICRYLIDVEGVRPDDAIELFNRCRGHCLERQNYIEDLQNGPIRKNWNSSVPRSSDFEDSAHLMQPVHNKPVKQGPRYNLHQIQGHSAPRHFHTQTQSLQQSVRKFSENPHVYQRHHLPPPGPPGEDYSHRRYSWNVKPNASRAAQDRRRWYPYNYSRLSYPACWEWTQ

SEQ ID NO: 95 - protein, 29 aa, Caenorhabditis elegans
PEKLMEQLKKLESAMDSVIETASDLVSLS

SEQ ID NO: 96 - protein, 7 aa, Caenorhabditis elegans
PEMKKIK

SEQ ID NO: 97 - protein, 7 aa, Caenorhabditis elegans
PKRRKFE

SEQ ID NO: 98 - protein, 7 aa, Caenorhabditis elegans
PLNKRKD

SEQ ID NO: 99 - protein, 7 aa, Caenorhabditis elegans
PRRSRTV

SEQ ID NO: 100 - protein, 29 aa, Caenorhabditis elegans
IQQLRDLNQKSIEDQERETRENDKIDDGE

SEQ ID NO: 101 - protein, 36 aa, Caenorhabditis elegans
PKQLTKEEEQFKKLQNDLLKQAKERLEALEMSEDME

SEQ ID NO: 102 - protein, 4 aa, Caenorhabditis elegans
KPRR

SEQ ID NO: 103 - protein, 1845 aa, Caenorhabditis elegans
MVRVRADLQCFNPRDYQVELLDKATKKNTIVQLGTGSGKTFIAVLLLKEYGVQLFAPLDQGGKRAFFVVEKVNLVEQQAIHIEVHTSFKVGQVHGQTSSGLWDSKEQCDQFMKRHHVVVITAQCLLDLIRHAYLKIEDMCVLIFDECHHALGSQHPYRSIMVDYKLLKKDKPVPRVLGLTASLIKAKVAPEKLMEQLKKLESAMDSVIETASDLVSLSKYGAKPYEVVIICKDFEIGCLGIPNFDTVIEIFDETVAFVNTTTEFHPDLDLDPRRPIKDSLKTTRAVFRQLGPWAAWRTAQVWEKELGKIIKSQVLPDKTLRFLNMAKTSMITIKRLLEPEMKKIKSIEALRPYVPQRVIRLFEILETFNPEFQKERMKLEKAEHLSAIIFVDQRYIAYSLLLMMRHIKSWEPKFKFVNPDYVVGASGRNLASSDSQGLHKRQTEVLRRFHRNEINCLIATSVLEEGVDVKQCNLVIKFDRPLDMRSYVQSKGRARRAGSRYVITVEEKDTAAYCSKLPSDIFTRLVPHNQIIPIEENGVTKYCAELLLPINSPIKHAIVLKNPMPNKKTAQMAVALEACRQLHLEGELDDNLLPKGRESIAKLLEHIDEEPDEYAPGIAAKVGSSKRKQLYDKKIARALNESFVEADKECFIYAFELERFREAELTLNPKRRKFEDPFNYEYCFGFLSAKEIPKIPPFPVFLRQGNMKVRLIVAPKKTTVTAAQLQEIQLFHNYLFTQVLQMCKTGNLEFDGTSNAPLNTLIVPLNKRKDDMSYTINMKYVSEVVANMENMPRIPKDEVRRQYKFNAEDYKDAIVMPWYRNLEQPVFYYVAEILPEWRPSSKFPDTHFETFNEYFIKKYKLEIYDQNQSLLDVDFTSTRLNLLQPRIQNQPRRSRTVSNSSTSNIPQASASDSKESNTSVPHSSQRQILVPELMDIHPISATLWNVIAALPSIFYRVNQLLLTDELRETILVKAFGKEKTKLDDNVEWNSLAYATEYEEKQTIIVKKIQQLRDLNQKSIEDQERETRENDKIDDGEELFNIGVWDPEEAVRIGVEISSRDDRMDGEDQDTVGLTQGLHDGNISDEDDELPFVMHDYTARLTSNRNGIGAWSGSESIVPSGWGDWDGPEPDNSPMPFQILGGPGGLNVQALMADVGRVFDPSTASSSLSQTVQESTVSPPKQLTKEEEQFKKLQNDLLKQAKERLEALEMSEDMEKPRRLEDTVNLEDYGDDQENQEDENTPTNFPKTIDEEIEELSIGARKKQEIDDNAAKTDVLERENCEVLPVAINEKSRSFSFEKESKAINGRLIRQRSEEYVSHIDSDIGLGVSPCLLLTALTTSNAADGMSLERFETIGDSFLKFATTDYLYHTLLDQHEGKLSFARSKEVSNCNLYRLGKKLGIPQLIVANKFDAHDSWLPPCYIPTCDFKAPNTDDAEEKDNEIERILDGQVIEEKPENKTGWDIGGDVSKSTTDGIETITFPKQARVGNDDISPLPYNLLTQQHISDKSIADAVEALIGVHLLTLGPNPTLKVMNWMGLKVIQKDQKSDVPSPLLRFIDTPTNPNASLNFLNNLWQQFQFTQLEEKIGYRFKERAYLVQAFTHASYINNRVTGCYQRLEFLGDAVLDYMITRYLFEDSRQYSPGVLTDLRSALVNNTIFASLAVKFEFQKHFIAMCPGLYHMIEKFVKLCSERNFDTNFNAEMYMVTTEEEIDEGQEEDIEVPKAMGDIFESVAGAIYLDSGRNLDTTWQVIFHMMRGTIELCCANPPRSPIRELMEFEQSKVRFSKMERILESGKVRVTVEVVNNMRFTGMGRNYRIAKATAAKRALKYLHQIEQQRRQSPSLTTV

SEQ ID NO: 104 - protein, 1922 aa, Homo sapiens
MKSPALQPLSMAGLQLMTPASSPMGPFFGLPWQQEAIHDNIYTPRKYQVELLEAALDHNTIVCLNTGSGKTFIAVLLTKELSYQIRGDFSRNGKRTVFLVNSANQVAQQVSAVRTHSDLKVGEYSNLEVNASWTKERWNQEFTKHQVLIMTCYVALNVLKNGYLSLSDINLLVFDECHLAILDHPYREIMKLCENCPSCPRILGLTASILNGKCDPEELEEKIQKLEKILKSNAETATDLVVLDRYTSQPCEIVVDCGPFTDRSGLYERLLMELEEALNFINDCNISVHSKERDSTLISKQILSDCRAVLVVLGPWCADKVAGMMVRELQKYIKHEQEELHRKFLLFTDTFLRKIHALCEEHFSPASLDLKFVTPKVIKLLEILRKYKPYERQQFESVEWYNNRNQDNYVSWSDSEDDDEDEEIEEKEKPETNFPSPFTNILCGIIFVERRYTAVVLNRLIKEAGKQDPELAYISSNFITGHGIGKNQPRNKQMEAEFRKQEEVLRKFRAHETNLLIATSIVEEGVDIPKCNLVVRFDLPTEYRSYVQSKGRARAPISNYIMLADTDKIKSFEEDLKTYKAIEKILRNKCSKSVDTGETDIDPVMDDDDVFPPYVLRPDDGGPRVTINTAIGHINRYCARLPSDPFTHLAPKCRTRELPDGTFYSTLYLPINSPLRASIVGPPMSCVRLAERVVALICCEKLHKIGELDDHLMPVGKETVKYEEELDLHDEEETSVPGRPGSTKRRQCYPKAIPECLRDSYPRPDQPCYLYVIGMVLTTPLPDELNFRRRKLYPPEDTTRCFGILTAKPIPQIPHFPVYTRSGEVTISIELKKSGFMLSLQMLELITRLHQYIFSHILRLEKPALEFKPTDADSAYCVLPLNVVNDSSTLDIDFKFMEDIEKSEARIGIPSTKYTKETPFVFKLEDYQDAVIIPRYRNFDQPHRFYVADVYTDLTPLSKFPSPEYETFAEYYKTKYNLDLTNLNQPLLDVDHTSSRLNLLTPRHLNQKGKALPLSSAEKRKAKWESLQNKQILVPELCAIHPIPASLWRKAVCLPSILYRLHCLLTAEELRAQTASDAGVGVRSLPADFRYPNLDFGWKKSIDSKSFISISNSSSAENDNYCKHSTIVPENAAHQGANRTSSLENHDQMSVNCRTLLSESPGKLHVEVSADLTAINGLSYNQNLANGSYDLANRDFCQGNQLNYYKQEIPVQPTTSYSIQNLYSYENQPQPSDECTLLSNKYLDGNANKSTSDGSPVMAVMPGTTDTIQVLKGRMDSEQSPSIGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNTDKWEKDEMTKDCMLANGKLDEDYEEEDEEEESLMWRAPKEEADYEDDFLEYDQEHIRFIDNMLMGSGAFVKKISLSPFSTTDSAYEWKMPKKSSLGSMPFSSDFEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTDREKALCPTRENFNSQQKNLSVSCAAASVASSRSSVLKDSEYGCLKIPPRCMFDHPDADKTLNHLISGFENFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVQFQLEKNEMQGMDSELRRSEEDEEKEEDIEVPKAMGDIFESLAGAIYMDSGMSLETVWQVYYPMMRPLIEKFSANVPRSPVRELLEMEPETAKFSPAERTYDGKVRVTVEVVGKGKFKGVGRSYRIAKSAAARRALRSLKANQPQVPNS

SEQ ID NO: 105 - protein, 1917 aa, Mus musculus
MNEKPCFAALSMAGLQLMTPASSPMGPFFGLPWQQEAIHDNIYTPRKYQVELLEAALDHNTIVCLNTGSGKTFIAVLLTKELAHQIRGDLNPHAKRTVFLVNSANQVCQQVSAVRTHSDLKVGEYSDLEVNASWTKERWSQEFTKHQVLIMTCYVALTVLKNGYLSLSDINLLVFDECHLAILDHPYREIMKLCESCPSCPRILGLTASILNGKCDPEELEEKIQKLERILRSDAETATDLVVLDRYTSQPCEIVVDCGPFTDRSGLYERLLMELEAALDFINDCNVAVYSKERDSTLISKQILSDCRAVLVVLGPWCADKVAGMMVRELQKYIKHEQEELHRKFLLFTDTLLRKIHALCEEYFSPASLDLKYVTPKVMKLLEILRKYKPYERQQFESVEWYNNRNQDNYVSWSDSEDDDDDEEIEEKEKPETNFPSPFTNILCGIIFVERRYTAVVLNRLIKEAGKQDPELAYISSNFITGHGIGKNQPRSKQMEAEFRKQEEVLRKFRAHETNLLIATSVVEEGVDIPKCNLVVRFDLPTEYRSYVQSKGRARAPISNYVMLADTDKIKSFEEDLKTYKAIEKILRNKCSKSADGAEADVHAGVDDEDAFPPYVLRPDDGGPRVTINTAIGHINRYCARLPSDPFTHLAPKCRTRELPDGTFYSTLYLPINSPLRASIVGPPMDSVRLAERVVALICCEKLHKIGELDEHLMPVGKETVKYEEELDLHDEEETSVPGRPGSTKRRQCYPKAIPECLRDSYPKPDQPCYLYVIGMVLTTPLPDELNFRRRKLYPPEDTTRCFGILTAKPIPQIPHFPVYTRSGEVTISIELKKSGFILSQQMLELITRLHQYIFSHILRLEKPALEFKPTGAESAYCVLPLNVVNDSGTLDIDFKFMEDIEKSEARIGIPSTKYSKETPFVFKLEDYQDAVIIPRYRNFDQPHRFYVADVYTDLTPLSKFPSPEYETFAEYYKTKYNLDLTNLNQPLLDVDHTSSRLNLLTPRHLNQKGKALPLSSAEKRKAKWESLQNKQILVPELCAIHPIPASLWRKAVCLPSILYRLHCLLTAEELRAQTASDAGVGVRSLPVDFRYPNLDFGWKKSIDSKSFISTCNSSLAESDNYCKHSTTVVPEHAAHQGATRPSLENHDQMSVNCKRLPAESPAKLQSEVSTDLTAINGLSYNKNLANGSYDLVNRDFCQGNQLNYFKQEIPVQPTTSYPIQNLYNYENQPKPSNECPLLSNTYLDGNANTSTSDGSPAVSTMPAMMNAVKALKDRMDSEQSPSVGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNSEKWEKDEMTKDCLLANGKLGEACEEEEDLTWRAPKEEAEDEDDFLEYDQEHIQFIDSMLMGSGAFVRKISLSPFSASDSAYEWKMPKKASLGSMPFASGLEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTSREKALDPAQENGSSQQKSLSGSCAAPVGPRSSAGKDLEYGCLKIPPRCMFDHPDAEKTLNHLISGFETFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVKFQLEKNEMQGMDSELRRSEEDEEKEEDIEVPKAMGDIFESLAGAIYMDSGMSLEVVWQVYYPMMQPLIEKFSANVPRSPVRELLEMEPETAKFSPAERTYDGKVRVTVEVVGKGKFKGVGRSYRIAKSAAARRALRSLKANQPQVPNS

SEQ ID NO: 106 - DNA, 33 bp, Caenorhabditis elegans
tcgccggcgggctcctcgccacttaaaatgtca

SEQ ID NO: 107 - protein, 56 aa, Caenorhabditis elegans
VYRRCRIVDVCRNNELLKVFFIDDAVIAWVQPECLGELDQHYMYYPWQAIQVSMFG

SEQ ID NO: 108 - protein, 53 aa, Caenorhabditis elegans
RCLILKNLELIEAARIFFIDSAVTANVSWKCLFQIDENLKFHPWQAMHCTLGR

SEQ ID NO: 109 - protein, 84 aa, Caenorhabditis elegans
FGNYGEQMVGYVLGTNKQGAVQQTSQEQQLTLDKFNNGRLKVIVATSVVEEGLDVTACNLIIEYNCSSGSAIQLVQQRGRARAK

SEQ ID NO: 110 - protein, 87 aa, Caenorhabditis elegans
ELLMLGIKSEWMSGLNKSTASSADISASKQKQMEKLKMFADGEIRILVSTSVAEEGLDVPECSLVIKYNYATNEIAHVQRRGRGRAL

SEQ ID NO: 111 - protein, 87 aa, Caenorhabditis elegans
ELAYISSNGITGHGIGKNQPRNKQMEAEFRKQEEVLRKFRAHETNLLIATSIVEEGVDIPKCHLVVRFDLPTEYRSYVQSKGRARAP

SEQ ID NO: 112 - protein, 77 aa, Caenorhabditis elegans
VVGASGRNLASSDSQGLHKRQTEVLRRFHRNEINCLIATSVLEEGVDVKQCNLVIKFDRPLDMRSYVQSKGRARRAG